(19) United States
(12) Patent Application Publication
(10) Pub. No.: US 2020/0255851 A1
(43) Pub. Date: Aug. 13, 2020

(54) COMPOSITIONS FOR TREATING CITRUS DISEASE AND PROMOTING YIELD INCREASE IN ROW CROPS

(71) Applicant: Spogen Biotech Inc., St. Louis, MO (US)

(72) Inventors: Brian Thompson, Creve Coeur, MO (US); Michelle Leslie, Webster Groves, MO (US)

(21) Appl. No.: 16/749,576

(22) Filed: Jan. 22, 2020

Related U.S. Application Data
(60) Provisional application No. 62/796,010, filed on Jan. 23, 2019.

(51) Int. Cl. C12N 15/82; C12N 9/64; C12N 9/24; C07K 14/32; C07K 14/415

(52) U.S. Cl. CPC C12N 15/8261; C12N 9/6408; C12N 9/2405; C12Y 302/01014; C07K 14/415; C12Y 305/99007; C12Y 302/01001; C07K 14/32

(57) ABSTRACT

Isolated bioactive priming peptides and bioactive priming compostions comprising bioactive priming polypeptides and/or inducer compounds are provided that are useful when applied to plants in agricultural formulations. Methods of using the isolated bioactive priming peptides and/or compositions are also provided which are applied exogenously to the surface of a plant or a plant cell membrane or endogenously to the interior of a plant or to a plant cell. The isolated bioactive priming peptides and/or bioactive priming compositions when applied to a plant, a plant part, or a plant growth medium or a rhizosphere in an area surrounding the plant or the plant part increase growth, yield, health, longevity, productivity, and/or vigor of a plant or a plant part and/or protect the plant or the plant part from disease, and/or increase the innate immune response of the plant or the plant part and/or improve the quality and/or quantity of juice obtained from a plant or plant part.

### CROSS REFERENCE TO RELATED APPLICATIONS

This application claims priority to U.S. Provisional Application No. 62/796,010, filed Jan. 23, 2019, the content of which is incorporated herein by reference in its entirety.

### FIELD OF THE INVENTION

Bioactive priming compositions which can be delivered in agricultural formulations are provided. The compositions comprise polypeptides and/or inducer compounds and can be applied to crops to achieve agronomically desirable outcomes such as enhanced phenotypes in plants (e.g., those that exhibit protection against pests, disease agents and abiotic stress), increased plant growth, productivity and yield. The compositions and methods described herein are particularly suited for improving the health and productivity of citrus, specialty, horticultural, row and vine crops.

### BACKGROUND OF THE INVENTION

Conventional methods to achieve desired agronomic phenotypes such as increased yield, disease prevention, disease resistance, and improved abiotic stress tolerance have utilized mostly selective breeding, grafting, transgenic and agrochemical approaches.

Bioactive Priming Polypeptides Involved in Plant Defense Responses

Plants possess an immune system that detects and protects against microbes that can cause disease. Antimicrobial peptides (AMPs) in plants are often the first line of defense against invading pathogens and are involved in the initiation of defense responses that can impart innate immunity to a plant. Many AMPs are generically active against various kinds of infectious agents. They are generally classified as antibacterial, anti-fungal, anti-viral and/or anti-parasitic.

The resistance of given plant species against certain pathogenic organisms that can contact a plant surface and colonize it, is based on highly specialized recognition systems for molecules produced only by certain microbes (for example, specific bacterial or fungal strains). Plants sense potential microbial invaders by using pattern-recognition receptors (PRRs) to recognize the pathogen-associated molecular patterns (PAMPs) associated with them.

Flagellin/Flagellin-Associated Polypeptides

Flagellins and flagellin-associated polypeptides derived from those flagellins have been reported to have functional roles in innate immune responses in plants. These polypeptides are derived from highly conserved domains of eubacterial flagellin. Flagellin is the main building block of the bacterial flagellum. The flagellin protein subunit building up the filament of bacterial flagellum can act as a potent elicitor in cells to mount defense-related responses in various plant species.

“Flagellin” is a globular protein that polymerizes to form the whip-like filament structure of of the bacterial flagellum. Flagellin is the principal substituent of bacterial flagellum and is present in flagellated bacteria. Plants can perceive, combat infection and mount defense signaling against bacterial microbes through the recognition of conserved epitopes, such as the stretch of 22 amino acids (Flg22) located in the N-terminus of a full length flagellin coding sequence. The elicitor activity of Flg22 polypeptide is attributed to this conserved domain within the N-terminus of the flagellin protein (Felix et al., 1999). Plants can perceive bacteria through pattern recognition receptors (PRRs) which include leucine-rich repeat receptor kinases located in the plasma membrane and available at the plant cell surface. In plants, Flg22 is recognized by the leucine-rich repeat receptor kinase FLAGELLIN SENSING 2 (FLS2), which is highly conserved in both monocot and dicot plants.

In Arabidopsis, the innate immune response to Flg22 involves a host recognition protein complex that contains the FLS2 leucine rich repeat (LRR) receptor kinase (Gómez-Gómez L. and Boller T., “FLS2: An LRR receptor-like kinase involved in the perception of the bacterial elicitor flagellin in Arabidopsis,” Molecular Cell 5: 1003-1011, 2000). In Arabidopsis thaliana, FLS2 is a PRR that determines flagellin perception and is specific for the binding of the flagellin-associated polypeptide(s). For example, the binding of Flg22 to the plasma membrane-bound receptor triggers a signaling cascade that is involved in the activation of pattern-triggered immunity (Chinchilla et al., “The Arabidopsis receptor kinase FLS2 binds Flg22 and determines the specificity of flagellin perception,” Plant Cell 18: 465-476, 2006). Thus, the binding of Flg22 to the Arabidopsis FLS2 membrane-bound receptor promotes the first step of activation in which the binding elicits an activation cascade for defense responses in the plant. The Flg22-FLS2 interaction can also lead to the production of reactive oxygen species (ROS) that contribute to the induction of an oxidative burst, cellular medium alkalinization, downstream induction of pathogen-responsive genes and defense-related responses which then can impart disease resistance to a plant (Felix G. et al., “Plants have a sensitive perception system for the most conserved domain of bacterial flagellin,” The Plant Journal 18: 265-276, 1999, Gómez-Gómez L. and Boller T., “FLS2: An LRR receptor-like kinase involved in the perception of the bacterial elicitor flagellin in Arabidopsis,” Molecular Cell 5: 1003-1011, 2000, Meindi et al., “The bacterial elicitor flagellin activates its receptor in tomato cells according to the address-message concept,” The Plant Cell 12: 1783-1794, 2000). In tomato, high affinity binding of Flg22 to a FLS receptor was observed using both intact cells as well as to microsomal membrane preparations. In this study, the binding of Flg22 to the FLS2 receptor(s) at the plasma membrane surface was nonreversible under physiological conditions, which reflects an uptake process of the Flg22 elicitor with import into the tomato cells (Meindi et al., “The bacterial elicitor flagellin activates its receptor in tomato cells according to the address-message concept,” The Plant Cell 12: 1783-1794, 2000). Recognition of Flg22 by FLS2 triggers both local and systemic plant immune responses. The Flg22-bound, activated FLS2 receptor complex is internalized into plant cells by endocytosis and Flg22 is shown to move systemically throughout the plant (Jelenska et al., “Flagellin peptide Flg22 gains access to long-distance trafficking in Arabidopsis via its receptor, FLS2,” Journal of Experimental Botany 68: 1769-1783, 2017), which may contribute towards systemic Flg22 immune responses.

Flagellin perception involving Flg22 is highly conserved across divergent plant taxa (Taki et al., “Analysis of flagellin perception mediated by Flg22 receptor OsFLS2 in rice,” Molecular Plant Microbe Interactions 21: 1635-1642, 2008). Submicromolar concentrations of synthetic polypeptides comprising between 15-22 or 28 amino acids from conserved domains of a flagellin protein, act as elicitors to initiate defense responses in a variety of plant species.

Generation of transgenic plants has been used to confirm the flagellin-specific PAMPs that bind to the flagellin-specific PRRs. Ectopic expression of FLS2 in Arabidopsis plants showed a direct correlation between the flagellin responses and FLS2 expression levels, which indicate that FLS2 is involved in the recognition of flagellin (a signal of bacterial presence) and leads to the activation of defense responses in plants (Gómez-Gómez L. and Boller T., “FLS2: An LRR receptor-like kinase involved in the perception of the bacterial elicitor flagellin in Arabidopsis,” Molecular Cell 5: 1003-1011, 2000). Transgenic plants expressing the flagellin binding receptor have shown efficacy against certain pathogens. Flagellin binding to FLS2 was involved in the initiation of expression of specific MAP kinase transcription factors that function downstream of the flagellin receptor FLS2. Mutant plants (fls2) lacking in the FLS2 receptor are insensitive to Flg22 (Gómez-Gómez L. and Boller T., “FLS2: An LRR receptor-like kinase involved in the perception of the bacterial elicitor flagellin in Arabidopsis,” Molecular Cell 5: 1003-1011, 2000), and impaired in Flg22 binding to the FLS2 receptor. Mutant plants (fls2) also exhibited enhanced susceptibility to infection and disease when treated with pathogenic bacteria (Zipfel et al., “Bacterial disease resistance in Arabidopsis through flagellin perception,” Nature 428: 764-767, 2004).

Traditionally, methods to improve disease resistance have capitalized on these and other such findings and have taken a transgenic approach. Transgenic plants and seeds transformed with a Flagellin-Sensing (FLS) receptor protein (WO2016007606A2 incorporated herein by reference in its entirety) or with transcription factors involved in downstream signaling of FLS (WO2002072782A2 incorporated herein by reference in its entirety) have produced plants that confer disease resistance to certain pathogenic microorganisms. In another example, transgenic plants expressing Flagellin-Sensing (FLS3) receptor also have exhibited enhanced resistance to disease compared to non-transgenic plants not expressing the FLS3 receptor (WO2016007606A2 incorporated herein by reference in its entirety).

Plant Defensins/Thionins

Plant defensins are also characterized as anti-microbial peptides (AMPs). Plant defensins contain several conserved cysteinyl residues that form disulfide bridges and contribute to their structural stability. Defensins are among the best characterized cysteine-rich AMPs in plants. Members of the defensin family have four disulfide bridges that fold into a globular structure. This highly conserved structure bestows highly specialized roles in protecting plants against microbial pathogenic organisms (Nawrot et al., “Plant antimicrobial peptides,” Folia Microbiology 59: 181-196, 2014). Thionins are cystine-rich plant AMPs classified in the defensin family and typically comprise 45-48 amino acid residues, in which 6-8 of these amino acids are cysteine that form 3-4 disulfide bonds in higher plants. Thionins have been found to be present in both monocot and dicot plants and their expression can be induced by infection with various microbes (Tam et. al., “Antimicrobial peptides from plants,” Pharmaceuticals 8: 711-757, 2015). Particular amino acids of thionins such as Lys1 and Tyr13, which are highly conserved, have been found to be vital to the functional toxicity of these AMPs.

Root Hair Promoting Polypeptide (RHPP)

Root hair promoting polypeptide (RHPP) is a 12 amino acid fragment derived from soybean Kunitz trypsin inhibitor (KTI) protein, which was detected from soybean meal that was subjected to degradation using an alkaline protease from Bacillus circulans HA₁₂ (Matsumiya Y. and Kubo M. “Soybean and Nutrition, Chapter 11: Soybean Peptide: Novel plant growth promoting peptide from soybean,” Agricultural and Biological Sciences, Sheny H. E. (editor), pgs. 215-230, 2011). When applied to soybean roots, RHPP was shown to accumulate in the roots and promote root growth through the stimulation of cell division and root hair differentiation in Brassica.

Citrus Greening and Other Citrus Diseases

Asian citrus greening disease is transmitted by the Asian citrus psyllid, Diaphorina citri or the two-spotted citrus psyllid, Trioza erytreae Del Guercio, which are both characterized as sap-sucking, hemipteran bug(s) in the family Psyllidae and have been implicated in the spread of citrus greening, a disease caused by a highly fastidious phloem-inhabiting bacteria, Candidatus Liberibacter asiaticus (Halbert, S. E. and Manjunath, K. L, “Asian citrus psyllids Sternorrhyncha: Psyllidae and greening disease of citrus: A literature review and assessment of risk in Florida,” Florida Entomologist 87: 330-353, 2004). Three separate species of the bacteria have been identified to cause HLB disease in citrus plants with Candidatus Liberbacter asiaticus (CLas) the most widespread in North America and responsible for the disease in Florida (Gottwald, TR., “Current epidemiological understanding of citrus Huanglongbing”, Annual Review of Phytopathology 48: 119-139, 2010).

Liberbacter infection in citrus trees is accompanied by callose deposition in the plasmodesmata pore units that connect the companion cells and sieve elements. This callose accumulation was shown to result in the impairment of movement or transport through the phloem in infected trees resulting in a delay in photoassimilate export in Liberibacter infected leaves (Koh et. al., “Callose deposition in the phloem plasmodesmata and inhibition of phloem transport in citrus leaves infected with Candidatus Liberibacter asiaticus” Protoplasma 249: 687-697, 2012). The symptoms of HLB disease include vein yellowing and an asymmetrical chlorosis of leaves termed blotchy mottle that occur as the bacteria clogs up the vascular system and is the most diagnostic symptom of the disease. Early symptoms of yellowing may appear on a single shoot or branch and with disease progression, the yellowing can spread over the entire tree. Infected trees are stunted and sparsely foliated and can have root loss. Overall tree appearance for citrus trees infected with HLB may exhibit yellow shoots with upright narrow leaves, shoot die back, sparse foliation, a thin canopy, stunting, off-season bloom, or an overall yellow appearance.

As HLB continues to infect a tree, there is the spread of yellow leaves, vein corking and green islands on the leaves. Fruit can show signs of HLB infection both inside and out. On the outside, fruits may be lopsided or oblong in shape, they may be smaller than normal fruits, and they may change color abnormally turning orange near the stem and staying green at the blossom end. Fruit of afflicted trees are often few in number, small, deformed (malformed) or lopsided and fail to color properly (discolored), remaining green at the end and display a yellow stain just beneath the peduncle (stem) on a cut fruit. HLB-diseased trees also produce fruit with aborted seeds. The fruit of diseased trees may be green, drop prematurely from the tree and have a low soluble acid content accompanied by a bitter taste, root loss and eventually tree death (International Research Conference on Huanglongbing; Proceedings of the Meeting 2009 Plant Management Network).

To date, there is no effective treatment for trees infected with HLB. Infected trees overtime become unproductive and are usually destroyed to minimize further spread of the bacteria. HLB disease is considered fatal for a citrus tree once the tree becomes infected. All commercially available citrus varieties are susceptible to HLB. Therefore, the demand for new treatments and methods of disease control for HLB is necessary.

HLB disease may also be graft transmitted when citrus rootstocks are selected for and grafted to scion varieties. Management of citrus greening disease has proven difficult and therefore current methods for control of HLB have taken a multi-tiered integrated disease and pest management approach using 1) the implementation of disease-free nursery stock and rootstock used in grafting, 2) the use of pesticides and systemic insecticides to control the psyllid vector, 3) the use of biological control agents such as antibiotics, 4) the use of beneficial insects, such as parasitic wasps that attack the psyllid, and 5) breeding for new citrus germplasm with increased resistance to the citrus greening causing bacteria (Candidatus Liberibacter spp.). The use of cultural and regulatory measures to prevent the spread of the disease is also part of the integrated management approach. Many aspects involved in the management of citrus greening are costly both monetarily and in respect to losses in citrus production.

Tissue sectioning of CLas-infected leaves and stems revealed increased deposition of callose and starch within the plant vasculature (Koh et al., “Callose deposition in the phloem plasmodesmata and inhibition of phloem transport in citrus leaves infected with “Candidatus Liberibacter asiaticus” Protoplasma 249: 687-697, 2012). The devastating symptoms of Citrus Greening Disease or HLB are likely caused in part by a blockage of the plant vasculature with callose, resulting in a failure to move photosynthates through the plant (from source leaves to sink tissues, such as fruit).

Those of skill in the art are able to test for infection by Ca. Liberibacter to identify which plants are infected with the bacteria. Treatment of such infected citrus plants using treatments that comprise a flagellin peptide (Flg22) or an antibiotic (oxytetracycline) provided in combination with inducer compounds and recovery mixtures and methods that use the compositions and mixture to prevent and treat HLB are provided herein.

In addition to HLB disease, other plant pathogens of citrus include the bacterium Xanthomonas citri causing citrus canker, Xanthomonas axonopodis pv. citrumelo causing citrus bacterial spot disease, and Xylellafastidiosa causing citrus variegated chlorosis; the pathogenic fungus Alternaria citri causing leaf and stem rot and spot, Phytophthora spp. causing serious and soil-borne diseases such as foot and root rot, and Guignardia citricarpa causing citrus black spot, all of which can result in economic crop loss, juice and fruit quality. Effective methods and compositions to treat these and other citrus plant pathogens are urgently needed.

### SUMMARY OF THE INVENTION

A composition is provided for bioactive priming of a plant or a plant part to increase growth, yield, health, longevity, productivity, and/or vigor of a plant or a plant part and/or protect the plant or the plant part from disease, and/or increase the innate immune response of the plant or the plant part. The composition comprises (A) at least one bioactive priming polypeptide and at least one inducer compound or (B) at least two bioactive priming polypeptides, optionally with at least one inducer compound; or (C) a callose synthase inhibitor and at least one inducer compound comprising a bacteriocide, an amino acid, a substituted or unsubstituted benzoic acid or derivative or salt thereof, a dicarboxylic acid or derivative or salt thereof, a betaine, a proline, a benzothiadiazole, or any combination thereof; or (D) a bacteriocide and at least one inducer compound comprising β-amino butyric acid (BABA), a betaine, a proline, a benzothiadiazole, salicylic acid, oxalic acid, or any combination thereof; wherein:

the bioactive priming polypeptide or polypeptides of (A) or (B) comprise:

(i) a flagellin or flagellin-associated polypeptide; or

(ii) a retro inverso flagellin or flagellin-associated polypeptide

(iii) a root hair promoting polypeptide (RHPP); or

(iv) a retro inverso root hair promoting polypeptide (RI RHPP); or

(v) a thionin or thionin-like polypeptide; or

(vi) a glucanase polypeptide; or

(vii) a serine protease polypeptide; or

(viii) an ACC deaminase polypeptide; or

(ix) an amylase; or

(x) a chitinase; or

(xi) any combination thereof;

with the provisos that:

the inducer compound comprises a callose synthase inhibitor, β-amino butyric acid (BABA), a betaine, a proline, salicylic acid, oxalic acid, a benzothiadiazole, or any combination thereof when the polypeptide of (A) comprises any polypeptide from groups (i) to (iv) but not polypeptides selected from the groups (v) to (x); and

the inducer compound comprises a bacteriocide, an amino acid or isomer thereof, a callose synthase inhibitor, a substituted or unsubstituted benzoic acid or derivative thereof, a dicarboxylic acid or derivative thereof, a betaine, a proline, a benzothiadiazole, or any combination thereof when the polypeptide of (A) comprises any polypeptide from groups (v) to (x);

and the composition comprises the inducer compound and the inducer compound comprises a callose synthase inhibitor, β-amino butyric acid (BABA), a betaine, proline, salicyclic acid, oxalic acid, a benzothiadiazole, or any combination thereof when the two or more polypeptides of (B) comprise polypeptides selected from groups (i)-(iv) but not polypeptides selected from the groups (v) to (x).

Another composition is provided for bioactive priming of a plant or a plant part to increase growth, yield, health, longevity, productivity, and/or vigor of a plant or a plant part and/or protect the plant or the plant part from disease, and/or increase the innate immune response of the plant or the plant part and/or improve the quality of a fruit, juice obtained from a fruit, or a harvest obtained from a plant or plant part, wherein the composition comprises bixafen and at least one free polypeptide comprising

(i) a flagellin or flagellin-associated polypeptide; or

(ii) a retro inverso flagellin or flagellin-associated polypeptide

(iii) a root hair promoting polypeptide (RHPP); or

(iv) a retro inverso root hair promoting polypeptide (RI RHPP); or

(v) a thionin or thionin-like polypeptide; or

(vi) a glucanase polypeptide; or

(vii) a serine protease polypeptide; or

(viii) an ACC deaminase (1-aminocyclopropane-1-carboxylate deaminase) polypeptide; or

(ix) an amylase; or

(x) a chitinase; or

(xi) any combination thereof;

wherein the free polypeptide is not bound to an exosporium of a Bacillus cereus family member or an intact Bacillus cereus family member spore.

An isolated peptide for bioactive priming of a plant or a plant part is provided, to increase growth, yield, health, longevity, productivity, and/or vigor of a plant or a plant part and/or decrease abiotic stress in the plant or plant part and/or protect the plant or the plant part from disease, insects and/or nematodes and/or increase the innate immune response of the plant or the plant part and/or change plant architecture, wherein the peptide comprises the amino acid sequence of any one of SEQ ID NOs: 732, 735, 746-755 and 757-778, or the peptide consists of the amino acid sequence of any one of SEQ ID NOs: 732, 735, and 745-778.

A method is provided for increasing growth, yield, health, longevity, productivity, and/or vigor of a plant or plant part and/or protecting the plant or plant part from disease and/or increase the innate immune response of the plant or the plant part, the method comprising applying a composition or an isolated polypeptide to a plant, plant part, or a plant growth medium in which the plant or plant part will be grown, or a rhizosphere in an area surrounding the plant or the plant part to increase growth, yield, health, longevity, productivity, and/or vigor of the plant or plant part and/or protect the plant or the plant part from disease and/or increase the innate immune response of the plant or plant part wherein the isolated polypeptide comprises: a β-1,3 glucanase and the β-1,3 glucanase is injected into the trunk of the citrus plant; or an amino acid sequence of the isolated polypeptide comprises any one of SEQ ID NOs: 732, 735, 746-755 and 757-778, or consists of any one of SEQ ID NOs: 732, 735, and 745-778; and the composition comprises a β-1,3 glucanase, or bixafen and at least one free polypeptide or (A) at least one bioactive priming polypeptide and at least one inducer compound or (B) at least two bioactive priming polypeptides, optionally with at least one inducer compound; or (C) a callose synthase inhibitor and at least one inducer compound comprising a bacteriocide, an amino acid, a substituted or unsubstituted benzoic acid or derivative or salt thereof, a dicarboxylic acid or derivative or salt thereof, a betaine, a proline, a benzothiadiazole, a or any combination thereof; or (D) a bacteriocide and at least one inducer compound comprising β-amino butyric acid (BABA), a betaine, a proline, a benzothiadiazole, salicylic acid, oxalic acid, or any combination thereof; wherein:
the bioactive priming polypeptide or polypeptides of (A) or (B) or the free polypeptide comprise:
(i) a flagellin or flagellin-associated polypeptide; or
(ii) a retro inverso flagellin or flagellin-associated polypeptide
(iii) a root hair promoting polypeptide (RHPP); or
(iv) a retro inverso root hair promoting polypeptide (RI RHPP); or
(v) a thionin or thionin-like polypeptide; or
(vi) a glucanase polypeptide; or
(vii) a serine protease polypeptide; or
(viii) an ACC deaminase polypeptide; or
(ix) an amylase; or
(x) a chitinase; or
(xi) any combination thereof;
with the provisos that:
the inducer compound comprises a callose synthase inhibitor, β-amino butyric acid (BABA), a betaine, a proline, salicylic acid, oxalic acid, a benzothiadiazole or any combination thereof when the polypeptide of (A) comprises any polypeptide from groups (i)-(v) but not polypeptides selected from the groups (vi) to (x); and
the inducer compound comprises a bacteriocide, an amino acid or isomer thereof, a callose synthase inhibitor, a substituted or unsubstituted benzoic acid or derivative thereof, a dicarboxylic acid or derivative thereof, a betaine, a proline, a benzothiadiazole, or any combination thereof when the polypeptide of (A) comprises any polypeptide from groups (vi) to (x); and
the composition comprises the inducer compound and the inducer compound comprises a callose synthase inhibitor, β-amino butyric acid (BABA), a betaine, proline, salicyclic acid, oxalic acid, a benzothiadiazole, or any combination thereof when the two or more polypeptides of (B) comprise polypeptides selected from groups (i)-(v) but not polypeptides selected from the groups (vi) to (x).

Another method is provided for increasing juice content and/or improving juice, sugar or acid content or improving a Brix:acid ratio of juice obtained from a plant, the method comprising applying a composition or an isolated polypeptide to the plant or plant part, or plant growth medium in which the plant will be grown, or a rhizosphere in an area surrounding the plant or plant part to increase juice content and/or improving juice, sugar or acid content or improve a Brix:acid ratio of juice obtained from the plant or plant part, the isolated polypeptide comprises a β-1,3 glucanase and the β-1,3 glucanase is injected into the trunk of the citrus plant; or an amino acid sequence of the isolated polypeptide comprises any one of SEQ ID NOs: 732, 735, 746-755 and 757-778, or consists of any one of SEQ ID NOs: 732, 735, and 745-778 and the composition comprising β-1,3-glucanase, bixafen and at least one free polypeptide, or (A) at least one polypeptide and an inducer compound; (B) at least two polypeptides, optionally, with an inducer compound; (C) a callose synthase inhibitor and at least one of an inducer compound comprising a bacteriocide, an amino acid or isomer thereof, a substituted or unsubstituted benzoic acid or derivative or salt thereof, a dicarboxylic acid or derivative or salt thereof, a benzothiadiazole, a betaine, a proline, or any combination thereof; or (D) a bacteriocide and at least one of an inducer compound comprising an amino acid or isomer thereof, a substituted or unsubstituted benzoic acid or derivative or salt thereof, a dicarboxylic acid or derivative or salt thereof, a benzothiadiazole, a betaine, a proline, or any combination thereof; wherein: the polypeptide or polypeptides of (A) or (B) or the free polypeptide comprise:

(i) a flagellin or flagellin-associated polypeptide; or

(ii) a retro inverso flagellin or flagellin-associated polypeptide

(iii) a root hair promoting polypeptide (RHPP); or

(iv) a retro inverso root hair promoting polypeptide (RI RHPP); or

(v) a thionin or thionin-like polypeptide; or

(vi) a glucanase polypeptide; or

(vii) a serine protease polypeptide; or

(viii) an ACC deaminase polypeptide; or

(ix) an amylase; or

(x) a chitinase; or

(ix) any combination thereof;

The features of the invention are further defined in the appended claims. Other objects and features will be in part apparent and in part pointed out hereinafter.

### BRIEF DESCRIPTION OF THE DRAWINGS

FIG. 1 shows the Bt.4Q7Flg22 bioactive priming polypeptide in its native L configuration (SEQ ID NO: 226) and the corresponding retro inverso or D configuration form (SEQ ID NO: 375).

### DEFINITIONS

When the articles “a,” “an,” “one,” “the,” and “said” are used herein, they mean “at least one” or “one or more” unless otherwise indicated.

The terms “comprising,” “including,” and “having” are intended to be inclusive and mean that there may be additional elements other than the listed elements.

“Abiotic stress” as used herein is defined as an environmental condition that can have a negative impact on a plant. Abiotic stress can include: temperature (high or low) stress, radiation stress (visible or UV), drought stress, cold stress, salt stress, osmotic stress, nutrient-deficient or high metal stress, or water stress that results in water deficit, flooding or anoxia. Other abiotic stress factors include dehydration, wounding, ozone, and high or low humidity.

“Bioactive priming” refers to an effect of the polypeptides and/or compositions as described herein to improve a plant or a plant part. Bioactive priming can increase growth, yield, quality, health, longevity, productivity, and/or vigor of a plant or a plant part and/or decrease abiotic stress in the plant or the plant part and/or protect the plant or the plant part from disease, insects and/or nematodes, and/or increase the innate immune response of the plant or the plant part and/or change plant architecture. Bioactive priming can be used to protect a plant or plant part from cosmetic damage due to bacterial or fungal growth on the surface of the plant or plant part or remove/cleanse bacteria and/or fungi from the surface of a plant or plant part. Bioactive priming can also improve the quality and/or quantity of a product obtained from a plant. For example, bioactive priming can improve juice quality or quantity obtained from a citrus plant.

A “bioactive priming polypeptide” as used herein may be used interchangeably with the term “priming agent(s)” and as described for the classes of polypeptides of the: flagellin and flagellin-associated polypeptides, thionins, root hair promoting polypeptide (RHPP), serine proteases, glucanases, and ACC deaminases as well as any retro inverso polypeptides thereof.

A “colorant” as used herein acts as a visual product identifier for product branding and application. Colorants can include, but are not limited to, dyes and pigments, inorganic pigments, organic pigments, polymeric colorants, and formulated pigment coating dispersions available in a variety of highly concentrated shades.

“Endogenously” applied as used herein refers to an application to the inside of a plant surface. Small bioactive priming polypeptides are particularly suited for signalling and communication within a plant. Inside a plant surface refers to a surface internal to any plant membrane or plant cell. Internal could be used to mean either extracellular or intracellular to a plant cell and is inclusive of xylem, phloem, tracheids, etc. Endogenous can refer to movement systemically or through a plant such as referring to cell to cell movement in a plant. Endogenous application can include delivery of bioactive priming polypeptides using recombinant endophytic bacteria or fungi, wherein the endophytic microorganism is delivered externally to the plant and through natural mechanisms moves internally to the plant.

“Exogenously” applied as used herein refers to an application to the outside of a plant surface. A plant surface can be any external plant surface, for example a plasma membrane, a cuticle, a trichome, a leaf, a root hair, seed coat, etc.

“-associated” or “-like” polypeptides as used herein refers to polypeptides derived from or structurally similar to the recited polypeptide but having an amino acid sequence and/or source distinct from the recited polypeptide. For example, the thionin-like protein from Brassica rapa (SEQ ID NO: 664) has a different sequence than thionin from Brassica napus (SEQ ID NO: 663) but is structurally and functionally similar.

A “foliar treatment” as used herein refers to a composition that is applied to the above ground parts or foliage of a plant or plant part and may have leaves, stems, flowers, branches, or any aerial plant part, for example, scion.

A “free polypeptide” as used herein refers to a peptide, polypeptide or protein (e.g., an enzyme) that is substantially free of intact cells. The term “free polypeptide” includes, but is not limited to, crude cell extracts containing a polypeptide, a partially purified, a substantially purified, or a purified polypeptide. Free polypeptides can optionally be immobilized on a chemical matrix or support to allow for controlled release of the polypeptide. Free polypeptide preparations preferably do not include polypeptides bound to an exosporium of a Bacillus cereus family member. Free polypeptides also preferably do not include polypeptides bound to exosporium of an intact Bacillus cereus family member spore.

“Injection” as described herein can be used interchangeably with vaccination or immunization and provides a process whereby the bioactive priming polypeptides are delivered endogenously to a plant or plant part.

“Inoculation” means to deliver-bacteria or living microorganisms that produce the priming polypeptide to a plant or plant part. Inoculation can also refer to the delivery of the priming polypeptide for passive entry through the stomata or any opening in or on a plant or plant part.

A “plant” refers to but is not limited to a monocot plant, a dicot plant, or a gymnosperm plant. The term “plant” as used herein includes whole plants, plant organs, progeny of whole plants or plant organs, embryos, somatic embryos, embryo-like structures, protocorms, protocorm-like bodies, and suspensions of plant cells. Plant organs comprise shoot vegetative organs/structures (e.g., leaves, stems and tubers), roots, flowers and floral organs/structures (e.g., bracts, sepals, petals, stamens, carpels, anthers and ovules), seed including embryo, endosperm, and seed coat and fruit (the mature ovary), plant tissue (e.g., phloem tissue, xylem tissue, vascular tissue, ground tissue, and the like) and cells (e.g., guard cells, egg cells, trichomes and the like). The class of plants that can be used in the methods described herein is generally as broad as the class of higher plants, specifically angio-sperms monocotyledonous (monocots) and dicotyledonous (dicots) plants and gymnosperms. It includes plants of a variety of ploidy levels, including aneuploid, polyploid, diploid, haploid, homozygous and hemizygous. The plants described herein can be monocot crops, such as, sorghum, maize, wheat, rice, barley, oats, rye, millet, and triticale. The plants described herein can also be dicot crops, such as apple, pear, peach, plum, orange, lemon, lime, grapefruit, kiwi, pomegranate, olive, peanut, tobacco, tomato, etc. Also, the plants can be horticultural plants such as rose, marigold, primrose, dogwood, pansy, geranium, etc. Also, the plant can be a citrus plant or a row crop. Other suitable plants are discussed in more detail in the specification below.

A plant “biostimulant” is any substance or microorganism applied to a plant or a plant part that is used to enhance nutrition efficiency, abiotic stress tolerance and/or any other plant quality trait(s).

A “plant cell” as used herein refers to any plant cell and can comprise a cell at the plant surface or internal to the plant plasma membrane, for example, an epidermal cell, a trichome cell, a xylem cell, a phloem cell, a sieve tube element, or a companion cell.

A “plant part” as described herein refers to a plant cell, a plant tissue (e.g., phloem tissue, xylem tissue, vascular tissue, ground tissue, and the like), a plant system (e.g., the vascular system), a leaf, a stem, a flower, a floral organ, a fruit, pollen, a vegetable, a tuber, a corm, a bulb, a pseudobulb, a pod, a root, a rhizome, a root ball, a root stock, a scion, or a seed.

A “polypeptide” as described herein refers to any protein, peptide or polypeptide. The polypeptide can comprise or consist of 100 amino acids or fewer, 90 amino acids or fewer, 80 amino acids or fewer, 70 amino acids or fewer, 60 amino acids or fewer, 50 amino acids or fewer, or 40 amino acids or fewer. The polypeptide can comprise or consist of 6 or more amino acids, 7 or more amino acids, 8 or more amino acids, 9 or more amino acids, or 10 or more amino acids. For example, the polypeptide can comprise or consist of from 6 to 50 amino acids, from 6 to 40 amino acids, from 6 to 35 amino acids, from 6 to 30 amino acids, from 7 to 30 amino acids, from 8 to 30 amino acids, from 9 to 30 amino acids, from 10 to 30 or from 15 to 30 amino acids. The polypeptide can comprise or consist of about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 amino acids.

Alternatively, the polypeptide can comprise a full-length protein and comprise or consist of from about 100 to about 500 amino acids, from about 100 to 400 amino acids, from about 200 to about 400 amino acids, from about 300 to about 500 amino acids, from about 300 to about 350 amino acids, from about 350 to 400 amino acids, from about 400 to 450 amino acids amino acids, from about 300 to about 310 amino acids, from about 320 to about 330 amino acids, from about 330 to about 340 amino acids, from about 340 to about 350 amino acids, from about 350 to about 360 amino acids, from about 360 to about 370 amino acids, from about 370 to about 380 amino acids, from about 380 to about 390 amino acids, from about 390 to about 400 amino acids, from about 400 to about 410 amino acids, from about 410 to about 420 amino acids, from about 420 to about 430 amino acids, from about 430 to about 440 amino acids, or from about 440 to about 450 amino acids.

“Priming” or “peptide priming” as used herein refers to a technique used to improve plant performance. In particular priming is a process whereby the bioactive priming polypeptides are applied either exogenously or endogenously to a plant, plant part, plant cell or to the intercellular space of a plant that results in outcomes that provide benefits to a plant, such as enhanced growth, productivity, abiotic stress tolerance, pest and disease tolerance or prevention.

A “retro-inverso” polypeptide as used herein refers to a polypeptide chain of a natural derived polypeptide from a normal-all-L chain reconfigured and built using non-naturally occurring D-amino acids in reverse order of the naturally occurring L-amino acids. The all-D-amino acid form and the parent chain containing all L-form are topological mirrorings of the protein structure.

A “seed treatment” as used herein refers to a substance or composition that is used to treat or coat a seed. Sample seed treatments include an application of biological organisms, chemical ingredients, inoculants, herbicide safeners, micronutrients, plant growth regulators, seed coatings, etc. provided to a seed to suppress, control or repel plant pathogens, insects, or other pests that attack seeds, seedlings or plants or any useful agent to promote plant growth and health.

A “synergistic” effect refers to an effect arising between the interaction or cooperation of two or more bioactive priming polypeptides, substances, compounds, or other agents to produce a combined effect greater than the sum of their separate effects.

A “synergistic effective concentration” refers to the concentration(s) of two or more bioactive priming polypeptides, substances, compounds or other agents that produces an effect greater than the sum of the individual effects.

The term “Citrus” or “citrus”, as used herein refers to any plant of the genus Citrus, family Ruttaceae, and include, but are not limited to: Sweet orange also known as Hamlin or Valencia orange (Citrus sinensis, Citrus maxima x Citrus reticulata), Bergamot Orange (Citrus bergamia, Citrus limetta x Citrus aurantium), Bitter Orange, Sour Orange, or Seville Orange (Citrus aurantium, Citrus maxima x Citrus reticulata), Blood Orange (Citrus sinensis), Orangelo or Chironja (Citrus paradisi x Citrus sinensis), Mandarin Orange (Citrus reticulate), Trifoliate Orange (Citrus trifoliata), Tachibana Orange (Citrus tachibana), Alemow (Citrus macrophylla), Clementine (Citrus clementina), Cherry Orange (Citrus kinokuni), Lemon (Citrus limon, or hybrids with Citrus maxima x Citrus medica) or Citrus limonia, Indian Wild Orange (Citrus indica), Imperial Lemon (Citrus limon, Citrus medica x Citrus paradisi), Lime (Citrus latifoli, Citrus aurantifolia), Meyer Lemon (Citrus meyeri); hybrids of Citrus x meyeri with Citrus maxima, Citrus medica, Citrus paradisi and/or Citrus sinensis), Rough Lemon (Citrus jambhiri), Volkamer Lemon (Citrus volkameriana), Ponderosa Lemon (Citrus limon x Citrus medica), Key Lime (Citrus aurantiifolia), Kaffir Lime (Citrus hystrix or Mauritius papeda), Sweet Lemon, Sweet Lime, or Mosambi (Citrus limetta), Persian Lime or Tahiti Lime (Citrus latifolia), Palestine Sweet Lime (Citrus limettioides), Winged Lime (Citrus longispina), Australian Finger Lime (Citrus australasica), Australian Round Lime (Citrus australis), Australian Desert or Outback Lime (Citrus glauca), Mount White Lime (Citrus garrawayae), Jambola (Citrus grandis), Kakadu Lime or Humpty Doo Lime (Citrus gracilis), Russel River Lime (Citrus inodora), New Guinea Wild Lime (Citrus warburgiana), Brown River Finger Lime (Citrus wintersii), Mandarin Lime (Citrus limonia; (hybrids with Citrus reticulata x Citrus maxima x Citrus medica), Carabao Lime (Citrus pennivesiculata), Blood Lime (Citrus australasica x Citrus limonia) Limeberry (Triphasia brassii, Triphasia grandifolia, Triphasia trifolia), Lemon hybrid or Lumia (Citrus medica x Citrus limon), Omani Lime (Citrus aurantiifolia, Citrus medica x Citrus micrantha), Sour Lime or Nimbuka (Citrus acida), Grapefruit (Citrus paradisi; Citrus maxima x Citrus x sinensis), Tangarine (Citrus tangerina), Tangelo (Citrus tangelo; Citrus reticulata x Citrus maxima or Citrus paradisi), Minneola Tangelo (Citrus reticulata x Citrus paradisi), Orangelo (Citrus paradisi x Citrus sinensis), Tangor (Citrus nobilis; Citrus reticulata x Citrus sinensis), Pummelo or Pomelo (Citrus maxima or Citrus retkulata), Citron (Citrus medica), Mountain Citron (Citrus halimii), Kumquat (Citrus japonica or Fortunella species), Kumquat hybrids (Calamondin, Fortunella japonica; Citranqequat, Citrus ichangensis; Limequat, Citrofortunella floridana; Orangequat, hybrid between Satsuma mandarin x Citrus japonica or Fortunella species; Procimequat, Fortunella hirdsiie; Sunquat, hybrid between Citrus meyeri and Citrus japonica or Fortunella species; Yuzuquat, hybrid between Citrus ichangensis and Fortunella margarita), Papedas (Citrus halimii, Citrus indica, Citrus macroptera, Citrus micrantha), Ichang Papeda (Citrus ichangensis), Celebes Papeda (Citrus celebica), Khasi Papeda (Citrus latipes), Melanesian Papeda (Citrus macroptera), Ichang Lemon (Citrus ichangensis x Citrus maxima), Yuzu (Citrus ichangensis x Citrus reticulata), Cam sành (Citrus reticulata x Citrus maxima), Kabosu (Citrus sphaerocarpa), Sudachi (Citrus sudachi), Alemow (Citrus macrophylla), Biasong (Citrus micrantha), Samuyao (Citrus micrantha), Kalpi (Citrus webberi), Mikan (Citrus unshiu), Hyuganatsu (Citrus tamurana), Manyshanyegan (Citrus mangshanensis), Lush (Citrus crenatifolia), Amanatsu or Natsumikan (Citrus natsudaidai), Kinnow (Citrus nobilis x Citrus deliciosa), Kiyomi (Citrus sinensis x Citrus unshiu), Oroblanco (Citrus maxima x Citrus paradisi), Ugh (Citrus reticulata x Citrus maxima and/or Citrus x paradisi), Calamondin (Citrus reticulata x Citrus japonica), Chinotto (Citrus myrtifoha, Citrus aurantium or Citrus pumila), Cleopatra Mandarin (Citrus reshni), Daidai (Citrus aurantium or Citrus daidai), Laraha (Citrus aurantium), Satsuma (Citrus unshiu), Naartjie (Citrus reticulata x Citrus nobilis), Rangpur (Citrus limonia; or hybrid with Citrus sinensis x Citrus maxima x Citrus reticulata), Djeruk Limau (Citrus amblycarpa), Iyokan, anadomikan (Citrus iyo), Odichukuthi (Citrus odichukuthi), Ougonkan (Citrus flaviculpus), Pompia (Citrus monstruosa), Tangerine (Citrus tangerine), Taiwan Tangerine (Citrus depressa), Shonan gold (Citrus flaviculpus or Citrus unshiu), Sunki (Citrus sunki), Mangshanyen (Citrus mangshanensis, Citrus nobilis), Clymenia (Clymenia platypoda, Clymenia polyandra), Jabara (Citrus jabara), Mandora (Mandora cyprus), Melogold (Citrus grandis x Citrus paradisii/Citrus maxima/Citrus grandis), Shangjuan (Citrus ichangensis x Citrus maxima), Nanfengmiju (Citrus reticulata), and Shīkwāsaī (Citrus depressa).

The term “Huanglongbing,” “Huanglongbing disease,” or “HLB,” as used herein, refers to a disease of plants caused by microorganisms of the Candidatus genus Liberibacter, such as L. asiaticus, L. africanus, and L. americanus. This disease, for example, can be found in citrus plants, or other plants in the genus Rutaceae. Symptoms of Huanglongbing disease include one or more of yellow shoots and mottling of the plant leaves, occasionally with thickening of the leaves, reduced fruit size, fruit greening, premature dropping of fruit from the plant, low fruit soluble acid content, fruit with a bitter or salty taste, or death of the plant.

The term “treating” or “treatment,” or its cognates, as used herein indicates any process or method which prevents, cures, diminishes, reduces, ameliorates, or slows the progression of a disease. Treatment can include reducing pathogen titer in plant tissue or the appearance of disease symptoms relative to controls which have not undergone treatment. Treatment can also be prophylactic (e.g., by preventing or delaying an infection in a plant).

The term “reduction of disease symptoms,” as used herein, refers to a measurable decrease in the number or severity of disease symptoms.

The term “treatment application,” as used herein, refers to any treatment that includes an injection treatment, such as the injection into a trunk of a tree or a plant part, any application to the foliage of a plant or the soil that a plant is growing in and any application to a seed of a plant or the area surrounding the seed of a plant.

As used herein, “cysteine” can comprise analogs, acids or salts of cysteine. Cysteine is a thiol-containing amino acid in the form of L-cysteine, D-cysteine, DL-cysteine, analogs of L-cysteine comprising: DL homocysteine, L-cysteine methyl ester, L-cysteine ethyl ester, N-carbamoyl cysteine, N-acetylcysteine, L-cysteine sodium salt, L-cysteine monosodium salt L-cysteine disodium salt, L-cysteine monohydrochloride, L-cysteine hydrochloride, L-cysteine ethyl ester hydrochloride, L-cysteine methyl ester hydrochloride, others selenocysteine, seleno-DL-cysteine, N-isobutyryl-L-cysteine, N-isobutyryl-L-cysteine or an acid of cysteine such as cysteine sulfinic acid.

As used herein, “betaine” refers to any betaine, betaine homolog, or betaine analog. The betaine can comprise glycine betaine, glycine betaine aldehyde, β-alanine betaine, betaine hydrochloride, cetyl betaine, proline betaine, choline-O-sulfate betaine, cocaamidopropyl betaine, oleyl betaine, sulfobetaine, lauryl betaine, octyl betaine, caprylamidopropyl betaine, lauramidopropyl betaine, isostearamidopropyl betaine, or a combination, homolog, or analog of any thereof. For example, the betaine can comprise glycine betaine, glycine betaine aldehyde, β-alanine betaine, betaine hydrochloride, cetyl betaine, choline-O-sulfate betaine, cocaamidopropyl betaine, oleyl betaine, sulfobetaine, lauryl betaine, octyl betaine, caprylamidopropyl betaine, lauramidopropyl betaine, isostearamidopropyl betaine, or a combination, homolog, or analog of any thereof. The betaine can be derived from a plant source such as wheat (e.g., wheat germ or wheat bran) or a plant of the genus Beta (e.g., Beta vulgaris (beet)). The betaine homolog or analog can comprise ectoine, choline, phosphatidylcholine, acetylcholine, cytidine disphosphate choline, dimethylethanolamine, choline chloride, choline salicylate, glycerophosphocholine, phosphocholine, a sphingomyelin, choline bitartrate, propio betaine, deanol betaine, homodeanol betaine, homoglycerol betaine, diethanol homobetaine, triethanol homobetaine, or a combination of any thereof.

As used herein, “proline” refers to any proline, proline homolog or proline analog. The proline can comprise L-proline, D-proline, hydroxyproline, hydroxyproline derivatives, proline betaine, or a combination, derivative, homolog, or analog of any thereof. The proline homolog or analog can comprise α-methyl-L-proline, α-benzyl-Lproline, trans-4-hydroxy-L-proline, cis-4-hydroxy-L-proline, trans-3-hydroxy-L-proline, cis-3-hydroxy-L-proline, trans-4-amino-L-proline, 3,4-dehydro-α-proline, (2S)-aziridine-2-carboxylic acid, (2S)-azetidine-2-carboxylic acid, L-pipecolic acid, proline betaine, 4-oxo-L-proline, thiazolidine-2-carboxylic acid, (4R)-thiazolidine-4-carboxylic acid, or a combination of any thereof. As used herein, the term “inducer compound” is any compound or substance that acts synergistically with another substance to improve the overall effect either substance would have on a plant or plant part alone. For example, an inducer compound can improve the bioactive priming ability of a bioactive priming polypeptide. Alternatively, two or more inducer compounds can be used in the absence of a polypeptide to exert a synergistic beneficial effect on the plant or plant part. The “beneficial effect” improved by the presence of the inducer can be measured by an increase in growth, yield, health, longevity, productivity, and/or vigor of a plant or a plant part and/or by an improvement in disease symptoms or in the innate immune response of the plant or plant part.

As used herein, the term “derivative” refers to any derivative, analog, salt or ester of the compound.

As used herein, the term “substituted’ refers to a compound having one or more of its carbon atoms or one or more hydrogen atoms bound to a carbon atom replaced with a heteroatom or other group, such as hydroxyl (—OH), alkylthio, phosphino, amido (—CON(R_{A})R_{B}), wherein R_{A} and R_{B} are independently hydrogen, alkyl, or aryl), amino (—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} is alkyl or aryl), or keto (—C(O)R_{A} wherein R_{A} is alkyl or aryl), or heterocyclo. Each substitution can comprise a substituted or unsubstituted alkyl, a substituted or unsubstituted aryl or heteroaryl, or a heteroatom. Suitable substituents include, but are not limited to, lower alkyls (e.g, methyl, ethyl, propyl, butyl), hydroxyls, amines, amides, and benzyls. For example, a “substituted benzoic acid” can comprise a benzoic acid bearing one or more substituents. In an example, one substituent can be a hydroxyl and the substituted benzoic acid can be salicylic acid.

### DESCRIPTION OF THE PREFERRED EMBODIMENTS

There is a growing need for bioactive compositions that act as “priming agents” to provide benefits to agriculture. The use of bioactive “priming” compositions in agricultural practices provides a paradigm shift for integrated crop management practices for example, to manage disease, abiotic stress and yield programs. Bioactive priming compositions herein can comprise bioactive priming polypeptides (naturally occurring, recombinant or synthetic) and/or an inducer compound. Compositions and methods of using the bioactive priming polypeptides and/or inducer compounds are described to supply a multi-tiered treatment regime to apply to crops to achieve agronomically desirable outcomes. Such desirable outcomes include enhanced phenotypes in plants such as those that exhibit protection against pest, disease agents and abiotic stress, as well as increased plant growth, productivity and yield. More specifically, the formulations of the bioactive priming polypeptides and/or inducer compounds described herein can be applied using various treatment regimes, exogenously and/or endogenously to a plant or plant part, and have been discovered to increase growth, yield, health, longevity, productivity, and/or vigor of a plant or a plant part and/or protect the plant or the plant part from disease, and/or increase the innate immune response of the plant or the plant part.

Specific classes of synthetically derived or naturally occurring bioactive priming polypeptides that can be included, alone or in combination, in the compositions herein include flagellins and flagellin-associated polypeptides (including those conserved among the Bacillus genera), thionins, root hair promoting polypeptide (RHPP), serine proteases, glucanases, amylases, chitinases, and ACC deaminases. Each of these classes of polypeptides were selected for their distinct modes of action and can be used individually or in combination with other polypeptides to accommodate the specific agricultural needs described above. For example, in certain cases, isolated polypeptides from these classes can be used individually to accommodate the specific agricultural needs described above. They can be used in the place of or in addition to commercially available agrochemicals, biostimulants, supplemental bioactives and/or pesticidal compounds.

Specific classes of inducer compounds include amino acids (particularly, isolated amino acids) and isomers thereof, certain acids (e.g., substituted or unsubstituted benzoic acids and dicarboxylic acids), bacteriocides, callose synthase inhibitors, succinate dehydrogenase inhibitors, benzothiazoles, and osmoprotectants (e.g., betaines or prolines). Specific inducers in these classes will be described below.

Isolated polypeptides and combinations of the bioactive priming polypeptides and/or the inducer compounds described herein have been found to have a synergistic effect on plant health, yield and disease prevention/treatment. Combinations described herein are particularly effective at treating citrus diseases and improving the yield and quality of a fruit and/or juice obtained from a citrus plant. Further, the compositions provide synergistic benefits to improve the yield and productivity of row crops.

### I. Compositions

Novel bioactive priming compositions are provided herein. More specifically, a composition is provided for bioactive priming of a plant or a plant part to increase growth, yield, health, longevity, productivity, and/or vigor of a plant or a plant part and/or protect the plant or the plant part from disease, and/or increase the innate immune response of the plant or the plant part and/or increase the quantity and/or quality of juice obtained from a citrus plant. The compositions can comprise a β-1,3-glucanase, or (A) at least one bioactive priming polypeptide and an inducer compound or (B) at least two bioactive priming polypeptides, optionally with an inducer compound or (C) at least two inducer compounds. The bioactive priming polypeptides and inducer compounds that can be used in these compositions and the specific methods where they can be used are described below.

Another composition is provided for bioactive priming of a plant or a plant part to increase growth, yield, health, longevity, productivity, and/or vigor of a plant or a plant part and/or protect the plant or the plant part from disease, and/or increase the innate immune response of the plant or the plant part and/or increase the quantity and/or quality of juice obtained from a plant. The compositions can comprise bixafen and a free polypeptide (i.e., not bound to an exosporium of a Bacillus cereus family member or an intact Bacillus cereus family member spore). The free polypeptide can comprise (i) a flagellin or flagellin-associated polypeptide; or (ii) a retro inverso flagellin or flagellin-associated polypeptide; or (iii) a root hair promoting polypeptide (RHPP); or (iv) a retro inverso root hair promoting polypeptide (RI RHPP); or (v) a thionin or thionin-like polypeptide; or (vi) a glucanase polypeptide; or (vii) a serine protease polypeptide; or (viii) an ACC deaminase (1-aminocyclopropane-1-carboxylate deaminase) polypeptide; or (ix) an amylase; or (x) a chitinase; or (xi) any combination thereof.

a. Polypeptides and Compositions Thereof

The compositions described herein can comprise one or more bioactive priming polypeptides or free polypeptides. The bioactive priming peptides and free polypeptides can comprise at least one flagellin or flagellin associated polypeptide, at least one retro-inverso flagellin or flagellin associated polypeptide, at least one root hair promoting polypeptide (RHPP), at least one retro inverso root hair promoting polypeptide (RI-RHPP), at least one thionin or thionin-like polypeptide, at least one glucanase polypeptide, at least one serine protease polypeptide, at least one amylase polypeptide, at least one chitinase polypeptide, at least one ACC deaminase polypeptide or any combination thereof.

The bioactive priming polypeptides and free polypeptides used in the compositions and methods described herein are provided as naturally occurring, recombinant or chemically synthesized forms derived from bacteria or plants. The bioactive priming polypeptides are provided in both the normal L and non-natural retro-inverso D amino-acid forms. In addition, bioactive priming polypeptides are provided that contain non-natural modifications, including N-terminal and C-terminal modifications, cyclization, β-amino and D-amino acid containing, and other chemical modifications that enhance stability or performance of the polypeptides. For example, flagellin and the Flg-associated polypeptides comprising 22 amino acids in length and derived from the full coding region of flagellin were initially isolated and identified from a proprietary genome assembled for bacterial strain, Bacillus thuringiensis 4Q7. These Flg22 derived polypeptides were provided in the standard (L) and retro-inverso (D) forms. They are described as Bt.4Q7Flg22 and retro-inverso (RI) Bt.4Q7Flg22. Other bacterial derived bioactive priming polypeptides are Ec.Flg22 (Escherichia coli), X.Flg22 (Xanthomonas sp.), and other Flg22 from other bacterial species, serine proteases (Bacillus subtilis and other bacterial species), ACC deaminases (Bacillus thuringinesis and other bacterial species), β-1,3-D-glucanases (Paenibacillus spp. and other bacterial species) and amylases (Bacillus subtilis and other bacterial species) while the plant derived polypeptides include thionins (Citrus spp. and other plant species), and RHPP (Glycine max).

The bioactive priming polypeptides and free polypeptides used in the compositions and methods described herein can include full-length proteins and are provided as naturally occurring, synthetic or recombinant forms derived from bacteria or plants. For example, flagellins, thionins, RHPPs, serine proteases, glucanases, amylases, chitinases, and ACC deaminases can all be delivered to plants.

The bioactive priming polypeptides and free polypeptides can also be delivered as fusion partners to other protein sequences, including protease cleavage sites, binding proteins, and targeting proteins to prepare formulations for specific delivery to plants or plant parts.

Also provided are signature, signal anchor sorting and secretion sequences that can be naturally or chemically synthesized and targeting sequences, such as phloem-targeting sequences that are produced along with the bioactive priming polypeptide(s) and free polypeptides using recombinant microorganisms and either used as fusion or assistance polypeptides with the bioactive priming polypeptides and free polypeptides as described herein.

Flagellins and Flagellin-Associated Polypeptides

The composition can comprise a flagellin or flagellin-associated polypeptide.

Flagellin is a globular protein that arranges itself in a hollow cylinder to form the filament in a bacterial flagellum identified from a proprietary bacterial strain of Bacillus thuringiensis strain 4Q7. Flagellin is the principal substituent of bacterial flagellum and is present in flagellated bacteria. Plants can perceive, combat infection and mount defense signaling against bacterial microbes through the recognition of conserved epitopes, such as the stretch of 22 amino acids (Flg22) located in the N-terminus of a full length flagellin coding sequence. The elicitor activity of Flg22 polypeptide is attributed to this conserved domain within the N-terminus of the flagellin protein (Felix et al., 1999). Plants can perceive bacterial flagellin through a pattern recognition receptor (PRR) at the plant's cell surface known as flagellin sensitive receptor, which is a leucine-rich repeat receptor kinase located in the plasma membrane and available at the plant cell surface. In plants, the best-characterized PRR is FLAGELLIN SENSING 2 (FLS2), which is highly conserved in both monocot and dicot plants. A Bt.4Q7Flg22Syn01 is a mutagenized form of the native version Bt.4Q7Flg22 that exhibits an increased activity using assays to the generation of reactive oxygen response which positively correlates to increases in plant immunity and disease resistance in plants.

Flagellin or flagellin-associated polypeptides are particularly useful in compositions for treating bacterial diseases in plants. Upon infection, Candidatus Liberbacter asiaticus (CLas) evades immune detection in part due to point mutations in the flagellin protein FliC that prevent either binding and/or activation of the plant immune receptor Flagellin-Sensing 2 (FLS2). Activation of FLS2 by flagellin protein fragments, such as Bt.4Q7Flg22 triggers production of antimicrobial reactive oxygen species (ROS), up-regulates the plant defense hormone salicylic acid, alters gene expression patterns, and promotes expression of antimicrobial proteins. While CLas flagellin evades detection by the plant, a 22-amino sequence of flagellin FliC from the non-pathogenic bacterium Bacillus thuringiensis strain 4Q7, Bt.Flg22, and the mutagenized form Bt.4Q7Flg22Syn01 are recognized by citrus plants. Bt.4Q7Flg22 or Bt.4Q7Flg22Syn01 treatment induces rapid ROS production, thus activating the plant immune system, leading to reduced CLas bacterial titer in the plant, thus promoting new foliar growth and flowering, which ultimately improves fruit yield.

The flagellin or flagellin-associated polypeptide can be derived from a Bacillus, a Lysinibacillus, a Paenibacillus, an Aneurinibacillus genus bacterium, or any combination thereof.

One of the main classes of bioactive priming polypeptides as described herein are the flagellin(s) and the flagellin-associated priming polypeptide(s). Conserved full and partial length amino acid flagellin coding sequences were identified from various species of Bacillus and non-Bacillus bacteria using methods as described herein.

Flagellin is a structural protein that forms the main portion of flagellar filaments from flagellated bacterial species that can show conservation in the N-terminal and C-terminal regions of the protein but can be variable in the central or mid part (Felix G. et al., “Plants have a sensitive perception system for the most conserved domain of bacterial flagellin,” The Plant Journal 18: 265-276, 1999). The N- and C-terminal conserved regions from flagellins that form the inner core of the flagellin protein may have roles in the polymerization of the protein into a filament, in the motility and transport of the protein and in the surface attachment of a peptide fragment to the plant cell membrane/cell surface receptors of a plant.

Full or partial flagellins (Tables 1-2) and the flagellin-associated polypeptides derived from those Bacillus and non-Bacillus flagellins (Tables 3 and 5) are provided.

The amino acid sequence of the flagellin or flagellin-associated polypeptide can comprise any one of SEQ ID NOs: 226, 1-225, 227-375, 526, 528, 530, 532, 534, 536, 538, 540, 541, or 572-603, or any combination thereof.

Flagellin-associated bioactive priming polypeptides are produced from flagellin coding polypeptides (such as the precursor proteins of Flg22). More specifically, a polypeptide or a cleaved fragment derived from the polypeptide is provided to achieve a bioactive priming Flg polypeptide that can be used to prime or treat a plant. The cleavage of the Flg22 fragment from larger precursors can be accomplished through introduction of proteolytic cleavage sites near the Flg22 to facilitate processing of the active biopeptide from the larger polypeptide.

The flagellin-associated bioactive priming polypeptides can be derived from full length flagellin proteins (or precursor proteins from Flg-associated polypeptides from a Bacillus, a Lysinibacillus, a Paenibacillus, or an Aneurinibacillus or other non-related genera bacterium). For example, PCR purified DNA from the flagellin-associated polypeptides such as Flg22 and FlgII-28 (Bacillus genera) and Flg15 and Flg22 (E. coli) are cloned into a recombinant vector, amplified to achieve adequate amounts of purified DNA that is then sequenced using conventional methods known and used by one of ordinary skill in the art. The same methods can be used with the flagellin coding or the flagellin partial sequences (Table 1), N- or C-terminal flagellin polypeptides (Table 2) and any of the Flg-associated polypeptides (Tables 3-5).

The flagellin or flagellin-associated polypeptide can be derived from any member of Eubacteria that contains the conserved 22 amino acid region that is recognized by the plants. Preferred flagellin or flagellin-associated polypeptides can be derived from a Bacillus, a Lysinibacillus, a Paenibacillus, an Aneurinibacillus genus bacterium, or any combination thereof. Additional preferred flagellin and Flg22 sequences can be obtained from the gammaproteobacteria, which contain conserved 22 amino acid sequences of >68% identity.

Conserved Flagellin Sequences from Bacillus

The flagellin-associated bioactive priming polypeptides correspond to the N-terminal conserved domains of Bacillus spp. and other Eubacterial flagellin and are provided as synthetic, recombinant or naturally occurring forms. The flagellin bioactive priming polypeptides of Flg22, Flg15 and FlgII-28 (Table 3) were identified and act as potent elicitors on a wide range of crops and vegetables to prevent and treat the spread of select disease(s) while synergistically stimulating and promoting growth responses in plants.

The flagellin and flagellin-associated bioactive priming polypeptides as described herein are provided for use in compositions either individually or in combination with other bioactive priming polypeptides as described herein, and include conserved full and partial flagellins from Bacillus (Table 1), conserved N- and C-terminal regions from flagellin polypeptides (Table 2), Bacillus derived Flg22 and FlgII-28-derived bioactive priming polypeptides (Table 3) and retro-inverso sequences that are mirror images derived from the Bacillus Flg22 and FlgII-28 (Table 4). The underlined portion of the sequences in Tables 1 and 3 represent identified signal anchor sorting or secretion sequences, and signal anchoring sequences, respectively. Other non-Bacillus derived polypeptide and proteins are also described that are functional equivalents and can be utilized in similar fashion (Table 5).

TABLE 1
Conserved flagellin sequences from Bacillus

| SEQ ID NO: | Full or Partial Flagellin Coding Sequence - Amino Acid |
|---|---|
| Flagellin | MRINTNINSMRTQEYMRQNQAKMSNAMDRLSSGKRINSASDD |
| SEQ ID NO: 1 | AAGLAIATRMKAREGGLNVAGRNTQDGMSLIRTADSALNSVS |
| Bacillus | NILLRMRDLANQSANGTNTKGNQASLQKEFAQLTEQIDYIAKN |
| thuringiensis strain | TQFNDQQLLGTADKKIKIQTLDTGSTNPAQIEITLNSVKSADLGL |
| 4Q7 | DVQIGDEGDAESTAAADPTSAKQAIDAIDAAITTVAGQRATLGA |
| | TLNRFEFNANNLKSQETSMADAASQIEDADMAKEMSEMTKFKI |
| | LNEAGISMLSQANQTPQMVSKLLQ |
| Flagellin | MRINTNINSMRTQEYMRQNQAKMSNAMDRLSSGKRINSASDD |
| SEQ ID NO: 2 | AAGLAIATRMKAREGGLNVAGRNTQDGMSLIRTADSALNSVS |
| Bacillus | NILLRMRDLANQSANGTNTKGNQASLQKEFAQLTEQIDYIAKN |
| thuringiensis, strain | TQFNDQQLLGTADKKIKIQTLDTGSTNPAQIEITLNSVKSADLGL |
| HD1002 | DVQIGDEGDAESTAAADPTSAKQAIDAIDAAITTVAGQRATLGA |
| | TLNRFEFNANNLKSQETSMADAASQIEDADMAKEMSEMTKFKI |
| | LNEAGISMLSQANQTPQMVSKLLQ |
| Flagellin | MRINTNINSMRTQEYMRQNQAKMSNAMDRLSSGKRINSASDD |
| SEQ ID NO: 3 | AAGLAIATRMKAREGGLNVAGRNTQDGMSLIRTADSALNSVS |
| Bacillus | NILLRMRDLANQSANGTNTKGNQASLQKEFAQLTEQIDYIAKN |
| thuringiensis, | TQFNDQQLLGTADKKIKIQTLDTGSTNPAQIEITLNSVKSADLGL |
| strain HD-789 | DVQIGDEGDAESTAAADPTSAKQAIDAIDAAITTVAGQRATLGA |
| | TLNRFEFNANNLKSQETSMADAASQIEDADMAKEMSEMTKFKI |
| | LNEAGISMLSQANQTPQMVSKLLQ |
| Flagellin | MRINTNINSMRTQEYMRQNQAKMSNAMDRLSSGKRINSASDD |
| SEQ ID NO: 4 | AAGLAIATRMKAREGGLNVAGRNTQDGMSLIRTADSALNSVS |
| Bacillus cereus | NILLRMRDLANQSANGTNTKGNQASLQKEFAQLTEQIDYIAKN |
| strain G9842 | TQFNDQQLLGTADKKIKIQTLDTGSTNPAQIEITLNSVKSADLGL |
| | DVQIGDEGDAESTAAADPTSAKQAIDAIDAAITTVAGQRATLGA |
| | TLNRFEFNANNLKSQETSMADAASQIEDADMAKEMSEMTKFKI |
| | LNEAGISMLSQANQTPQMVSKLLQ |
| Flagellin | MRIGTNVLSMNARQSLYENEKHMNVAMEHLATGKKLNNASD |
| SEQ ID NO: 5 | NPANIAIVTRMHARASGMRVAIRNNEDAISMLRTAEAALQTVT |
| Bacillus | NILQRMRDLAVQSANGTNSNKNRHSLNKEFQSLTEKIGYIGETT |
| thuringiensis | EFNDLSVFEGQNRPITLDDIGHTINMMKHIPPSPTQHDIKISTEQE |
| serovarindiana | ARAAILKIEDALQSVSLHRADLGAMINRLQFNIENLNSQSMALT |
| strain HD521 | DAASLIEDADMAQEMSDFLKFKLLTEVALSMVSQANQIPQMVS |
| | KLLQS |
| Flagellin | MRINTNINSMRTQEYMRQNQAKMSNSMDRLSSGKRINNASDD |
| SEQ ID NO: 6 | AAGLAIATRMRARESGLGVAADNTQNGMSLIRTADSAMNSVS |
| Bacillus | NILLRMRDIANQSANGTNTNENKSALQKEFAQLQKQITYIAENT |
| thuringiensis strain | QFNDKNLLNEDSEVKIQTLDSSKGEQQITIDLKAVTLEKLNIKDI |
| CTC | AIGKADAADKPVTPGATVDQKDLDSVTDKIAALTETSSKADID |
| | AIQSSLDNFKASMTPEDVKTLEDALKGFKTGQANPADAGVDAI |
| | QDALSKVKLPTATAAAPAADADKSDALAAIAAIDAALTKVAD |
| | NRATLGATLNRLDFNVNNLKSQSSSMASAASQIEDADMAKEM |
| | SEMTKFKILNEAGISMLSQANQTPQMVSKLLQ |
| Flagellin | MTGITINLEIDFFAYYRFSICRKVNIKKWGFLNMRINTNINSMRT |
| SEQ ID NO: 7 | QEYMRQNQAKMSNAMDRLSSGKRINNASDDAAGLAIATRMR |
| Bacillus | ARENGLGVAANNTQDGMSLIRTADSAMNSVSNILLRMRDLAN |
| thuringiensis | QSANGTNTDDNQKALDKEFSALKEQIDYISKNTEFNDKKLLNG |
| serovaryunnanensis | ENKTIAIQTLDNADTTKQININLADSSTSALQIDKLTISGKTTDTT |
| strain IEB C- T20001 | KTETITVTDDEIKAAKTDIDEFNDAKKALADLKAETSAGKADGS |
| | TDDEIKTAVSNFTKSFEKIQKFMNDSDIKTVQTEIEKFDAAAPAL |
| | DKAKGMGIAFTSAMDPKAGTITKAATRQNASDAIKSIDAALETI |
| | ASNRATLGATLNRLDFNVNNLKSQSSSMAAAASQIEDADMAK |
| | EMSEMTKFKILNEAGISMLSQANV |
| Flagellin | MRINTNINSMRTQEYMRQNQAKMSNAMDRLSSGKRINNASDD |
| SEQ ID NO: 8 | AAGLAIATRMRARENGLGVAANNTQDGMSLIRTADSALQSVS |
| Bacillus | NILLRMRDLANQSANGTNTDENKAAMEKEFGQLKDQIKYITDN |
| thuringiensis | TQFNDKNLLDAASGTTKSIAIQTLDSDQASTQIEIKIAGSSLAAL |
| serovar tolworthi | GLDKVQIGQETVAQKDLDVLTKAMGRLAAPDADATTRDLDVQ |
| | VAKDAFDKVKGFIADPAQAKAVERAFEDYTAAEAGKEEDAAK |
| | AIDAAYKKVTGLTAGTTGTVDAHNAVNKIDAALKTVADNRAT |
| | LGATLNRLDFNVNNLKSQSASMASAASQIEDADMAKEMSEMT |
| | KFKILNEAGISMLSQANQTPQMVSKLLQ |
| Flagellin | MRINTNINSMRTQEYMRQNQAKMSNSMDRLSSGKRINNASDD |
| SEQ ID NO: 9 | AAGLAIATRMRARESGLGVAANNTQDGMSLIRTADSAMNSVS |
| Bacillus cereus | NILLRMRDIANQSANGTNTDKNQVALQKEFGELQKQIDYIAKN |
| strain FM1 | TQFNDKNLLSGKAGAPDQALEINIQTLDSSDPNQQIKISLDSVST |
| | AQLGVKDLQIGSSSITQQQLDTLDNAMKRLETASTTAAVRDQD |
| | VADAKAAFENVKGFFSEGNVDSINRAFTDFANETTNKDDKAEA |
| | IYALYNNATLITKPTPDASNPASVDPANAIKKIDQAIEKIASSRAT |
| | LGATLNRLDFNVNNLKSQQSSMASAASQVEDADMAKEMSEMT |
| | KFKILNEAGISMLSQANQTPQMVSKLLQ |
| Flagellin | MRIGTNVLSMNARQSFYENEKRMNVAIEHLATGKKLNHASDN |
| SEQ ID NO: 10 | PANVAIVTRMHARTSGIHVAIRNNEDAISMLRTAEAALQTVTNI |
| Bacillus cereus | LQRMRDVAVQSANGTNSNKNRDSLNKEFQSLTEQIGYIDETTEF |
| strain FM1 | NDLSVFDRQNCPVTLDDIGHTVNVTKHIPPSPTQHDINTSTEQEA |
| | RAAIRKIEETLQNVSLHRADLGAMINQLQFNIENLNSQSTALTD |
| | AASRIEDADMAQEMSDFLKFKLLTEVALSMVSQANQIPQMVY |
| | KLLQS |
| Flagellin | MDRLSSGKRINNASDDAAGLAIATRMRARESGLGVAANNTQD |
| SEQ ID NO: 11 | GMSLIRTADSALNSVSNILLRMRDIANQSANGTNTADNQQALQ |
| Bacillus | KEFGQLKEQISYIADNTEFNDKTLLKADNSVKIQTLDSADTNKQ |
| thuringiensis strain | ISIDLKGVTLNQLGLDTVNIGSEKLSAESLNVAKATMARLVKAD |
| MC28 | QNADPSTFALDVNTAKESFDKIKGFIANKTNVQNVENAFNDYA |
| | VADPADKADKADAIQAAFNTAITGLTAGTPNTSNPSSAVDSIDA |
| | ALKTVASNRATLGATLNRLDFNVNNLKSQSASMASAASQIEDA |
| | DMAKEMSEMTKFKILNEAGISMLSQANQTPQMVSKLLQ |
| Flagellin | MRINTNINSMRTQEYMRQNQAKMSNSMDRLSSGKRINNASDD |
| SEQ ID NO: 12 | AAGLAIATRMRSREGGLNVAARNTEDGMSLIRTADSALNSVSN |
| Bacillus | ILLRMRDLANQSASGTNTDKNQAAMQKEFDQLKEQIQYIADNT |
| bombysepticus | EFNDKKLLDGSNSTINTQTLDSHDKNKQITISLDSASLKNLDIKD |
| strain Wang | LAIGSATINQTDLDTATNSMKRLATPATDGKVLAQDIADAKAA |
| | FNKVQSAYTPAEVDKIQDAFKAYDKLAADPASKATDIADAAK |
| | NVNTVFGTLATPTATKFDPSSAVEKIDKAIETIASSRATLGATLN |
| | RLDFNVTNLKSQENSMAASASQIEDADMAKEMSEMTKFKILNE |
| | AGISMLSQANQTPQMVSKLLQ |
| Flagellin | MTGITINLEIDFFAYYRFSICRKVNIKKWGFLNMRINTNINSMRT |
| SEQ ID NO: 13 | QEYMRQNQAKMSNSMDRLSSGKRINNASDDAAGLAIATRMRS |
| Bacillus | REGGLNVAARNTEDGMSLIRTADSALNSVSNILLRMRDLANQS |
| thuringiensis | ASGTNTDKNQAAMQKEFDQLKEQIQYIADNTEFNDKKLLDGSN |
| serovar kenyae | STINTQTLDSHDKNKQITISLDSASLKNLDIKDLAIGSATINQTDL |
| | DTATNSMKRLATPATDGKVLAQDIADAKAAFNKVQSAYTPAE |
| | VDKIQDAFKAYDKLAADPASKDTDIADAAKNVNTVFGTLATPT |
| | ATKFDPSSAVEKIDKAIETIASSRATLGATLNRLDFNVTNLKSQE |
| | NSMAASASQIEDADMAKEMSEMTKFKILNEAGISMLSQANQTP |
| | QMVSKLLQ |
| Flagellin | MRINTNINSMRTQEYMRQNQAKMSNSMDRLSSGKRINNASDD |
| SEQ ID NO: 14 | AAGLAIATRMRSREGGLNVAARNTEDGMSLIRTADSALNSVSN |
| Bacillus | ILLRMRDLANQSASGTNTDKNQAAMQKEFDQLKEQIQYIADNT |
| thuringiensis | EFNDKKLLDGSNSTINTQALDSHDKNKQITISLDSASLKNLDIKD |
| serovar kenyae | LAIGSATINQTDLDTATNSMKRLATPATDGKVLAQDIADAKAA |
| | FNKVQSAYTPAEVDKIQDAFKAYDKLAADPASKDTDIADAAK |
| | NVNTVFGTLATPTATKFDPSSAVEKIDKAIETIASSRATLGATLN |
| | RLDFNVTNLKSQENSMAASASQIEDADMAKEMSEMTKFKILNE |
| | AGISMLSQANQTPQMVSKLLQ |
| Flagellin (A-type) | MRINTNINSMRTQEYMRQNQAKMSNAMDRLSSGKRINNASDD |
| SEQ ID NO: 15 | AAGLAIATRMRARENGLGVAANNTQDGMSLIRTADSALNSVS |
| Bacillus cereus | NILLRMRDLANQSANGTNTGDNQKALDKEFSALKEQIDYISKN |
| | TEFNDKKLLNGDNKTIAIQTLDNADTSKQININLADSSTSALKIE |
| | KLTISGSTAIAGKTEKVTITAEDIKAAEEDIKAFTQAQEGLANLV |
| | KEVKDTDGSVKTPGSTPDDIKKAVTAFTESFEKMKKFMNDEDI |
| | TKVEEKIKAFDAASPDLDAAKEMGTAFTAAMKPAAGEITKAA |
| | MKPNASDAIKSIDEALETIASNRATLGATLNRLDFNVNNLKSQS |
| | SSMASAASQIEDADMAKEMSEMTKFKILNEAGISMLSQANQTP |
| | QMVSKLLQ |
| Flagellin (A-type) | MRIGTNVLSMNARQSLYENEKRMNVAMEHLATGKKLNNASD |
| SEQ ID NO: 16 | NPANIAIVTRMHARASGMRLAIRNNEDTISMLRTAEAALQTLTN |
| Bacillus cereus | ILQRMRDLAVQSANGTNSNKNRDSLNKEFQSLTEQIGYIGETTE |
| | FNDLSVFDGQNRPVTLDDIDHTINMTKHIPPSPTQHDIKISTEQE |
| | ARAAILKIEEALQSVSIHRADLGSMINRLQFNIENLNSQSMALTD |
| | AASRIEDADMAQEMSDFLKFKLLTEVALSMVSQANQIPQMVSK |
| | LLQS |
| Flagellin | MRIGTNVLSMNARQSLYENEKRMNVAMEHLATGKKLNHASD |
| SEQ ID NO: 17 | NPANVAIVTRMHARASGMRVAIRNNEDAISMLRTAEAALQTVT |
| Bacillus | NVLQRMRDVAVQSANGTNLNKNRDSLNNEFQSLTEQIGYIDET |
| thuringiensis | TAFNDLSVFDGQNRPVTLDDIGHTVNVTKHISPSPTQHDINTSTE |
| serovar finitimus | QEARAAIRKIEEALQNVSLYRADLGAMINRLQFNIENLNSQSTA |
| strain YBT-020 | LTDAASRIEDADMAQEMSDFLKFKLLTEVALSMVSQANQIPQM |
| | VYKLLQS |
| Flagellin | MRINTNINSMRTQEYMRQNQAKMSNAMDRLSSGKRINNASDD |
| SEQ ID NO: 18 | AAGLAIATRMRARESGLNVAADNTQNGMSLIRTADSAMNSVS |
| Bacillus | NILLRMRDIANQSANGTNTDSNKSALQKEFAELQKQITYIADNT |
| thuringiensis | QFNDKNLLKEDSEVKIQTLDSSKGEQQIGIDLKAVTLEKLGINNI |
| serovar finitimus | SIGKADGTTEGTKADLTALQAAAKKLEKPDTGTMEKDVKDAK |
| strain YBT-020 | EEFDKVKASLSDEDVKKIEAAFGEFDKDKTNTTKASDIFNAIKD |
| | VKLADKAAAAPAPADLTKFKAALDKLQTPNAGTMVDDVKDA |
| | KDEFEKIKGSLSDADAQKIQAAFEEFEKANTDDSKASAIYNLAK |
| | DVKVNATDTTTGTDKDTTTSTDKDAALAAIAAIDAALTKVADN |
| | RATLGATLNRLDFNVNNLKSQSSSMASAASQIEDADMAKEMSE |
| | MTKFKILNEAGISMLSQANQTPQMVSKLLQ |
| Flagellin | MRINTNINSMRTQEYMRQNQAKMSNAMDRLSSGKRINNASDD |
| SEQ ID NO: 19 | AAGLAIATRMRARESGLGVAANNTQDGMSLIRTADSALNSVSN |
| Bacillus cereus | ILLRMRDLANQSANGTNTAENKAAMQKEFGELKDQIKYISENT |
| stain B4264 | QFNDQHLLNAAKGSTNEIAIQTLDSDSSSKQIKITLQGASLDSLD |
| | IKDLQIGSGSTVSQTDLDVLDATMTRVKTATGATRDVDVQAAK |
| | SAFDKVKGLMTKPAEVKAIERAFEDYNAGKTDALATAIEAAYT |
| | ANKTGLPAPAAAAGTVDALGAITKIDAALKTVADNRATLGATL |
| | NRLDFNVNNLKSQSASMASAASQIEDADMAKEMSEMTKFKILN |
| | EAGISMLSQANQTPQMVSKLLQ |
| Flagellin | MRINTNINSMRTQEYMRQNQAKMSNAMDRLSSGKRINNASDD |
| SEQ ID NO: 20 | AAGLAIATRMRARESGLGVAANNTQDGMSLIRTADSALNSVSN |
| Bacillus | ILLRMRDIANQSANGTNTSDNQKALDKEFSALKEQIDYISKNTE |
| thuringiensis | FNDKKLLNGDNKSIAIQTLDNADTTKQININLADSSTTALNIDKL |
| serovar nigeriensis | SIEGTGNKTITLTAADIAKDKANIDAVGTAKTALAGLTGTPAAA |
| | AINSAVADFKTAFAKADKNLMSDAQIKAVTDAITAFEADATPD |
| | LTKAKAIGTAYTAPAAGDITKASPNASEAIKSIDAALDTIASNRA |
| | TLGATLNRLDFNVNNLKSQSSSMASAASQIEDADMAKEMSEM |
| | TKFKILNEAGISMLSQANQTPQMVSKLLQ |
| Flagellin | MRINTNINSMRTQEYMRQNQAKMSNAMDRLSSGKRINNASDD |
| SEQ ID NO: 21 | AAGLAIATRMRARESGLGVAANNTQDGMSLIRTADSALNSVSN |
| Bacillus | ILLRMRDIANQSANGTNTADNQQALQKEFGQLKEQISYIADNTE |
| thuringiensis | FNDKTLLKADNSVKIQTLDSADTNKQISIDLKGVTLNQLGLDTV |
| | NIGSETLSAESLNVAKATMARLVKADQNADPSTFALDVNTAKE |
| | SFDKIKGFITNKTNVQNVENAFNDYTVADPADKADKADAIQAA |
| | FNTAITGLTAGTPNTSNPSSAVDAIDAALKTVASNRATLGATLN |
| | RLDFNVNNLKSQSASMASAASQIEDADMAKEMSEMTKFKILNE |
| | AGISMLSQANQTPQMVSKLLQ |
| Flagellin | MRIGTNVLSMNARQSLYENEKRMNVAMEHFATGKKLNHASD |
| SEQ ID NO: 22 | NPANVAIVTRMHARASGMRVAIRNNEDAISMLRTAEAALQTV |
| Bacillus | MNILQRMRDLAVQSANGTNSNKNRDSLNKEFQSLTEQIGYIGE |
| thuringiensis | TTEFNDLSVFDGQNRPVTLDDIGHTVNVTKHTSPSPTKHDIKIST |
| serovar konkukian | EQEARAAIRKIEEALQNVSLHRADFGAMINRLQFNIENLNSQSM |
| strain 97-27 | ALTDAASRIEDADMAQEMSDFLKFKLLTEVALSMVSQANQIPQ |
| | MVSKLLQS |
| Flagellin | MRINTNINSMRTQEYMRQNQAKMSNAMDRLSSGKRINNASDD |
| SEQ ID NO: 23 | AAGLAIATRMRARESGLGVAANNTQDGMSLIRTADSALNSVSN |
| Bacillus | ILLRMRDIANQSANGTNTADNQQALQKEFGQLKEQISYIADNTE |
| thuringiensis | FNDKTLLKADNSVKIQTLDSADTNKQISIDLKGVTLNQLGLDTV |
| serovar konkukian | NIGSETLSAESLNVAKATMARLVKADQNADPSTFALDVNTAKE |
| strain 97-27 | SFDKIKGFITNKTNVQNVENAFNDYTVADPADKADKADAIQAA |
| | FNTAITGLTAGTPNTSNPSSAVDAIDAALKTVASNRATLGATLN |
| | RLDFNVNNLKSQSASMASAASQIEDADMAKEMSEMTKFKILNE |
| | AGISMLSQANQTPQMVSKLLQ |
| Flagellin protein | MRIGTNVLSMNARQSLYENEKRIVINVAMEHLATGKKLNHASD |
| FlaA | NPANIVIVTRMYARASGMRVAIRNNEDAISMLRTAEAALQTVT |
| SEQ ID NO: 24 | NILQHMRDFAIQSANGTNSNTNRDSLNKEFQSLTEPIGYIGETTE |
| Bacillus | FNDLSVFDGQNRPITLDDIGHTINMTKHIPPSPTQHDIKISTEQEA |
| thuringiensis | RAAIRKIEEALQNVSLHRADLGSMINRLQFNIENLNSQSMALIDT |
| serovar | ASQVEDADMAQEISDFLKFKLLTAVALSVVSQANQIPQIVSKLL |
| thuringiensis strain | QS |
| IS5056 | |
| Flagellin protein | MRINTNINSMRTQEYMRQNQAKMSNAMDRLSSGKRINNASDD |
| FlaA | AAGLAIATRMRARESGLGVAANNTQDGMSLIRTADSAMNSVS |
| SEQ ID NO: 25 | NILLRMRDISNQSANGTNTDKNQSALDKEFAALKDQIDYISKNT |
| Bacillus | EFNDQKLLDGSKKSIAIQTLDNADTNKQIDIQLSNVSTKELKLDT |
| thuringiensis | LSIEGSSSKTFTITADDMLAVGTANATAKAKAGTLKGLNVTTG |
| serovar | DLTAAKTDVQDFRAAFDKVKGFMGSTEVTNIEKALTKFDGDQS |
| thuringiensis strain | LANAKAIGDALTSDLATTIAKDQTYSKNVSNASSAIASIDAALES |
| IS5056 | IASNRATLGATLNRLDFNVNNLKSQSSSMASAASQIEDADMAK |
| | EMSEMTKFKILNEAGISMLSQANQTPQMVSKLLQ |
| Flagellin B | MRINTNINSMIRTQEYMRQNQAKMSNAMDRLSSGKRINNASDD |
| SEQ ID NO: 26 | AAGLAIATRMRARESGLGVAANNTQDGMSLIRTADSAMNSVS |
| Bacillus | NILLRMRDISNQSANGTNTDKNQSALDKEFAALKDQIDYISKNT |
| thuringiensis | EFNDQKLLDGSKKSIAIQTLDNADTNKQIDIQLSNVSTKELKLDT |
| strain Bt407 | LSIEGSSSKTFTITADDMLAVGTANATAKAKAGTLKGLNVTTG |
| | DLTAAKTDVQDFRAAFDKVKGFMGSTEVTNIEKALTKFDGDQS |
| | LANAKAIGDALTSDLATTIAKDQTYSKNVSNASSAIASIDAALES |
| | IASNRATLGATLNRLDFNVNNLKSQSSSMASAASQIEDADMAK |
| | EMSEMTKFKILNEAGISMLSQANQTPQMVSKLLQ |
| Flagellin | MRINTNINSMIRTQEYMRQNQAKMSNAMDRLSSGKRINNASDD |
| SEQ ID NO: 27 | AAGLAIATRMRARESGLGVAANNTQDGMSLIRTADSAMNSVS |
| Bacillus | NILLRMRDISNQSANGTNTDKNQSALDKEFAALKDQIDYISKNT |
| thuringiensis | EFNDQKLLDGSKKSIAIQTLDNADTNKQIDIQLSNVSTKELKLDT |
| serovar chinensis | LSIEGSSSKTFTITADDMLAVGTANATAKAKAGTLKGLNVTTG |
| CT-43 | DLTAAKTDVQDFRAAFDKVKGFMGSTEVTNIEKALTKFDGDQS |
| | LANAKAIGDALTSDLATTIAKDQTYSKNVSNASSAIASIDAALES |
| | IASNRATLGATLNRLDFNVNNLKSQSSSMASAASQIEDADMAK |
| | EMSEMTKFKILNEAGISMLSQANQTPQMVSKLLQ |
| Flagellin | MTGITINLEIDFFAYYRFSICRKVNIKKWGFLNMRINTNINSMRT |
| SEQ ID NO: 28 | QEYMIRQNQAKMSNAMDRLSSGKRINNASDDAAGLAIATRMR |
| Bacillus | ARESGLGVAANNTQDGISLIRTADSAMNSVSNILLRMRDLANQ |
| thuringiensis | SANGTNTNENQAALNKEFDALKEQIDYISTNTEFNDKKLLDGS |
| serovar canadensis | NKTIAVQTLDNADTSKQININLSNVSTKELGLDTLSIGTDKVEKT |
| | VYDATTKAFADLGAKTGADKAAFDADVTAAMKEFDKVKPFM |
| | SADDVKKIETKLEDYNKANDAGAQTAAQALGKEFATLTKLETT |
| | DLKANASGAIASIDTALKNIASNRATLGATLNRLDFNVNNLKSQ |
| | SSSMASAASQIEDADMAKEMSEMTKFKILNEAGISMLSQANQT |
| | PQMVSKLLQ |
| Flagellin | MTGITINLEIDFFAYYRFSICRKVNIKKWGFLNMRINTNINSMRT |
| SEQ ID NO: 29 | QEYMIRQNQAKMSNAMDRLSSGKRINNASDDAAGLAIATRMR |
| Bacillus | ARESGLGVAANNTQDGISLIRTADSAMNSVSNILLRMRDLANQ |
| thuringiensis | SANGTNTNENQAALNKEFDALKEQIDYISTNTEFNDKKLLDGS |
| serovar galleriae | NKTIAVQTLDNADTSKQININLSNVSTKELGLSTLSIGTDKVEKT |
| | VYDATTKAFADLGAKTGTDKAAFAADVTAAMKEFDKVKPFM |
| | SADDVKKIETKLEDYNKANDAGAEAAAQALGKEFATLTKLETT |
| | DLKANASGAIASIDTALKNIASNRATLGATLNRLDFNVNNLKSQ |
| | SSSMASAASQIEDADMAKEMSEMTKFKILNEAGISMLSQANQT |
| | PQMVSKLLQ |
| Flagellin N-terminal | MRINTNINSMIRTQEYMIRQNQAKMSNAMDRLSSGKRINNASDD |
| helical region | AAGLAIATRMRARESGLSVAANNTQDGMSLIRTADSAMNSVSN |
| SEQ ID NO: 30 | ILLRMRDLSNQSANGTNTDENQQALNKEFAALKDQIDYISKNTE |
| Bacillus | FNDKKLLDGSNKSIAIQTLDNADTTKQINIDLSNVSTDTLNISGL |
| weihenstephanensis | TINGKKDITVTISDKDIANAATDIGKATSAQQGLADLTDTTPAVP |
| | DTPAVIGTGTAGNPQFPAVKGTPEIPGSSPAEIAKAVDDFKQAF |
| | NKVKGLMSDSAVSAMEQKFATFEKDKSLANAKDIGTAFSAPIA |
| | GNITKGEQNASGAIKSIDAALEKIASNRATLGATLNRLDFNVNN |
| | LKSQSSSMASAASQIEDADMAKEMSEMTKFKILNEAGISMLSQ |
| | ANQTPQMVSKLLQ |
| Flagellin | MTGITINLEIDFFAYYRFSICRKVNIKKWGFLNMRINTNINSMRT |
| SEQ ID NO: 31 | QEYMRQNQAKMSNAMDRLSSGKRINNASDDAAGLAIATRMR |
| Bacillus | ARESGLGVAANNTQDGMSLIRTADSALNSVSNILLRMRDIANQ |
| thuringiensis | SANGTNTGDNQKALDKEFSALKEQIDYISKNTEFNDKKLLNGD |
| serovar ostriniae | NKSIAIQTLDNADTAKQININLADSSTKALNIDTLSIAGTTDKTIT |
| | ITAKDLTDNKTTLDALKTAKDDLAKLDDKSDQATIDKAVDAFK |
| | TAFNNVDKNLLSDKAIEGITEKMTAFDGTHTAAAAIGAAYTEPT |
| | AADIKKSAPNASGAIKSIDAALETIASNRATLGATLNRLDFNVN |
| | NLKSQSSSMASAASQIEDADMAKEMSEMTKFKILNEAGISMLS |
| | QANQTPQMVSKLLQ |
| Flagellin | MRIGTNVLSMNARQSLYENEKRMNVAMEHLATGKKLNHASD |
| SEQ ID NO: 32 | NPANVAIVTRMHARASGMRVAIRNNEDALSMLRTAEATLQTV |
| Bacillus | ANILQRMRDLAVQSSNDTNSNKNRDSLNKEFQSLTEQISYIGET |
| thuringiensis | TEFNDLSVFDGQNRPVTLDDIGHTVNVTKHISPSPTQHDIKISTE |
| | QEARAAIRKIEEALQNVLLHRADLGAMINRLQFNIENLNSQSMA |
| | LTDAASRIEDADMAQEMSDFLKFKLLSEVALSMVSQANQIPQM |
| | VSELLQS |
| Flagellin | MRINTNINSMRTQEYMRQNQTKMSNAMDRLSSGKRINNASDD |
| SEQ ID NO: 33 | AAGLAIATRMRARENGLGVAANNTQDGMSLIRTADSAMNSVS |
| Bacillus | NILLRMRDLANQSANGTNTDDNQKALDKEFSALKEQIDYISKN |
| thuringiensis | TEFNDKKLLNGENKTIAIQTLDNADTTKQININLADSSTSALQID |
| | KLTISGKTTDTTKTQTITVTDDEIKAAKTDIDEFNDAKKALADL |
| | KAESAPSKGDGSSDDEIKEAVSNFKKSFEKIQKFMNDSDIKTVQ |
| | TEIEKFDAAAPALDKAKGMGIAFTSAMDPKAGTITKAATRQNA |
| | SDAIKSIDAALETIASNRATLGATLNRLDFNVNNLKSQSSSMAA |
| | AASQIEDADMAKEMSEMTKFKILNEAGISMLSQANQTPQMVSK |
| | LLQ |
| Flagellin | MTGITINLEIDFFAYYRFSICRKVNIKKWGFLIMRINTNINSMRTQ |
| SEQ ID NO: 34 | EYMRQNQTKMSNAMDRLSSGKRINNASDDAAGLAIATRMRAR |
| Bacillus | ENGLGVAANNTQDGMSLIRTADSAMNSVSNILLRMRDLANQS |
| thuringiensis | ANGTNTDDNQKALDKEFSALKEQIDYISKNTEFNDKKLLNGEN |
| serovar | KTIAIQTLDNADTTKQININLADSSTSALQIDKLTISGKTTDTTKT |
| pondicheriensis | QTITVTDDEIKAAKTDIDEFNDAKKALADLKAESAPSKGDGSSD |
| | DEIKEAVSNFKKSFEKIQKFMNDSDIKTVQTEIEKFDAAAPALD |
| | KAKGMGIAFTSAMDPKAGTITKAATRQNASDAIKSIDAALETIA |
| | SNRATLGATLNRLDFNVNNLKSQSSSMAAAASQIEDADMAKE |
| | MSEMTKFKILNEAGISMLSQANQTPQMVSKLLQ |
| Flagellin B | MSIMRIGTNVLSMNARQSLYENEKRMNVAMEHLATGKKLNHA |
| SEQ ID NO: 35 | SDNPANIVIVTRMYARASGMRVAIRNNEDAISMLRTAEAALQT |
| Bacillus | VTNILQHMRDFAIQSANGTNSNTNRDSLNKEFQSLTEPIGYIGET |
| thuringiensis | TEFNDLSVFDGQNRPITLDDIGHTINMTKHIPPSPTQHDIKISTEQ |
| serovar Berliner | EARAAIRKIEEALQNVSLHRADLGSMINRLQFNIENLNSQSMALI |
| | DTASQVEDADMAQEISDFLKFKLLTAVALSVVSQANQIPQIVSK |
| | LLQS |
| Flagellin A | MARITINLEIDFFAYYRFSICRKVNIKKWGFLNMRINTNINSMRT |
| SEQ ID NO: 36 | QDYMRQNQAKMSNAMDRLSSGKRINNASDDAAGLAIATRMR |
| Bacillus | ARESGLGVAANNTQDGMSLIRTADSAMNSVSNILLRMRDISNQ |
| thuringiensis | SANGTNTDKNQSALDKEFAALKDQIDYISKNTEFNDQKLLDGS |
| serovar Berliner | KKSIAIQTLDNADTNKQIDIQLSNVSTKELKLDTLSIEGSSSKTFT |
| | ITADDMLAVGTANATAKAKAGTLKGLNVTTGDLTAAKTDVQD |
| | FRAAFDKVKGFMGSTEVTNIEKALTKFDGDQSLANAKAIGDAL |
| | TSDLATTIAKDQTYSKNVSNASSAIASIDAALESIASNRATLGAT |
| | LNRLDFNVNNLKSQSSSMASAASQIEDADMAKEMSEMTKFKIL |
| | NEAGISMLSQANQTPQMVSKLLQ |
| Flagellin | MRIGTNVLSMNARQSLYENEKRMNVAMEHLATGKKLNHASN |
| SEQ ID NO: 37 | NPANVAIVTRMHARASGMRVAIRNNEDAISMLRTAEAALQTVT |
| Bacillus cereus | NVLQRMRDVAVQSANGTNSSKNRDSLNKEFQSLTEQIGYIDET |
| strain Q1 | TEFNDLSVFDGQNRTVTLDDIGHTVNVTKHIPPSPTQHDINISTE |
| | QEARAAIRKIEEALQNVSLHRADLGAMINRLQFNIENLNSQSTA |
| | LTDAASRIEDADMAQEMSDFLKFKLLTEVALSMVSQANQIPQM |
| | VSKLLQS |
| Flagellin | MRINTNINSMRTQEYMRQNQAKMSNAMDRLSSGKRINNASDD |
| SEQ ID NO: 38 | AAGLAIATRMRARESGLSVAADNTQNGMSLIRTADSAMNSVSN |
| Bacillus cereus | ILLRMRDIANQSANGTNTDKNQVALQKEFAALKEQITYIADNT |
| strain Q1 | QFNDKNLLNGNQTINTQTLDSHDSTKQIGIDLKSATLEALGIKDL |
| | TVGAVGSTEAKNYVDAKEALAKNVAANEFIDAKKALDGNAIA |
| | KGYVEAKTAFDDAKPEVKALVSNYTDALAALAKDDTNDDLK |
| | KDVADTKALMDANTVAKTYFEAKTAHDGADQAIKDIVTTYDS |
| | KLGALDDAANKAISDFDKAKAAFDESPAAKELVKTMDDAKQA |
| | ATQNNTANAYLVAKAAAELAPNDADKKAELENATKALEKDD |
| | TAKGLVKTYENAKEALNPANAMPLDAVKQIDAALKTVADNRA |
| | TLGATLNRLDFNVNNLKSQSSAMAASASQIEDADMAKEMSEM |
| | TKFKILNEAGISMLSQANQTPQMVSKLLQ |
| Flagellin | MRIGTNFLSMNARQSLYENEKRMNVAMEHLATGKKLNHASDN |
| SEQ ID NO: 39 | PANIAIVTRMHARANGMRVAIRNNEDAISMLRTAEAALQTVMN |
| Bacillus | ILQRMRDLAIQSANSTNSNKNRDSLNKEFQSLTEQISYIGETTEF |
| thuringiensis | NDLSVFDGQNRPVTLDDIGHTVHISKSIPPPSPTQHDIKISTEQEA |
| serovar morrisoni | RAAILKIEEALQSVSLHRADLGAMINRLHFNIENLNSQSMALTD |
| | AASRIEDADMAQEMSDFLKFKLLTEVALSMVSQANQIPQMVSK |
| | LLQS |
| Flagellin | MRINTNINSMRTQEYMRQNQTKMSNAMDRLSSGKRINNASDD |
| SEQ ID NO: 40 | AAGLAIATRMRARENGLGVAANNTQDGMSLIRTADSALNSVS |
| Bacillus | NILLRMRDIANQSANGTNTSDNQKALDKEFSALKEQIDYISKNT |
| thuringiensis | EFNDKKLLNGDNKSIAIQTLDNADTTKQININLADSSTSALNIDK |
| serovar | LSIEGTGNKTITLTAADIAKDKTNIDAVGTAKTALAGLTGTPAA |
| neoleonensis | AAINSAVADFKTAFAKADKNLMSDAQIKSVTDAITAFEADATP |
| | DLTKAKAIGTAYTAPAAGDITKASPNASEAIKSIDAALDTIASNR |
| | ATLGATLNRLDFNVNNLKSQSSSMASAASQIEDADMAKEMSE |
| | MTKFKILNEAGISMLSQANQTPQMVSKLLQ |
| Flagellin | MTGITINLEIDFFAYYRFSICRKVNIKKWGFLNMRINTNINSMRT |
| SEQ ID NO: 41 | QEYMRQNQAKMSNAMDRLSSGKRINNASDDAAGLAIATRMR |
| Bacillus | ARESGLGVAANNTQDGMSLIRTADSALNSVSNILLRMRDIANQ |
| thuringiensis | SANGTNTGDNQKALDKEFSALKEQIDYISKNTEFNDKKLLNGD |
| serovar morrisoni | NKSIAIQTLDNADTAKQININLADSSTKALNIDTLSIAGTTDKTIT |
| | ITAKDLTDNKATLDALKTAKADLAKLDDKSDQATIDKAVDAF |
| | KTAFNNVDKNLLSDKAIEGITDKMTAFDGTHTAAAAIGTAYTE |
| | PTAGDITKSAPNASGAIKSIDAALETIASNRATLGATLNRLDFNV |
| | NNLKSQSSSMASAASQIEDADMAKEMSEMTKFKILNEAGISML |
| | SQANQTPQMVSKLLQ |
| Flagellin | MRINTNINSMRTQEYMRQNQAKMSNAMDRLSSGKRINNASDD |
| SEQ ID NO: 42 | AAGLAIATRMRARESGLGVAANNTQDGMSLIRTADSALNSVSN |
| Bacillus | ILLRMRDIANQSANGTNTGDNQKALDKEFSALKEQIDYISKNTE |
| thuringiensis | FNDKKLLNGDNKSIAIQTLDNADTAKQININLADSSTKALNIDTL |
| serovar morrisoni | SIAGTTDKTITITAKDLTDNKATLDALKTAKADLAKLDDKSDQ |
| | ATIDKAVDAFKTAFNNVDKNLLSDKAIEGITDKMTAFDGTHTA |
| | AAAIGTAYTEPTAGDITKSAPNASGAIKSIDAALETIASNRATLG |
| | ATLNRLDFNVNNLKSQSSSMASAASQIEDADMAKEMSEMTKF |
| | KILNEAGISMLSQANQTPQMVSKLLQ |
| Flagellin | MRINTNINSMRTQEYMRQNQAKMSNAMDRLSSGKRINNASDD |
| SEQ ID NO: 43 | AAGLAIATRMRARESGLGVAANNTQDGMSLIRTADSAMNSVS |
| Bacillus | NILLRMRDIANQSANGTNTNGNQAALNKEFDALKQQINYISTNT |
| thuringiensis | EFNDKKLLDGSNKTIAIQTLDNADTSKKIDIQLADVSTKSLNIDK |
| serovar jegathesan | LKIGGVSKETTDAVGDTFTKLSTTATTDMGALKIEVEAAMKEF |
| | DKVKGAMSAEDAKAVTDKLDAFNTAAAATNDAATIAAAKAL |
| | GAAFDKTKVEMADPNASVAAIDSALENIASNRATLGATLNRLD |
| | FNVNNLKSQQSSMASAASQIEDADMAKEMSEMTKFKILNEAGI |
| | SMLSQANQTPQMVSKLLQ |
| Flagellin | MRINTNINSMRTQEYMRQNQAKMSNAMDRLSSGKRINNASDD |
| SEQ ID NO: 44 | AAGLAIATRMRARESGLGVAANNTQDGMALIRTADSAMNSVS |
| Bacillus cereus stain | NILLRMRDIANQSANGTNTDKNQAALQKEFGELQKQIDYIAGN |
| ATCC 10987 | TQFNDKNLLDGSNPSISIQTLDSADQSKQISIDLKSATLEALGIKD |
| | LTVGATENTLAKATITAKDAFDAAKDASDAAKKEIDAAAKDTP |
| | SKNDAQLAKEYIEAKATLATLKPTDATYAAKAAELDAATTALN |
| | DNAKVLVDGYEKKLTTTKTKEAEYTAAKEQSTKSTAAADLVT |
| | KYETAKSNALGNDIAKEYLEAKTAYEANKNDISSKSRFEAAETE |
| | LNKDITANKAAKVLVETYEKAKTAGTTEKSLVAVDKIDEALKT |
| | IADNRATLGATLNRLDFNVNNLKSQSASMASAASQIEDADMAK |
| | EMSEMTKFKILNEAGISMLSQANQTPQMVSKLLQ |
| Flagellin | MTGITINLEIDFFAYYRFSICRKVNIKKWGFLIMRINTNINSMRTQ |
| SEQ ID NO: 45 | EYMRQNQAKMSNAMDRLSSGKRINNASDDAAGLAIATRMRAR |
| Bacillus | ESGLGVAANNTQDGMSLIRTADSAMNSVSNILLRMRDLANQSA |
| thuringiensis | NGTNTNENQAALNKEFDALKEQINYISTNTEFNDKKLLDGSNK |
| serovar monterrey | TIAIQTLDNADTSKKIDIKLADVSTESLKIDKLKIGGVSKETTDA |
| | VSETFTKLSTTKTTDKDALKAEVEAAMKEFDKVKGAMSTEDA |
| | KAVTDKLGLFNTAAAGTDDTAIATAAKNLGAAFDKTKVNMAD |
| | PNASVAAIDSALENIASNRATLGATLNRLDFNVNNLKSQQSSM |
| | ASAASQIEDADMAKEMSEMTKFKILNEAGISMLSQANQTPQMV |
| | SKLLQ |
| Flagellin | MRIGTNVLSLNARQSLYENEKRMNVAMEHLATGKKLNNASDN |
| SEQ ID NO: 46 | PANIAIVTRMHARASSMRVAIRNNEDAISMLRTAEAALQTVTN |
| Bacillus cereus | VLQRMRDLAVQSANDTNSNKNRDSLNKEFQSLTEQIGYIDETT |
| strain NC 7401 | DFNDLSVFDGQNRTVTLDDIGHTVNVTKHIPPSPTQHDINISTEQ |
| | EARAAIRKIEEALQNVSLHRADLGAMINRLQFNIENLNSQSTAL |
| | TDAASRIEDADMAQEMSDFLKFKLLTEVALSMVSQANQIPQMV |
| | SKLLQS |
| Flagellin | MRINTNINSMRTQEYMRQNQAKMSNAMDRLSSGKRINNASDD |
| SEQ ID NO: 47 | AAGLAIATRMRARESGLGVASNNTQDGMSLIRTADSALNSVSN |
| Bacillus cereus | ILLRMRDLANQSANGTNTNENKAAMQKEFGELKEQIKYIAENT |
| strain NC 7401 | QFNDQHLLNADKGITKEIAIQTLDSDSDSKQIKIKLQGSSLEALDI |
| | KDLQIGNTELAQKDLDLLNATMDRLDATVPGTRDVDVQAAKD |
| | AFDKVKGFYTNSDSVKAIERAFEDYATASTAGTAKADAATAIK |
| | AAFDLAANKVGKPATGGAQGSANSLGAITKIDAALKTVADNR |
| | ATLGATLNRLDFNVNNLKSQASSMAAAASQVEDADMAKEMSE |
| | MTKFKILNEAGISMLSQANQTPQMVSKLLQ |
| Flagellin (A-type) | MRINTNINSLRTQEYMRQNQAKMSNSMDRLSSGKRINNASDDA |
| SEQ ID NO: 48 | AGLAIATRMRARESGLNVAANNTQDGMSLIRTADSALGSVSNI |
| Bacillus cereus | LLRMRDLANQSANGTNTSDNQAAMQKEFAELQKQITYIADNT |
| strain AH820 | QFNDKNLLQSNSSINIQTLDSSDGNQQIGIELKSASLKSLGIEDLA |
| | IGASVNPLAKATVEASEAYDKAKADTAAFAKSIADTAATGTGA |
| | AKADAAAVDAYIKEADPTAKGNLYTGLTADQKKLADEHNTLK |
| | AAEDGKKAELTMATTKSTADGTAKGLVDAYDNAKSDAMNDP |
| | KAKAYLEAKMAYEKDTSNVANKQKLDSTKEAMEKDPASKDL |
| | VVKLDAAKAAATNGTPLDAVSKIDAALKTVADNRATLGATLN |
| | RLDFNVNNLKSQSSSMASAASQIEDADMAKEMSEMTKFKILNE |
| | AGISMLSQANQTPQMVSKLLQ |
| Flagellin | MRINTNINSMRTQEYMRQNQAKMSNAMDRLSSGKRINNASDD |
| SEQ ID NO: 49 | AAGLAIATRMRARESGLGVASNNTQDGMSLIRTADSALNSVSN |
| Bacillus cereus | ILLRMRDLANQSANGTNTNENKAAMQKEFGELKEQIKYIAENT |
| AH187 | QFNDQHLLNADKGITKEIAIQTLDSDSDSKQIKIKLQGSSLEALDI |
| | KDLQIGNTELAQKDLDLLNATMDRLDATVPGTRDVDVQAAKD |
| | AFDKVKGFYTNSDSVKAIERAFEDYATASTAGTAKADAATAIK |
| | AAFDLAANKVGKPATGGAQGSANSLGAITKIDAALKTVADNR |
| | ATLGATLNRLDFNVNNLKSQASSMAAAASQVEDADMAKEMSE |
| | MTKFKILNEAGISMLSQANQTPQMVSKLLQ |
| Flagellin | MDFFAYYRFSICRKVNIKKWGFFYMRINTNINSMRTQEYMRQN |
| SEQ ID NO: 50 | QAKMSNAMDRLSSGKRINNASDDAAGLAIATRMRARESGLGV |
| Bacillus cereus | ASNNTQDGMSLIRTADSALNSVSNILLRMRDLANQSANGTNTN |
| | ENKAAMQKEFGELKEQIKYIAENTQFNDQHLLNADKGITKEIAI |
| | QTLDSDSDSKQIKIKLQGSSLEALDIKDLQIGNTELAQKDLDLLN |
| | ATMDRLDATVPGTRDVDVQAAKDAFDKVKGFYTNSDSVKAIE |
| | RAFEDYATASTAGTAKADAATAIKAAFDLAANKVGKPATGGA |
| | QGSANSLGAITKIDAALKTVADNRATLGATLNRLDFNVNNLKS |
| | QASSMAAAASQVEDADMAKEMSEMTKFKILNEAGISMLSQAN |
| | QTPQMVSKLLQ |
| Flagellin protein F1a | MRINTNINSMRTQEYMRQNQAKMSNAMDRLSSGKRINNASDD |
| SEQ ID NO: 51 | AAGLAIATRMRARESGLGVAANNTQDGMSLIRTADSALNSVSN |
| Bacillus cereus | ILLRMRDIANQSANGTNTGDNQKALDKEFSALKEQIDYISKNTE |
| | FNDKKLLNGENTSIAIQTLDSADTAKQININLADSSTSALLIDKLS |
| | ISGAGAGTALAGVATADINAAGTKQAALSGLTGSKTTDELDDA |
| | VKEFKTEFDKVKSGLSAENADKITAAMDKYTNNKTLDNAKAIG |
| | DLYKTMAPADSTVVGTAGTKGQALIDLNATATGDTAQKRQVA |
| | VDAFKDDFDKIKGGLNAQDAAKVTAALDKFNKADGSGNTLEN |
| | AQEIGKVFAEVAAGSTKSNASDAIKSIDKALETIASNRATLGATL |
| | NRLDFNVNNLKSQSSSMASAASQIEDADMAKEMSEMTKFKILN |
| | EAGISMLSQANQTPQMVSKLLQ |
| Flagellin | MRINTNINSMRTQEYMRQNQTKMSNAMDRLSSGKRINNASDD |
| SEQ ID NO: 52 | AAGLAIATRMRSREGGLNVAARNTEDGMSLIRTADSALNSVSN |
| Bacillus | ILLRMRDLANQSASETNTSKNQAAMQKEFDQLKEQIQYIADNT |
| thuringiensis | EFNDKKLLDGSNSTINTQTLDSHDKNKQITISLDSASLKNLDITDL |
| Strain HD-771 | AIGSNTVNKNDLDTLNNSMKRLETAAADAAVQAQDVTDAKN |
| [51] | AFNKVKSGYTPAEVEKMEDAFKAYDKVVADPAKTDALLKAA |
| | AEKINTEFKTLTAPTATAFDPSSSVEKIDKAIETIASSRATLGATL |
| | NRLDFNVTNLKSQENSMAASASQIEDADMAKEMSEMTKFKILN |
| | EAGISMLSQANQTPQMVSKLLQ |
| Flagellin | MRINTNINSMRTQEYMRQNQTKMSNAMDRLSSGKRINNASDD |
| SEQ ID NO: 53 | AAGLAIATRMRSREGGLNVAARNTEDGMSLIRTADSALNSVSN |
| Bacillus | ILLRMRDLANQSASETNTSKNQAAMQKEFDQLKEQIQYIADNT |
| thuringiensis | EFNDKKLLDGSNSTINTQTLDSHDKNKQITISLDSASLKNLDITDL |
| serovar sotto | AIGSNTVNKNDLDTLNNSMKRLETAAADAAVQAQDVTDAKN |
| [52] | AFNKVKSGYTPAEVEKMEDAFKAYDKVVADPAKTDALLKAA |
| | AEKINTEFKTLTAPTATAFDPSSSVEKIDKAIETIASSRATLGATL |
| | NRLDFNVTNLKSQENSMAASASQIEDADMAKEMSEMTKFKILN |
| | EAGISMLSQANQTPQMVSKLLQ |
| Flagellin | MGVLNMRINTNINSMRTQEYMRQNQAKMSNAMDRLSSGKRIN |
| SEQ ID NO: 54 | NASDDAAGLAIATRMRARENGLGVAANNTQDGMSLIRTADSA |
| Bacillus | LNSVSNILLRMRDIANQSANGTNTGDNQKALDKEFSALKEQID |
| thuringiensis | YISKNTEFNDKKLLNGDNKSIAIQTLDNADTSKQINIDLANTSTS |
| serovar Novosibirsk | SLKIDKLSIEGKGNQTIAITAADIAKDTNIAALTSAQGKLAALTG |
| | TPAPAALTTAVDEFKAAFEKVDKNLMSDTQITGIENAIKAYDG |
| | ATTKTLALAQAVGTAYTAPTPGDITKELPNASSSIKSIDAALETI |
| | ASNRATLGATLNRLDFNVNNLKSQASSMASAASQIEDADMAK |
| | EMSEMTKFKILNEAGISMLSQANQTPQMVSKLLQ |
| Flagellin | MGVLNMRINTNINSMRTQEYMRQNQAKMSNAMDRLSSGKRIN |
| SEQ ID NO: 55 | NASDDAAGLAIATRMRARESGLGVAANNTQDGISLIRTADSAM |
| Bacillus | NSVSNILLRMRDLANQSANGTNTSENQAALDKEFGALKEQINYI |
| thuringiensis | STNTEFNDKKLLDGSNETIAIQTLDNADEGKKIDIKLANVSTDSL |
| serovar londrina | KIDKLTIGGAAQKTVDAVADKFNALKTTTTTDKAAIQTEVDAV |
| | MKEFDKVKGSMSAEDAKVITDKLKDYNDAADTDTAKATAAK |
| | DLGAAFDKTKVNIANPNAAVAAIDSALENIASNRATLGATLNR |
| | LDFNVNNLKSQSSSMASAASQIEDADMAKEMSEMTKFKILNEA |
| | GISMLSQANQTPQMVSKLLQ |
| Flagellin | MRIGTNVLSMNARQSLYENEKRMNVAMEHLATGKKLNHASN |
| SEQ ID NO: 56 | NPANIAIVTRMHARASGMRVAIRNNEDALSMLRTAEAALQTVT |
| Bacillus cereus | NILQRMRDLAVQSANVTNSNKNRNSLNKEFQSLTEQISYIGETT |
| strain E3 3L | EFNDLSVFDGQNRPVTLDDIGYTVNVTKHTPPSPTQHDIKISTEQ |
| | EARAAIRKIEEALQNVSLHRADLGSMMNRLQFNIENLNSQSMA |
| | LTDAASRIEDADMAQEMSDFLKFKLLTEVALSMVSQANQIPQM |
| | VSKLLQS |
| Flagellin | MRINTNINSMRTQEYMRQNQAKMSTAMDRLSSGKRINNASDD |
| SEQ ID NO: 57 | AAGLAIATRMRARESGLGVAANNTQDGISLIRTADSAMNSVSNI |
| Bacillus cereus | LLRMRDLANQSANGTNTDKNQGALDKEFAALKEQIDYISKNTE |
| strain E3 3L | FNDKKLLDGSNKAIAIQTLDSDDKGKQIDISLSDTSTTALKINNL |
| | SIAANGLGIGSGKELVGVADNTIANASAEALKKLDGTTGDTDV |
| | KRSNAVKAFTDQYKDLKVAMNAKDVETIDAAIKKFEGANTLE |
| | NAQAIGAAFEGAAKATLTTDINNATLTSKALSDLDTDSTTETRK |
| | AAMKDFVAAFDKVKGSMNSSDVTKISDAIDRFSKTDDSGNTLE |
| | AARAIGDAFKAATTNGKTSTATDANSAIKAIDEALETIASNRAT |
| | LGATLNRLDFNVNNLKNQASSMASAASQVEDADMAKEMSEM |
| | TKFKILNEAGISMLSQANQTPQMVSKLLQ |
| Flagellin | MRINTNINSMRTQEYMRQNQAKMSTAMDRLSSGKRINNASDD |
| SEQ ID NO: 58 | AAGLAIATRMRARESGLGVAANNTQDGISLIRTADSAMNSVSNI |
| Bacillus cereus | LLRMRDLANQSANGTNTDKNQAALDKEFNALKEQIDYISKNTE |
| strain FRI-35 | FNDKKLLDGSNKSIAVQTLDNADTSKQININLSNTSTKALEINSL |
| | TISGTTPIAGKNETSKITAEQMTAASDALEKFKTAQEGLANLTEP |
| | TKGSDGKPEAGTGSSNEDIVKAVKAFKEAFKNIQPLMSDTDITT |
| | VQNKIDLFDEDAPDLSAAKLIGTTFEESMKPVADKEITKAAVKP |
| | NASDAIAAIDAALTKVADNRATLGATLNRLDFNVNNLKSQASS |
| | MASAASQVEDADMAKEMSEMTKFKILNEAGISMLSQANQTPQ |
| | MVSKLLQ |
| Flagellin | MRIGTNVLSLNARQSLYENEKRMNVAMEHLATGKKLNNASDN |
| SEQ ID NO: 59 | PANIAIVTRMHARASGMRVAIRNNEDAISMLRTAEAALQTVTN |
| Bacillus cereus | VLQRMRDLAVQSANGTNSNKNRDSLNKEFQSLTEQIGYIDETT |
| strain FRI-35 | EFNNLSVFDGQNRPVTLDDIGHTVNVTKHIPPFPTQHDINISTEQ |
| | EARAAIRKIEEALQNVSLHRADLGAMINRLQFNIENLNSQSTAL |
| | TDAASRIEDADMAQEMSDFLKFKLLTEVALSMVSQANQVPQM |
| | VSKLLQS |
| Flagellin | MRINTNINSMRTQEYMRQNQAKMSNAMDRLSSGKRINNASDD |
| SEQ ID NO: 60 | AAGLAIATRMRAHESGLSVAARNTSDGISLIRTADSALQSVSNIL |
| Bacillus | LRMRDIANQTANGTNKDTDIEALGKEFAALKEQITYVSDNTKF |
| thuringiensis | NGRELLKGGDDINIQTYDGSDESQQIKIKISELDLSSLDTGEVTD |
| | SDTARGTVSTLDDAITNIASKRAELGATLNRLDYNTQNVNSEA |
| | ASMAASASQIEDADMAKEMSEMTKFKILSEAGISMLSQANQTP |
| | QMVSKLLQ |
| Flagellin | MRINTNINSMRTQEYMRQNQAKMSNAMDRLSSGKRINNASDD |
| SEQ ID NO: 61 | AAGLAIATRMRAHESGLSVAARNTSDGISLIRTADSALQSVSNIL |
| Bacillus cereus | LRMRDIANQTANGTNKDTDIEALGKEFAALKEQITYVSDNTKF |
| strain ATCC 4342 | NGRELLKGGDDINIQTYDGSDESQQIKIKISELDLSSLDTGEVTD |
| | SDTARGTVSTLDDAITNIASKRAELGATLNRLDYNTQNVNSEA |
| | ASMAASASQIEDADMAKEMSEMTKFKILSEAGISMLSQANQTP |
| | QMVSKLLQ |
| Flagellin | MRIGTNFLSMNARQSLYENEKRMNVAMEHLATGKKLNHASDN |
| SEQ ID NO: 62 | PANIAIVTRMHARANGMRVAIRNNEDAISMLRTAEAALQTVMN |
| Bacillus | ILQRMRDLAIQSANSTNSNKNRDSLNKEFQSLTEQISYIGETTEF |
| thuringiensis | NDLSVFDGQNRPVTLDDIGHTVHISKSIPPPSPTQHDIKISTEQEA |
| | RAAILKIEEALQSVSLHRADLGAMINRLHFNIENLNSQSMALTD |
| | AASRIEDADMAQEMSDFLKFKLLTEVALSMVSQANQIPQMVSK |
| | LLQS |
| Flagellin | MRINTNINSMRTQEYMRQNQAKMSNAMDRLSSGKRINNASDD |
| SEQ ID NO: 63 | AAGLAIATRMRARESGLGVAANNTQDGMSLIRTADSALNSVSN |
| Bacillus | ILLRMRDIANQSANGTNTGDNQKALDKEFSALKEQIDYISKNTE |
| thuringiensis | FNDKKLLNGDNKSIAIQTLDNADTAKQININLADSSTKALNIDTL |
| | SIAGTTDKTITITAKDLTDNKATLDALKTAKADLAKLDDKSDQ |
| | ATIDKAVDAFKTAFNNVDKNLLSDKAIEGITDKMTAFDGTHTA |
| | AAAIGTAYTEPTAGDITKSAPNASGAIKSIDAALETIASNRATLG |
| | ATLNRLDFNVNNLKSQSSSMASAASQIEDADMAKEMSEMTKF |
| | KILNEAGISMLSQANQTPQMVSKLLQ |
| Flagellin | MRINHNITALNTYRQFNNANNAQAKSMEKLSSGQRINSASDDA |
| SEQ ID NO: 64 | AGLAISEKMRGQIRGLDQASRNAQDGVSLIQTAEGALNETHDIL |
| Bacillus aryabhattai | QRMRELVVQAGNGTNKTEDLDAIQDEIGSLIEEIGGETDSKGISD |
| | RAQFNGRNLLDGSLDITLQVGANAGQQVNLKIGDMSAGALGA |
| | DTDSDGAADAFVNSINVKDFATTSFDDQLAIIDGAINQVSEQRS |
| | GLGATQNRLDHTINNLSTSSENLTASESRIRDVDYALAA |
| Flagellin | MRINTNINSMRTQEYMRQNQDKMNTSMNRLSSGKQINSASDD |
| SEQ ID NO: 65 | AAGLAIATRMRAKEGGLNVGAKNTQDGMSALRTMDSALNSVS |
| Bacillus | NILLRMRDLATQSATGTNQGNDRESLDLEFQQLTEEITHIAEKT |
| manliponensis | NFNGNALLSGSGSAINVQLSDAAEDKLTIAAIDATASTLLKGAV |
| | DVKTEDKADAAITKIDQAIQDIADNRATYGSQLNRLDHNLNNV |
| | NSQATNMAAAASQIEDADMAKEMSEMTKFKILSEAGVSMLSQ |
| | ANQTPQMVSKLLQ |
| Flagellin | MRIGSWTATGMSIVNEIMNRNAVNAASKSMLRLSSGYRINSAAD |
| SEQ ID NO: 66 | DAAGLAISEKMRGQIRGLTMASKNIMDGVSLIQTAEGALNETH |
| Lysinibacillus sp. | AIVQRMRELAVQAATDTNTDDDRAKLDLEFQELKKEIDRISTDT |
| strain BF-4 | EFNTRTLLNGDYKDNGLKIQVGANSGQAIEVKIGDAGLAGIGLS |
| | TESIATREGANAALGKLDEATKNVSMERSRLGAYQNRLEHAYN |
| | VAENTAINLQDAESRIRDVDIAKEMMNMVKSQILAQVGQQVLA |
| | MHMQQAQGILRLLG |
| Flagellin | MKIGSWTATGMSIVNHMNRNWNAASKSMLRLSSGYRINSAAD |
| SEQ ID NO: 67 | DAAGLAISEKMRGQIRGLTMASKNIMDGVSLIQTAEGALNETH |
| Lysinibacillus sp. | AIVQRMRELAVQAATDTNTDDDRAKLDLEFQELKKEIDRISTDT |
| strain 13S34_air | AFNTRTLLNGDYKDNGLKIQVGANSGQAIEVKIGDAGLAGIGLS |
| | TESIATREGANAALGKLDEATKNVSMERSRLGAYQNRLEHAYN |
| | VAENTAINLQDAESRIRDVDIAKEMMHMVKSQILAQVGQQVLA |
| | MHIQQAQGILRLLG |
| Flagellin | MIISHNLTALNTMNKLKQKDLAVSKSLGKLSSGLRINGASDDA |
| SEQ ID NO: 68 | AGLAISEKMRGQIRGLNQASRNIQDGISLIQVADGAMQEIHSML |
| Paenibacillus sp. | QRMNELAVQASNGTYSGSDRLNIQSEVEQLIEEIDEIAGNTGFN |
| strain HW567 | GIKLLNGNNEKTEKTEKTGSVVSVNNPPNNKLITISSPVGTSVSE |
| | ILNNLLTVFNEAKNGQVGDSDSKRVSSKFTLSINNDELSIVCDTG |
| | DGFLLSGGSPNLFYQGYIGGSYKYKFTEFINENDFINIMDIGGAN |
| | GGDTLKFNFSSISKEPEEQKEQKGLTLQIGANSGETLNIKLPNVT |
| | TSAIGISSIDVSTIPNAESSLSSISAAIDKVSAERARMGAYQNRLE |
| | HSRNNVVTYAENLTAAESRIRDVDMAKEMMELMKNQIFTQAG |
| | QAMLLQTNTQPQAILQLLK |
| Flagellin | MRINTNINSMRTQEYMRQNQAKMSNAMDRLSSGKRINNASDD |
| SEQ ID NO: 69 | AAGLAIATRMRARESGLGVAANNTQDGMSLIRTADSAMNSVS |
| Bacillus anthracis | NILLRMRDLANQSANGTNTKENQDALDKEFGALKEQIDYISKN |
| | TEFNDKKLLNGDNKSIAIQTLDNADTAKQININLADSSTKALNID |
| | SLTISGSKDATITITAEDITAASAEITAAKGARTALANLKDTPADP |
| | TKDPAASTPAEIKAAVDDFKGKFEKIKGLMNDTDVKAVEEKIK |
| | EFETTSTLAKAQAIGTAFTTGMEPKAGNITKNVPAASSSIKAIDS |
| | ALETIASNRATLGATLNRLDFNVNNLKSQSSAMASAASQIEDAD |
| | MAKEMSEMTKFKILNEAGISMLSQANQTPQMVSKLLQ |
| Flagellin | MQKSQYKKMGVLKMRINTNINSMRTQEYMRQNQDKMNVSM |
| SEQ ID NO: 70 | NRLSSGKRINSAADDAAGLAIATRMRARQSGLEKASQNTQDG |
| Bacillus anthracis | MSLIRTAESAMNSVSNILTRMRDIAVQSSNGTNTAENQSALQKE |
| | FAELQEQIDYIAKNTEFNDKNLLAGTGAVTIGSTSISGAEISIETL |
| | DSSATNQQITIKLANTTAEKLGIDATTSNISISGAASALAAISALN |
| | TALNTVAGNRATLGATLNRLDRNVENLNNQATNMASAASQIE |
| | DADMAKEMSEMTKFKILNEAGISMLSQANQTPQMVSKLLQ |
| Flagellin | MRINTNINSMRTQEYMRQNQDKMNVSMNRLSSGKRINSAADD |
| SEQ ID NO: 71 | AAGLAIATRMRARQSGLEKASQNTQDGMSLIRTAESAMNSVSN |
| Bacillus anthracis | ILTRMRDIAVQSSNGTNTAENQSALQKEFAELQEQIDYIAKNTE |
| | FNDKNLLAGTGAVTIGSTSISGAEISIETLDSSATNQQITIKLANT |
| | TAEKLGIDATTSNISISGAASALAAISALNTALNTVAGNRATLGA |
| | TLNRLDRNVENLNNQATNMASAASQIKDADKAKEMSEMTKFK |
| | ILNEAGISMLSQANQTPQMVSKLLQ |
| Flagellin | MRINTNINSMRTQEYMRQNQDKMNVSMNRLSSGKRINSAADD |
| SEQ ID NO: 72 | AAGLAIATRMRARQSGLEKASQNTQDGMSLIRTAESAMNSVSN |
| Bacillus anthracis | ILTRMRDIAVQSSNGTNTAENQSALQKEFAELQEQIDYIAKNTE |
| | FNDKNLLAGTGAVTIGSTSISGAEISIETLDSSATNQQITIKLANT |
| | TAEKLGIDATTSNISISGAASALAAISALNTALNTVAGNRATLGA |
| | TLNRLDRNVENLNNQATNMASAASQIEDADMAKEMSEMTKFK |
| | ILNEAGISMLSQANQTPQMV |
| Flagellin | MNVSMNRLSSGKRINSAADDAAGLAIATRMRARQSGLEKASQ |
| SEQ ID NO: 73 | NTQDGMSLIRTAESAMNSVSNILTRMRDIAVQSSNGTNTAENQS |
| Bacillus anthracis | ALQKEFAELQEQIDYIAKNTEFNDKNLLAGTGAVTIGSTSISGAE |
| strain H9401 | ISIETLDSSATNQQITIKLANTTAEKLGIDATTSNISISGAASALAA |
| | ISALNTALNTVAGNRATLGATLNRLDRNVENLNNQATNMASA |
| | ASQIEDADMAKEMSEMTKFKILNEAGISMLSQANQTPQMVSKL |
| | LQ |
| Flagellin | MRINHNITALNTYRQFNNANNAQAKSMEKLSSGQRINSASDDA |
| SEQ ID NO: 74 | AGLAISEKMRGQIRGLDQASRNAQDGVSLIQTAEGALNETHDIL |
| Bacillus megaterium | QRMRELVVQAGNGTNKTEDLDAIQDEIGSLIEEIGGEADSKGIS |
| strain WSH-002 | DRAQFNGRNLLDGSLDITLQVGANAGQQVNLKIGDMSAGALG |
| | ADTNSDGAADAFVNSINVKDFTATSFDDQLAIIDGAINQVSEQR |
| | SGLGATQNRLDHTINNLSTSSENLTASESRIRDVDYALAA |
| Flagellin | MRINHNLPALNAYRNLAQNQIGTSKILERLSSGYRINRASDDAA |
| SEQ ID NO: 75 | GLAISEKMRGQIRGLEQGQRNTMDGVSLIQTAEGALQEIHEML |
| Aneurinibacillus sp. | QRMRELAVQAANGTYSDKDKKAIEDEINQLTAQIDQIAKTTEF |
| XH2 | NGIQLIGDSDSTSLQDVKIQYGPKKEDSLTLELTTQPEADPPFAA |
| | GCKADKASLKIDNVDVISDPEGAIETFKAAIDQVSRIR SYFGAIQ |
| | NRLEHVVNNLSNYTENLTGAESRIRDADMAKEMTEFTRFNIINQ |
| | SATAMLAQANQLPQGVLQLLKG |

N- and C-Terminal Conserved Regions of Flagellin

The flagellin or flagellin-associated polypeptide used in the compositions and methods herein can comprise a truncated N-terminal polypeptide and an amino acid sequence of the truncated N-terminal polypeptide can comprise SEQ ID NO: 76, 78, 80, 82, 84, 86, 88, 90, 92, 94, 96, 98, 100, 102, 104, 106, 108, 109, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 590, or any combination thereof.

The flagellin or flagellin-associated polypeptide used in the compositions and methods herein can comprise a truncated C-terminal polypeptide and an amino acid sequence of the truncated C-terminal polypeptide can comprise SEQ ID NO: 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 141, 143, 145, 147, 149, 151, 153, 155, 157, 159, 161, 163, 165, 167, 169, 171, 173, 175, 177, 179, 181, 183, 185, 187, 189, 191, 201, 203, 205, 207, 209, 211, 213, 215, 217, 219, 221, 223, 225, or any combination thereof.

N-terminal and C-terminal conserved regions were identified from full length flagellin sequences from diverse strains of Bacillus spp. and other Eubacteria (Table 2). Conserved N- and C-terminal domains were identified using BLAST multiple alignment software and assigned functional annotations based on individual hits searching against Bacillus and other Eubacterial bacterial databases. The start site for the N-terminal region of the coding sequences is bolded methionine (M). The conserved domains are provided as amino acid sequences N-terminus (left column) and C-terminus (right column).

TABLE 2
N- and C-terminal conserved regions of flagellins

| SEQ ID NO: | Conserved N-terminus | Conserved C-terminus |
|---|---|---|
| Flagellin | GFLNMRINTNINSMRTQEYMRQ | IDAAITTVAGQRATLGA |
| N-SEQ ID NO: 76 | NQAKMSNAMDRLSSGKRINSAS | TLNRFEFNANNLKSQET |
| C-SEQ ID NO: 77 | DDAAGLAIATRMKAREGGLNV | SMADAASQIEDADMAK |
| Bacillus thuringensis | AGRNTQDGMSLIRTADSALNSV | EMSEMTKFKILNEAGIS |
| strain 4Q7 | SNILLRMRDLANQSANGTNTKG | MLSQANQTPQMVSKLL |
| [CDS of SEQ ID NO: 1] | NQASLQKEFAQLTEQIDYIAKNT | Q |
| | QFNDQQLLGTADKKIKIQTL | |
| Flagellin | GFLNMRINTNINSMRTQEYMRQ | IDAAITTVAGQRATLGA |
| N-SEQ ID NO: 78 | NQAKMSNAMDRLSSGKRINSAS | TLNRFEFNANNLKSQET |
| C-SEQ ID NO: 79 | DDAAGLAIATRMKAREGGLNV | SMADAASQIEDADMAK |
| Bacillus thuringiensis, | AGRNTQDGMSLIRTADSALNSV | EMSEMTKFKILNEAGIS |
| strain | SNILLRMRDLANQSANGTNTKG | MLSQANQTPQMVSKLL |
| HD1002 | NQASLQKEFAQLTEQIDYIAKNT | Q |
| [CDS of SEQ ID NO: 2] | QFNDQQLLGTADKKIKIQTL | |
| Flagellin | GFLNMRINTNINSMRTQEYMRQ | IDAAITTVAGQRATLGA |
| N-SEQ ID NO: 80 | NQAKMSNAMDRLSSGKRINSAS | TLNRFEFNANNLKSQET |
| C-SEQ ID NO: 81 | DDAAGLAIATRMKAREGGLNV | SMADAASQIEDADMAK |
| Bacillus thuringiensis, | AGRNTQDGMSLIRTADSALNSV | EMSEMTKFKILNEAGIS |
| strain HD-789 | SNILLRMRDLANQSANGTNTKG | MLSQANQTPQMVSKLL |
| [CDS of SEQ ID NO: 3] | NQASLQKEFAQLTEQIDYIAKNT | Q |
| | QFNDQQLLGTADKKIKIQTL | |
| Flagellin | GFLNMRINTNINSMRTQEYMRQ | IDAAITTVAGQRATLGA |
| N-SEQ ID NO: 82 | NQAKMSNAMDRLSSGKRINSAS | TLNRFEFNANNLKSQET |
| C-SEQ ID NO: 83 | DDAAGLAIATRMKAREGGLNV | SMADAASQIEDADMAK |
| Bacillus cereus | AGRNTQDGMSLIRTADSALNSV | EMSEMTKFKILNEAGIS |
| strain G9842 | SNILLRMRDLANQSANGTNTKG | MLSQANQTPQMVSKLL |
| [CDS of SEQ ID NO: 4] | NQASLQKEFAQLTEQIDYIAKNT | Q |
| | QFNDQQLLGTADKKIKIQTL | |
| Flagellin | GFLNMRINTNINSMRTQEYMRQ | QLDAALTKVADNRATL |
| N-SEQ ID NO: 84 | NQAKMSNSMDRLSSGKRINSAA | GATLNRLDFNVNNLKS |
| C-SEQ ID NO: 85 | DDAAGLAIATRMKAREGGLNV | QENSMAASASQIEDAD |
| Bacillus thuringiensis | AARNTQDGMSLIRTADSALNSV | MAKEMSEMTKFKILNE |
| serovar indiana strain | SNILLRMRDLANQSATGTNTTK | AGISMLSQANQTPQMV |
| HD521 | NQVALNKEFAALKEQITYIADN | SKLLQ |
| [CDS of SEQ ID NO:5] | TQFNDKNLLKSTQEIKIQTL | |
| Flagellin | WGFLIMRINTNINSMRTQEYMR | AIAAIDAALTKVADNR |
| N-SEQ ID NO: 86 | QNQAKMSNSMDRLSSGKRINN | ATLGATLNRLDFNVNN |
| C-SEQ ID NO: 87 | ASDDAAGLAIATRMRARESGLG | LKSQSSSMASAASQIED |
| Bacillus thuringiensis | VAADNTQNGMSLIRTADSAMN | ADMAKEMSEMTKFKIL |
| strain CTC | SVSNILLRMRDIANQSANGTNT | NEAGISMLSQANQTPQ |
| [CDS of SEQ ID NO:6] | NENKSALQKEFAQLQKQITYIAE | MVSKLLQ |
| | NTQFNDKNLLNEDSEVKIQTLD | |
| | S | |
| Flagellin | GFLNMRINTNINSMRTQEYMRQ | RATLGATLNRLDFNVN |
| N-SEQ ID NO: 88 | NQAKMSNAMDRLSSGKRINNA | NLKSQSSSMAAAASQIE |
| C-SEQ ID NO: 89 | SDDAAGLAIATRMRARENGLG | DADMAKEMSEMTKFKI |
| Bacillus | VAANNTQDGMSLIRTADSAMN | LNEAGISMLSQAN |
| thuringiensis | SVSNILLRIVIRDLANQSANGTNT | |
| serovar yunnanensis | DDNQKALDKEFSALKEQIDYIS | |
| strain IEBC-T20001 | KNTEFNDKKLL | |
| [CDS of SEQ ID NO:7] | | |
| Flagellin | GFLNMRINTNINSMRTQEYMRQ | IDAALKTVADNRATLG |
| N-SEQ ID NO: 90 | NQAKMSNAMDRLSSGKRINNA | ATLNRLDFNVNNLKSQ |
| C-SEQ ID NO: 91 | SDDAAGLAIATRMRARENGLG | SASMASAASQIEDADM |
| Bacillus thuringiensis | VAANNTQDGMSLIRTADSALQS | AKEMSEMTKFKILNEA |
| serovar tolworthi | VSNILLRMRDLANQSANGTNTD | GISMLSQANQTPQMVS |
| [CDS of SEQ ID NO:8] | ENKAAMEKEFGQLKDQIKYITD | KLLQ |
| | NTQFNDKNLLDA | |
| Flagellin | MGVLNMRINTNINSMRTQEYM | RATLGATLNRLDFNVN |
| N-SEQ ID NO: 92 | RQNQAKMSNSMDRLSSGKRIN | NLKSQQSSMASAASQV |
| C-SEQ ID NO: 93 | NASDDAAGLAIATRMRARESGL | EDADMAKEMSEMTKF |
| Bacillus cereus strain | GVAANNTQDGMSLIRTADSAM | KILNEAGISMLSQANQT |
| FM1 | NSVSNILLRMRDIANQSANGTN | PQMVSKLLQ |
| [CDS of SEQ ID NO: 9] | TDKNQVALQKEFGELQKQIDYI | |
| | AKNTQFND | |
| Flagellin | MGVLNMRIGTNVLSMNARQSF | RADLGAMINQLQFNIE |
| N-SEQ ID NO: 94 | YENEKRMNVAIEHLATGKKLN | NLNSQSTALTDAASRIE |
| C-SEQ ID NO: 95 | HASDNPANVAIVTRMHARTSGI | DADMAQEMSDFLKFKL |
| Bacillus cereus strain | HVAIRNNEDAISMLRTAEAALQ | LTEVALSMVSQANQIP |
| FM1 | TVTNILQRMRDVAVQSANGTNS | QMVYKLLQ |
| [CDS of SEQ ID NO: | NKNRDSLNKEFQSLTEQIGYIDE | |
| 10] | TTEFND | |
| Flagellin | GFLNMRINTNINSMRTQEYMRQ | AVDSIDAALKTVASNR |
| N-SEQ ID NO: 96 | NQAKMSNAMDRLSSGKRINNA | ATLGATLNRLDFNVNN |
| C-SEQ ID NO: 97 | SDDAAGLAIATRMRARESGLGV | LKSQSASMASAASQIED |
| Bacillus thuringiensis | AANNTQDGMSLIRTADSALNSV | ADMAKEMSEMTKFKIL |
| strain MC28 | SNILLRMRDIANQSANGTNTAD | NEAGISMLSQANQTPQ |
| [CDS of SEQ ID NO: | NQQALQKEFGQLKEQISYIADN | MVSKLLQ |
| 11] | TEFNDKTLL | |
| Flagellin | GFLNMRINTNINSMRTQEYMRQ | LGATLNRLDFNVTNLK |
| N-SEQ ID NO: 98 | NQAKMSNSMDRLSSGKRINNAS | SQENSMAASASQIEDA |
| C-SEQ ID NO: 99 | DDAAGLAIATRMRSREGGLNV | DMAKEMSEMTKFKILN |
| Bacillus bombysepticus | AARNTEDGMSLIRTADSALNSV | EAGISMLSQANQTPQM |
| strain Wang | SNILLRMRDLANQSASGTNTDK | VSKLLQ |
| [CDS of SEQ ID NO: | NQAAMQKEFDQLKEQIQYI | |
| 12] | | |
| Flagellin | GFLNMRINTNINSMRTQEYMRQ | RATLGATLNRLDFNVT |
| N-SEQ ID NO: 100 | NQAKMSNSMDRLSSGKRINNAS | NLKSQENSMAASASQIE |
| C-SEQ ID NO: 101 | DDAAGLAIATRMRSREGGLNV | DADMAKEMSEMTKFKI |
| Bacillus thuringiensis | AARNTEDGMSLIRTADSALNSV | LNEAGISMLSQANQTP |
| serovar kenyae | SNILLRMRDLANQSASGTNTDK | QMVSKLLQ |
| [CDS of SEQ ID NO: | NQAAMQKEFDQLKEQIQYI | |
| 13] | | |
| Flagellin | GFLNMRINTNINSMRTQEYMRQ | RATLGATLNRLDFNVT |
| N-SEQ ID NO: 102 | NQAKMSNSMDRLSSGKRINNAS | NLKSQENSMAASASQIE |
| C-SEQ ID NO: 103 | DDAAGLAIATRMRSREGGLNV | DADMAKEMSEMTKFKI |
| Bacillus thuringiensis | AARNTEDGMSLIRTADSALNSV | LNEAGISMLSQANQTP |
| serovar kenyae | SNILLRMRDLANQSASGTNTDK | QMVSKLLQ |
| [CDS of SEQ ID NO: | NQAAMQKEFDQLKEQIQYI | |
| 14] | | |
| Flagellin (A-type) | GFLNMRINTNINSMRTQEYMRQ | RATLGATLNRLDFNVN |
| N-SEQ ID NO: 104 | NQAKMSNAMDRLSSGKRINNA | NLKSQSSSMASAASQIE |
| C-SEQ ID NO: 105 | SDDAAGLAIATRMRARENGLG | DADMAKEMSEMTKFKI |
| Bacillus cereus | VAANNTQDGMSLIRTADSALNS | LNEAGISMLSQANQTP |
| [CDS of SEQ ID NO: | VSNILLRMRDLANQSANGTNTG | QMVSKLLQ |
| 15] | DNQKALDKEFSALKEQIDYISK | |
| | NTEFNDKKLL | |
| Flagellin (A-type) | GFLNMRIGTNVLSMNARQSLYE | RADLGSMINRLQFNIEN |
| N-SEQ ID NO: 106 | NEKRMNVAMEHLATGKKLNN | LNSQSMALTDAASRIED |
| C-SEQ ID NO: 107 | ASDNPANIAIVTRMHARASGMR | ADMAQEMSDFLKFKLL |
| Bacillus cereus | LAIRNNEDTISMLRTAEAALQTL | TEVALSMVSQANQIPQ |
| [CDS of SEQ ID NO: | TNILQRMRDLAVQSANGTNSNK | MVSKLLQ |
| 16] | NRDSLNKEFQSLTEQIGYIGETT | |
| | EFND | |
| Flagellin | GVLNMRINTNINSMRTQEYMR | AIDAALTKVADNRATL |
| N-SEQ ID NO: 108 | QNQAKMSNAMDRLSSGKRINN | GATLNRLDFNVNNLKS |
| C-SEQ ID NO: 109 | ASDDAAGLAIATRMRARESGLN | QSSSMASAASQIEDAD |
| Bacillus thuringiensis | VAADNTQNGMSLIRTADSAMN | MAKEMSEMTKFKILNE |
| serovar finitimus | SVSNILLRMRDIANQSANGTNT | AGISMLSQANQTPQMV |
| strain YBT-020 | DSNKSALQKEFAELQKQITYIAD | SKLLQ |
| [CDS of SEQ ID NO: | NTQFNDKNLLKEDSEVKIQTLD | |
| 17] | S | |
| Flagellin | GVLNMRINTNINSMRTQEYMR | AAIDAALTKVADNRAT |
| N-SEQ ID NO: 110 | QNQAKMSNAMDRLSSGKRINN | LGATLNRLDFNVNNLK |
| C-SEQ ID NO: 111 | ASDDAAGLAIATRMRARESGLN | SQSSSMASAASQIEDAD |
| Bacillus thuringiensis | VAADNTQNGMSLIRTADSAMN | MAKEMSEMTKFKILNE |
| serovar finitimus | SVSNILLRMRDIANQSANGTNT | AGISMLSQANQTPQMV |
| strain YBT-020 | DSNKSALQKEFAELQKQITYIAD | SKLLQ |
| [CDS of SEQ ID NO: | NTQFNDKNLLKEDSEVKIQTLD | |
| 18] | S | |
| Flagellin | GFLNMRINTNINSMRTQEYMRQ | TVADNRATLGATLNRL |
| N-SEQ ID NO: 112 | NQAKMSNAMDRLSSGKRINNA | DFNVNNLKSQSASMAS |
| C-SEQ ID NO: 113 | SDDAAGLAIATRMRARESGLGV | AASQIEDADMAKEMSE |
| Bacillus cereus | AANNTQDGMSLIRTADSALNSV | MTKFKILNEAGISMLSQ |
| stain B4264 | SNILLRMRDLANQSANGTNTAE | ANQTPQMVSKLLQ |
| [CDS of SEQ ID NO: | NKAAMQKEFGELKDQIKYISEN | |
| 19] | TQFNDQHLL | |
| Flagellin | GFLNMRINTNINSMRTQEYMRQ | AIKSIDAALDTIASNRA |
| N-SEQ ID NO: 114 | NQAKMSNAMDRLSSGKRINNA | TLGATLNRLDFNVNNL |
| C-SEQ ID NO: 115 | SDDAAGLAIATRMRARESGLGV | KSQSSSMASAASQIEDA |
| Bacillus thuringiensis | AANNTQDGMSLIRTADSALNSV | DMAKEMSEMTKFKILN |
| serovar nigeriensis | SNILLRMRDIANQSANGTNTSD | EAGISMLSQANQTPQM |
| [CDS of SEQ ID NO: | NQKALDKEFSALKEQIDYISKNT | VSKLLQ |
| 20] | EFNDKKLL | |
| Flagellin | WGFLIMRINTNINSMRTQEYMR | AVDAIDAALKTVASNR |
| N-SEQ ID NO: 116 | QNQAKMSNAMDRLSSGKRINN | ATLGATLNRLDFNVNN |
| C-SEQ ID NO: 117 | ASDDAAGLAIATRMRARESGLG | LKSQSASMASAASQIED |
| Bacillus thuringiensis | VAANNTQDGMSLIRTADSALNS | ADMAKEMSEMTKFKIL |
| [CDS of SEQ ID NO: | VSNILLRMRDIANQSANGTNTA | NEAGISMLSQANQTPQ |
| 21] | DNQQALQKEFGQLKEQISYIAD | MVSKLLQ |
| | NTEFND | |
| Flagellin | WGFLIMRINTNINSMRTQEYMR | AVDAIDAALKTVASNR |
| N-SEQ ID NO: 118 | QNQAKMSNAMDRLSSGKRINN | ATLGATLNRLDFNVNN |
| C-SEQ ID NO: 119 | ASDDAAGLAIATRMRARESGLG | LKSQSASMASAASQIED |
| Bacillus thuringiensis | VAANNTQDGMSLIRTADSALNS | ADMAKEMSEMTKFKIL |
| serovar konkukian | VSNILLRMetRDIANQSANGTNT | NEAGISMLSQANQTPQ |
| strain 97-27 | ADNQQALQKEFGQLKEQISYIA | MVSKLLQ |
| [CDS of SEQ ID NO: | DNTEFNDKTLL | |
| 22] | | |
| Flagellin | WGFLIMRINTNINSMRTQEYMR | AIASIDAALESIASNRAT |
| N-SEQ ID NO: 120 | QNQAKMSNAMDRLSSGKRINN | LGATLNRLDFNVNNLK |
| C-SEQ ID NO: 121 | ASDDAAGLAIATRMRARESGLG | SQSSSMASAASQIEDAD |
| Bacillus thuringiensis | VAANNTQDGMSLIRTADSAMN | MAKEMSEMTKFKILNE |
| serovar konkukian | SVSNILLRMRDISNQSANGTNTD | AGISMLSQANQTPQMV |
| strain 97-27 | KNQSALDKEFAALKDQIDYISK | SKLLQ |
| [CDS of SEQ ID NO: | NTEFNDQKLL | |
| 23] | | |
| Flagellin protein FlaA | GFLNMRINTNINSMRTQEYMRQ | AIASIDAALESIASNRAT |
| N-SEQ ID NO: 122 | NQAKMSNAMDRLSSGKRINNA | LGATLNRLDFNVNNLK |
| C-SEQ ID NO: 123 | SDDAAGLAIATRMRARESGLGV | SQSSSMASAASQIEDAD |
| Bacillus thuringiensis | AANNTQDGMSLIRTADSAMNS | MAKEMSEMTKFKILNE |
| serovar thuringiensis | VSNILLRMRDISNQSANGTNTD | AGISMLSQANQTPQMV |
| strain IS5056 | KNQSALDKEFAALKDQIDYISK | SKLLQ |
| [CDS of SEQ ID NO: | NTEFNDQKLL | |
| 24] | | |
| Flagellin protein FlaA | GFLNMRINTNINSMRTQEYMRQ | AIASIDAALESIASNRAT |
| N-SEQ ID NO: 124 | NQAKMSNAMDRLSSGKRINNA | LGATLNRLDFNVNNLK |
| C-SEQ ID NO: 125 | SDDAAGLAIATRMRARESGLGV | SQSSSMASAASQIEDAD |
| Bacillus thuringiensis | AANNTQDGMSLIRTADSAMNS | MAKEMSEMTKFKILNE |
| serovar thuringiensis | VSNILLRMRDISNQSANGTNTD | AGISMLSQANQTPQMV |
| strain IS5056 | KNQSALDKEFAALKDQIDYISK | SKLLQ |
| [CDS of SEQ ID NO: | NTEFNDQKLL | |
| 25] | | |
| Flagellin B | GFLNMRINTNINSMRTQEYMRQ | AIASIDAALESIASNRAT |
| N-SEQ ID NO: 126 | NQAKMSNAMDRLSSGKRINNA | LGATLNRLDFNVNNLK |
| C-SEQ ID NO: 127 | SDDAAGLAIATRMRARESGLGV | SQSSSMASAASQIEDAD |
| Bacillus thuringiensis | AANNTQDGMSLIRTADSAMNS | MAKEMSEMTKFKILNE |
| strain Bt407 | VSNILLRMRDISNQSANGTNTD | AGISMLSQANQTPQMV |
| [CDS of SEQ ID NO: | KNQSALDKEFAALKDQIDYISK | SKLLQ |
| 26] | NTEFNDQKLL | |
| Flagellin | GFLNMRINTNINSMRTQEYMRQ | AIASIDAALESIASNRAT |
| N-SEQ ID NO: 128 | NQAKMSNAMDRLSSGKRINNA | LGATLNRLDFNVNNLK |
| C-SEQ ID NO: 129 | SDDAAGLAIATRMRARESGLGV | SQSSSMASAASQIEDAD |
| Bacillus thuringiensis | AANNTQDGMSLIRTADSAMNS | MAKEMSEMTKFKILNE |
| serovar chinensis CT-43 | VSNILLRMRDISNQSANGTNTD | AGISMLSQANQTPQMV |
| [CDS of SEQ ID NO: | KNQSALDKEFAALKDQIDYISK | SKLLQ |
| 27] | NTEFNDQKLL | |
| Flagellin | GFLNMRINTNINSMRTQEYMRQ | RATLGATLNRLDFNVN |
| N-SEQ ID NO: 130 | NQAKMSNAMDRLSSGKRINNA | NLKSQSSSMASAASQIE |
| C-SEQ ID NO: 131 | SDDAAGLAIATRMRARESGLGV | DADMAKEMSEMTKFKI |
| Bacillus thuringiensis | AANNTQDGISLIRTADSAMNSV | LNEAGISMLSQANQTP |
| serovar Canadensis | SNILLRMRDLANQSANGTNTNE | QMVSKLLQ |
| [CDS of SEQ ID NO: | NQAALNKEFDALKEQIDYISTNT | |
| 28] | EFNDKKLL | |
| Flagellin | GFLNMRINTNINSMRTQEYMRQ | RATLGATLNRLDFNVN |
| N-SEQ ID NO: 132 | NQAKMSNAMDRLSSGKRINNA | NLKSQSSSMASAASQIE |
| C-SEQ ID NO: 133 | SDDAAGLAIATRMRARESGLGV | DADMAKEMSEMTKFKI |
| Bacillus thuringiensis | AANNTQDGISLIRTADSAMNSV | LNEAGISMLSQANQTP |
| serovar galleriae | SNILLRMRDLANQSANGTNTNE | QMVSKLLQ |
| [CDS of SEQ ID NO: | NQAALNKEFDALKEQIDYISTNT | |
| 29] | EFNDKKLL | |
| Flagellin N-terminal | GVLNMRINTNINSMRTQEYMR | RATLGATLNRLDFNVN |
| helical region | QNQAKMSNAMDRLSSGKRINN | NLKSQSSSMASAASQIE |
| N-SEQ ID NO: 134 | ASDDAAGLAIATRMRARESGLS | DADMAKEMSEMTKFKI |
| C-SEQ ID NO: 135 | VAANNTQDGMSLIRTADSAMN | LNEAGISMLSQANQTP |
| Bacillus | SVSNILLRMRDLSNQSANGTNT | QMVSKLLQ |
| weihenstephanensis | DENQQALNKEFAALKDQIDYIS | |
| [CDS of SEQ ID NO: | KNTEFNDKKLL | |
| 30] | | |
| Flagellin | GFLNMRINTNINSMRTQEYMRQ | IDAALETIASNRATLGA |
| N-SEQ ID NO: 136 | NQAKMSNAMDRLSSGKRINNA | TLNRLDFNVNNLKSQS |
| C-SEQ ID NO: 137 | SDDAAGLAIATRMRARESGLGV | SSMASAASQIEDADMA |
| Bacillus thuringiensis | AANNTQDGMSLIRTADSALNSV | KEMSEMTKFKILNEAGI |
| serovar ostriniae | SNILLRMRDIANQSANGTNTGD | SMLSQANQTPQMVSKL |
| [CDS of SEQ ID NO: | NQKALDKEFSALKEQIDYISKNT | LQS |
| 31] | EFNDKKLL | |
| Flagellin | WGFLIMRINTNINSMRTQEYMR | LGATLNRLDFNVNNLK |
| N-SEQ ID NO: 138 | QNQTKMSNAMDRLSSGKRINN | SQSSSMAAAASQIEDA |
| C-SEQ ID NO: 139 | ASDDAAGLAIATRMRARENGL | DMAKEMSEMTKFKILN |
| Bacillus thuringiensis | GVAANNTQDGMSLIRTADSAM | EAGISMLSQANQTPQM |
| [CDS of SEQ ID NO: | NSVSNILLRMRDLANQSANGTN | VSKLLQ |
| 32] | TDDNQKALDKEFSALKEQIDYIS | |
| | KNTEFNDKKLL | |
| Flagellin | WGFLIMRINTNINSMRTQEYMR | LGATLNRLDFNVNNLK |
| N-SEQ ID NO: 140 | QNQTKMSNAMDRLSSGKRINN | SQSSSMAAAASQIEDA |
| C-SEQ ID NO: 141 | ASDDAAGLAIATRMRARENGL | DMAKEMSEMTKFKILN |
| Bacillus thuringiensis | GVAANNTQDGMSLIRTADSAM | EAGISMLSQANQTPQM |
| [CDS of SEQ ID NO: | NSVSNILLRMRDLANQSANGTN | VSKLLQ |
| 33] | TDDNQKALDKEFSALKEQIDYIS | |
| | KNTEFNDKKLL | |
| Flagellin | WGFLIMRINTNINSMRTQEYMR | RATLGATLNRLDFNVN |
| N-SEQ ID NO: 142 | QNQTKMSNAMDRLSSGKRINN | NLKSQSSSMAAAASQIE |
| C-SEQ ID NO: 143 | ASDDAAGLAIATRMRARENGL | DADMAKEMSEMTKFKI |
| Bacillus thuringiensis | GVAANNTQDGMSLIRTADSAM | LNEAGISMLSQANQTP |
| serovar pondicheriensis | NSVSNILLRMRDLANQSANGTN | QMVSKLLQ |
| [CDS of SEQ ID NO: | TDDNQKALDKEFSALKEQIDYIS | |
| 34] | KNTEFNDKKLL | |
| Flagellin B | GFLNMRINTNINSMRTQDYMRQ | AIASIDAALESIASNRAT |
| N-SEQ ID NO: 144 | NQAKMSNAMDRLSSGKRINNA | LGATLNRLDFNVNNLK |
| C-SEQ ID NO: 145 | SDDAAGLAIATRMRARESGLGV | SQSSSMASAASQIEDAD |
| Bacillus thuringiensis | AANNTQDGMSLIRTADSAMNS | MAKEMSEMTKFKILNE |
| serovar Berliner | VSNILLRMRDISNQSANGTNTD | AGISMLSQANQTPQMV |
| [CDS of SEQ ID NO: | KNQSALDKEFAALKDQIDYISK | SKLLQ |
| 35] | NTEFNDQKLL | |
| Flagellin A | GFLNMARITINLEIDFFAYYRFSI | AIASIDAALESIASNRAT |
| N-SEQ ID NO: 146 | CRKVNIKKWGFLNMRINTNINS | LGATLNRLDFNVNNLK |
| C-SEQ ID NO: 147 | MRTQDYMRQNQAKMSNAMDR | SQSSSMASAASQIEDAD |
| Bacillus thuringiensis | LSSGKRINNASDDAAGLAIATR | MAKEMSEMTKFKILNE |
| serovar Berliner | MRARESGLGVAANNTQDGMSL | AGISMLSQANQTPQMV |
| [CDS of SEQ ID NO: | IRTADSAMNSVSNILLRMRDISN | SKLLQ |
| 36] | QSANGTNTDKNQSALDKEFAAL | |
| | KDQIDYISKNTEFNDQKLL | |
| Flagellin | GVLYMRINTNINSMRTQEYMR | TVADNRATLGATLNRL |
| N-SEQ ID NO: 148 | QNQAKMSNAMDRLSSGKRINN | DFNVNNLKSQSSAMAA |
| C-SEQ ID NO: 149 | ASDDAAGLAIATRMRARE | SGLSSASQIEDADMAKEMSE |
| Bacillus cereus strain Q1 | VAADNTQNGMSLIRTADSAMN | MTKFKILNEAGISMLSQ |
| [CDS of SEQ ID NO: | SVSNILLRMRDIANQSANGTNT | ANQTPQMVSKLLQ |
| 37] | DKNQVALQKEFAALKEQITYIA | |
| | DNTQFNDKNLLNGNQTINIQTL | |
| | DSHDST | |
| Flagellin | GVLYMRINTNINSMRTQEYMR | TVADNRATLGATLNRL |
| N-SEQ ID NO: 150 | QNQAKMSNAMDRLSSGKRINN | DFNVNNLKSQSSAMAA |
| C-SEQ ID NO: 151 | ASDDAAGLAIATRMRARE | SGLSSASQIEDADMAKEMSE |
| Bacillus cereus strain Q1 | VAADNTQNGMSLIRTADSAMN | MTKFKILNEAGISMLSQ |
| [CDS of SEQ ID NO: | SVSNILLRMRDIANQSANGTNT | ANQTPQMVSKLLQ |
| 38] | DKNQVALQKEFAALKEQITYIA | |
| | DNTQFNDKNLLNGNQTINIQTL | |
| | DSHDST | |
| Flagellin | GFLNMRINTNINSMRTQEYMRQ | LGATLNRLDFNVNNLK |
| N-SEQ ID NO: 152 | NQAKMSNAMDRLSSGKRINNA | SQSSSMASAASQIEDAD |
| C-SEQ ID NO: 153 | SDDAAGLAIATRMRARESGLGV | MAKEMSEMTKFKILNE |
| Bacillus thuringiensis | AANNTQDGMSLIRTADSALNSV | AGISMLSQANQTPQMV |
| serovar morrisoni | SNILLRMRDIANQSANGTNTGD | SKLLQ |
| [CDS of SEQ ID NO: | NQKALDKEFSALKEQIDYISKNT | |
| 39] | EFNDKKLL | |
| Flagellin | GFLNMRINTNINSMRTQEYMRQ | AIKSIDAALDTIASNRA |
| N-SEQ ID NO: 154 | NQTKMSNAMDRLSSGKRINNAS | TLGATLNRLDFNVNNL |
| C-SEQ ID NO: 155 | DDAAGLAIATRMRARENGLGV | KSQSSSMASAASQIEDA |
| Bacillus thuringiensis | AANNTQDGMSLIRTADSALNSV | DMAKEMSEMTKFKILN |
| serovar neoleonensis | SNILLRMRDIANQSANGTNT | SDEAGISMLSQANQTPQM |
| [CDS of SEQ ID NO: | NQKALDKEFSALKEQIDYISKNT | VSKLLQ |
| 40] | EFNDKKLL | |
| Flagellin | GFLNMRINTNINSMRTQEYMRQ | RATLGATLNRLDFNVN |
| N-SEQ ID NO: 156 | NQAKMSNAMDRLSSGKRINNA | NLKSQSSSMASAASQIE |
| C-SEQ ID NO: 157 | SDDAAGLAIATRMRARESGLGV | DADMAKEMSEMTKFKI |
| Bacillus thuringiensis | AANNTQDGMSLIRTADSALNSV | LNEAGISMLSQANQTP |
| serovar morrisoni | SNILLRMRDIANQSANGTNTGD | QMVSKLLQ |
| [CDS of SEQ ID NO: | NQKALDKEFSALKEQIDYISKNT | |
| 41] | EFNDKKLL | |
| Flagellin | GFLNMRINTNINSMRTQEYMRQ | RATLGATLNRLDFNVN |
| N-SEQ ID NO: 158 | NQAKMSNAMDRLSSGKRINNA | NLKSQSSSMASAASQIE |
| C-SEQ ID NO: 159 | SDDAAGLAIATRMRARESGLGV | DADMAKEMSEMTKFKI |
| Bacillus thuringiensis | AANNTQDGMSLIRTADSALNSV | LNEAGISMLSQANQTP |
| serovar morrisoni | SNILLRMRDIANQSANGTNTGD | QMVSKLLQ |
| [CDS of SEQ ID NO: | NQKALDKEFSALKEQIDYISKNT | |
| 42] | EFNDKKLL | |
| Flagellin | GFLNMRINTNINSMRTQEYMRQ | LGATLNRLDFNVNNLK |
| N-SEQ ID NO: 160 | NQAKMSNAMDRLSSGKRINNA | SQQSSMASAASQIEDA |
| C-SEQ ID NO: 161 | SDDAAGLAIATRMRARESGLGV | DMAKEMSEMTKFKILN |
| Bacillus thuringiensis | AANNTQDGMSLIRTADSAMNS | EAGISMLSQANQTPQM |
| serovar jegathesan | VSNILLRMRDIANQSANGTNTN | VSKLLQ |
| [CDS of SEQ ID NO: | GNQAALNKEFDALKQQINYIST | |
| 43] | NTEFNDKKLLDGSNKTIAIQTLD | |
| Flagellin | GVLNMRINTNINSMRTQEYMR | DKIDEALKTIADNRATL |
| N-SEQ ID NO: 162 | QNQAKMSNAMDRLSSGKRINN | GATLNRLDFNVNNLKS |
| C-SEQ ID NO: 163 | ASDDAAGLAIATRMRARESGLG | QSASMASAASQIEDAD |
| Bacillus cereus stain | VAANNTQDGMALIRTADSAMN | MAKEMSEMTKFKILNE |
| ATCC 10987 | SVSNILLRRDIANQSANGTNTDK | AGISMLSQANQTPQMV |
| [CDS of SEQ ID NO: | NQAALQKEFGELQKQIDYIAGN | SKLLQ |
| 44] | TQFNDK | |
| Flagellin | WGFLIMRINTNINSMRTQEYMR | RATLGATLNRLDFNVN |
| N-SEQ ID NO: 164 | QNQAKMSNAMDRLSSGKRINN | NLKSQQSSMASAASQIE |
| C-SEQ ID NO: 165 | ASDDAAGLAIATRMRARESGLG | DADMAKEMSEMTKFKI |
| Bacillus thuringiensis | VAANNTQDGMSLIRTADSAMN | LNEAGISMLSQANQTP |
| serovar monterrey | SVSNILLRMRDLANQSANGTNT | QMVSKLLQ |
| [CDS of SEQ ID NO: | NENQAALNKEFDALKEQINYIST | |
| 45] | NTEFNDKKLL | |
| Flagellin | WGFFYMRINTNINSMRTQEYM | TVADNRATLGATLNRL |
| N-SEQ ID NO: 166 | RQNQAKMSNAMDRLSSGKRIN | DFNVNNLKSQASSMAA |
| C-SEQ ID NO: 167 | NASDDAAGLAIATRMRARESGL | AASQVEDADMAKEMS |
| Bacillus cereus strain | GVASNNTQDGMSLIRTADSALN | EMTKFKILNEAGISMLS |
| NC 7401 | SVSNILLRMRDLANQSANGTNT | QANQTPQMVSKLLQ |
| [CDS of SEQ ID NO: | NENKAAMQKEFGELKEQIKYIA | |
| 46] | ENTQFNDQHLL | |
| Flagellin | WGFFYMRINTNINSMRTQEYM | TVADNRATLGATLNRL |
| N-SEQ ID NO: 168 | RQNQAKMSNAMDRLSSGKRIN | DFNVNNLKSQASSMAA |
| C-SEQ ID NO: 169 | NASDDAAGLAIATRMRARESGL | AASQVEDADMAKEMS |
| Bacillus cereus strain | GVASNNTQDGMSLIRTADSALN | EMTKFKILNEAGISMLS |
| NC 7401 | SVSNILLRMRDLANQSANGTNT | QANQTPQMVSKLLQ |
| [CDS of SEQ ID NO: | NENKAAMQKEFGELKEQIKYIA | |
| 47] | ENTQFNDQHLL | |
| Flagellin (A-type) | GVLNMRINTNINSLRTQEYMRQ | IDAALKTVADNRATLG |
| N-SEQ ID NO: 170 | NQAKMSNSMDRLSSGKRINNAS | ATLNRLDFNVNNLKSQ |
| C-SEQ ID NO: 171 | DDAAGLAIATRMRARESGLNV | SSSMASAASQIEDADM |
| Bacillus cereus strain | AANNTQDGMSLIRTADSALGSV | AKEMSEMTKFKILNEA |
| AH820 | SNILLRMRDLANQSANGTNTSD | GISMLSQANQTPQMVS |
| [CDS of SEQ ID NO: | NQAAMQKEFAELQKQITYIADN | KLLQ |
| 48] | TQFNDKNLL | |
| Flagellin | WGFFYMRINTNINSMRTQEYM | TVADNRATLGATLNRL |
| N-SEQ ID NO: 172 | RQNQAKMSNAMDRLSSGKRIN | DFNVNNLKSQASSMAA |
| C-SEQ ID NO: 173 | NASDDAAGLAIATRMRARESGL | AASQVEDADMAKEMS |
| Bacillus cereus AH187 | GVASNNTQDGMSLIRTADSALN | EMTKFKILNEAGISMLS |
| [CDS of SEQ ID NO: | SVSNILLRMRDLANQSANGTNT | QANQTPQMVSKLLQ |
| 49] | NENKAAMQKEFGELKEQIKYIA | |
| | ENTQFNDQHLL | |
| Flagellin | WGFFYMRINTNINSMRTQEYM | TVADNRATLGATLNRL |
| N-SEQ ID NO: 174 | RQNQAKMSNAMDRLSSGKRIN | DFNVNNLKSQASSAAA |
| C-SEQ ID NO: 175 | NASDDAAGLAIATRMRARESGL | ASQVEDADMAKEMSE |
| Bacillus cereus | GVASNNTQDGMSLIRTADSALN | MTKFKILNEAGISMLSQ |
| [CDS of SEQ ID NO: | SVSNILLRMRDLANQSANGTNT | ANQTPQMVSKLLQ |
| 50] | NENKAAMQKEFGELKEQIKYIA | |
| | ENTQFNDQHLL | |
| Flagellin protein Fla | GFLNMRINTNINSMRTQEYMRQ | LGATLNRLDFNVNNLK |
| N-SEQ ID NO: 176 | NQAKMSNAMDRLSSGKRINNA | SQSSSMASAASQIEDAD |
| C-SEQ ID NO: 177 | SDDAAGLAIATRMRARESGLGV | MAKEMSEMTKFKILNE |
| Bacillus cereus | AANNTQDGMSLIRTADSALNSV | AGISMLSQANQTPQMV |
| [CDS of SEQ ID NO: | SNILLRMRDIANQSANGTNTGD | SKLLQ |
| 51] | NQKALDKEFSALKEQIDYISKNT | |
| | EFNDKKLL | |
| Flagellin | GFLNMRINTNINSMRTQEYMRQ | RATLGATLNRLDFNVT |
| N-SEQ ID NO: 178 | NQTKMSNAMDRLSSGKRINNAS | NLKSQENSMAASASQIE |
| C-SEQ ID NO: 179 | DDAAGLAIATRMRSREGGLNV | DADMAKEMSEMTKFKI |
| Bacillus thuringiensis | AARNTEDGMSLIRTADSALNSV | LNEAGISMLSQANQTP |
| Strain HD-771 | SNILLRMRDLANQSASETNTSK | QMVSKLLQ |
| [CDS of SEQ ID NO: | NQAAMQKEFDQLKEQIQYI | |
| 52] | | |
| Flagellin | GFLNMRINTNINSMRTQEYMRQ | RATLGATLNRLDFNVT |
| N-SEQ ID NO: 180 | NQTKMSNAMDRLSSGKRINNAS | NLKSQENSMAASASQIE |
| C-SEQ ID NO: 181 | DDAAGLAIATRMRSREGGLNV | DADMAKEMSEMTKFKI |
| Bacillus thuringiensis | AARNTEDGMSLIRTADSALNSV | LNEAGISMLSQANQTP |
| serovar sotto | SNILLRMRDLANQSASETNTSK | QMVSKLLQ |
| [CDS of SEQ ID NO: | NQAAMQKEFDQLKEQIQYI | |
| 53] | | |
| Flagellin | MGVLNMRINTNINSMRTQEYM | AIKAIDEALETIASNRAT |
| N-SEQ ID NO: 182 | RQNQAKMSTAMDRLSSGKRIN | LGATLNRLDFNVNNLK |
| C-SEQ ID NO: 183 | NASDDAAGLAIATRMRARESGL | NQASSMASAASQVEDA |
| Bacillus thuringiensis | GVAANNTQDGISLIRTADSAMN | DMAKEMSEMTKFKILN |
| serovar Novosibirsk | SVSNILLRMRDLANQSANGTNT | EAGISMLSQANQTPQM |
| [CSD of SEQ ID NO: | DKNQGALDKEFAALKEQIDYIS | VSKLLQ |
| 54] | KNTEFNDKKLL | |
| Flagellin | MGVLNMRINTNINSMRTQEYM | AIDSALENIASNRATLG |
| N-SEQ ID NO: 184 | RQNQAKMSNAMDRLSSGKRIN | ATLNRLDFNVNNLKSQ |
| C-SEQ ID NO: 185 | NASDDAAGLAIATRMRARESGL | SSSMASAASQIEDADM |
| Bacillus thuringiensis | GVAANNTQDGISLIRTADSAMN | AKEMSEMTKFKILNEA |
| serovar Londrina | SVSNILLRMRDLANQSANGTNT | GISMLSQANQTPQMVS |
| [CDS of SEQ ID NO: | SENQAALDKEFGALKEQINYIST | KLLQ |
| 55] | NTEFNDKKLL | |
| Flagellin | MGVLNMRINTNINSMRTQEYM | LGATLNRLDFNVNNLK |
| N-SEQ ID NO: 186 | RQNQAKMSTAMDRLSSGKRIN | NQASSMASAASQVEDA |
| C-SEQ ID NO: 187 | NASDDAAGLAIATRMRARESGL | DMAKEMSEMTKFKILN |
| Bacillus cereus strain | GVAANNTQDGISLIRTADSAMN | EAGISMLSQANQTPQM |
| E33L | SVSNILLRMRDLANQSANGTNT | VSKLLQ |
| [CDS of SEQ ID NO: | DKNQGALDKEFAALKEQIDYIS | |
| 56] | KNTEFNDKKLL | |
| Flagellin | MGVLNMRINTNINSMRTQEYM | ATLNRLDFNVNNLKNQ |
| N-SEQ ID NO: 188 | RQNQAKMSTAMDRLSSGKRIN | ASSMASAASQVEDAD |
| C-SEQ ID NO: 189 | NASDDAAGLAIATRMRARESGL | MAKEMSEMTKFKILNE |
| Bacillus cereus strain | GVAANNTQDGISLIRTADSAMN | AGISMLSQANQTPQMV |
| E33L | SVSNILLRMRDLANQSANGTNT | SKLLQ |
| [CDS of SEQ ID NO: | DKNQGALDKEFAALKEQIDYIS | |
| 57] | KNTEFNDKKLL | |
| Flagellin | WGFFYMRINTNINSMRTQEYM | AIAAIDAALTKVADNR |
| N-SEQ ID NO: 190 | RQNQAKMSTAMDRLSSGKRIN | ATLGATLNRLDFNVNN |
| C-SEQ ID NO: 191 | NASDDAAGLAIATRMRARESGL | LKSQASSMASAASQVE |
| Bacillus cereus | GVAANNTQDGISLIRTADSAMN | DADMAKEMSEMTKFKI |
| strain FRI-35 | SVSNILLRMRDLANQSANGTNT | LNEAGISMLSQANQTP |
| [CDS of SEQ ID NO: | DKNQAALDKEFNALKEQIDYIS | QMVSKLLQ |
| 58] | KNTEFNDKKL | |
| Flagellin | WGFFYMRIGTNVLSLNARQSLY | AIRKIEEALQNVSLHRA |
| N-SEQ ID NO: 192 | ENEKRMNVAMEHLATGKKLNN | DLGAMINRLQFNIENLN |
| C-SEQ ID NO: 193 | ASDNPANIAIVTRMHARASGMR | SQSTALTDAASRIEDAD |
| Bacillus cereus | VAIRNNEDAISMLRTAEAALQT | MAQEMSDFLKFKLLTE |
| strain FRT-35 | VTNVLQRMRDLAVQSANGTNS | VALSMVSQANQVPQM |
| [CDS of SEQ ID NO: | NKNRDSLNKEFQSLTEQIGYIDE | VSKLLQ |
| 59] | TTEFNN | |
| Flagellin | LVPFAVWLAMSRIRRRILDTDC | MAASASQIEDADMAKE |
| N-SEQ ID NO: 194 | KAESAVRIKEIPSDVLRAATERP | MSEMTKFKILSEAGISM |
| C-SEQ ID NO: 195 | LSCARIRVAIARPAASSEALLIRL | LSQANQTPQMVSKLLQ |
| Bacillus thuringiensis | PLDKRSIALLILAWFWRMYSCV | |
| [CDS of SEQ ID NO: | RMLLMFVLILMLRTP | |
| 60] | | |
| Flagellin | AVWLAMSRIRRRILDTDCKAES | SMAASASQIEDADMAK |
| N-SEQ ID NO: 196 | AVRIKEIPSDVLRAATERPLSCA | EMSEMTKFKILSEAGIS |
| C-SEQ ID NO: 197 | RIRVAIARPAASSEALLIRLPLDK | MLSQANQTPQMVSKLL |
| Bacillus cereus strain | RSIALLILAWFWRMYSCVRMLL | Q |
| ATCC 4342 | MFVLILMLRTP | |
| [CDS of SEQ ID NO: | | |
| 61] | | |
| Flagellin | GFLNMRIGTNFLSMNARQSLYE | LGAMINRLHFNIENLNS |
| N-SEQ ID NO: 198 | NEKRMNVAMEHLATGKKLNH | QSMALTDAASRIEDAD |
| C-SEQ ID NO: 199 | ASDNPANIAIVTRMHARANGMR | MAQEMSDFLKFKLLTE |
| Bacillus thuringiensis | VAIRNNEDAISMLRTAEAALQT | VALSMVSQANQIPQMV |
| [CDS of SEQ ID NO: | VMNILQRMRDLAIQSANSTNSN | SKLLQ |
| 62] | KNRDSLNKEFQSLTEQISYI | |
| Flagellin | GFLNMRINTNINSMRTQEYMRQ | LGATLNRLDFNVNNLK |
| N-SEQ ID NO: 200 | NQAKMSNAMDRLSSGKRINNA | SQSSSMASAASQIEDAD |
| C-SEQ ID NO: 201 | SDDAAGLAIATRMRARESGLGV | MAKEMSEMTKFKILNE |
| Bacillus thuringiensis | AANNTQDGMSLIRTADSALNSV | AGISMLSQANQTPQMV |
| [CDS of SEQ ID NO: | SNILLRMRDIANQSANGTNTGD | SKLLQ |
| 63] | NQKALDKEFSALKEQIDYI | |
| Flagellin | MRINHNITALNTYRQFNNANNA | IDGAINQVSEQRSGLGA |
| N-SEQ ID NO: 202 | QAKSMEKLSSGQRINSASDDAA | TQNRLDHTINNLSTSSE |
| C-SEQ ID NO: 203 | GLAISEKMRGQIRGLDQASRNA | NLTASESRIRDVDYALA |
| Bacillus aryabhattai | QDGVSLIQTAEGALNETHDILQR | A |
| [CDS of SEQ ID NO: | MRELVVQAGNGTNKTEDLDAI | |
| 64] | QDEIGSLIEEIGGETDSKGISDRA | |
| | QFNGRNLLDGSLDITLQVGA | |
| Flagellin | MRINTNINSMRTQEYMRQNQD | IDQAIQDIADNRATYGS |
| N-SEQ ID NO: 204 | KMNTSMNRLSSGKQINSASDDA | QLNRLDHNLNNVNSQA |
| C-SEQ ID NO: 205 | AGLAIATRMRAKEGGLNVGAK | TNMAAAASQIEDADM |
| Bacillus manliponensis | NTQDGMSALRTMDSALNSVSNI | AKEMSEMTKFKILSEA |
| [CDS of SEQ ID NO: | LLRMRDLATQSATGTNQGNDR | GVSMLSQANQTPQMVS |
| 65] | ESLDLEFQQLTEEITHIAEKTNFN | KLLQ |
| | GNALLSGSGSAINVQLS | |
| Flagellin | MRIGSWTATGMSIVNUMNRNW | LDEATKNVSMERSRLG |
| N-SEQ ID NO: 206 | NAASKSMLRLSSGYRINSAADD | AYQNRLEHAYNVAENT |
| C-SEQ ID NO: 207 | AAGLAISEKMRGQIRGLTMASK | AINLQDAESRIRDVDIA |
| Lysinibacillus sp. strain | NIMDGVSLIQTAEGALNETHAIV | KEMMNMVKSQILAQV |
| BF-4 | QRMRELAVQAATDTNTDDDRA | GQQVLAMHMQQAQGI |
| [CDS of SEQ ID NO: | KLDLEFQELKKEIDRISTDTEFN | LRLLG |
| 66] | TRTLLNGDYKDNGLKIQVG | |
| Flagellin | MKIGSWTATGMSIVNHMNRNW | LDEATKNVSMERSRLG |
| N-SEQ ID NO: 208 | NAASKSMLRLSSGYRINSAADD | AYQNRLEHAYNVAENT |
| C-SEQ ID NO: 209 | AAGLAISEKMRGQIRGLTMASK | AINLQDAESRIRDVDIA |
| Lysinibacillus sp. strain | NIMDGVSLIQTAEGALNETHAIV | KEMMHMVKSQILAQV |
| 13S34 air | QRMRELAVQAATDTNTDDDRA | GQQVLAMHIQQAQGIL |
| [CDS of SEQ ID NO: | KLDLEFQELKKEIDRISTDTAFN | RLLG |
| 67] | TRTLLNGDYKDNGLKIQVG | |
| Flagellin | MIISHNLTALNTMNKLKQKDLA | ISAAIDKVSAERARMG |
| N-SEQ ID NO: 210 | VSKSLGKLSSGLRINGASDDAA | AYQNRLEHSRNNVVTY |
| C-SEQ ID NO: 211 | GLAISEKMRGQIRGLNQASRNIQ | AENLTAAESRIRDVDM |
| Paenibacillus sp. strain | DGISLIQVADGAMQEIHSMLQR | AKEMMELMKNQIFTQA |
| HW567 | MNELAVQASNGTYSGSDRLNIQ | GQAMLLQTNTQPQAIL |
| [CDS of SEQ ID NO: | SEVEQLIEEIDEIAGNTGFNGIKL | QLLK |
| 68] | LNGNNEKTEKTEK | |
| Flagellin | MRINTNINSMRTQEYMRQNQA | IDSALETIASNRATLGA |
| N-SEQ ID NO: 212 | KMSNAMDRLSSGKRINNASDD | TLNRLDFNVNNLKSQS |
| C-SEQ ID NO: 213 | AAGLAIATRMRARESGLGVAA | SAMASAASQIEDADMA |
| Bacillus anthracis | NNTQDGMSLIRTADSAMNSVSN | KEMSEMTKFKILNEAGI |
| [CDS of SEQ ID NO: | ILLRMRDLANQSANGTNTKENQ | SMLSQANQTPQMVSKL |
| 69] | DALDKEFGALKEQIDYISKNTEF | LQ |
| | NDKKLLNGDNKSIAIQTL | |
| Flagellin | MQKSQYKKMGVLKMRINTNIN | ALNTVAGNRATLGATL |
| N-SEQ ID NO: 214 | SMRTQEYMRQNQDKMNVSMN | NRLDRNVENLNNQATN |
| C-SEQ ID NO: 215 | RLSSGKRINSAADDAAGLAIATR | MASAASQIEDADMAKE |
| Bacillus anthracis | MRARQSGLEKASQNTQDGMSLI | MSEMTKFKILNEAGISM |
| [CDS of SEQ ID NO: | RTAESAMNSVSNILTRMRDIAV | LSQANQTPQMVSKLLQ |
| 70] | QSSNGTNTAENQSALQKEFAEL | |
| | QEQIDYIAKNTEFNDKNLLAGT | |
| | GAVTIGSTSISGAEISIETL | |
| Flagellin | MRINTNINSMRTQEYMRQNQD | ALNTVAGNRATLGATL |
| N-SEQ ID NO: 216 | KMNVSMNRLSSGKRINSAADD | NRLDRNVENLNNQATN |
| C-SEQ ID NO: 217 | AAGLAIATRMRARQSGLEKASQ | MASAASQIKDADKAKE |
| Bacillus anthracis | NTQDGMSLIRTAESAMNSVSNI | MSEMTKFKILNEAGISM |
| [CDS of SEQ ID NO: | LTRMRDIAVQSSNGTNTAENQS | LSQANQTPQMVSKLLQ |
| 71] | ALQKEFAELQEQIDYIAKNTEFN | |
| | DKNLLAGTGAVTIGSTSISGAEIS | |
| | IETL | |
| Flagellin | MRINTNINSMRTQEYMRQNQD | ALNTVAGNRATLGATL |
| N-SEQ ID NO: 218 | KMNVSMNRLSSGKRINSAADD | NRLDRNVENLNNQATN |
| C-SEQ ID NO: 219 | AAGLAIATRMRARQSGLEKASQ | MASAASQIEDADMAKE |
| Bacillus anthracis | NTQDGMSLIRTAESAMNSVSNI | MSEMTKFKILNEAGISM |
| [CDS of SEQ ID NO: | LTRMRDIAVQSSNGTNTAENQS | LSQANQTPQMV |
| 72] | ALQKEFAELQEQIDYIAKNTEFN | |
| | DKNLLAGTGAVTIGSTSISGAEIS | |
| | IETL | |
| Flagellin | MNVSMNRLSSGKRINSAADDA | LNTALNTVAGNRATLG |
| N-SEQ ID NO: 220 | AGLAIATRMRARQSGLEKASQN | ATLNRLDRNVENLNNQ |
| C-SEQ ID NO: 221 | TQDGMSLIRTAESAMNSVSNILT | ATNMASAASQIEDADM |
| Bacillus anthracis strain | RMRDIAVQSSNGTNTAENQSAL | AKEMSEMTKFKILNEA |
| H9401 | QKEFAELQEQIDYIAKNTEFNDK | GISMLSQANQTPQMVS |
| [CDS of SEQ ID NO: | NLLAGTGAVTIGSTSISGAEISIE | KLLQ |
| 73] | TL | |
| Flagellin | MRINHNITALNTYRQFNNANNA | IIDGAINQVSEQRSGLG |
| N-SEQ ID NO: 222 | QAKSMEKLSSGQRINSASDDAA | ATQNRLDHTINNLSTSS |
| C-SEQ ID NO: 223 | GLAISEKMRGQIRGLDQASRNA | ENLTASESRIRDVDYAL |
| Bacillus megaterium | QDGVSLIQTAEGALNETHDILQR | AA |
| strain WSH-002 | MRELVVQAGNGTNKTEDLDAI | |
| [CDS of SEQ ID NO: | QDEIGSLIEEIGGEADSKGISDRA | |
| 74] | QFNGRNLLDGSLDITLQVGA | |
| Flagellin | MRINHNLPALNAYRNLAQNQIG | FKAAIDQVSRIRSYFGAI |
| N-SEQ ID NO: 224 | TSKILERLSSGYRINRASDDAAG | QNRLEHVVNNLSNYTE |
| C-SEQ ID NO: 225 | LAISEKMRGQIRGLEQGQRNTM | NLTGAESRIRDADMAK |
| Aneurinibacillus sp. XH2 | DGVSLIQTAEGALQEIHEMLQR | EMTEFTRFNIINQSATA |
| [CDS of SEQ ID NO: | MRELAVQAANGTYSDKDKKAI | MLAQANQLPQGVLQLL |
| 75] | EDEINQLTAQIDQIAKTTEFNGIQ | KG |
| | LIGDSDSTSLQDVK | |

The amino acid sequence of the flagellin or flagellin-associated polypeptide used in the compositions and methods herein can comprise any one of SEQ ID NOs: 226-300, or any combination thereof.

The amino acid sequence of the flagellin or flagellin-associated polypeptide used in the compositions and methods herein can comprise SEQ ID NO: 226 or 571.

The amino acid sequence of the flagellin or flagellin-associated polypeptide used in the compositions and methods herein can comprise SEQ ID NO: 590.

The amino acid sequence of the flagellin or flagellin-associated polypeptide used in the compositions and methods herein can comprise any one of SEQ ID NOs: 301-375, and 587 or any combination thereof.

The amino acid sequence of the flagellin or flagellin-associated polypeptide used in the compositions and methods herein can comprise SEQ ID NO: 301.

The flagellin-derived polypeptide sequence for Bt4Q7Flg22 (SEQ ID NO: 226) was identified from a proprietary “in house” library from Bacillus thuringiensis (Bt.) strain 4Q7. Conserved primers to full length flagellin from E. coli were used to screen the Bt.4Q7 strain library and identify a functional flagellin-associated bioactive priming Flg22 polypeptide.

TABLE 3
Flagellin polypeptides Flg22 and FlgII-28 identified
from Bacillus spp.

| SEQ ID NO: | Peptide Flg22 |
|---|---|
| Flg22-Bt.4Q7 | DRLSSGKRINSASDDAAGLAIA |
| SEQ ID NO: 226 | |
| Bacillus thuringiensis | |
| strain 4Q7 | |
| Flg22 | DRLSSGKRINSASDDAAGLAIA |
| SEQ ID NO: 227 | |
| Bacillus thuringiensis, strain | |
| HD1002 | |
| Flg22 | DRLSSGKRINSASDDAAGLAIA |
| SEQ ID NO: 228 | |
| Bacillus thuringiensis, strain | |
| HD-789 | |
| Flg22 | DRLSSGKRINSASDDAAGLAIA |
| SEQ ID NO: 229 | |
| Bacillus cereus | |
| strain G9842 | |
| Flg22 | EHLATGKKLNNASDNPANIAIV |
| SEQ ID NO: 230 | |
| Bacillus thuringiensis serovar indiana | |
| strain HD521 | |
| Flg22 | DRLSSGKRINNASDDAAGLAIAT |
| SEQ ID NO: 231 | |
| Bacillus thuringiensis strain CTC | |
| Flg22 | DRLSSGKRINNASDDAAGLAIA |
| SEQ ID NO: 232 | |
| Bacillus thuringiensis serovar | |
| yunnanensis strain IEBC-T20001 | |
| Flg22 | DRLSSGKRINNASDDAAGLAIA |
| SEQ ID NO: 233 | |
| Bacillus thuringiensis serovar tolworthi | |
| Flg22 | DRLSSGKRINNASDDAAGLAIA |
| SEQ ID NO: 234 | |
| Bacillus cereus strain FM1 | |
| Flg22 | EHLATGKKLNHASDNPANVAIV |
| SEQ ID NO: 235 | |
| Bacillus cereus strain FM1 | |
| Flg22 | DRLSSGKRINNASDDAAGLAIA |
| SEQ ID NO: 236 | |
| Bacillus thuringiensis strain MC28 | |
| Flg22 | DRLSSGKRINNASDDAAGLAIA |
| SEQ ID NO: 237 | |
| Bacillus bombysepticus | |
| strain Wang | |
| Flg22 | DRLSSGKRINNASDDAAGLAIA |
| SEQ ID NO: 238 | |
| Bacillus thuringiensis serovar kenyae | |
| Flg22 | DRLSSGKRINNASDDAAGLAIA |
| SEQ ID NO: 239 | |
| Bacillus thuringiensis serovar kenyae | |
| Flg22 | DRLSSGKRINNASDDAAGLAIA |
| SEQ ID NO: 240 | |
| Bacillus cereus | |
| Flg22 | EHLATGKKLNNASDNPANIAIV |
| SEQ ID NO: 241 | |
| Bacillus cereus | |
| Flg22 | EHLATGKKLNHASDNPANVAIV |
| SEQ ID NO: 242 | |
| Bacillus thuringiensis serovar finitimus | |
| strain YBT-020 | |
| Flg22 | DRLSSGKRINNASDDAAGLAIA |
| SEQ ID NO: 243 | |
| Bacillus thuringiensis serovar finitimus | |
| strain YBT-020 | |
| Flg22 | DRLSSGKRINNASDDAAGLAIA |
| SEQ ID NO: 244 | |
| Bacillus cereus stain B4264 | |
| Flg22 | DRLSSGKRINNASDDAAGLAIA |
| SEQ ID NO: 245 | |
| Bacillus thuringiensis serovar nigeriensis | |
| Flg22 | DRLSSGKRINNASDDAAGLAIA |
| SEQ ID NO: 246 | |
| Bacillus thuringiensis | |
| Flg22 | EHFATGKKLNHASDNPANVAIV |
| SEQ ID NO: 247 | |
| Bacillus thuringiensis serovar konkukian | |
| strain 97-27 | |
| Flg22 | DRLSSGKRINNASDDAAGLAIA |
| SEQ ID NO: 248 | |
| Bacillus thuringiensis serovar konkukian | |
| strain 97-27 | |
| Flg22 | EHLATGKKLNHASDNPANIVIV |
| SEQ ID NO: 249 | |
| Bacillus thuringiensis serovar | |
| thuringiensis strain IS5056 | |
| Flg22 | DRLSSGKRINNASDDAAGLAIA |
| SEQ ID NO: 250 | |
| Bacillus thuringiensis serovar | |
| thuringiensis strain IS5056 | |
| Flg22 | DRLSSGKRINNASDDAAGLAIA |
| SEQ ID NO: 251 | |
| Bacillus thuringiensis | |
| strain Bt407 | |
| Flg22 | DRLSSGKRINNASDDAAGLAIA |
| SEQ ID NO: 252 | |
| Bacillus thuringiensis serovar chinensis | |
| CT-43 | |
| Flg22 | DRLSSGKRINNASDDAAGLAIA |
| SEQ ID NO: 253 | |
| Bacillus thuringiensis serovar canadensis | |
| Flg22 | DRLSSGKRINNASDDAAGLAIA |
| SEQ ID NO: 254 | |
| Bacillus thuringiensis serovar galleriae | |
| Flg22 | DRLSSGKRINNASDDAAGLAIA |
| SEQ ID NO: 255 | |
| Bacillus weihenstephanensis | |
| Flg22 | DRLSSGKRINNASDDAAGLAIA |
| SEQ ID NO: 256 | |
| Bacillus thuringiensis serovar ostriniae | |
| Flg22 | EHLATGKKLNHASDNPANVAIV |
| SEQ ID NO: 257 | |
| Bacillus thuringiensis | |
| Flg22 | DRLSSGKRINNASDDAAGLAIA |
| SEQ ID NO: 258 | |
| Bacillus thuringiensis | |
| Flg22 | DRLSSGKRINNASDDAAGLAIA |
| SEQ ID NO: 259 | |
| Bacillus thuringiensis serovar | |
| pondicheriensis | |
| Flg22 | EHLATGKKLNHASDNPANIVIV |
| SEQ ID NO: 260 | |
| Bacillus thuringiensis serovar Berliner | |
| Flg22 | DRLSSGKRINNASDDAAGLAIA |
| SEQ ID NO: 261 | |
| Bacillus thuringiensis serovar Berliner | |
| Flg22 | EHLATGKKLNHASNNPANVAIV |
| SEQ ID NO: 262 | |
| Bacillus cereus strain Q1 | |
| Flg22 | DRLSSGKRINNASDDAAGLAIA |
| SEQ ID NO: 263 | |
| Bacillus cereus strain Q1 | |
| Flg22 | EHLATGKKLNHASDNPANIAIV |
| SEQ ID NO: 264 | |
| Bacillus thuringiensis serovar morrisoni | |
| Flg22 | DRLSSGKRINNASDDAAGLAIA |
| SEQ ID NO: 265 | |
| Bacillus thuringiensis serovar neoleonensis | |
| Flg22 | DRLSSGKRINNASDDAAGLAIA |
| SEQ ID NO: 266 | |
| Bacillus thuringiensis serovar morrisoni | |
| Flg22 | DRLSSGKRINNASDDAAGLAIA |
| SEQ ID NO: 267 | |
| Bacillus thuringiensis serovar morrisoni | |
| Flg22 | DRLSSGKRINNASDDAAGLAIA |
| SEQ ID NO: 268 | |
| Bacillus thuringiensis serovar jegathesan | |
| Flg22 | DRLSSGKRINNASDDAAGLAIA |
| SEQ ID NO: 269 | |
| Bacillus cereus stain ATCC 10987 | |
| Flg22 from Flagellin A | DRLSSGKRINNASDDAAGLAIA |
| SEQ ID NO: 270 | |
| Bacillus thuringiensis serovar monterrey | |
| Flg22 | EHLATGKKLNNASDNPANIAIV |
| SEQ ID NO: 271 | |
| Bacillus cereus strain NC7401 | |
| Flg22 | DRLSSGKRINNASDDAAGLAIA |
| SEQ ID NO: 272 | |
| Bacillus cereus strain NC7401 | |
| Flg22 | DRLSSGKRINNASDDAAGLAIA |
| SEQ ID NO: 273 | |
| Bacillus cereus strain AH820 | |
| Flg22 | DRLSSGKRINNASDDAAGLAIA |
| SEQ ID NO: 274 | |
| Bacillus cereus AH187 | |
| Flg22 | DRLSSGKRINNASDDAAGLAIA |
| SEQ ID NO: 275 | |
| Bacillus cereus | |
| Flg22 | DRLSSGKRINNASDDAAGLAIA |
| SEQ ID NO: 276 | |
| Bacillus cereus | |
| Flg22 | DRLSSGKRINNASDDAAGLAIA |
| SEQ ID NO: 277 | |
| Bacillus thuringiensis | |
| Strain HD-771 [51] | |
| Flg22 | DRLSSGKRINNASDDAAGLAIA |
| SEQ ID NO: 278 | |
| Bacillus thuringiensis serovar sotto [52] | |
| Flg22 | DRLSSGKRINNASDDAAGLAIA |
| SEQ ID NO: 279 | |
| Bacillus thuringiensis serovar Novosibirsk | |
| Flg22 | DRLSSGKRINNASDDAAGLAIA |
| SEQ ID NO: 280 | |
| Bacillus thuringiensis serovar londrina | |
| Flg22 | EHLATGKKLNHASNNPANIAIV |
| SEQ ID NO: 281 | |
| Bacillus cereus strain E33L | |
| Flg22 | DRLSSGKRINNASDDAAGLAIA |
| SEQ ID NO: 282 | |
| Bacillus cereus strain E33L | |
| Flg22 | DRLSSGKRINNASDDAAGLAIA |
| SEQ ID NO: 283 | |
| Bacillus cereus | |
| strain FRI-35 | |
| Flg22 | EHLATGKKLNNASDNPANIAIV |
| SEQ ID NO: 284 | |
| Bacillus cereus strain FRI-35 | |
| Flg22 | DRLSSGKRINNASDDAAGLAIA |
| SEQ ID NO: 285 | |
| Bacillus thuringiensis | |
| Flg22 | DRLSSGKRINNASDDAAGLAIA |
| SEQ ID NO: 286 | |
| Bacillus cereus strain ATCC 4342 | |
| Flg22 | EHLATGKKLNHASDNPANIAIV |
| SEQ ID NO: 287 | |
| Bacillus thuringiensis | |
| Flg22 | DRLSSGKRINNASDDAAGLAIA |
| SEQ ID NO: 288 | |
| Bacillus thuringiensis | |
| Flg22 | EKLSSGQRINSASDDAAGLAIS |
| SEQ ID NO: 289 | |
| Bacillus aryabhattai | |
| Flg22 | NRLSSGKQINSASDDAAGLAIA |
| SEQ ID NO: 290 | |
| Bacillus manliponensis | |
| Flg22 | LRLSSGYRINSAADDAAGLAIS |
| SEQ ID NO: 291 | |
| Lysinibacillus sp. strain BF-4 | |
| Flg22 | LRLSSGYRINSAADDAAGLAIS |
| SEQ ID NO: 292 | |
| Lysinibacillus sp. strain 13S34_air | |
| Flg22 | GKLSSGLRINGASDDAAGLAIS |
| SEQ ID NO: 293 | |
| Paenibacillus sp. strain HWS67 | |
| Flg22 | DRLSSGKRINNASDDAAGLAIA |
| SEQ ID NO: 294 | |
| Bacillus anthracis | |
| Flg22 | NRLSSGKRINSAADDAAGLAIA |
| SEQ ID NO: 295 | |
| Bacillus anthracis | |
| Flg22 | NRLSSGKRINSAADDAAGLAIA |
| SEQ ID NO: 296 | |
| Bacillus anthracis | |
| Flg22 | NRLSSGKRINSAADDAAGLAIA |
| SEQ ID NO: 297 | |
| Bacillus anthracis | |
| Flg22 | NRLSSGKRINSAADDAAGLAIA |
| SEQ ID NO: 298 | |
| Bacillus anthracis strain H9401 | |
| Flg22 | EKLSSGQRINSASDDAAGLAIS |
| SEQ ID NO: 299 | |
| Bacillus megaterium strain WSH-002 | |
| Flg22 | ERLSSGYRINRASDDAAGLAIS |
| SEQ ID NO: 300 | |
| Aneurinibacillus sp. XH2 | |
| SEQ ID NO: | Peptide Flg15 |
| Flg15-Bt4Q7 | RINSAKDDAAGLAIA |
| SEQ ID NO: 590 | |
| Modified FLG15-Bt4Q7; Syn01 | |
| Bacillus thuringiensis strain 4Q7 | |
| SEQ ID NO: | Peptide FglI-28 |
| FlgII-28-Bt.4Q7 | SVSNILLRMRDLANQSANGTNTKGNQAS |
| SEQ ID NO: 301 | |
| Bacillus thuringiensis | |
| strain 4Q7 | |
| FlgII-28 | SVSNILLRMRDLANQSANGTNTKGNQAS |
| SEQ ID NO: 302 | |
| Bacillus thuringiensis, strain | |
| HD1002 | |
| FlgII-28 | SVSNILLRMRDLANQSANGTNTKGNQAS |
| SEQ ID NO: 303 | |
| Bacillus thuringiensis, strain | |
| HD-789 | |
| FlgII-28 | SVSNILLRMRDLANQSANGTNTKGNQAS |
| SEQ ID NO: 304 | |
| Bacillus cereus | |
| strain G9842 | |
| FlgII-28 | TVTNILQRMRDLAVQSANGTNSNKNRHS |
| SEQ ID NO: 305 | |
| Bacillus thuringiensis serovar indiana | |
| strain HD521 | |
| FlgII-28 | SVSNILLRMRDIANQSANITNTNENKSA |
| SEQ ID NO: 306 | |
| Bacillus thuringiensis strain CTC | |
| FlgII-28 | SVSNILLRMRDLANQSANGTNTDDNQKA |
| SEQ ID NO: 307 | |
| Bacillus thuringiensis | |
| serovar yunnanensis strain IEBC-T20001 | |
| FlgII-28 | SVSNILLRMRDLANQSANGTNTDENKAA |
| SEQ ID NO: 308 | |
| Bacillus thuringiensis serovar tolworthi | |
| FlgII-28 | SVSNILLRMRDIANQSANGTNTDKNQVA |
| SEQ ID NO: 309 | |
| Bacillus cereus strain FM1 | |
| FlgII-28 | TVTNILQRMRDVAVQSANGTNSNKNRDS |
| SEQ ID NO: 310 | |
| Bacillus cereus strain FM1 | |
| FlgII-28 | SVSNILLRMRDIANQSANGTNTADNQQA |
| SEQ ID NO: 311 | |
| Bacillus thuringiensis strain MC28 | |
| FlgII-28 | SVSNILLRMRDLANQSASGTNTDKNQAA |
| SEQ ID NO: 312 | |
| Bacillus bombysepticus strain Wang | |
| FlgII-28 | SVSNILLRMRDLANQSASGTNTDKNQAA |
| SEQ ID NO: 313 | |
| Bacillus thuringiensis serovar kenyae | |
| FlgII-28 | SVSNILLRMRDLANQSASGTNTDKNQAA |
| SEQ ID NO: 314 | |
| Bacillus thuringiensis serovar kenyae | |
| FlgII-28 | SVSNILLRMRDLANQSANGTNTGDNQKA |
| SEQ ID NO: 315 | |
| Bacillus cereus | |
| FlgII-28 | TNILQRMRDLAVQSANGTNSNKNRDSLN |
| SEQ ID NO: 316 | |
| Bacillus cereus | |
| FlgII-28 | TNVLQRMRDVAVQSANGTNLNKNRDSLN |
| SEQ ID NO: 317 | |
| Bacillus thuringiensis serovar finitimus | |
| strain YBT-020 | |
| FlgII-28 | SVSNILLRMRDIANQSANGTNTDSNKSA |
| SEQ ID NO: 318 | |
| Bacillus thuringiensis serovar finitimus | |
| strain YBT-020 | |
| FlgII-28 | SVSNILLRMRDLANQSANGTNTAENKAA |
| SEQ ID NO: 319 | |
| Bacillus cereus stain B4264 | |
| FlgII-28 | SVSNILLRMRDIANQSANGTNTSDNQKA |
| SEQ ID NO: 320 | |
| Bacillus thuringiensis serovar nigeriensis | |
| FlgII-28 | SVSNILLRMRDIANQSANGTNTADNQQA |
| SEQ ID NO: 321 | |
| Bacillus thuringiensis | |
| FlgII-28 | TVMNILQRMRDLAVQSANGTNSNKNRDS |
| SEQ ID NO: 322 | |
| Bacillus thuringiensis serovar konkukian | |
| strain 97-27 | |
| FlgII-28 | SVSNILLRMRDIANQSANGTNTADNQQA |
| SEQ ID NO: 323 | |
| Bacillus thuringiensis serovar konkukian | |
| strain 97-27 | |
| FlgII-28 | TVTNILQHMRDFAIQSANGTNSNTNRDS |
| SEQ ID NO: 324 | |
| Bacillus thuringiensis serovar thuringiensis | |
| strain IS5056 | |
| FlgII-28 | SVSNILLRMRDISNQSANGTNTDKNQSA |
| SEQ ID NO: 325 | |
| Bacillus thuringiensis serovar thuringiensis | |
| strain IS5056 | |
| FlgII-28 | SVSNILLRMRDISNQSANGTNTDKNQSA |
| SEQ ID NO: 326 | |
| Bacillus thuringiensis | |
| strain Bt407 | |
| FlgII-28 | SVSNILLRMRDISNQSANGTNTDKNQSA |
| SEQ ID NO: 327 | |
| Bacillus thuringiensis serovar chinensis | |
| CT-43 | |
| FlgII-28 | SVSNILLRMRDLANQSANGTNTNENQAA |
| SEQ ID NO: 328 | |
| Bacillus thuringiensis serovar canadensis | |
| FlgII-28 | SVSNILLRMRDLANQSANGTNTNENQAA |
| SEQ ID NO: 329 | |
| Bacillus thuringiensis serovar galleriae | |
| FlgII-28 | SVSNILLRMRDLSNQSANGTNTDENQQA |
| SEQ ID NO: 330 | |
| Bacillus weihenstephanensis | |
| FlgII-28 | SVSNILLRMRDIANQSANGTNTGDNQKA |
| SEQ ID NO: 331 | |
| Bacillus thuringiensis serovar ostriniae | |
| FlgII-28 | TVANILQRMRDLAVQSSNDTNSNKNRDS |
| SEQ ID NO: 332 | |
| Bacillus thuringiensis | |
| FlgII-28 | SVSNILLRMRDLANQSANGTNTDDNQKA |
| SEQ ID NO: 333 | |
| Bacillus thuringiensis | |
| FlgII-28 | SVSNILLRMRDLANQSANGTNTDDNQKA |
| SEQ ID NO: 334 | |
| Bacillus thuringiensis serovar | |
| pondicheriensis | |
| FlgII-28 | TVTNILQHMRDFAIQSANGTNSNTNRDS |
| SEQ ID NO: 335 | |
| Bacillus thuringiensis serovar Berliner | |
| FlgII-28 | SVSNILLRMRDISNQSANGTNTDKNQSA |
| SEQ ID NO: 336 | |
| Bacillus thuringiensis serovar Berliner | |
| FlgII-28 | TVTNVLQRMRDVAVQSANGTNSSKNRDS |
| SEQ ID NO: 337 | |
| Bacillus cereus strain Q1 | |
| FlgII-28 | SVSNILLRMRDIANQSANGTNTDKNQVA |
| SEQ ID NO: 338 | |
| Bacillus cereus strain Q1 | |
| FlgII-28 | TVMNILQRMRDLAIQSANSTNSNKNRDS |
| SEQ ID NO: 339 | |
| Bacillus thuringiensis serovar morrisoni | |
| FlgII-28 | SVSNILLRMRDIANQSANGTNTSDNQKA |
| SEQ ID NO: 340 | |
| Bacillus thuringiensis serovar neoleonensis | |
| FlgII-28 | SVSNILLRMRDIANQSANGTNTGDNQKA |
| SEQ ID NO: 341 | |
| Bacillus thuringiensis serovar morrisoni | |
| FlgII-28 | SVSNILLRMRDIANQSANGTNTGDNQKA |
| SEQ ID NO: 342 | |
| Bacillus thuringiensis serovar morrisoni | |
| FlgII-28 | SVSNILLRMRDIANQSANGTNTNGNQAA |
| SEQ ID NO: 343 | |
| Bacillus thuringiensis serovar jegathesan | |
| FlgII-28 | SVSNILLRMRDIANQSANGTNTDKNQAA |
| SEQ ID NO: 344 | |
| Bacillus cereus stain ATCC 10987 | |
| FlgII-28 from Flagellin A | SVSNILLRMRDLANQSANGTNTNENQAA |
| SEQ ID NO: 345 | |
| Bacillus thuringiensis serovar monterrey | |
| FlgII-28 | TVTNVLQRMRDLAVQSANDTNSNKNRDS |
| SEQ ID NO: 346 | |
| Bacillus cereus strain NC7401 | |
| FlgII-28 | SVSNILLRMRDLANQSANGTNTNENKAA |
| SEQ ID NO: 347 | |
| Bacillus cereus strain NC7401 | |
| FlgII-28 | SVSNILLRMRDLANQSANGTNTSDNQAA |
| SEQ ID NO: 348 | |
| Bacillus cereus strain AH820 | |
| FlgII-28 | SVSNILLRMRDLANQSANGTNTNENKAA |
| SEQ ID NO: 349 | |
| Bacillus cereus AH187 | |
| FlgII-28 | SVSNILLRMRDLANQSANGTNTNENKAA |
| SEQ ID NO: 350 | |
| Bacillus cereus | |
| FlgII-28 | SVSNILLRMRDIANQSANGTNTGDNQKA |
| SEQ ID NO: 351 | |
| Bacillus cereus | |
| FlgII-28 | SVSNILLRMRDLANQSASETNTSKNQAA |
| SEQ ID NO: 352 | |
| Bacillus thuringiensis | |
| Strain HD-771 [51] | |
| FlgII-28 | SVSNILLRMRDLANQSASETNTSKNQAA |
| SEQ ID NO: 353 | |
| Bacillus thuringiensis serovar sotto [52] | |
| FlgII-28 | SVSNILLRMRDIANQSANGTNTGDNQKA |
| SEQ ID NO: 354 | |
| Bacillus thuringiensis serovar Novosibirsk | |
| FlgII-28 | SVSNILLRMRDLANQSANGTNTSENQAA |
| SEQ ID NO: 355 | |
| Bacillus thuringiensis serovar londrina | |
| FlgII-28 | TVTNILQRMRDLAVQSANVTNSNKNRNS |
| SEQ ID NO: 356 | |
| Bacillus cereus strain E33L | |
| FlgII-28 | SVSNILLRMRDLANQSANGTNTDKNQGA |
| SEQ ID NO: 357 | |
| Bacillus cereus strain E33L | |
| FlgII-28 | SVSNILLRMRDLANQSANGTNTDKNQAA |
| SEQ ID NO: 358 | |
| Bacillus cereus | |
| strain FRI-35 | |
| FlgII-28 | TVTNVLQRMRDLAVQSANGTNSNKNRDS |
| SEQ ID NO: 359 | |
| Bacillus cereus strain FRI-35 | |
| FlgII-28 | SVSNILLRMRDIANQTANGTNKDTDIEA |
| SEQ ID NO: 360 | |
| Bacillus thuringiensis | |
| FlgII-28 | SVSNILLRMRDIANQTANGTNKDTDIEA |
| SEQ ID NO: 361 | |
| Bacillus cereus strain ATCC 4342 | |
| FlgII-28 | TVMNILQRMRDLAIQSANSTNSNKNRDS |
| SEQ ID NO: 362 | |
| Bacillus thuringiensis | |
| FlgII-28 | SVSNILLRMRDIANQSANGTNTGDNQKA |
| SEQ ID NO: 363 | |
| Bacillus thuringiensis | |
| FlgII-28 | ETHDILQRMRELVVQAGNGTNKTEDLDA |
| SEQ ID NO: 364 | |
| Bacillus aryabhattai | |
| FlgII-28 | SVSNILLRMRDLATQSATGTNQGNDRES |
| SEQ ID NO: 365 | |
| Bacillus manliponensis | |
| FlgII-28 | ETHAIVQRMRELAVQAATDTNTDDDRAK |
| SEQ ID NO: 366 | |
| Lysinibacillus sp. strain BF-4 | |
| FlgII-28 | ETHAIVQRMRELAVQAATDTNTDDDRAK |
| SEQ ID NO: 367 | |
| Lysinibacillus sp. strain 13S34_air | |
| FlgII-28 | EIHSMLQRMNELAVQASNGTYSGSDRLN |
| SEQ ID NO: 368 | |
| Paenibacillus sp. strain HW567 | |
| FlgII-28 | SVSNILLRMRDLANQSANGTNTKENQDA |
| SEQ ID NO: 369 | |
| Bacillus anthracis | |
| FlgII-28 | SVSNILTRMRDIAVQSSNGTNTAENQSA |
| SEQ ID NO: 370 | |
| Bacillus anthracis | |
| FlgII-28 | SVSNILTRMRDIAVQSSNGTNTAENQSA |
| SEQ ID NO: 371 | |
| Bacillus anthracis | |
| FlgII-28 | SVSNILTRMRDIAVQSSNGTNTAENQSA |
| SEQ ID NO: 372 | |
| Bacillus anthracis | |
| FlgII-28 | SVSNILTRMRDIAVQSSNGTNTAENQSA |
| SEQ ID NO: 373 | |
| Bacillus anthracis strain H9401 | |
| FlgII-28 | ETHDILQRMRELVVQAGNGTNKTEDLDA |
| SEQ ID NO: 374 | |
| Bacillus megaterium strain WSH-002 | |
| FlgII-28 | EIHEMLQRMRELAVQAANGTYSDKDKKA |
| SEQ ID NO: 375 | |
| Aneurinibacillus sp. XH2 | |

Retro-Inverso Flagellin-Associated Polypeptides

Bioactive Flg polypeptide(s) useful for the bioactive priming compositions or methods herein can be created in a non-natural isomeric or retro-inverso (RI) form and used in the compositions and methods herein.

The retro-inverso Flg polypeptides can exhibit enhanced binding affinity for the FLS receptor protein(s). Plant flagellin receptors, like FLS2, can recognize a retro inverso Flg polypeptide fragment such as either Flg22 or FlgII-28 located within the N-terminal conserved domain of flagellin. The retro-inverso forms of these Flg polypeptides are provided as biologically active forms, which can recognize and interact with the Flg-associated or FLS receptor protein on the surface of the plant cell membrane.

Retro-inverso Flg polypeptides can possess an increased activity and stability to proteolytic degradation at the plant membrane surface. For example, retro inverso forms of Bacillus Flg22 or FlgII-28 polypeptides can increase activity and stability of the Flg polypeptide(s) and increase protection against proteolytic degradation at the plant surface or root surface. The retro inverso forms also exhibit enhanced stability when applied in a field, or on or in a soil.

Retro-inverso polypeptides are topological mirror images of the native structures of the parent polypeptide. Retro inverso synthetic forms of the polypeptide sequences are created by reversing the polypeptide sequences and using retro-all-D or retro-enantio-peptides. The all D-chain amino acid Flg polypeptide(s) adopts a “mirror image” of the three-dimensional structure of its related L-peptide or L-chain amino.

This is further accomplished by creating a retro-inverso alteration of any of the parent Flg polypeptide derived from Bacillus or other Eubacteria in Table 3. Retro-inverso polypeptides that were designed to the Flg22 (RI Flg22: SEQ ID NOs: 376-450), and FlgII-28 (RI-FlgII-28: SEQ ID NOs: 451-525) are provided in Table 4. Retro inverso forms of Ec.Flg22 (SEQ ID NO: 526) and Ec.Flg15 (SEQ ID NO: 529) as provided in Table 5 were also created from E. coli derived sequences.

Any of the flagellin-associated bioactive priming polypeptides comprising Bacillus or from other Eubacteria Flg22 or FlgII-28 polypeptides in Table 3 can be used in their retro-inversed forms (Table 4) in the compositions and methods herein.

Retro inverso forms of the Flg bioactive priming polypeptides as referenced herein can be provided in any of three forms where the inversion of amino acid chirality contains the normal-all-D (inverso), all-L (retro) and/or retro-all-D (retro-inverso) or a combination of these forms to achieve the desired phenotypes in a plant.

The Bacillus-derived L-Flg22 and L-FlgII-28 polypeptides in Table 3 and the E.c. native L-Flg22 and L-Flg15 polypeptides in Table 5 were synthetically generated via retro-inverso engineering to form retro-inverso D-Flg22 polypeptide (SEQ ID NO: 376-450), D-FlgII-28 (SEQ ID NO: 451-525), and E.c. D-Flg22 polypeptide (SEQ ID NO: 527, 529).

The inversion of amino acid chirality (all-L to all-D) for Bt.4Q7 Flg22 (SEQ ID NO: 376), which is provided as a small linear polypeptide fragment and is referred to as a retro inverso modification was achieved by a reversal of the direction of the polypeptide backbone and described below.

(DADIADLDGDADADDDDDSDADSDNDIDRDKDGDSDSDLDRDD)

The retro inverso all D-chain amino acid Flg22 polypeptide adopts a “mirror image” of the three-dimensional structure of its related native L-Bt.4Q7Flg 22 polypeptide and this all L-chain has an equivalent mirror image to the all D Bt.4Q7Flg22 polypeptide. All L-amino acid residues are replaced by their D-enantiomers leading to all D-peptides or retro all D-isomer-peptides containing amide linkages. The native L-amino acid chain form of Bt.4Q7 Flg22 polypeptide chain reversed to generate the retro-inverso synthetic all-D confirmation that is prepared by replacing all the L-amino acid residues with their corresponding D-enantiomers.

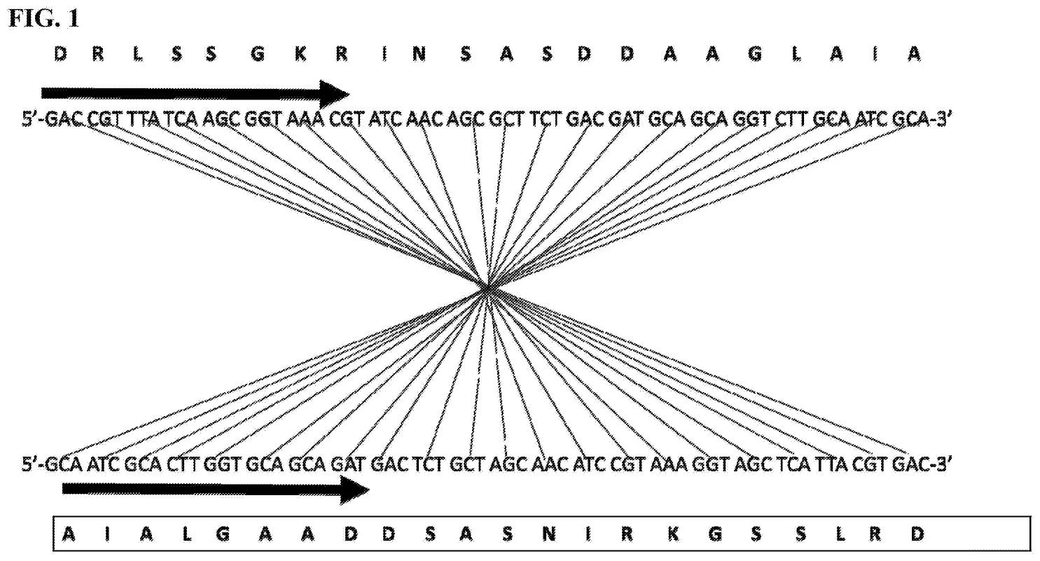

FIG. 1 provides a diagrammatic representation of a natural (all L) Bt.4Q7 Flg22 and its retro inverso or mirror image to form an all D Bt.4Q7 Flg22 enantiomeric polypeptide. The retro-inverso Flg polypeptide that corresponds to Bt.4Q7 Flg22 (SEQ ID NO: 226) is described as SEQ ID NO: 376.

In the case of short polypeptides, such as Flg22, Flg15 and FlgII-28, the mirroring of the side chain positions in a conformational change from L-to-D conversion states results in a mirroring of symmetry transformations of the side chains as well.

Retro-all-D analogues have been found to possess biological activity (Guptasarma, “Reversal of peptide backbone direction may result in mirroring of protein structure, FEBS Letters 310: 205-210, 1992). The retro-inverso D-Flg polypeptide(s) can assume a side chain topology in its extended conformation that is similar to a corresponding native L-Flg polypeptide sequence, thus emulating biological activities of the native L-parent molecule while fully resistant to proteolytic degradation thus increasing stability when the polypeptide contacts the plant or the surrounding environment.

Retro-inverso Flg bioactive priming polypeptides are described in Table 4 or Table 5. Retro inverso Flg-associated bioactive priming polypeptides provided in Table 4 were selected for their enhanced activity and stability and their ability to survive under varying conditions and environments. Based on their D enantiomer nature, they are more resistant to proteolytic degradation and can survive and exist in harsher environmental conditions.

TABLE 4
Retro-inverso flagellin polypeptides from Flg22
and FlgII-28 from Bacillus

| SEQ ID NO: | Peptide Flg22 |
|---|---|
| RI Bt.4Q7Flg22 | AIALGAADDSASNIRKGSSLRD |
| SEQ ID NO: 376 | |
| Bacillus thuringiensis | |
| strain 4Q7 | |
| RI Flg22 | AIALGAADDSASNIRKGSSLRD |
| SEQ ID NO: 377 | |
| Bacillus thuringiensis, strain | |
| HD1002 | |
| RI Flg22 | AIALGAADDASNIRKGSSLRD |
| SEQ ID NO: 378 | |
| Bacillus thuringiensis, strain | |
| HD-789 | |
| RI Flg22 | AIALGAADDSASNIRKGSSLRD |
| SEQ ID NO: 379 | |
| Bacillus cereus | |
| strain G9842 | |
| RI Flg22 | VIANAPNDSANNLKKGTALHE |
| SEQ ID NO: 380 | |
| Bacillus thuringiensis serovar indiana | |
| strain HD521 | |
| RI Flg22 | TAIAGAADDSANNIRKGSSLRD |
| SEQ ID NO: 381 | |
| Bacillus thuringiensis strain CTC | |
| RI Flg22 | AIALGAADDSANNIRKGSSLRD |
| SEQ ID NO: 382 | |
| Bacillus thuringiensis | |
| serovaryunnanensis strain IEBC-T20001 | |
| RI Flg22 | AIALGAADDSANNIRKGSSLRD |
| SEQ ID NO: 383 | |
| Bacillus thuringiensis serovar tolworthi | |
| RI Flg22 | AIALGAADDSANNIRKGSSLRD |
| SEQ ID NO: 384 | |
| Bacillus cereus strain FM1 | |
| RIFlg22 | VIAVNAPNDSAHNLKKGTALHE |
| SEQ ID NO: 385 | |
| Bacillus cereus strain FM1 | |
| RI Flg22 | AIALGAADDSANNIRKGSSLRD |
| SEQ ID NO: 386 | |
| Bacillus thuringiensis strain MC28 | |
| RI Flg22 | AIALGAADDSANNIRKGSSLRD |
| SEQ ID NO: 387 | |
| Bacillus bombysepticus | |
| strain Wang | |
| RI Flg22 | AIALGAADDSANNIRKGSSLRD |
| SEQ ID NO: 388 | |
| Bacillus thuringiensis serovar kenyae | |
| RI Flg22 | AIALGAADDSANNIRKGSSLRD |
| SEQ ID NO: 389 | |
| Bacillus thuringiensis serovar kenyae | |
| RI Flg22 | AIALGAADDSANNIRKGSSLRD |
| SEQ ID NO: 390 | |
| Bacillus cereus | |
| RI Flg22 | VIAINAPNDASNNLKKGTALHE |
| SEQ ID NO: 391 | |
| Bacillus cereus | |
| RI Flg22 | VIANAPNDSAHNLKKGTALHE |
| SEQ ID NO: 392 | |
| Bacillus thuringiensis serovar finitimus | |
| strain YBT-020 | |
| RI Flg22 | AIALGAADDSANNIRKGSSLRD |
| SEQ ID NO: 393 | |
| Bacillus thuringiensis serovar finitimus | |
| strain YBT-020 | |
| RI Flg22 | AIALGAADDSANNIRKGSSLRD |
| SEQ ID NO: 394 | |
| Bacillus cereus stain B4264 | |
| RI Flg22 | AIALGAADDSANNIRKGSSLRD |
| SEQ ID NO: 395 | |
| Bacillus thuringiensis serovar nigeriensis | |
| RI Flg22 | AIALGAADDSANNIRKGSSLRD |
| SEQ ID NO: 396 | |
| Bacillus thuringiensis | |
| RI Flg22 | VIANAPNDSAHNLKKGTAFHE |
| SEQ ID NO: 397 | |
| Bacillus thuringiensis serovar konkukian | |
| strain 97-27 | |
| RI Flg22 | AIALGAADDSANNRKGSSLRD |
| SEQ ID NO: 398 | |
| Bacillus thuringiensis serovar konkukian | |
| strain 97-27 | |
| RI Flg22 | VIVINAPNDSAHNLKKGTALHE |
| SEQ ID NO: 399 | |
| Bacillus thuringiensis serovar thuringiensis | |
| strain IS5056 | |
| RI Flg22 | AIALGAADDSANNIRKGSSLRD |
| SEQ ID NO: 400 | |
| Bacillus thuringiensis serovar thuringiensis | |
| strain IS5056 | |
| RI Flg22 | AIALGAADDSANNIRKGSSLRD |
| SEQ ID NO: 401 | |
| Bacillus thuringiensis | |
| strain Bt407 | |
| RI Flg22 | AIALGAADDSANNIRKGSSLRD |
| SEQ ID NO: 402 | |
| Bacillus thuringiensis serovar chinensis | |
| CT-43 | |
| RI Flg22 | AIALGAADDSANNIRKGSSLRD |
| SEQ ID NO: 403 | |
| Bacillus thuringiensis serovar canadensis | |
| RI Flg22 | AIALGAADDSANNIRKGSSLRD |
| SEQ ID NO: 404 | |
| Bacillus thuringiensis serovar galleriae | |
| RI Flg22 | AIALGAADDSANNIRKGSSLRD |
| SEQ ID NO: 405 | |
| Bacillus weihenstephanensis | |
| RI Flg22 | AIALGAADDSANNIRKGSSLRD |
| SEQ ID NO: 406 | |
| Bacillus thuringiensis serovar ostriniae | |
| RI Flg22 | VIANAPNDSAHNLKKGTALHE |
| SEQ ID NO: 407 | |
| Bacillus thuringiensis | |
| RI Flg22 | AIALGAADDSANNIRKGSSLRD |
| SEQ ID NO: 408 | |
| Bacillus thuringiensis | |
| RI Flg22 | AIALGAADDSANNIRKGSSLRD |
| SEQ ID NO: 409 | |
| Bacillus thuringiensis serovar | |
| pondicheriensis | |
| RI Flg22 | VIVINAPNDASHNLKKGTALHE |
| SEQ ID NO: 410 | |
| Bacillus thuringiensis serovar Berliner | |
| RI Flg22 | AIALGAADDSANNIRKGSSLRD |
| SEQ ID NO: 411 | |
| Bacillus thuringiensis serovar Berliner | |
| RI Flg22 | VIAVANPNNSAHNLKKGTALHE |
| SEQ ID NO: 412 | |
| Bacillus cereus strain Q1 | |
| RI Flg22 | AIALGAADDSANNIRKGSSLRD |
| SEQ ID NO: 413 | |
| Bacillus cereus strain Q1 | |
| RI Flg22 | VIANAPNDSAHNLKKGTALHE |
| SEQ ID NO: 414 | |
| Bacillus thuringiensis serovar morrisoni | |
| RI Flg22 | AIALGAADDSANNIRKGSSLRD |
| SEQ ID NO: 415 | |
| Bacillus thuringiensis serovar neoleonensis | |
| RI Flg22 | AIALGAADDSANNIRKGSSLRD |
| SEQ ID NO: 416 | |
| Bacillus thuringiensis serovar morrisoni | |
| RI Flg22 | AIALGAADDSANNIRKGSSLRD |
| SEQ ID NO: 417 | |
| Bacillus thuringiensis serovar morrisoni | |
| RI Flg22 | AIALGAADDSANNIRKGSSLRD |
| SEQ ID NO: 418 | |
| Bacillus thuringiensis serovar jegathesan | |
| RI Flg22 | AIALGAADDSANNIRKGSSLRD |
| SEQ ID NO: 419 | |
| Bacillus cereus stain ATCC 10987 | |
| RI Flg22 from Flagellin A | AIALGAADDASNNIRKGSSLRD |
| SEQ ID NO: 420 | |
| Bacillus thuringiensis serovar monterrey | |
| RI Flg22 | VIANAPNDSANNLKKGTALHE |
| SEQ ID NO: 421 | |
| Bacillus cereus strain NC7401 | |
| RI Flg22 | AIALGAADDSANNIRKGSSLRD |
| SEQ ID NO: 422 | |
| Bacillus cereus strain NC7401 | |
| RI Flg22 | AIALGAADDSANNIRKGSSLRD |
| SEQ ID NO: 423 | |
| Bacillus cereus strain AH820 | |
| RI Flg22 | AIALGAADDSANNIRKGSSLRD |
| SEQ ID NO: 424 | |
| Bacillus cereus AH187 | |
| RI Flg22 | AIALGAADDSANNIRKGSSLRD |
| SEQ ID NO: 425 | |
| Bacillus cereus | |
| RI Flg22 | AIALGAADDSANNIRKGSSLRD |
| SEQ ID NO: 426 | |
| Bacillus cereus | |
| RI Flg22 | AIALGAADDSANNIRKGSSLRD |
| SEQ ID NO: 427 | |
| Bacillus thuringiensis | |
| Strain HD-771 [51] | |
| RI Flg22 | AIALGAADDANNIRKGSSLRD |
| SEQ ID NO: 428 | |
| Bacillus thuringiensis serovar sotto [52] | |
| RI Flg22 | AIALGAADDSANNIRKGSSLRD |
| SEQ ID NO: 429 | |
| Bacillus thuringiensis serovar Novosibirsk | |
| RI Flg22 | AIALGAADDSANNIRKGSSLRD |
| SEQ ID NO: 430 | |
| Bacillus thuringiensis serovar londrina | |
| RI Flg22 | VIAINAPNNSAHNLKKGTALHE |
| SEQ ID NO: 431 | |
| Bacillus cereus strain E33L | |
| RI Flg22 | AIALGAADDSANNIRKGSSLRD |
| SEQ ID NO: 432 | |
| Bacillus cereus strain E33L | |
| RI Flg22 | AIALGAADDSANNIRKGSSLRD |
| SEQ ID NO: 433 | |
| Bacillus cereus | |
| strain FRI-35 | |
| RI Flg22 | VIAINAPNDSANNLKKGTALHE |
| SEQ ID NO: 434 | |
| Bacillus cereus strain FRI-35 | |
| RI Flg22 | AIALGAADDSANNIRKGSSLRD |
| SEQ ID NO: 435 | |
| Bacillus thuringiensis | |
| RI Flg22 | AIALGAADDANNIRKGSSLRD |
| SEQ ID NO: 436 | |
| Bacillus cereus strain ATCC 4342 | |
| RI Flg22 | VIANAPNDSAHNLKKGTALHE |
| SEQ ID NO: 437 | |
| Bacillus thuringiensis | |
| RI Flg22 | AIALGAADDSANNIRKGSSLRD |
| SEQ ID NO: 438 | |
| Bacillus thuringiensis | |
| RI Flg22 | SIALGAADDSASNIRQGSSLKE |
| SEQ ID NO: 439 | |
| Bacillus aryabhattai | |
| RI Flg22 | AIALGAADDSASNIQKGSSLRN |
| SEQ ID NO: 440 | |
| Bacillus manliponensis | |
| RI Flg22 | SIALGAADDAASNIRYGSSLRL |
| SEQ ID NO: 441 | |
| Lysinibacillus sp. strain BF-4 | |
| RI Flg22 | SIALGAADDAASNIRYGSSLRL |
| SEQ ID NO: 442 | |
| Lysinibacillus sp. strain 13S34_air | |
| RI Flg22 | SIAGLAADDSAGNIRLGSSLKG |
| SEQ ID NO: 443 | |
| Paenibacillus sp. strain HW567 | |
| RI Flg22 | AIALGAADDSANNIRKGSSLRD |
| SEQ ID NO: 444 | |
| Bacillus anthracis | |
| RI Flg22 | AIALGAADDAASNIRKGSSLRN |
| SEQ ID NO: 445 | |
| Bacillus anthracis | |
| RI Flg22 | AIALGAADDAASNIRKGSSLRN |
| SEQ ID NO: 446 | |
| Bacillus anthracis | |
| RI Flg22 | AIALGAADDAASNIRKGSSLRN |
| SEQ ID NO: 447 | |
| Bacillus anthracis | |
| RI Flg22 | AIALGAADDAASNIRKGSSLRN |
| SEQ ID NO: 448 | |
| Bacillus anthracis strain H9401 | |
| RI Flg22 | SIALGAADDSASNIRQGSSLKE |
| SEQ ID NO: 449 | |
| Bacillus megaterium strain WSH-002 | |
| RI Flg22 | SIALGAADDSARNIRYGSSLRE |
| SEQ ID NO: 450 | |
| Aneurinibacillus sp. XH2 | |
| SEQ ID NO: | Peptide Flg15 |
| RI Flg15-Bt4Q7 | AIALGAADDKASNIR |
| SEQ ID NO: 586 | |
| Modified FLG15-Bt4Q7; Syn01 | |
| Bacillus thuringiensis strain 4Q7 | |
| SEQ ID NO: | Peptide FlgII-28 |
| RI FlgII-28-Bt.4Q7 | SAQNGKTNTGNASQNALDRMRLLINSVS |
| SEQ ID NO: 451 | |
| Bacillus thuringiensis | |
| strain 4Q7 | |
| RI FlgII-28 | SAQNGKTNTGNASQNALDRMRLLINSVS |
| SEQ ID NO: 452 | |
| Bacillus thuringiensis, strain | |
| HD1002 | |
| RI FlgII-28 | SAQNGKTNTGNASQNALDRMRLLINSVS |
| SEQ ID NO: 453 | |
| Bacillus thuringiensis, strain | |
| HD-789 | |
| RI FlgII-28 | SAQNGKTNTGNASQNALDRMRLLINSVS |
| SEQ ID NO: 454 | |
| Bacillus cereus | |
| strain G9842 | |
| RI FlgII-28 | SHRNKNSNTGNASQVALDRMRQLINTVT |
| SEQ ID NO: 455 | |
| Bacillus thuringiensis serovar indiana | |
| strain HD521 | |
| RI FlgII-28 | ASKNENTNTGNASQNAIDRMRLLINSVS |
| SEQ ID NO: 456 | |
| Bacillus thuringiensis strain CTC | |
| RI FlgII-28 | AKQNDDTNTGNASQNALDRMRLLINSVS |
| SEQ ID NO: 457 | |
| Bacillus thuringiensis | |
| serovaryunnanensis strain IEBC-T20001 | |
| RI FlgII-28 | AAKNEDTNTGNASQNALDRMRLLINSVS |
| SEQ ID NO: 458 | |
| Bacillus thuringiensis serovar tolworthi | |
| RI FlgII-28 | LAVQNKDTNTGNASQNAIDRMRLLINSVS |
| SEQ ID NO: 459 | |
| Bacillus cereus strain FM1 | |
| RI FlgII-28 | SDRNKNSNTGNASQVAVDRMRQLINTVT |
| SEQ ID NO: 460 | |
| Bacillus cereus strain FM1 | |
| RI FlgII-28 | AQQNDATNTGNASQNAIDRMRLLINSVS |
| SEQ ID NO: 461 | |
| Bacillus thuringiensis strain MC28 | |
| RI FlgII-28 | AAQNKDTNTGSASQNALDRMRLLINSVS |
| SEQ ID NO: 462 | |
| Bacillus bombysepticus strain Wang | |
| RI FlgII-28 | AAQNKDTNTGSASQNALDRMRLLINSVS |
| SEQ ID NO: 463 | |
| Bacillus thuringiensis serovar kenyae | |
| RI FlgII-28 | AAQNKDTNTGSASQNALDRMRLLINSVS |
| SEQ ID NO: 464 | |
| Bacillus thuringiensis serovar kenyae | |
| RI FlgII-28 | AKQNDGTNTGNASQNALDRMRLLINSVS |
| SEQ ID NO: 465 | |
| Bacillus cereus | |
| RI FlgII-28 | NLSDRNKNSNTGNASQVALDRMRQLINT |
| SEQ ID NO: 466 | |
| Bacillus cereus | |
| RI FlgII-28 | NLSDRNKNLNTGNASQVAVDRMRQLVNT |
| SEQ ID NO: 467 | |
| Bacillus thuringiensis serovar finitimus | |
| strain YBT-020 | |
| RI FlgII-28 | ASKNSDTNTGNASQNAIDRMRLLINSVS |
| SEQ ID NO: 468 | |
| Bacillus thuringiensis serovar finitimus | |
| strain YBT-020 | |
| RI FlgII-28 | AAKNEATNTGNASQNALDRMRLLINSVS |
| SEQ ID NO: 469 | |
| Bacillus cereus stain B4264 | |
| RI FlgII-28 | AKQNDSTNTGNASQNAIDRMRLLINSVS |
| SEQ ID NO: 470 | |
| Bacillus thuringiensis serovar nigeriensis | |
| RI FlgII-28 | AQQNDATNTGNASQNAIDRMRLLINSVS |
| SEQ ID NO: 471 | |
| Bacillus thuringiensis | |
| RI FlgII-28 | SDRNKNSNTGNASQVALDRMRQLINMVT |
| SEQ ID NO: 472 | |
| Bacillus thuringiensis serovar konkukian | |
| strain 97-27 | |
| RI FlgII-28 | AQQNDATNTGNASQNAIDRMRLLINSVS |
| SEQ ID NO: 473 | |
| Bacillus thuringiensis serovar konkukian | |
| strain 97-27 | |
| RI FlgII-28 | SDRNTNSNTGNASQIAFDRMHQLINTVT |
| SEQ ID NO: 474 | |
| Bacillus thuringiensis serovar thuringiensis | |
| strain IS5056 | |
| RI FlgII-28 | ASQNKDTNTGNASQNSIDRMRLLINSVS |
| SEQ ID NO: 475 | |
| Bacillus thuringiensis serovar thuringiensis | |
| strain IS5056 | |
| RI FlgII-28 | ASQNKDTNTGNASQNSIDRMRLLINSVS |
| SEQ ID NO: 476 | |
| Bacillus thuringiensis | |
| strain Bt407 | |
| RI FlgII-28 | ASQNKDTNTGNASQNSISRMRLLINSVS |
| SEQ ID NO: 477 | |
| Bacillus thuringiensis serovar chinensis | |
| CT-43 | |
| RI FlgII-28 | AAQNENTNTGNASQNALDRMRLLINSVS |
| SEQ ID NO: 478 | |
| Bacillus thuringiensis serovar canadensis | |
| RI FlgII-28 | AQQNEDTNTGNASQNSLDRMRLLINSVS |
| SEQ ID NO: 479 | |
| Bacillus thuringiensis serovar galleriae | |
| RI FlgII-28 | AQQNEDTNTGNASQNSLDRMRLLINSVS |
| SEQ ID NO: 480 | |
| Bacillus weihenstephanensis | |
| RI FlgII-28 | AKQNDGTNTGNASQNAIDRMRLLINSVS |
| SEQ ID NO: 481 | |
| Bacillus thuringiensis serovar ostriniae | |
| RI FlgII-28 | SDRNKNSNTDNSSQVALDRMRQLINAVT |
| SEQ ID NO: 482 | |
| Bacillus thuringiensis | |
| RI FlgII-28 | AKQNDDTNTGNASQNALDRMRLLINSVS |
| SEQ ID NO: 483 | |
| Bacillus thuringiensis | |
| RI FlgII-28 | AKQNDDTNTGNASQNALDRMRLLINSVS |
| SEQ ID NO: 484 | |
| Bacillus thuringiensis serovar | |
| pondicheriensis | |
| RI FlgII-28 | SDRNTNSNTGNASQIAFDRMHQLINTVT |
| SEQ ID NO: 485 | |
| Bacillus thuringiensis serovar Berliner | |
| RI FlgII-28 | ASQNKDTNTGNASQNSIDRMRLLINSVS |
| SEQ ID NO: 486 | |
| Bacillus thuringiensis serovar Berliner | |
| RI FlgII-28 | SDRNKSSNTGNASQVAVDRMRQLVNTVT |
| SEQ ID NO: 487 | |
| Bacillus cereus strain Q1 | |
| RI FlgII-28 | AVQKDTNTGNASQNAIDRMRLLINSVS |
| SEQ ID NO: 488 | |
| Bacillus cereus strain Q1 | |
| RI FlgII-28 | SDRNKNSNTSNASQIALDRMRQLINMVT |
| SEQ ID NO: 489 | |
| Bacillus thuringiensis serovar morrisoni | |
| RI FlgII-28 | AKQNDSTNIGNASQNAIDRMRLLINSVS |
| SEQ ID NO: 490 | |
| Bacillus thuringiensis serovar neoleonensis | |
| RI FlgII-28 | AKQNDGTNTFNASQNAIDRMRLLINSVS |
| SEQ ID NO: 491 | |
| Bacillus thuringiensis serovar morrisoni | |
| RI FlgII-28 | AKQNDGTNTFNASQNAIDRMRLLINSVS |
| SEQ ID NO: 492 | |
| Bacillus thuringiensis serovar morrisoni | |
| RI FlgII-28 | AAQNGNTNTFNASQNAIDRMRLLINSVS |
| SEQ ID NO: 493 | |
| Bacillus thuringiensis serovar jegathesan | |
| RI FlgII-28 | AAQNKDTNTGNASQNAIDRMRLLINSVS |
| SEQ ID NO: 494 | |
| Bacillus cereus stain ATCC 10987 | |
| RI FlgII-28 from Flagellin A | AAQNENTNTGNASQNALDRMRLLINSVS |
| SEQ ID NO: 495 | |
| Bacillus thuringiensis serovar monterrey | |
| RI FlgII-28 | SDRNKNSNTDNASQVALDRMRQLVNTVT |
| SEQ ID NO: 496 | |
| Bacillus cereus strain NC7401 | |
| RI FlgII-28 | AAKNENTNTGNASQNALDRMRLLINSVS |
| SEQ ID NO: 497 | |
| Bacillus cereus strain NC7401 | |
| RI FlgII-28 | AAQNDSTNTGNASQNALDRMRLLINSVS |
| SEQ ID NO: 498 | |
| Bacillus cereus strain AH820 | |
| RI FlgII-28 | AAKNENTNTGNASQNALDRMRLLINSVS |
| SEQ ID NO: 499 | |
| Bacillus cereus AH187 | |
| RI FlgII-28 | AAKNENTNTGNASQNALDRMRLLINSVS |
| SEQ ID NO: 500 | |
| Bacillus cereus | |
| RI FlgII-28 | AKQNDGTNTGNASQNAIDRMRLLINSVS |
| SEQ ID NO: 501 | |
| Bacillus cereus | |
| RI FlgII-28 | AAQNKSTNTESASQNALDRMRLLINSVS |
| SEQ ID NO: 502 | |
| Bacillus thuringiensis | |
| Strain HD-771 [51] | |
| RI FlgII-28 | AAQNKSTNTESASQNALDRMRLLINSVS |
| SEQ ID NO: 503 | |
| Bacillus thuringiensis serovar sotto [52] | |
| RI FlgII-28 | AKQNDGTNTGNASQNAIDRMRLLINSVS |
| SEQ ID NO: 504 | |
| Bacillus thuringiensis serovar Novosibirsk | |
| RI FlgII-28 | AAQNESTNTGNAQNALDRMRLLINSVS |
| SEQ ID NO: 505 | |
| Bacillus thuringiensis serovar londrina | |
| RI FlgII-28 | SNRNKNSNTVNASQVALDRMRQLINTVT |
| SEQ ID NO: 506 | |
| Bacillus cereus strain E33L | |
| RI FlgII-28 | AGQNKDTNTNASQNALDRMRLLINSVS |
| SEQ ID NO: 507 | |
| Bacillus cereus strain E33L | |
| RI FlgII-28 | AAQNKDTNTGNASQNALDRMRLLINSVS |
| SEQ ID NO: 508 | |
| Bacillus cereus | |
| strain FRI-35 | |
| RI FlgII-28 | SDRNKNSNTGNASQVALDRMRQLVNTVT |
| SEQ ID NO: 509 | |
| Bacillus cereus strain FRI-35 | |
| RI FlgII-28 | AEIDTDKNTGNATQNAIDRMRLLINSVS |
| SEQ ID NO: 510 | |
| Bacillus thuringiensis | |
| RI FlgII-28 | AEIDTDKNTGNATQNAIDRMRLLINSVS |
| SEQ ID NO: 511 | |
| Bacillus cereus strain ATCC 4342 | |
| RI FlgII-28 | SDRNKNSNTSNASQIALDRMRQLINMT |
| SEQ ID NO: 512 | |
| Bacillus thuringiensis | |
| RI FlgII-28 | AKQNDGTNTGNASQNAIDRMRLLINSVS |
| SEQ ID NO: 513 | |
| Bacillus thuringiensis | |
| RI FlgII-28 | ADLDETKNTGNGAQVVLERMRQLIDHTE |
| SEQ ID NO: 514 | |
| Bacillus aryabhattai | |
| RI FlgII-28 | SERDNGQNTGTAQTALDRMRLLINSVS |
| SEQ ID NO: 515 | |
| Bacillus manliponensis | |
| RI FlgII-28 | KARDDDTNTDTAAQVALERMRQVIAHTE |
| SEQ ID NO: 516 | |
| Lysinibacillus sp. strain BF-4 | |
| RI FlgII-28 | KARDDDTNTDTAAQVALERMRQVIAHTE |
| SEQ ID NO: 517 | |
| Lysinibacillus sp. strain 13S34_air | |
| RI FlgII-28 | NLRDSGSYTGNSAQVALENMRQLMSHIE |
| SEQ ID NO: 518 | |
| Paenibacillus sp. strain HW567 | |
| RI FlgII-28 | ADQNEKTNTGNASQNALDRMRLLINSVS |
| SEQ ID NO: 519 | |
| Bacillus anthracis | |
| RI FlgII-28 | ASQNEATNTGNSSQVAIDRMRTLINSVS |
| SEQ ID NO: 520 | |
| Bacillus anthracis | |
| RI FlgII-28 | ASQNEATNTGNSSQVAIDRMRTLINSVS |
| SEQ ID NO: 521 | |
| Bacillus anthracis | |
| RI FlgII-28 | ASQNEATNTGNSSQVAIDRMRTLINSVS |
| SEQ ID NO: 522 | |
| Bacillus anthracis | |
| RI FlgII-28 | ASQNEATNTGNSSQVIADRMRTLINSVS |
| SEQ ID NO: 523 | |
| Bacillus anthracis strain H9401 | |
| RI FlgII-28 | ADLDETKNTGNGAQVVLERMRQLIDHTE |
| SEQ ID NO: 524 | |
| Bacillus megaterium strain WSH-002 | |
| RI FlgII-28 | AKKDKSYTGNAAQVALERMRQLMEHIE |
| SEQ ID NO: 525 | |
| Aneurinibacillus sp. XH2 | |

Flg Sequences from Various Organisms

TABLE 5
Flagellin-associated Flg22 and Flg15 polypeptides and RI-polypeptides thereof
from other organisms

| SEQ ID NO: | Peptide - Amino Acid |
|---|---|
| Flagellin (Flg22) | ERLSSGLRINSAKDDAAGQAIA |
| SEQ ID NO: 526 | |
| Escherichia coli | |
| Flagellin (Retro-Inverso Flg22) | AIAQGAADDKASNIRLGSSLRE |
| SEQ ID NO: 527 | |
| Escherichia coli | |
| Flagellin (Flg15) | RINSAKDDAAGQAIA |
| SEQ ID NO: 528 | |
| Escherichia coli | |
| Flagellin (Retro-Inverso Flg15) | AIAQGAADDKASNIR |
| SEQ ID NO: 529 | |
| Escherichia coli | |
| Flagellin (Flg22) | QRLSTGSRINSAKDDAAGLQIA |
| SEQ ID NO: 530 | |
| Pseudomonas aeruginosa | |
| Flagellin (Retro Inverso Flg22) | AIQLGAADDKASNIRSGTSLRQ |
| SEQ ID NO: 531 | |
| Pseudomonas aeruginosa | |
| Flagellin (Flg22) | QRLSSGLRINSAKDDAAGLAIS |
| SEQ ID NO: 532 | |
| Xanthomonas spp. | |
| X. campestris & X. citri | |
| Flagellin (Retro Inverso Flg22) | SIALGAADDKASNIRLGSSLRQ |
| SEQ ID NO: 533 | |
| Xanthomonas spp. | |
| X. campestris & X. citri | |
| Flagellin (Flg22) | QRLSSGLRINSAKDDAAGQAIS |
| SEQ ID NO: 534 | |
| Erwinia amylovora | |
| Flagellin (Retro Inverso Flg22) | SIAQGAADDKASNIRLGSSLRQ |
| SEQ ID NO: 535 | |
| Erwinia amylovora | |
| Flagellin (Flg22) | TRLSSGKRINSAADDAAGLAIS |
| SEQ ID NO: 536 | |
| Burkholderia phytofirmans | |
| Flagellin (Retro Inverso Flg22) | SIALGAADDAASNIRKGSSLRT |
| SEQ ID NO: 537 | |
| Burkholderia phytofirmans | |
| Flagellin (Flg22) | NRLSSGKRINTAADDAAGLAIS |
| SEQ ID NO: 538 | |
| Burkholderia ubonensis | |
| Flagellin (Retro Inverso Flg22) | SIALGAADDAATNIRKGSSLRN |
| SEQ ID NO: 539 | |
| Burkholderia ubonensis | |
| Flagellin (Flg22) | TRLSSGLKINSAKDDAAGLQIA |
| SEQ ID NO: 540 | |
| Pseudomonas syringae | |
| Flagellin (Retro Inverso Flg22) | AIQLGAADDKASNIKLGSSLRT |
| SEQ ID NO: 541 | |
| Pseudomonas syringae | |
| Flagellin (FlgII-28) | ESTNILQRMRELAVQSRNDSNSATDREA |
| (SEQ ID NO: 587) | |
| Pseudomonas syringae | |
| Flagellin (Retro Inverso FlgII-28) | AERDTASNSDNRSQVALERMRQLINTSE |
| (SEQ ID NO: 588) | |
| Pseudomonas syringae | |

The composition can comprise at least one retro-inverso flagellin or flagellin associated polypeptide.

The retro-inverso flagellin or flagellin associated polypeptide can be a retro inverso Flg22 polypeptide. An amino acid sequence of the retro-inverso Flg22 polypeptide can comprise any one of SEQ ID NOs: 376-450, 527, 531, 533, 535, 537 and 539.

The retro-inverso flagellin or flagellin associated polypeptide can be a retro inverso FlgII-28 polypeptide. An amino acid sequence of the retro-inverso FlgII-28 polypeptide can comprise any one of SEQ ID NOs: 451-525, or 588.

The retro-inverso flagellin or flagellin associated polypeptide can be a retro inverso Flg15 polypeptide. An amino acid sequence of the retro-inverso Flg15 polypeptide can comprise any one of SEQ ID NOs: 529 or 586.

Sequences that Assist in Directing Flagellins or Flagellin-Associated Polypeptides to the Plant

The signature, signal anchor sorting and secretion sequences can be used separately or together in combination with any of the flagellin or flagellin-associated polypeptides as described herein. These assistance sequences are useful for the efficient delivery of the flagellin polypeptides to the plant cell membrane surface. Other assistance sequences can also assist with the translocation of the Flg polypeptide fragment across the plasma membrane. Delivery of flagellins and flagellin-associated polypeptides to the plasma membrane surface of a plant (or plant part) can contribute to downstream signalling processes and result in beneficial outcomes to a plant or a plant part, such as enhanced plant health and productivity.

The polypeptide in the compositions or methods herein can further comprise an assistance polypeptide.

The assistance polypeptide can comprise a signature polypeptide, and an amino acid sequence of the signature polypeptide can comprise any one of SEQ ID NOs: 542-548, listed in Table 6, or any combination thereof. For example, the amino acid sequence of the signature polypeptide can comprise SEQ ID NO: 542.

The assistance polypeptide can comprise a signal anchor sorting polypeptide, and an amino acid sequence of the signal anchor sorting polypeptide can comprise any one of SEQ ID NOs: 549-562, listed in Table 7, or any combination thereof. For example, the amino acid sequence of the signal anchor sorting polypeptide can comprise SEQ ID NO: 549.

The flagellin or flagellin-associated polypeptide can be produced recombinantly by a microorganism. For example, the microorganism can comprise a Bacillus, a Pseudomonas, a Paenibacillus, Aneurinibacillus or a Lysinibacillus.

The assistance polypeptide can comprise a secretion polypeptide, and an amino acid sequence of the secretion polypeptide can comprise any one of SEQ ID NOs: 563-570, or any combination thereof. For example, the amino acid sequence of the secretion polypeptide can comprise SEQ ID NO: 563.

These three types of assistance sequences are further described in Table 6 (N-terminal signature sequences), Table 7 (signal anchor sorting sequences) and Table 8 (secretion sequences).

Also provided are “assistance” sequences having conserved signature (Table 6; SEQ ID NOs: 542-548), signal anchor sorting (Table 7; SEQ ID NOs: 549-562) and secretion (Table 8; SEQ ID NOs: 563-570) sequences in combination with any of the flagellin-associated polypeptides as described herein. Particularly useful are combinations of the signature, signal anchor sorting and secretion assistance sequences with the native L-Flg polypeptides (Table 3. SEQ ID NOs: 226-375) or any of the retro inverso Flg22 polypeptides (Table 4. SEQ ID NOs: 376-525) for providing efficient delivery of the Flg polypeptides to the extracellular plant membrane surface, such as the surface of a plant or plant part.

N-terminal Signature Sequences

Amino acid “signature” sequences conserved within Bacillus, Lysinibacillus, Paenibacillus or Aneurinibacillus bacteria (genera) and other Eubacterial generas can function in targeting flagellin polypeptides to the appropriate Flg-associated receptor protein(s), such as FLS receptors that have an exposed binding site at the plant cell membrane surface and can be used to enhance Flg polypeptide-receptor binding leading to an increased activation potential of the Flg-associated receptor(s). Flagellin signature sequences as identified in Table 6 are useful for targeting and stably delivering the Flg polypeptides for binding to the FLS or FLS-like receptor(s) therefore increasing the contact and binding between the membrane receptor and the Flg polypeptide.

Conserved N-terminal signature sequences (SEQ ID NO: 542-548) can be used in combination with any of the flagellin-associated polypeptides as described herein. Of particular utility are the signature sequences used in combination with the native L-Flg polypeptides (L-Flg22 SEQ ID NOs: 226-300; L-FlgII-28 SEQ ID NOs: 301-375) or any of the retro inverso D-Flg polypeptides (D-Flg22 SEQ ID NOs: 376-450; FlgII-28 SEQ ID NO: 451-525) or any of the other Flg-associated sequences provided in Table 5 (SEQ ID NOs: 526-541) to provide efficient delivery of the Flg-associated polypeptides to the plant membrane surface.

Signature sequences assist with Flg22 and FlgII-28 bioactive priming polypeptide sequences in binding to the appropriate Flg-associated receptor(s) in order to activate the receptor(s) making it functionally active.

TABLE 6
Flagellin-associated N-terminal signature
sequences

| | Flagellin Signature |
|---|---|
| SEQ ID NO: | Sequences |
| SEQ ID NO: 542 | GFLN |
| SEQ ID NO: 543 | WGFLI |
| SEQ ID NO: 544 | MGVLN |
| SEQ ID NO: 545 | GVLN |
| SEQ ID NO: 546 | WGFFY |
| SEQ ID NO: 547 | LVPFAVWLA |
| SEQ ID NO: 548 | AVWLA |

N-terminal Signal Anchor Sorting Sequences

Amino acid “signal anchor sorting” sequences conserved within Bacillus, Lysinibacillus, Aneurinibacillus and Paenibacillus genera and other Eubacterial generas' bacteria can function in anchoring and localizing the flagellin-associate polypeptides to the plant cell membrane surface and assist in high affinity binding to the appropriate Flg-associated receptor(s) thereby increasing the activation potential of the bound receptor(s).

Conserved signal anchor sequences (SEQ ID NO: 549-562; Table 7) are located downstream of the pre-cleaved or full-length coding or partial coding flagellin sequences, for example, as described herein (SEQ ID NOs: 1-75; Table 1).

The signal anchor sorting domains as described herein are useful in membrane attachment. They can be used to aid in the localization and binding of Flg-associated polypeptides to a surface membrane receptor and have some functional similarity at the amino acid level to proteins that are endosomal (vesicular) trafficked or destined for targeting to the secretory pathway. Such signal anchor sorting sequences as described herein that are useful for anchoring the Flg bioactive priming polypeptides to the plant cell membrane are also used to enhance the membrane integration of the bioactive priming Flg polypeptides into the plant cell.

Such sequences as described in Table 7 may further be functionally annotated as import receptor signal anchor sequences, which can be used to improve targeting or delivery and efficient membrane anchoring of Flg-associated polypeptides to a plant and assist with membrane integration into the cytosol of the plant cell.

Combining the signal anchor sequences (SEQ ID NOs: 549-562; Table 7) with any of the flagellins or flagellin-associated bioactive priming polypeptides as described herein is useful to facilitate the attachment and import of these flagellin-associated polypeptide(s) into the plant.

Such signal anchor sorting sequences can be used in combination with the Flg-associated polypeptides, and are useful for targeting, efficient membrane anchoring, membrane integration and Golgi-to-lysosomal/vacuolar trafficking. The signal anchor sorting sequences are used to stably deliver the Flg polypeptides to the plant membrane surface and integrally incorporate them into the plant.

Such sequences as described herein contain di-leucine amino acids that are referenced to confer endocytosis functionalities in plant systems (Pond et al. 1995, “A role for acidic residues in di-leucine motif-based targeting to the endocytic pathway”, Journal of Biological Chemistry 270: 19989-19997, 1995).

Such signal anchor sorting sequences as described can also be used to efficiently deliver systemic signals to infection sites and stimulate a plant's innate immunity in plant cells.

TABLE 7
Flagellin-associated signal anchor sorting
sequences

| | Signal Anchor |
|---|---|
| SEQ ID NO: | Sequence |
| SEQ ID NO: 549 | LLGTADKKIKIQ |
| SEQ ID NO: 550 | LLKSTQEIKIQ |
| SEQ ID NO: 551 | LLNEDSEVKIQ |
| SEQ ID NO: 552 | LGVAANNTQ |
| SEQ ID NO: 553 | LLRMRDLANQ |
| SEQ ID NO: 554 | LQRMRDVAVQ |
| SEQ ID NO: 555 | LLRMRDISNQ |
| SEQ ID NO: 556 | LLRMRDIANQ |
| SEQ ID NO: 557 | LQKQIDYIAGNTQ |
| SEQ ID NO: 558 | LLIRLPLD |
| SEQ ID NO: 559 | QRMRELAVQ |
| SEQ ID NO: 560 | TRMRDIAVQ |
| SEQ ID NO: 561 | TRMRDIAVQ |
| SEQ ID NO: 562 | QRMRELVVQ |

C-Terminal Secretion Sequences

Conserved sequences located in the C-terminus of flagellin(s) are further described as secretion sequences (SEQ ID NO: 563-570; Table 8).

Conserved sequences were identified in the C-terminus of the Bacillus, Lysinibacillus, and Paenibacillus bacteria (genera) and other Eubacterial genera derived flagellin proteins and comprise 6 amino acids, for example LGATLN, LGSMIN, or LGAMIN. These sequences were functionally annotated using BLAST against the bacterial databases as motifs that have highest homology to secretion polypeptides. The 6 amino acid conserved polypeptides identified were found most similar to those found in type III secretion systems in E. coli. Type III export systems have been cited to be involved in the translocation of polypeptides across the plant cell membrane. The filament assembly of flagellin is dependent on the availability of flagellins to be secreted and may require chaperones that assist in the secretory process.

These secretion polypeptides as described herein can be used in combination with any of the flagellin-associated polypeptides as described herein to deliver these polypeptides/peptides into the cytosol of the host plant thus providing beneficial outcomes to a plant.

TABLE 8
C-terminal flagellin-associated secretion
sequences

| | Flagellin Secretion |
|---|---|
| SEQ ID NO: | polypeptides |
| SEQ ID NO: 563 | LGATLN |
| SEQ ID NO: 564 | LGATQN |
| SEQ ID NO: 565 | LAQANQ |
| SEQ ID NO: 566 | LGAMIN |
| SEQ ID NO: 567 | LGSMIN |
| SEQ ID NO: 568 | MGAYQN |
| SEQ ID NO: 569 | LGAYQN |
| SEQ ID NO: 570 | YGSQLN |

The signature (SEQ ID NO: 542-548; Table 6), signal anchor sorting (SEQ ID NO: 549-562; Table 7) and secretion (SEQ ID NO: 563-570; Table 8) sequences as provided herein can be used with any of the flagellin polypeptides or the flagellin-associated polypeptides to promote growth and provide health and protective benefits to a plant or a plant part.

Modification of Flg polypeptide Sequences Function

Any of the L or D Flg-associated sequences provided in Tables 3, 4 or 5 can be similarly modified as fused to any of the assistance sequences as described in Table 6-8. For one example, fusion of any of these assistance sequences will present a modification to the Bt.4Q7Flg22 bioactive priming polypeptide sequence identified as SEQ ID NO: 226.

Mutations to Flg-Associated Polypeptides to Increase Responsiveness to Reactive Oxygen Species or Polypeptide Stability

The flagellin or flagellin associated polypeptide useful in the compositions and methods herein can comprise a mutant flagellin or flagellin-associated polypeptide.

The mutant flagellin or flagellin-associated polypeptide can be derived from a Bacillus, a Lysinibacillus, a Paenibacillus, or an Aneurinibacillus genus bacterium. Other polypeptides from other Eubacterial classes, including Enterobacteraciae, can also be used in the same fashion. Other generas of interest include Pseudomonas, Escherichia, Xanthomonas, Burkholderia, Erwinia, and others.

The amino acid sequence of the flagellin or flagellin-associated polypeptide can comprise any one of SEQ ID NOs: 226, 289, 290, 291, 293, 294, 295, 300, 437, 526, 532, 534, 536, 538, 540, 571-585 and 587-603. For example, the amino acid sequence of the flagellin or flagellin-associated polypeptide can comprise any one of SEQ ID NOs: 226, 293, 295, 300, 540, 571-579, and 589-590, or any combination thereof. For example, the amino acid sequence of the flagellin or flagellin-associated polypeptide can comprise SEQ ID NO: 226, 571, 590 or any combination thereof. The amino acid sequence of the flagellin or flagellin-associated polypeptide can comprise SEQ ID NO: 226. The amino acid sequence of the flagellin or flagellin-associated polypeptide used in the compositions and methods herein can comprise SEQ ID NO: 590. The amino acid sequence of the flagellin or flagellin-associated polypeptide used in the compositions and methods herein can comprise SEQ ID NO: 571. The amino acid sequence of the flagellin or flagellin-associated polypeptide can comprise any one of SEQ ID NOs: 591-603.

The flagellin or flagellin-associated polypeptide can be modified chemically on its N or C terminus. Common modification of the N and C-termini include: acetylation, lipid addition, urea addition, pyroglutamyl addition, carbamate addition, sulfonamide addition, alkylamide addition, biotinylation, phosphorylation, glycosylation, PEGylation, methylation, biotinylation, acid addition, amide addition, ester addition, aldehyde addition, hydrazide addition, hydroxyamic acid addition, chloromethyl ketone addition, or addition of purification tags. These tags can increase activity of the polypeptides, increase stability, add protease inhibitor abilities to the polypeptides, block proteases directly, allow for tracking, and help in binding to plant tissues.

The flagellin or flagellin-associated polypeptide can be modified via crosslinking or cyclization. Crosslinking can bind polypeptides either to each other or to a secondary surface or moiety to help in delivery or stability of the polypeptides. Cyclization can be performed, for example, to both increase activity of the polypeptide as well as prevent protease interaction with the polypeptide.

Sequence modifications or mutations can be made to any amino acid sequence(s) as described in Tables 4 and 5 and replaced with any of the 20 standard amino acid sequences known in nature or replaced with a nonstandard or non-canonical amino acid sequence, such as selenocysteine, pyrrolysine, N-formylmethione, etc. For example, modifications or mutations can be made to the internal sequences as shown in SEQ ID NO: 571, to the C-terminis as shown in SEQ ID NO: 572 or SEQ ID NO: 589, or to the N terminus as shown in SEQ ID NO: 573 to produce Flg polypeptides with enhanced ROS activates and increased functionality in a plant or plant part. Modified polypeptides also can be truncated at the N or C terminus as shown in SEQ ID NO: 590 (N-terminus truncation) to further increase functionality in a plant or plant part. Table 9 summarizes flagellin polypeptides identified that provide modified ROS activity.

TABLE 9
Flagellin polypeptides Flg22 identified from Bacillus or other bacteria with
mutations that provide modified ROS activity

| SEQ ID NO: | Peptide Flg22 |
|---|---|
| Flg22-Bt4Q7 | DRLSSGKRINSAKDDAAGLAIA |
| SEQ ID NO:- 571 | |
| Bacillus thuringiensis | |
| strain 4Q7 | |
| Modified FLG22-Bt4Q7 (S13K); | |
| Syn01 | |
| Flg22-Bt4Q7 | DRLSSGKRINSASDDAAGLQIA |
| SEQ ID NO: 572 | |
| Bacillus thuringiensis | |
| strain 4Q7 | |
| Modified FLG22-Bt4Q7 (A20Q); | |
| Syn02 | |
| Flg22-Bt4Q7 | QRLSSGKRINSASDDAAGLAIA |
| SEQ ID NO: 573 | |
| Bacillus thuringiensis | |
| strain 4Q7 | |
| Modified FLG22-Bt4Q7 (D1Q); | |
| Syn03 | |
| Flg22-Bt4Q7 | NRLSSGKRINSASDDAAGLAIA |
| SEQ ID NO: 574 | |
| Bacillus thuringiensis | |
| strain 4Q7 | |
| Modified FLG22-Bt4Q7 (D1N); | |
| Syn06 | |
| Caballeronia megalochromosomata | TRLSSGKRINSASDDAAGLAIA |
| SEQ ID NO: 575 | |
| Flg22-Bt4Q7 | DRLSSGYRINSASDDAAGLAIA |
| SEQ ID NO: 576 | |
| Bacillus thuringiensis | |
| strain 4Q7 | |
| Modified FLG22-Bt4Q7 (K7Y); | |
| Syn07 | |
| Flg22-Bt4Q7 | DRLSSGFRINSASDDAAGLAIA |
| SEQ ID NO: 577 | |
| Bacillus thuringiensis | |
| strain 4Q7 | |
| Modified FLG22-Bt4Q7 (K7F); Syn08 | |
| Flg22-Bt4Q7 | DRLSSGKRINSASDDPAGLAIA |
| SEQ ID NO: 578 | |
| Bacillus thuringiensis | |
| Modified FLG22-Bt4Q7 (A16P); | |
| Syn05 | |
| Flg22-Bt4Q7 | DRLSSGQRINSASDDAAGLAIA |
| SEQ ID NO: 579 | |
| Bacillus thuringiensis | |
| strain 4Q7 | |
| Modified FLG22-Bt4Q (K7Q); | |
| Syn09 | |
| Flg22-Bt4Q7 | DRLSSGKRINSASDPAAGLAIA |
| SEQ ID NO: 589 | |
| Bacillus thuringiensis | |
| strain 4Q7 | |
| Modified FLG22-Bt4Q7 (D15P); | |
| Syn04 | |
| Flg15-Bt4Q7 | RINSAKDDAAGLAIA |
| SEQ ID NO: 590 | |
| Bacillus thuringiensis | |
| N-term Truncated Syn01 | |
| Bm.Flg22-B1 | NRLSSGKQINSASDDAAGLAIA |
| Bacillus manliponensis | |
| SEQ ID NO: 290 | |
| Ba.Flg22-B2 | NRLSSGKRINSAADDAAGLAIA |
| Bacillus anthracis | |
| SEQ ID NO: 295 | |
| Bc.Flg22-B3 | DRLSSGKRINNASDDAAGLAIA |
| Bacillus cereus | |
| SEQ ID NO: 294 | |
| A. spp.Flg22-B4 | ERLSSGYRINRASDDAAGLAIS |
| Aneurinibacillus spp. XH2 | |
| SEQ ID NO: 300 | |
| Ba.Flg22-B5 | EKLSSGQRINSASDDAAGLAIS |
| Bacillus aryabhattai | |
| SEQ ID NO: 289 | |
| P spp.Flg22-B6 | GKLSSGLRINGASDDAAGLAIS |
| Paenibacillus spp. strain HW567 | |
| SEQ ID NO: 293 | |
| L spp.Flg22-L1 | LRLSSGYRINSAADDAAGLAIS |
| Lysinibacillus spp. | |
| SEQ ID NO: 291 | |
| L spp.Flg22-L2 | EKLSSGLRINRAGDDAAGLAIS |
| Lysinibacillus spp. | |
| SEQ ID NO: 580 | |
| L spp.Flg22-L3 | EKLSSGYKINRASDDAAGLAIS |
| Lysinibacillus spp. | |
| SEQ ID NO: - 581 | |
| L spp.Flg22-L4 | LRISSGYRINSAADDPAGLAIS |
| Lysinibacillus spp. SG9 | |
| SEQ ID NO: 582 | |
| Lf.Flg22-L5 | LRISTGYRINSAADDPAGLAIS |
| Lysinibacillus fusiformis | |
| SEQ ID NO: 583 | |
| Lm.Flg22-L6 | EKLSSGFRINRAGDDAAGLAIS |
| Lysinibacillus macroides | |
| SEQ ID NO: 584 | |
| Lx.Flg22-L6 | EKLSSGYKINRAGDDAAGLAIS |
| Lysinibacillus | |
| xylanilyticus | |
| SEQ ID NO: 585 | |
| Pa.Flg22 | QRLSTGSRINSAKDDAAGLQIA |
| Pseudomonas aeruginosa | |
| SEQ ID NO: 530 | |
| Ec.Flg22 | ERLSSGLRINSAKDDAAGQAIA |
| Escherichia coli | |
| SEQ ID NO: 526 | |
| Xcc.Flg22 | QRLSSGLRINSAKDDAAGLAIS |
| Xanthomonas campestris pv | |
| campestris strain 305 or | |
| (Xanthomonas citri pv. citri) | |
| SEQ ID NO: 532 | |
| Ea.Flg22 | QRLSSGLRINSAKDDAAGQAIS |
| Erwinia amylovora | |
| SEQ ID NO: 534 | |
| Bp.Flg22 | TRLSSGKRINSAADDAAGLAIS |
| Burkholderia phytofirmans strain | |
| Ps.IN | |
| SEQ ID NO: 536 | |
| Bu.Flg22 | NRLSSGKRINTAADDAAGLAIS |
| Burkholderia ubonensis | |
| SEQ ID NO: 538 | |
| Ps.Flg22 | TRLSSGLKINSAKDDAAGLQIA |
| Pseudomonas syringae pv. actinidiae | |
| ICMP 19096 | |
| SEQ ID NO: 540 | |

### Core Active Domain of Flg22

The underlined portions of the sequences in Table 9 represent the core active domain of Flg22. This core domain comprises, for example, SEQ ID NO: 591 with up to one, two or three amino acid substitutions (represented by SEQ ID NOs 592-603) that can promote growth, disease reduction and/or prevention in crops, horticultural, and ornamental plants. For ease of reference, this core domain is represented as the consensus sequence having the SEQ ID NO: 603. The various native and mutant Flg22 polypeptides comprising SEQ ID NOs 591-603 are described along with the consensus sequence in Table 10, below. Therefore, the polypeptides used in the compositions and methods herein can further comprise a core sequence. The core sequence can comprise any one of SEQ ID NOs 591-603.

The polypeptide used in any of the compositions or methods herein can also comprise any polypeptide comprising any one of SEQ ID NOs 1-590, 604-778 and 794-796 wherein the polypeptide further comprises the core sequence comprising any one of SEQ ID NOs: 591-603. The inclusion of the core sequence in the polypeptide or full-length protein of dissimilar function can increase the bioactive priming activity of the polypeptide, and any composition comprising the polypeptide.

TABLE 10
Flg22 core sequence with variants

| | | Polypeptides comprising |
|---|---|---|
| SEQ ID NO: | FLG22 core sequence | core sequence |
| SEQ ID NO: 591 | RINSASDD | SEQ ID NO: 226-229 |
| | | SEQ ID NO: 289 |
| | | SEQ ID NO: 299 |
| | | SEQ ID NO: 536 |
| | | SEQ ID NO: 572-579 |
| SEQ ID NO: 592 | RINNASDD | SEQ ID NO: 231-234 |
| | | SEQ ID NO: 236-240 |
| | | SEQ ID NO: 243-246 |
| | | SEQ ID NO: 248 |
| | | SEQ ID NO: 250-256 |
| | | SEQ ID NO: 258-259 |
| | | SEQ ID NO: 261 |
| | | SEQ ID NO: 263 |
| | | SEQ ID NO: 265-270 |
| | | SEQ ID NO: 272-280 |
| | | SEQ ID NO: 282-283 |
| | | SEQ ID NO: 285-286 |
| | | SEQ ID NO: 288 |
| | | SEQ ID NO: 294 |
| SEQ ID NO: 593 | QINSASDD | SEQ ID NO: 290 |
| SEQ ID NO: 594 | RINSAADD | SEQ ID NO: 291-292 |
| | | SEQ ID NO: 295-298 |
| | | SEQ ID NO: 582-583 |
| | | SEQ ID NO: 536 |
| | | SEQ ID NO: 582-583 |
| SEQ ID NO: 595 | RINGASDD | SEQ ID NO: 293 |
| SEQ ID NO: 596 | RINRASDD | SEQ ID NO: 300 |
| SEQ ID NO: 597 | RINSAKDD | SEQ ID NO: 526 |
| | | SEQ ID NO: 528 |
| | | SEQ ID NO: 530 |
| | | SEQ ID NO: 532 |
| | | SEQ ID NO: 534 |
| | | SEQ ID NO: 571 |
| | | SEQ ID NO: 586 |
| SEQ ID NO: 598 | RINTAADD | SEQ ID NO: 538 |
| SEQ ID NO: 599 | KINSAKDD | SEQ ID NO: 540 |
| SEQ ID NO: 600 | RINRAGDD | SEQ ID NO: 580 |
| | | SEQ ID NO: 584 |
| SEQ ID NO: 601 | KINRASDD | SEQ ID NO: 581 |
| SEQ ID NO: 602 | KINRAGDD | SEQ ID NO: 585 |
| SEQ ID NO: 603 | (R/Q/K)IN(S/N/G/R/T)A(S/A/K/G)DD | Consensus of SEQ ID NO: |
| | | 591-602 (sequences |
| | | identified in this Table) |

Root Hair Promoting Polypeptide (RHPP)

The composition can comprise at least one RHPP.

The amino acid sequence of the RHPP can comprise any one of SEQ ID NO: 604, 607, 608, and 745-755. For example, the amino acid sequence of the RHPP can comprise SEQ ID NO: 604.

A combination of the polypeptide comprising an RHPP and a polypeptide comprising a flagellin or flagellin associated polypeptide is also provided. The flagellin or flagellin associated polypeptide can comprise any one of SEQ ID NO: 226, 590, and 571. In some instances, the composition comprises an RHPP comprising SEQ ID NO: 604 and a flagellin comprising SEQ ID NO: 226. In other instances, the composition comprises an RHPP comprising SEQ ID NO: 604 and a flagellin comprising SEQ ID NO: 571.

Additional RHPP bioactive priming polypeptides can be derived from the full length Kunitz Trypsin Inhibitor protein from Glycine max comprising SEQ ID NO: 606 or can be obtained from additional species (Table 12). The RHPP polypeptide can be modified via C-terminal amidation, N-terminal acetylation or other modification. The RHPP bioactive priming polypeptide can be obtained through addition of crude protease digest of kunitz trypsin inhibitor and/or soybean meal.

RHPP can be provided, for example, as a foliar application to produce beneficial phenotypes in corn, soybean and other vegetables or in citrus plants. For example, foliar application of RHPP can increase row crop and vegetable yield and/or improve disease symptoms and/or improve juice quality and crop yield in citrus plants.

TABLE 11
Amino acid sequence for RHPP forward and retro-inverso sequences

| SEQ ID NO: | Peptide Sequence Amino Acid |
|---|---|
| Root Hair Promoting Peptide | GGIRAAPTGNER |
| (RHPP) | |
| SEQ ID NO: 604 | |
| Glycine max | |
| MW 1198.20 Da | |
| Root Hair Promoting Peptide | RENGTPAARIGG |
| (Retro Inverso RHPP) | |
| SEQ ID NO:605 | |
| Glycine max | |
| MW 1198.20 Da | |
| KunItz Trypsin Inhibitor | MKSTIFFALFLFCAFTTSYLPSAIADFVLDNEGNPLENGG |
| SEQ ID NO: 606 | TYYILSDITAFGGIRAAPTGNERCPLTVVQSRNELDKGIET |
| Glycine Max | IISSPYRIRFIAEGHPLSLKFDSFAVIMLCVGIPTEWSVVED |
| | LPEGPAVKIGENKDAMDGWFRLERVSDDEFNNYKLVFC |
| | PQQAEDDKCGDIGISIDHDDGTRRLVVSKNKPLVVQFQK |
| | LDKESLAKKNHGLSRSE |

TABLE 12
Homologs and Variants of RHPP

| SEQ ID NO: | Peptide Sequence Amino Acid |
|---|---|
| Homolog RHPP | GGIRATPTENER |
| SEQ ID NO: 607 | |
| Glycine max | |
| Homolog RHPP | GGIRVAATGKER |
| SEQ ID NO: 608 | |
| Glycine max Glycine soja | |
| Root Hair Promoting Peptide (RHPP)-Pp | GGIRAAPT |
| SEQ ID NO: 745 | |
| Physcomitrella patens | |
| Root Hair Promoting Peptide (RHPP)-Mc | GIRDAPAGNE |
| SEQ ID NO: 746 | |
| Macleaya cordata | |
| Root Hair Promoting Peptide (RHPP)-Bd | GGARAAPAGEER |
| SEQ ID NO: 747 | |
| Brachypodium distachyon | |
| Root Hair Promoting Peptide (RHPP)-Va | GGIRTAITGNE |
| SEQ ID NO: 748 | |
| Vigna angularis | |
| Root Hair Promoting Peptide (RHPP)-Ls | GGISASPTGN |
| SEQ ID NO: 749 | |
| Lactuca sativa | |
| Root Hair Promoting Peptide (RHPP)-Vr | GGIRRARTGNE |
| SEQ ID NO: 750 | |
| Vigna radiata | |
| Syn01 Root Hair Promoting Peptide (RHPP) | GGIRRAPTGNER |
| SEQ ID NO: 751 | |
| Syn02 Root Hair Promoting Peptide (RHPP) | GGIRDAPTGNER |
| SEQ ID NO: 752 | |
| Syn03 Root Hair Promoting Peptide (RHPP) | GGIRAARTGNER |
| SEQ ID NO: 753 | |
| Syn04 Root Hair Promoting Peptide (RHPP) | GGIRAAPTGKER |
| SEQ ID NO: 754 | |
| Syn05 Root Hair Promoting Peptide (RHPP) | GGIRASPTGNER |
| SEQ ID NO: 755 | |

The polypeptide can comprise at least one retro inverso (RI) RHPP.

The retro inverso RHPP can have any amino acid sequence that comprises any one of SEQ ID NOs: 605, 609, 610 or 756-766 (Table 13).

The retro inverso (RI) RHPP can be modified via C-terminal amidation or N-terminal acetylation.

TABLE 13
Retro inverso amino acid sequences for homologs and variants of RHPP.

| SEQ ID NO: | Peptide Sequence Amino Acid |
|---|---|
| Retro-Inverso Homolog RHPP | RENETPTARIGG |
| SEQ ID NO: 609 | |
| Glycine max | |
| Retro-Inverso Homolog RHPP | REKGTAAVRIGG |
| SEQ ID NO: 610 | |
| Glycine max Glycine soja | |
| Retro-Inverso Root Hair Promoting Peptide | TPAARIGG |
| (RHPP)-Pp | |
| SEQ ID NO: 756 | |
| Physcomitrella patens | |
| Retro-Inverso Root Hair Promoting Peptide | ENGAPADRIG |
| (RHPP)-Mc | |
| SEQ ID NO: 757 | |
| Macleaya cordata | |
| Retro-Inverso Root Hair Promoting Peptide | REEGAPAARAGG |
| (RHPP)-Bd | |
| SEQ ID NO: 758 | |
| Brachypodium distachyon | |
| Retro-Inverso Root Hair Promoting Peptide | ENGTIATRIGG |
| (RHPP)-Va | |
| SEQ ID NO: 759 | |
| Vigna angularis | |
| Retro-Inverso Root Hair Promoting Peptide | NGTPSASIGG |
| (RHPP)-Ls | |
| SEQ ID NO: 760 | |
| Lactuca sativa | |
| Retro-Inverso Root Hair Promoting Peptide | ENGTRARRIGG |
| (RHPP)-Vr | |
| SEQ ID NO: 761 | |
| Vigna radiate | |
| Retro-Inverso Syn01 Root Hair Promoting | RENGTPARRIGG |
| Peptide (RHPP) | |
| SEQ ID NO: 762 | |
| Retro-Inverso Syn02 Root Hair Promoting | RENGTPADRIGG |
| Peptide (RHPP) | |
| SEQ ID NO: 763 | |
| Retro-Inverso Syn03 Root Hair Promoting | RENGTRAARIGG |
| Peptide (RHPP) | |
| SEQ ID NO: 764 | |
| Retro-Inverso Syn04 Root Hair Promoting | REKGTPAARIGG |
| Peptide (RHPP) | |
| SEQ ID NO: 765 | |
| Retro-Inverso Syn05 Root Hair Promoting | RENGTPSARIGG |
| Peptide (RHPP) | |
| SEQ ID NO: 766 | |

The RHPP and RI-RHPPs described in Tables 11 to 13 can also be provided as isolated polypeptides. Accordingly, an isolated polypeptide is provided wherein the polypeptide has an amino acid sequence comprising or consisting of any one of SEQ ID NOs: 745-766.

For example, the amino acid sequence of the isolated polypeptide can consist of any one of SEQ ID NOs: 745-766.

The amino acid sequence of the isolated polypeptide can comprise or consist of any one of SEQ ID NOs: 746-755 and 757-766.

The amino acid sequence of the isolated polypeptide can comprise or consist of any one of SEQ ID NOs: 746-755 and 757-766.

The amino acid sequence of the isolated polypeptide can comprise or consist of any one of SEQ ID NOs: 746-750 and 757-761.

The amino acid sequence of the isolated polypeptide can comprise or consist of any one of SEQ ID NOs: 746, 748, 749, 750, 757, 759, 760, and 761.

The amino acid sequence of the isolated polypeptide can comprise or consist of any one of SEQ ID NOs: 747 and 758.

### Thionins and Thionin-Targeting Polypeptides

The composition can comprise at least one thionin or thionin-like polypeptide.

The thionin or thionin-like polypeptide can be fused to a phloem targeting sequence to form a fused polypeptide The amino acid sequence of the phloem targeting sequence can comprise any one of SEQ ID NOs: 611-619, or any combination thereof, for delivering the fused polypeptide to vascular tissue or cells and/or phloem or phloem-associated tissue or cells in the plant or plant part.

The amino acid sequence of the phloem targeting sequence can comprise SEQ ID NO: 611.

More specifically, targeting sequences useful for targeting AMP polypeptides, such as thionins or Flg polypeptides to the vascular tissues (xylem and phloem) can be extremely useful for treating diseases that colonize restricted tissues involved in the transport of fluids and nutrients (e.g., water soluble nutrients, sugars, amino acids, hormones, etc.). Vascular tissues such as the xylem transport and store water and water-soluble nutrients and the phloem cells transport sugars, proteins, amino acids, hormones and other organic molecules in plants.

Preferred vascular/phloem targeting polypeptides useful for targeting the thionins and flagellin-associated polypeptides as described herein are provided in Table 14.

TABLE 14
Phloem targeting polypeptides

| SEQ ID NO: | Vascular/Phloem targeting polypeptides |
|---|---|
| Phloem targeting peptide | MSTATFVDIIIAILLPPLGVFLRFGCGVE |
| Synthetic | FWICLVLTLLGYIPGIIYAIYVLTK |
| SEQ ID NO: 611 | |
| Salt stress induced targeting peptide | MGSETFLEVILAILLPPVGVFLRYGCGV |
| Citrus clementina | EFWICLLLTVLGYIPGIIYAIYVLVG |
| SEQ ID NO: 612 | |
| Hypothetical protein CICLE | MGTATCVDIILAVILPPLGVFLKFGCKA |
| Citrus trifoliata | EFWICLLLTILGYIPGIIYAVYVITK |
| SEQ ID NO: 613 | |
| Hypothetical protein CICLE | MADEGTATCIDIILAIILPPLGVFLKFGC |
| Citrus sinensis | KVEFWICLLLTIFGYIPGIIYAVYAITKN |
| SEQ ID NO: 614 | |
| Low temperature and salt responsive protein | MADGSTATCVDILLAVILPPLGVFLKFG |
| Citrus sinensis | CKAEFWICLLLTILGYIPGIIYAVYAITK |
| SEQ ID NO: 615 | K |
| Hypothetical protein CICLE | FYKQKYQVQITKAVTQNPKHFFNQSSC |
| Citrus | FLTLNFILFHFTLFKNQSKMADGSTATC |
| clementina | VDILLAVILPPLGVFLKFGCKAEFWICL |
| SEQ ID NO: 616 | LLTILGYIPGIIYAVYAITKK |
| Low temperature and salt responsive protein | MSTATFVDIIIAILLPPLGVFLRFGCGVE |
| Arabidopsis thaliana | FWICLVLTLLGYIPGIIYAIYVLTK |
| SEQ ID NO: 617 | |
| Cold-inducible protein | MSTATFVDIIIAVLLPPLGVFLRFGCGV |
| Camelina sativa | EFWICLVLTLLGYIPGIIYAIYVLTK |
| SEQ ID NO:618 | |
| Low temperature and salt responsive protein | MGTATCVDIIIAILLPPLGVFLRFGCGVE |
| Arabidopsis lyrata | FWICLVLTLLGYIPGILYALYVLTK |
| SEQ ID NO: 619 | |

A synthetic version of a phloem targeting polypeptide (SEQ ID NO: 611) is particularly useful in targeting anti-microbial polypeptides to the phloem sieve tube and companion cells.

Anti-microbial thionin polypeptides are also provided (Table 15) and are utilized with the phloem targeting sequences provided in Table 14 for targeting the thionin sequences into the phloem tissues of citrus as well as other plants.

The amino acid sequence of the thionin or thionin-like polypeptide can comprise any one of SEQ ID NOs: 620-719, such as SEQ ID NO: 620.

TABLE 15
Thionin and thionin-like sequences

| SEQ ID NO: | Thionin or Thionin-like Sequences- Amino Acid |
|---|---|
| Thionin-like protein | RTCESQSHRFKGPCSRDSNCATVCLTEGFSG |
| Synthetic | GDCRGFRRRCRCTRPCVFDEK |
| SEQ ID NO: 620 | |
| Thionin-like protein | RVCQSQSHHFHGACFSHEINCAFVCRNEGFS |
| Citrus sinensis | GGKCRGVRRRCFCSKLC |
| SEQ ID NO: 621 | |
| Thionin-like protein | KSCCKDIMARNCYNVCRIPGTPRPVCATTCR |
| Avena sativa | CKIISGNKCPKDYPK |
| SEQ ID NO: 622 | |
| Thionin-like protein | RTCESQSHRFKGPCSRDSNCATVCLTEGFSG |
| Synthetic | GDCRGFRRRCRCTRPCVFDEK |
| SEQ ID NO: 623 | |
| Thionin-like protein | MDSRSFGLLPLLLLILLTSQMTVLQTEARLCE |
| Citrus sinensis | SQSHRFHGTCVRSHNCDLVCRTEGFTGGRC |
| SEQ ID NO: 624 | RGFRRRCFCTRIC |
| Proteinase inhibitor se60-like protein | MKSFFGIFLLLLILFASQEIMVPAEGRVCQSQ |
| Citrus paradise | SHHFHGACFSHEINCAFVCRNEGFSGGKCRG |
| SEQ ID NO: 625 | VRRRCFCSKLC |
| Defensin precursor | MKSFFGIFLLLLILFASQMMVPAEGRVCQSQ |
| Citrus clementina | SHHFHGACFSHEINCAFVCRNEGFSGGKCRG |
| SEQ ID NO: 626 | ARRRCFCSKLC |
| defensin precursor | MKSFFGIFLLLLILFASQEMMVPAEGRVCQS |
| Citrus clementina | QSHHFHGACFSHEINCAFVCRNEGFSGGKCR |
| SEQ ID NO: 627 | GARRRCFCSKLC |
| Thionin-like protein | MKSFFGIFLLLLILFASQMMVPAEGRVCQSQ |
| Citrus clementina | SHHFHGACFSHEINCAFVCRNEGFSGGKCRG |
| SEQ ID NO: 628 | ARRRCFCSKLC |
| Thionin-like peptide | MANSMRFFATVLLLALLVMATEMGPMTIAE |
| Nicotiana benthamiana | ARTCESQSHRFKGPCSRDSNCATVCLTEGFS |
| SEQ ID NO: 629 | GGDCRGFRRRCFCTRPC |
| Thionin-like protein | MAKSMRFFATVLLLALLVMATEMGPTTIAE |
| Nicotiana sylvestris | ARTCESQSHRFKGPCSRDSNCATVCLTEGFS |
| SEQ ID NO: 630 | GGDCRGFRRRCFCTRPC |
| Thionin-like protein | MANSMRFFATVLLLTLLVMATEMGPMTIAE |
| Nicotiana tabaccum | ARTCESQSHRFKGPCSRDSNCATVCLTEGFS |
| SEQ ID NO: 631 | GGDCRGFRRRCFCTRPC |
| Thionin-like protein | MANSMRFFATVLLIALLVMATEMGPMTIAE |
| Nicotiana tomentosiformis | ARTCESQSHRFKGPCSRDSNCATVCLTEGFS |
| SEQ ID NO: 632 | GGDCRGFRRRCFCTRPC |
| Thionin-like protein | MANSMRFFATVLLIALLVTATEMGPMTIAEA |
| Nicotiana tabaccum | RTCESQSHRFKGPCSRDSNCATVCLTEGFSG |
| SEQ ID NO: 633 | GDCRGFRRRCFCTRPC |
| Defensin class I | MANSMRFFATVLLLTLLFMATEMGPMTIAE |
| Nicotiana alata | ARTCESQSHRFKGPCARDSNCATVCLTEGFS |
| SEQ ID NO: 634 | GGDCRGFRRRCFCTRPC |
| Leaf thionin | MGSIKGLKSVVICVLVLGIVLEQVQVEGKSC |
| Avena sativa | CKDIMARNCYNVCRIPGTPRPVCATTCRCKII |
| SEQ ID NO: 635 | SGNKCPKDYPKLHGDPD |
| Leaf thionin | MGSIKGLKSVVICVLVLGIVLEHVQVEGKSC |
| Avena sativa | CKDTTARNCYNVCRIPGTPRPVCATTCRCKII |
| SEQ ID NO: 636 | SGNKCPKDYPKLHGDLD |
| Thionin Class I | LGLVVAQTQVDAKSCCPSTAARNCYNVCRF |
| Tulipa gesneriana | PGTPRPVCAATCGCKIITGTKCPPDYPKLGW |
| SEQ ID NO: 637 | STFQNSDVADKALDVVDEALHVAKEVMKE |
| | AVERCNNACSEVCTKGSYAVTA |
| Thionin-like protein Class I | MERKSLGFFFFLLLILLASQEMVVPSEARVCE |
| Vitis vinifera | SQSHKFEGACMGDHNCALVCRNEGFSGGKC |
| SEQ ID NO: 638 | KGLRRRCFCTKLC |
| Thionin-like protein Class I | MERKSLGFFFFLLLILLASQMVVPSEARVCES |
| Vitis vinifera | QSHKFEGACMGDHNCALVCRNEGFSGGKC |
| SEQ ID NO: 639 | KGLRRRCFCTKLC |
| defensin Ec-AMP-D1 | MERSVRLFSTVLLVLLLLASEMGLRAAEARI |
| Citrus sinensis | CESQSHRFKGPCVSKSNCAAVCQTEGFHGG |
| SEQ ID NO: 640 | HCRGFRRRCFCTKRC |
| Antimicrobial Protein 1 (Ah- Amp1) | LCNERPSQTWSGNCGNTAHCDKQCQDWEK |
| Aesculus hippocastanum | ASHGACHKRENHWKCFCYFNC |
| SEQ ID NO: 641 | |
| hypothetical protein DCAR | MAKNSTSPVSLFAISLIFFLLANSGSITEVDGK |
| Dacus carota | VCEKPSLTWSGKCGNTQHCDKQCQDWEGA |
| SEQ ID NO: 642 | KHGACHSRGGWKCFCYFEC |
| Cysteine-rich antimicrobial protein | NLCERASLTWTGNCGNTGHCDTQCRNWES |
| Clitoria ternatea | AKHGACHKRGNWKCFCYFNC |
| SEQ ID NO: 643 | |
| hypothetical protein DCAR | MAKKSSSFCLSAIFLVLLLVANTGMVREVDG |
| Dacus carota | ALCEKPSLTWSGNCRNTQHCDKQCQSWEG |
| SEQ ID NO: 644 | AKHGACHKRGNWKCFCYHAC |
| Thionin-like | MAKKLNAVTVSAIFLVVFLIASYSVGAAKEA |
| Bupleurum kaoi | GAEGEVVFPEQLCERASQTWSGDCKNTKNC |
| SEQ ID NO: 645 | DNQCIQWEKARHGACHKRGGKWMCFCYFD |
| | KC |
| defensin Dm-AMP1 = cysteine-rich | ELCEKASKTWSGNCGNTGHCDNQCKSWEG |
| antimicrobial protein | AAHGACHVRNGKHMCFCYFNC |
| Dahlia merckii | |
| SEQ ID NO: 646 | |
| Thionin-like | MAKISVAFNAFLLLLFVLAISEIGSVKGELCE |
| Helianthus annuus | KASQTWSGTCGKTKHCDDQCKSWEGAAHG |
| SEQ ID NO: 647 | ACHVRDGKHMCFCYFNCSKAQKLAQDKLR |
| | AEELAKEKIEPEKATAKP |
| Thionin | MAKNSVAFFALLLLICILTISEFAVVKGELCE |
| Cynara cardunculus var. scolymus | KASKTWSGNCGNTRHCDDQCKAWEGAAH |
| SEQ ID NO: 648 | GACHTRNKKHMCFCYFNCPKAEKLAQDKL |
| | KAEELARDKVEAKEVPHFKHPIEPIHHP |
| Thionin | MAKQWVSFFALAFIVFVLAISETQTVKGELC |
| Cynara cardunculus var. scolymus | EKASKTWSGNCGNTKHCDDQCKSWEGAAH |
| SEQ ID NO: 649 | GACHVRNGKHMCFCYFNSCAEADKLSEDQI |
| | EAGKLAFEKAEKLDRDVKKAVPNVDHP |
| defensin-like protein 1 - DCAR-like | MAQKVNSALIFSAIFVLFLVASYSVTVAEGA |
| Daucus carota subsp. Sativus | RAGAEGEVVYPEALCERASQTWTGKCQHTD |
| SEQ ID NO: 650 | HCDNQCIQWENARHGACHKRGGNWKCFCY |
| | FDHC |
| low-molecular-weight cysteine-rich | MASSYTLMLFLCLSIFLIASTEMMAVEARICE |
| defensin | RRSKTWTGFCGNTRGCDSQCKSWERASHGA |
| Arabidopsis lyrata | CHAQFPGFACFCYFNC |
| SEQ ID NO: 651 | |
| Thionin-like protein | MAKSSTSYLVFLLLVLVVAISEIASVNGKVC |
| Parthenium hysterophorus | EKPSKTWFGNCKDTEKCDKRCMEWEGAKH |
| SEQ ID NO: 652 | GACHQRESKYMCFCYFDCDP |
| putative defensin AMP1 protein | MASSYTLMLFLCLSIFLIASTEMMAVEGRICE |
| Arabidopsis thaliana | RRSKTWTGFCGNTRGCDSQCKRWERASHG |
| SEQ ID NO: 653 | ACHAQFPGFACFCYFNC |
| Thionin-like | MASSYTLLLFVCLSIFFIASTEMMMVEGRVC |
| Eutrema salsugineum | ERRSKTWTGFCGNTRGCDSQCKRWERASHG |
| SEQ ID NO: 654 | ACHAQFPGFACFCYFNC |
| defensin-like | MAKLLGYLLSYALSFLTLFALLVSTEMVML |
| Vitis vinifera | EAKVCQRPSKTWSGFCGSSKNCDRQCKNWE |
| SEQ ID NO: 655 | GAKHGACHAKFPGVACFCYFNC |
| Knottin | MAKSLSSFATFLALLCLFFLLSTPNEMKMAE |
| Corchorus olitorius | AKICEKRSQTWSGWCGNSSHCDRQCKNWE |
| SEQ ID NO: 656 | NARHGSCHADGLGWACFCYFNC |
| Knottin | MEMKMAEGKICEKRSQTWSGWCGNSSHCD |
| Corchorus olitorius | RQCKNWENARHGSCHADGLGWACFCYFNC |
| SEQ ID NO: 657 | |
| Thionin-like protein Camelina sativa | MASSLKLMLFLCLSIFLIASTEMMTVEGRTC |
| SEQ ID NO: 658 | ERRSKTWTGFCGNTRGCDSQCRSWEGASHG |
| | ACHAQFPGFACFCYFNC |
| Thionin-like protein Cucumis sativus | MAKVVGNSAKMIVALLFLLALMLSMNEKQ |
| SEQ ID NO: 659 | GVVEAKVCERRSKTWSGWCGNTKHCDRQC |
| | KNWEGATHGACHAQFPGRACFCYFNC |
| Thionin-like protein | MIDAFNYKQFSTVKGKICEKPSKTWFGKCQ |
| Cynara cardunculus var. scolymus | DTTKCDKQCIEWEDAKHGACHERESKLMCF |
| SEQ ID NO: 660 | CYYNCGPPKNTPPGTPPSPP |
| Thionin-like | MASSYKLILFLCLSIFLIASFEMMAVEGRICQ |
| Capsella rubella | RRSKTWTGFCGNTRGCDSQCKRWERASHG |
| SEQ ID NO: 661 | ACHAQFPGFACFCYFNC |
| Thionin | MMAVEGRICERRSKTWTGFCGNTRGCDSQC |
| Arabidopsis thaliana | KRWERASHGACHAQFPGFACFCYFNC |
| SEQ ID NO: 662 | |
| Thionin | MASSYTRLLLLCLSIFLIASTEVMMVEGRVC |
| Brassica napus | QRRSKTWTGFCGNTRGCDSQCKRWERASH |
| SEQ ID NO: 663 | GACHAQFPGFACFCYFNC |
| Thionin-like protein Brassica rapa | MASSYARLLLLCLSIFLIASTEVNINIVEGRVC |
| SEQ ID NO: 664 | QRRSKTWTGFCGNTRGCDSQCKRWERASH |
| | GACHAQFPGFACFCYFNC |
| Thionin-like protein Camelina sativa | MASSLKLMLFLCLSIFLIASTEMMTVEGRTC |
| SEQ ID NO: 665 | ERRSKTWTGFCGNTRGCDSQCRRWEHASHG |
| | ACHAQFPGFACFCYFNC |
| defensin-like protein Brassica napus | MASYTRLLLLCLSIFLIASTEVNINIVEGRVCQ |
| SEQ ID NO: 666 | RRSKTWTGFCGNTRGCDSQCKRWERASHG |
| | ACHAQFPGFACFCYFNC |
| Thionin-like protein Vitis vinifera | MVMLEAKVCQRPSKTWSGFCGSSKNCDRQ |
| SEQ ID NO: 667 | CKNWEGAKHGACHAKFPGVACFCYFNC |
| Thionin-like protein | MTKSFILVALLCICFILLSPTEMRLTLNACLK |
| Brassica napus | LAEAKICEKYSQTWSGRCTKTSHCDRQCIN |
| SEQ ID NO: 668 | WEDARHGACHQDKHGRACFCYFNCKK |
| Thionin-like protein | MASSYTVFLLLCLSIFLIASTEVMMVEGRVC |
| Raphanus sativus | QRRSKTWTGFCGNTRGCDSQCKRWEHASH |
| SEQ ID NO: 669 | GACHAQFPGFACFCYFNC |
| Thionin-like | MASSYTLLLFLCLSIFLIVSTEMNINIVEGRICE |
| Arabis alpine | RRSKTWTGFCANTRGCDSQCKRWERASHG |
| SEQ ID NO: 670 | ACHAQFPGVACFCYFNC |
| Thionin-like protein | MAKVVGNSAKMIVAFLFLLALTLSMNEKQG |
| Cucumis melo | VVEAKVCERRSKTWSGWCGDTKHCDRQCK |
| SEQ ID NO: 671 | NWEGAKHGACHAQFPGRACFCYFNC |
| Thionin-like protein | MAASLVYRLSSVILIVLLLFIMLNNEVMVVE |
| Erythranthe guttate | SRLCERRSKTWTGFCGSSNNCNNQCRNWER |
| SEQ ID NO: 672 | ASHGACHAQFPGFACFCYFNC |
| Thionin-like protein Sesamum indicum | MAKFQVSSTIFFALFFCFLLLASNEAKICQRNI |
| SEQ ID NO: 673 | SKTWSGVCLNSGNCDRQCRNWERAQHGAC |
| | HRRGLGFACLCYFKC |
| Thionin-like protein | MAKNSVAFFAFLLILFVLAISEIGSVKGELCE |
| Eclipta prostrata | KASQTWSGTCRITSHCDNQCKSWEGAAHGA |
| SEQ ID NO: 674 | CHVRGGKHMCFCYFSHCAKAEKLTQDKLK |
| | AGHLVNEKSEADQKVPVTP |
| Gamma thionin Cynara cardunculus var. | MAKNTKVSAFLFVFLFVFFLVVHSVTAFAIR |
| scolymus | FKCFDTDMLLKVIADMVVGMKGIEKVCRRR |
| SEQ ID NO: 675 | SKTWSGYCGDSKHCDQQCREWEGAEHGAC |
| | HHEGLGRACFCYFNC |
| Art v 1 precursor Ambrosia artemisiifolia | MAAGLLVFVLAISEIASVKGKLCEKPSVTWS |
| SEQ ID NO: 676 | GKCKVKQTDKCDKRCIEWEGAKHGACHKR |
| | DSKASCFCYFDCDPTKNPGPPPGAPKGKAPA |
| | PSPPSGGGGEGGGEGGGER |
| Art v 1 precursor Ambrosia | MAAGLLVFVLAISEIASVKGKLCEKPSLTWS |
| artemi679siifolia | GKCKVKQTDKCDKRCIEWEGAKHGACHKR |
| SEQ ID NO: 677 | DSKATCFCYFDCDPTKNPGPPPGAPKGKAPA |
| | PSPPSGGGAPPPSGGEGGER |
| Thionin-like protein Jatropha curcas | MAKLHSSALCFLIIFLFLLVSKEMAVTEAKLC |
| SEQ ID NO: 678 | QRRSKTWSGFCGDPGKCNRQCRNWEGASH |
| | GACHAQFPGFACFCYFKC |
| Thionin-like protein Nelumbo nucifera | MAKAPKSVSYFAFFFILFLLASSEIQKTKKLC |
| SEQ ID NO: 679 | ERRSKTWSGRCTKTQNCDKQCKDWEYAKH |
| | GACHGSWFNKKCYCYFDC |
| Thionin-like protein Pyrus x | MAKLLSRLSIPLIVFVFLLILLASTEVAMVEA |
| bretschneideri | RICQRRSKTWSGFCANTGNCNRQCTNWEGA |
| SEQ ID NO: 680 | LHGACHAQFPGVACFCYFRC |
| Low-molecular-weight cysteine-rich | MAKLHFPTLLCLFIFLFLLVSTEMQVTQAKV |
| protein LCR78 precursor | CQRRSKTWSGFCGSTKNCDRQCKNWEGAL |
| Ricinus communi | HGACHAQFPGVACFCYFKCGGER |
| SEQ ID NO: 681 | |
| homologue of Art v 1 precursor | KLCEKPSVTWSGKCKVKQTDKCDKRCIEWE |
| Ambrosia artemisiifolia | GAKHGACHKRDSKASCFCYFDCDPTKNPGP |
| SEQ ID NO: 682 | PPGAPKGKAPAPSPPSGGGAPPPSGGEGGGD |
| homologue of Art v 1 precursor | KLCEKPSVTWSGNKVKQTDKCDKRCIEWEG |
| Ambrosia artemisiifolia | AKHGACHKRDSKASCFCYFDCDPTKNPGPP |
| SEQ ID NO: 683 | PGAPKGKAPAPSPPSGGGAPPPSGGEGGGDG |
| | GGGRR |
| Thionin-like protein | MAKLLSHLLFYPILFLFLFIFLASTEVAILEARI |
| Prunus mume | CQRRSKTWSGFCGNTRNCNRQCRNWEGAL |
| SEQ ID NO: 684 | RGACHAQFPGFACFCYFRC |
| Knottin | MAKTLQLFALFFIVILLANQEIPVAEAKLCQK |
| Corchorus olitorius | RSKTWTGICIKTKNCDNQCKKWEKAEHGAC |
| SEQ ID NO: 685 | HRQGIGFACFCYFNQKKC |
| Knottin | MAKFVSTVALLFALFILLASFDEGMMPMAE |
| Corchorus olitorius | AKVCSKRSKTWSGFCNSSANCNKQCREWED |
| SEQ ID NO: 686 | AKHGACHFEFPGFACFCYFNC |
| Thionin-like protein Solanum pennellii | MNSKVILALLVCFLLIASNEMQGGEAKVCG |
| SEQ ID NO: 687 | RRSSTWSGLCLNTGNCNTQCIKWEHASSGA |
| | CHRDGFGFACFCYFNC |
| Thionin-like protein | MAKLLGYHLVYPILFLFIFLLLASTEMGMLE |
| Fragaria vesca subsp. Vesca | ARICQRRSKTWTGLCANTGNCHRQCRNWE |
| SEQ ID NO: 688 | GAQRGACHAQFPGFACFCYFNC |
| Knottin | MAKFVSVALLLALFILVASFDEGMVPMAEA |
| Corchorus capsularis | KLCSKRSKTWSGFCNSSANCNRQCREWEDA |
| SEQ ID NO: 689 | KHGACHFEFPGFACFCYFDC |
| Thionin-like protein Solanum tuberosum | MQGGEARVCERRSSTWSGPCFDTGNCNRQC |
| SEQ ID NO: 690 | INWEHASSGACHREGIGSACFCYFNC |
| Defensin 1.2-like protein PDF1.2-1 | MAKTLKSVQFFALFFLVILLAGSEMTAVEAL |
| Dimocarpus longan | CSKRSKTWSGPCFITSRCDRQCKRWENAKH |
| SEQ ID NO: 691 | GACHRSGWGFACFCYFNKC |
| Thionin-like protein Camelina sativa | MAKAATIVTLLFAALVFFAALETPTMVEAQ |
| SEQ ID NO: 692 | KLCERPSGTWSGVCGNSNACKNQCINLEKA |
| | RHGSCNYVFPAHKCICYFPC |
| Thionin-like | MAKFASIIAFLFAALVLFASFEAPTMVEAQK |
| Arabis alpine | YCEKPSGTWSGVCGNSNACNNQCINLEGAR |
| SEQ ID NO: 693 | HGSCNYVFPYYRCICYFQC |
| Thionin-like | MAMSLKSVHFFALFFIVVLLANQEMPVAEA |
| Theobroma cacao | KLCQKRSKTWTGPCIKTKNCDHQCRKWEK |
| SEQ ID NO: 694 | AQHGACHWQWPGFACFCYVNC |
| Thionin-like | MAKLVSPKAFFVFLFVFLLISASEFSGSEAKL |
| Amborella trichopoda | CQKRSRTWSGFCANSNNCSRQCKNLEGARF |
| SEQ ID NO: 695 | GACHRQRIGLACFCYFNC |
| low-molecular-weight cysteine-rich 67 | MAKSATIVTLFFAALVFFAALEAPMVVEAQ |
| Arabidopsis thaliana | KLCERPSGTWSGVCGNSNACKNQCINLEKA |
| SEQ ID NO: 696 | RHGSCNYVFPAHKCICYFPC |
| Thionin-like | MAKFASIITLLFAALVLFASLEAPTMVEAQK |
| Arabis alpine | LCQRPSGTWSGVCGNNGACKNQCINLEKAR |
| SEQ ID NO: 697 | HGSCNYVFPYHRCICYFPC |
| Thionin-like | MAKVASIIALLFAALVLFAAFEAPTMVEAQK |
| Brassica juncea | LCERPSGTWSGVCGNNNACKNQCINLEKAR |
| SEQ ID NO: 698 | HGSCNYVFPAHKCICYFPC |
| Thionin-like | MAKFASIIALLFAALVLFAALEAPTMVEAQK |
| Brassica oleracea var. oleracea | LCERPSGTWSGVCGNNNACKNQCINLEKAR |
| SEQ ID NO: 699 | HGSCNYVFPAHKCICYFPC |
| Thionin-like | MAKPATIVTLLFAALVFFAALETPTMVEAQK |
| Camelina sativa | LCERPSGTWSGVCGNNNACKNQCINLEKAR |
| SEQ ID NO: 700 | HGSCNYVFPAHKCICYFPC |
| Thionin-like | MAKSATIVTLLFAALVFFAALETPTMVEAQK |
| Camelina sativa | LCERPSGTWSGVCGNNNACKNQCINLEKAR |
| SEQ ID NO: 701 | HGSCNYVFPAHKCICYFPC |
| Thionin-like | MAKFASIIAPLFAVLVLFAAFEAPTMVEAQK |
| Brassica napus | LCERPSGTWSGVCGNNNACKNQCINLEKAR |
| SEQ ID NO:702 | HGSCNYVFPAHKCICYFPC |
| Thionin-like | MAKFASIITLLFAALVLFAVFEGPTMVEAQK |
| Eutrema salsugineum | LCERPSGTWSGVCGNNNACKNQCINLEKAR |
| SEQ ID NO: 703 | HGSCNYVFPAHKCICYFPC |
| Cysteine-rich antifungal protein | MAKFASIIALLFAALVLFAAFEAPTMVEAQK |
| Raphanus sativus | LCERPSGTWSGVCGNNNACKNQCINLEKAR |
| SEQ ID NO: 704 | HGSCNYVFPAHKCICYFPC |
| Thionin-like protein 1 Raphanus sativus | MAKFASIVSLLFAALVLFTAFEAPAMVEAQK |
| SEQ ID NO: 705 | LCERPSGTWSGVCGNNNACKNQCINLEKAR |
| | HGSCNYVFPAHKCICYFPC |
| Thionin-like protein 1 Raphanus sativus | MNTKVILALLFCFLLVASNEMQVGEAKVCQ |
| SEQ ID NO: 706 | RRSKTWSGPCINTGNCSRQCKQQEDARFGA |
| | CHRSGFGFACFCYFKC |
| Thionin-like | MAKFASIIAPLFAALVLFAAFEAPTMVEAQK |
| Brassica rapa | LCERPSGTWSGVCGNNNACKNQCINLEKAR |
| SEQ ID NO: 707 | HGSCNYVFPAHKCICYFPC |
| Thionin-like | MNTKLILALMFCFLLIASNEMQVGEAKVCQ |
| Solanum pennellii | RRSKTWSGPCINTGNCSRQCKQQEDARFGA |
| SEQ ID NO: 708 | CHRSGFGFACFCYFKC |
| Thionin-like | MAKFTTTFALLFAFFILFAAFDVPMAEAKVC |
| Citrus clementina | QRRSKTWSGLCLNTGNCSRQCKQQEDARFG |
| SEQ ID NO: 709 | ACHRQGIGFACFCYFKC |
| Thionin-like | MAKFTSIIVLLFAALVLFAGFEAPTMVEAQK |
| Brassica rapa | LCERPSGTWSGVCGNNNACKNQCIRLEKAR |
| SEQ ID NO: 710 | HGSCNYVFPARKCICYFPC |
| Thionin-like | MAKFASIITLLFAALVLFATFAPTMVEAKLC |
| Eutrema salsugineum | ERPSGTWSGVCGNNNACKSQCQRLEGARHG |
| SEQ ID NO: 711 | SCNYVFPAHKCICYFPC |
| Thionin-like | MAKFASIITLLFAALVLFATFEAPTMVEAKL |
| Eutrema salsugineum | CERPSGTWSGVCGNNNACKSQCQRLEGARH |
| SEQ ID NO: 712 | GSCNYVFPAHKCICYFPC |
| Thionin-like | MAKFASIIAFFFAALVLFAAFEAPTIVEAQKL |
| Heliophila coronopifolia | CERPSGTWSGVCGNNNACRNQCINLEKARH |
| SEQ ID NO: 713 | GSCNYVFPAHKCICYFPC |
| Thionin-like | MAKVASIVALLFPALVIFAAFEAPTMVEAQK |
| Brassica oleracea | LCERPSGTWSGVCGNNNACKNQCIRLEKAR |
| SEQ ID NO: 714 | HGSCNYVFPAHKCICYFPC |
| Thionin-like | MSKFYTVFMFLCLALLLISSWEVEAKLCQRR |
| Cicer arietinum | SKTWSGPCIITGNCKNQCKNVEHATFGACHR |
| SEQ ID NO: 715 | QGFGFACFCYFNCH |
| Thionin-like | MAKSVASITTAFALIFAFFILFASFGVPMAEA |
| Citrus clementina | KVCQRRSKTWSGPCLNTGKCSRQCKQQEYA |
| SEQ ID NO: 716 | RYGACYRQGAGYACYCYFNC |
| Thionin-like | MAKSVASITTAFALIFAFFILFASFEVPMAEA |
| Citrus sinensis | KVCQRRSKTWSGPCLNTGKCSRHCKQQEDA |
| SEQ ID NO: 717 | RYGACYRQGTGYACFCYFEC |
| Thionin-like | MAKFTTTFALLFAFFILFAAFDVPMAEAKVC |
| Citrus sinensis | QLRSKTWSGLCLNTGNCSRQCKQQEDARFG |
| SEQ ID NO: 718 | ACHRQGIGFACFCYFKC |
| Ec-AMP -D1 | MERSVRLFSTVLLVLLLLASEMGLRAAEARI |
| Citrus sinensis | CESQSHRFKGPCVSKSNCAAVCQTEGFHGG |
| SEQ ID NO: 719 | HCRGFRRRCFCTKRC |

The composition can comprise a fusion protein.

Table 16 (SEQ ID NO: 720) describes the sequences used to make a translational fusion using the nucleotide sequence that encodes the synthetic phloem targeting polypeptide (SEQ ID NO: 611) with a synthetic thionin polypeptide (SEQ ID NO: 620). The upper case (not bold) font sequence identifies the phloem targeting sequence, the upper case bold font identifies the thionin polypeptide. Table 16 depicts SEQ ID NO: 720 which represents the fusion of these two peptide sequences resulting in a phloem targeted bioactive priming polypeptide.

TABLE 16
Translational fusion of a phloem targeting sequence with a thionin derived
polypeptide
Translational fusion phloem targeting sequence with thionin polypeptide (synthetic):
SEQ ID NO: 720
MSTATFVDIIIAILLPPLGVFLRFGCGVEFWICLVLTLLGYIPGIIYAIYVLTKRTCESQS
HRFKGPCSRDSNCATVCLTEGFSGGDCRGFRRRCRCTRPCVFDEK

Serine Proteases

The composition can comprise at least one serine protease. The serine proteases provided herein comprise proteins or catalytic domains of proteins that belongs to the serine protease family. The full lengthproteins (e.g., SEQ ID NOs: 722 or 795) contain a type II transmembrane domain, a receptor class A domain, a scavenger receptor cysteine-rich domain and a protease domain. Serine proteases can inhibit other proteases in plants and function in protecting the plant against herbivorous insects by inhibiting digestive proteases. Compositions prepared herein with serine proteases can be particularly effective at protecting against HLB disease causing psyllids. Serine proteases can also be effective for disrupting bacterial biofilms through cleavage of protein components, thereby reducing bacterial survival and reducing spread of bacteria within or on a plant, or plant part.

For ease of reference, illustrative serine protease amino acid sequences are provided in Table 17 below, together with their SEQ ID NOs. The compositions herein can comprise a serine protease having an amino acid sequence comprising any one of SEQ ID NOs: 721, 722, and 794-796. The compositions herein can comprise a serine protease having an amino acid sequence comprising SEQ ID NO: 722 or 795. The compositions herein can comprise a serine protease having an amino acid sequence comprising SEQ ID NO: 794 or 796.

The serine protease can comprise a truncated version of SEQ ID NO: 722 comprising the catalytic domain of the full-length protein. For example, the amino acid sequence of the serine protease can comprise SEQ ID NO: 794 (Table 17). Accordingly, the compositions herein can comprise a serine protease having an amino acid sequence comprising SEQ ID NO: 794.

The amino acid sequence of serine protease 2 (SEQ ID NO: 795) provided in Table 17 was cloned from a proprietary library from Bacillus subtilis and comprises four amino acid substitutions relative to the native sequence (SEQ ID NO: 722), which confer a polypeptide with serine protease activity. In some compositions, the serine protease can comprise a truncated version of SEQ ID NO: 795 comprising the catalytic domain of the full-length protein. For example, the amino acid sequence of the serine protease can comprise SEQ ID NO: 796 (Table 17). Accordingly, the compositions herein can comprise a serine protease having an amino acid sequence comprising SEQ ID NO: 796.

The native amino acid sequence of the serine protease of SEQ ID NO: 722 includes the signal peptide MKKGIIRFLLVSFVLFFALSTGITGVQAAPA (SEQ ID NO: 797) at the amino-terminus of the sequence, immediately preceding the first amino acid of SEQ ID NO: 722. This signal peptide is not included in SEQ ID NO: 722. However, the signal peptide of SEQ ID NO: 797, or another signal peptide, can optionally be included at the amino terminus of the serine proteases of any of SEQ ID NO: 721-722, 794-796, or at the amino-terminus of any of the other peptides described herein.

TABLE 17
Serine Proteases

| SEQ ID NO: | Serine Protease Sequences- Amino Acid |
|---|---|
| Protease 1 (Bsub 168 aprX) | MFGYSMVQMVRANAHKLDWPLRETVLQLYKPFKWTPCF |
| (Bacillus subtilis subsp. | LHKFFETKLQNRKKMSVIIEFEEGCHETGFQMAGEVLQKE |
| subtilis str. 168) | KRSKLKSRFNKINCCSAEVTPSALHSLLSECSNIRKVYLNR |
| SEQ ID NO: 721 | EVKALLDTATEASHAKEVVRNGQTLTGKGVTVAVVDTGI |
| | YPHPDLEGRIIGFADMVNQKTEPYDDNGHGTHCAGDVAS |
| | SGASSSGQYRGPAPEANLIGVKVLNKQGSGTLADIIEGVE |
| | WCIQYNEDNPDEPIDIMSMSLGGDALRYDHEQEDPLVRAV |
| | EEAWSAGIVVCVAAGNSGPDSQTIASPGVSEKVITVGALD |
| | DNNTASSDDDTVASFSSRGPTVYGKEKPDILAPGVNIISLR |
| | SPNSYIDKLQKSSRVGSQYFTMSGTSMATPICAGIAALILQ |
| | QNPDLTPDEVKELLKNGTDKWKDEDPNIYGAGAVNAENS |
| | VPGQ |
| Serine Protease 2 | SSKTSADLEKAEVFGDIDMTTSKKTTVIVELKEKSLAEAKE |
| Bacillus subtilis subsp. | AGESQSKSKLKTARTKAKNKAIKAVKNGKVNREYEQVFS |
| Substilis str. 168, native | GFSMKLPANEIPKLLAVKDVKAVYPNVTYKTDNMKDKD |
| SEQ ID NO: 722 | VTISEDAVSPQMDDSAPYIGANDAWDLGYTGKGIKVAIID |
| | TGVEYNHPDLKKNFGQYKGYDFVDNDYDPKETPTGDPRG |
| | EATDHGTHVAGTVAANGTIKGVAPDATLLAYRVLGPGGS |
| | GTTENVIAGVERAVQDGADVMNLSLGNSLNNPDWATSTA |
| | LDWAMSEGVVAVTSNGNSGPNGWTVGSPGTSREAISVGA |
| | TQLPLNEYAVTFGSYSSAKVMGYNKEDDVKALNNKEVEL |
| | VEAGIGEAKDFEGKDLTGKVAVVKRGSIAFVDKADNAKK |
| | AGAIGMVVYNNLSGEIEANVPGMSVPTIKLSLEDGEKLVS |
| | ALKAGETKTTFKLTVSKALGEQVADFSSRGPVMDTWMIK |
| | PDISAPGVNIVSTIPTHDPDHPYGYGSKQGTSMASPHIAGA |
| | VAVIKQAKPKWSVEQIKAAIMNTAVTLKDSDGEVYPHNA |
| | QGAGSARIMNAIKADSLVSPGSYSYGTFLKENGNETKNET |
| | FTIENQSSIRKSYTLEYSFNGSGISTSGTSRVVIPAHQTGKA |
| | TAKVKVNTKKTKAGTYEGTVIVREGGKTVAKVPTLLIVK |
| | EPDYPRVTSVSVSEGSVQGTYQIETYLPAGAEELAFLVYDS |
| | NLDFAGQAGIYKNQDKGYQYFDWDGTINGGTKLPAGEY |
| | YLLAYAANKGKSSQVLTEEPFTVE |
| Serine Protease 2 native | DDSAPYIGANDAWDLGYTGKGIKVAIIDTGVEYNHPDLKK |
| (truncated) | NFGQYKGYDFVDNDYDPKETPTGDPRGEATDHGTHVAGT |
| Bacillus subtilis | VAANGTIKGVAPDATLLAYRVLGPGGSGTTENVIAGVERA |
| SEQ ID NO: 794 | VQDGADVMNLSLGNSLNNPDWATSTALDWAMSEGVVA |
| | VTSNGNSGPNGWTVGSPGTSREAISVGATQLPLNEYAVTF |
| | GSYSSAKVMGYNKEDDVKALNNKEVELVEAGIGEAKDFE |
| | GKDLTGKVAVVKRGSIAFVDKADNAKKAGAIGMVVYNN |
| | LSGEIEANVPGMSVPTIKLSLEDGEKLVSALKAGETKTTFK |
| | LTVSKALGEQVADFSSRGPVMDTWMIKPDISAPGVNIVSTI |
| | PTHDPDHPYGYGSKQGTSMASPHIAGAVAVIKQAKPKWS |
| | VEQIKAAIMNTAVTLKDSDGEVYPHNAQGAGSARIMNAI |
| | KAD |
| Serine Protease 2 mutant | SSKTSADLEKAEVFGDIDMTTSKKTTVIVELKEKSLAEAKE |
| (Bacillus subtilis subsp. | AGESQSKSKLKTARTQAKNKAIKAVKNGKVNREYEQVFS |
| subtilis str. 168, with | GFSMKLPANEIPKLLAVKDVKAVYPNVTYKTDNMKDKD |
| mutations) | VTISEDAVSPQMDDSAPYIGANDAWDLGYTGKGIKVAIID |
| SEQ ID NO: 795 | TGVEYNHPDLKKNFGQYKGYDFVDNDYDPKETPTGDPRG |
| | EATDHGTHVAGTVAANGTIKGVAPDATLLAYRVLGPGGS |
| | GTTENVIAGVERAVQDGADVMNLSLGNSLNNPDWATSTA |
| | LDWAMSEGVVAVTSNGNSGPNGWTVGSPGTSREAISVGA |
| | TQLPLDEYAVTFGSYSSAKVMGYNKEDDVKALNNKEVEL |
| | VEAGIGEAKDFEGKDLTGKVAVVKRGSIAFVDKADNAKK |
| | AGAIGMVVYNNLSGEIEANVPGMSVPTIKLSLEDGEKLVS |
| | ALKAGETKTTFKLTVSKALGEQVADFSSRGPVMDTWMIK |
| | PDISAPGVNIVSTIPTHDPDHPYGYGSKQGTSMASPHIAGA |
| | VAVIKQAKPKWSVEQIKAAIMNTAVTLKDSDGEVYPHNA |
| | QGAGSARIMNAIKADSLVSPGSCSYGTFLKENGNETKNET |
| | FTIENQSSIRKSYTLEYSFNGSGISTSGTSRVVIPAHQTGKA |
| | TARVKVNTKKTKAGTYEGTVIVREGGKTVAKVPTLLIVKE |
| | PDYPRVTSVSVSEGSVQGTYQIETYLPAGAEELAFLVYDS |
| | NLDFAGQAGIYKNQDKGYQYFDWDGTINGGTKLPAGEY |
| | YLLAYAANKGKSSQVLTEEPFTVE |
| Serine Protease 2 mutant- | DDSAPYIGANDAWDLGYTGKGIKVAIIDTGVEYNHPDLKK |
| truncated | NFGQYKGYDFVDNDYDPKETPTGDPRGEATDHGTHVAGT |
| (Bacillus subtilis subsp. | VAANGTIKGVAPDATLLAYRVLGPGGSGTTENVIAGVERA |
| subtilis str. 168, with | VQDGADVMNLSLGNSLNNPDWATSTALDWAMSEGVVA |
| mutations) | VTSNGNSGPNGWTVGSPGTSREAISVGATQLPLDEYAVTF |
| SEQ ID NO: 796 | GSYSSAKVMGYNKEDDVKALNNKEVELVEAGIGEAKDFE |
| | GKDLTGKVAVVKRGSIAFVDKADNAKKAGAIGMVVYNN |
| | LSGEIEANVPGMSVPTIKLSLEDGEKLVSALKAGETKTTFK |
| | LTVSKALGEQVADFSSRGPVMDTWMIKPDISAPGVNIVSTI |
| | PTHDPDHPYGYGSKQGTSMASPHIAGAVAVIKQAKPKWS |
| | VEQIKAAIMNTAVTLKDSDGEVYPHNAQGAGSARIMNAI |
| | KAD |

ACC Deaminase

The composition can comprise at least one ACC deaminase (1-aminocyclopropane-1-carboxylate deaminase) polypeptide. Preferably the composition comprises a polypeptide having ACC deaminase activity.

As explained in greater detail below, mutations can be made in polypeptides that exhibit D-cysteine desulfhydrase and/or ACC deaminase activity in order to increase the ACC deaminase activity of the polypeptides. All plants make ACC and respond to ethylene, and thus such modified ACC deaminase polypeptides have broad applicability.

For ease of reference, illustrative D-cysteine desulfhydrase and 1-aminocyclopropane-1-carboxylate deaminase (ACC deaminase) amino acid sequences are provided in Table 18 below, together with their SEQ ID NOs. Mutation of certain amino acids in a wild-type D-cysteine desulfhydrase or ACC deaminase enzyme can result in a polypeptide having increased ACC deaminase activity as compared to the ACC deaminase activity of the wild-type polypeptide (e.g., enzyme) under the same conditions.

In Table 18, SEQ ID NOs. 723-726 are amino acid sequences for wild-type enzymes that exhibit both ACC deaminase and D-cysteine desulfhydrase activity, and SEQ ID NOs. 727-730 are amino acid sequences for the corresponding versions of these enzymes having two amino acid substitutions relative to the wild-type sequence that result in increased enzyme activity. Thus, SEQ ID NO: 723 is a wild-type sequence and SEQ ID NO: 727 provides the amino acid sequence for the same enzyme having the two amino acid substitutions relative to the wild-type sequence. SEQ ID NOs. 724 and 728, 725 and 729, and 726 and 730 are related to one another in the same manner. The substituted amino acids are shown in SEQ ID NOs. 727-730 in Table 18 in bold and underlined text.

The compositions described herein can comprise a polypeptide having ACC deaminase activity. Preferably, the polypeptide has an amino acid sequence comprising at least one amino acid substitution relative to the sequence of a wild-type D-cysteine desulfhydrase or ACC deaminase enzyme from a Bacillus genus bacterium. The amino acid sequence of an exemplary ACC deaminase polypeptide that can be used in the compositions and methods herein can comprise SEQ ID NOs 723-730 (Table 18). Preferably, the amino acid sequence of the ACC deaminase polypeptide comprises SEQ ID NO: 730.

TABLE 18
ACC Deaminase Polypeptides

| Enzyme (SEQ ID NO) | Amino acid sequence |
|---|---|
| D-Cysteine Desulfhydrase | MNLAKFPRKKYTESYTPIEKLNNFSEALGGPTIYFKRDDLL |
| (ACC deaminase native 1b) | GLTAGGNKTRKLEFLVADAEAKGADTLITAGGIQSNHCRL |
| Wild-type | TLAAAVKEKMKCILVLEEGLEPEEKPDFNGNYFLYHLLGA |
| Bacillus thuringiensis | ENVIVVPNGADLMEEMHKVAKEVSEKGNTPYVIPVGGSNP |
| (SEQ ID NO: 723) | TGAMGYVACAQEIMAQSFDQGIDFSTVVCVSGSAGMHAG |
| | LITGFAGTQSHIPVIGINVSRGKAEQEEKVAKLVDETSAHV |
| | GIPNFIPRDAVTCFDEYVGPGYALPTPEMVEAVQLLAKTEG |
| | ILLDPVYTGKAVAGLIDLIKKGTFNKEDNILFVHSGGSPAL |
| | YANTSLFA |
| D-Cysteine Desulfhydrase | MNLAKFPRKKYTESYTPIEKLNHFSEVLGGPSIYFKRDDLL |
| (ACC deaminase native 2b) | GLTAGGNKTRKLEFLVADAQAKGVDTLITAGGIQSNHCRL |
| Wild-type | TLAAAVKEKMKCILVLEEGLEPEEKPDFNGNYFLYHLLGA |
| Bacillus pseudomycoides | ENVIVVPNGTDLMDEMQKVAKEVTEKGHTPYVIPVGGSNP |
| (SEQ ID NO: 724) | TGAMGYIACAEEIMAQSFEQGIDFNAVVCVSGSGGMHAGL |
| | ITGFYGRQTGIPIIGMNVSRGKAEQEEKVCKLVQETSAHVG |
| | IPNSIPREAVTCFDEYVGPGYALPTPEMVEAVQLLAKTEGIL |
| | LDPVYTGKAVAGLIDIIRKGTFKKEDNILFVHSGGSPALYA |
| | NTSLFS |
| D-Cysteine Desulfhydrase | MNLAKFPRKKYTESYTPIEKLNNFSEVLGGPTIYFKRDDLL |
| (ACC deaminase native 3b) | GLTAGGNKTRKLEFLVADAQAKGADTLITAGGIQSNHCRL |
| Wild-type | TLAAAVKEKMKCILVLEEGLEPEEKPDFNGNYFLYHLLGA |
| Bacillus thuringiensis | ENVIVVPNGADLMEEMHKVAKEVSEKGNTPYVIPVGGSNP |
| (SEQ ID NO: 725) | TGAMGYVACAQEIMAQSFEQGIDFSSVVCVSGSGGMHAG |
| | LITGFAGTQSHIPVIGINVSRGKAEQEEKVAKLVDETSAHV |
| | GIPNFISRDAVTCFDQYVGPGYALPTQEMVEAVQLLAKTE |
| | GILLDPVYTGKAVAGLIDLIKKGTFNKEDNILFVHSGGSPA |
| | LYANTSLFA |
| D-Cysteine Desulfhydrase | MNLAKFPRKKYTESYTPIEKLNNFSEALGGPTIYFKRDDLL |
| (ACC deaminase) | GLTAGGNKTRKLEFLVADAQEKGADTLITAGGIQSNHCRL |
| Bacillus thuringiensis | TLAAAVKEKMKCILVLEEGLEPEEKRDFNGNYFLYHLLGA |
| Wild-type | ENVIVVPNGADLMEEMNKVAKEVSEKGSTPYVIPVGGSNP |
| (SEQ ID NO: 726) | TGAMGYVACAQEIMAQSFEQGIDFSSVVCVSGSGGMHAG |
| | LITGFSGTQSHIPVIGINVSRGKAEQEEKVAKLVDETSAHVG |
| | IPNFISRDAVTCFDEYVGPGYALPTPEMVEAVQLLAKTEGI |
| | LLDPVYEGKAVAGLIDLIRKGKFNKEDNILFVHLGGSPALY |
| | ANTSLFA |
| D-Cysteine Desulfhydrase | MNLAKFPRKKYTESYTPIEKLNNFSEALGGPTIYFKRDDLL |
| (ACC deaminase native 1b) | GLTAGGNKTRKLEFLVADAEAKGADTLITAGGIQSNHCRL |
| With mutations | TLAAAVKEKMKCILVLEEGLEPEEKPDFNGNYFLYHLLGA |
| Bacillus thuringiensis | ENVIVVPNGADLMEEMHKVAKEVSEKGNTPYVIPVGGSNP |
| (SEQ ID NO: 727) | TGAMGYVACAQEIMAQSFDQGIDFSTVVCVSGSAGMHAG |
| | LITGFAGTQSHIPVIGINVSRGKAEQEEKVAKLVDETSAHV |
| | GIPNFIPRDAVTCFDEYVGPGYALPTPEMVEAVQLLAKTEG |
| | ILLDPVYEGKAVAGLIDLIKKGTFNKEDNILFVHLGGSPAL |
| | YANTSLFA |
| D-Cysteine Desulfhydrase | MNLAKFPRKKYTESYTPIEKLNHFSEVLGGPSIYFKRDDLL |
| (ACC deaminase native 2b) | GLTAGGNKTRKLEFLVADAQAKGVDTLITAGGIQSNHCRL |
| With mutations | TLAAAVKEKMKCILVLEEGLEPEEKPDFNGNYFLYHLLGA |
| Bacillus pseudomycoides | ENVIVVPNGTDLMDEMQKVAKEVTEKGHTPYVIPVGGSNP |
| (SEQ ID NO: 728) | TGAMGYIACAEEIMAQSFEQGIDFNAVVCVSGSGGMHAGL |
| | ITGFYGRQTGIPIIGMNVSRGKAEQEEKVCKLVQETSAHVG |
| | IPNSIPREAVTCFDEYVGPGYALPTPEMVEAVQLLAKTEGIL |
| | LDPVYEGKAVAGLIDIIRKGTFKKEDNILFVHLGGSPALYA |
| | NTSLFS |
| D-Cysteine Desulfhydrase | MNLAKFPRKKYTESYTPIEKLNNFSEVLGGPTIYFKRDDLL |
| (ACC deaminase native 3b) | GLTAGGNKTRKLEFLVADAQAKGADTLITAGGIQSNHCRL |
| With mutations | TLAAAVKEKMKCILVLEEGLEPEEKPDFNGNYFLYHLLGA |
| Bacillus thuringiensis | ENVIVVPNGADLMEEMHKVAKEVSEKGNTPYVIPVGGSNP |
| (SEQ ID NO: 729) | TGAMGYVACAQEIMAQSFEQGIDFSSVVCVSGSGGMHAG |
| | LITGFAGTQSHIPVIGINVSRGKAEQEEKVAKLVDETSAHV |
| | GIPNFISRDAVTCFDQYVGPGYALPTQEMVEAVQLLAKTE |
| | GILLDPVYEGKAVAGLIDLIKKGTFNKEDNILFVHLGGSPA |
| | LYANTSLFA |
| ACC deaminase | MNLAKFPRKKYTESYTPIEKLNNFSEALGGPTIYFKRDDLL |
| (D-Cysteine Desulfhydrase) | GLTAGGNKTRKLEFLVADAQEKGADTLITAGGIQSNHCRL |
| Bacillus thuringiensis, with | TLAAAVKEKMKCILVLEEGLEPEEKRDFNGNYFLYHLLGA |
| mutations) | ENVIVVPNGADLMEEMNKVAKEVSEKGSTPYVIPVGGSNP |
| (SEQ ID NO: 730) | TGAMGYVACAQEIMAQSFEQGIDFSSVVCVSGSGGMHAG |
| | LITGFSGTQSHIPVIGINVSRGKAEQEEKVAKLVDETSAHVG |
| | IPNFISRDAVTCFDEYVGPGYALPTPEMVEAVQLLAKTEGI |
| | LLDPVYEGKAVAGLIDLIRKGKFNKEDNILFVHLGGSPALY |
| | ANTSLFA |

Glucanase, Amylase and Chitinase Polypeptides

The composition can comprise a glucanase polypeptide.

Glucanases use water to break chemical bonds between individual glucose molecules in glucans, which are long chain polysaccharides. Glucans can be broken down into two types, alpha glucan, consisting of primarily alpha chains of glucose molecules, and beta glucans, consisting of primarily beta chains of glucose molecules. Common alpha glucans include dextrans, glycogens, pullalans, and starch. Alpha glucans generally include combinations of alpha 1,4; alpha 1,6, and/or alpha 1,3 glucans and branches. Glucanases that are specific for cleaving alpha linkages are called alpha-glucanases. Beta glucanases are specific to beta linkages between glucans. Common beta glucans include cellulose, laminarin, lichenin, zymosan. Beta glucans are commonly found with b1,3; b1,4, and/or b1,6 linkages between glucose molecules. Glucanases can be either “exo” or “endo” depending on the location of the cleavage of the polysaccharide. Endo-glucanases (particularly β-1,3-D-glucanases) and amylases are particularly effective in the therapeutic and yield promoting compositions described herein.

The amino acid sequence of illustrative glucanase polypeptides that can be used in the compositions and methods herein can comprise any one of SEQ ID NOs 731-735 or 767-776 as described in Table 19. The glucanase polypeptide can comprise a β-1,3-D-glucanase having an amino acid sequence comprising, for example, any one of SEQ ID NOs: 731-733 or 767-776. For example, the glucanase polypeptide can comprise a β-1,3-D-glucanase having an amino acid sequence comprising SEQ ID NO: 772. For example, the glucanase polypeptide can comprise a β-1,3-D-glucanase having an amino acid sequence comprising SEQ ID NO: 732.

The composition can comprise an amylase polypeptide.

Amylases are specific alpha-glucanases that breakdown starch. Amylases are enzymes that hydrolytically cleave α-1,4-glycosidic bonds between individual glucose moieties in the backbone of amylose and amylopectin. Amylose and amylopectin are the components of starch, which are plant-derived storage polysaccharides. Amylose is an unbranched polysaccharide consisting of α-1,4-glycosidic-linked glucose monomers. In the structurally related branched polysaccharide amylopectin several α-1,4-glucan chains are linked to each other by α-1,6-glycosidic bonds.

The amino acid sequence of illustrative amylase polypeptides that can be used in the compositions and methods herein can comprise SEQ ID NO: 734 or SEQ ID NO: 735.

The composition can comprise a chitinase polypeptide.

Chitinases are enzymes that hydrolytically cleave β-1,4-glycosidic bonds between individual N-acetylglucosamine moieties in the backbone of chitin molecules. Chitin is an unbranched structural polysaccharide consisting of β-1,4-glycosidic linked N-acetylglucosamine moieties, which is of high occurrence in the cell walls of many fungi and the exoskeleton of many arthropods.

The amino acid sequence of illustrative chitinase polypeptides that can be used in the compositions and methods herein can comprise SEQ ID NO: 777 or SEQ ID NO: 778.

In some instances, the compositions and methods herein comprise two or more glucanase, amylase or chitinase polypeptides (e.g., a β-1,3-glucanase and an amylase or a β-1,3-glucanase and a chitinase). For example, a composition can comprise an amylase having an amino acid sequence comprising at least one of SEQ ID NO: 734 or 735 and a β-1,3-D-glucanase having an amino acid sequence comprising any one of SEQ ID NO: 731-735 or 767-776. As an additional example, a composition can comprise a chitinase having an amino acid sequence comprising at least one of SEQ ID NO: 777 or 778 and a β-1,3-D-glucanase having an amino acid sequence comprising any one of SEQ ID NO: 731-735 or 767-776. In any of these combinations, the β-1,3-D-glucanase can have an amino acid sequence comprising SEQ ID NO: 772. In any of these combinations, the β-1,3-D-glucanase can have an amino acid sequence comprising SEQ ID NO: 732.

TABLE 19
Illustrative Glucanases, Amylases and Chitinases

| SEQ ID NO: | Glucanase Sequences- Amino Acid |
|---|---|
| β-1,3-D-glucanase | GVCYGVIGNNLPSRSDVVQLYRSKGINGMRIYFADGQALS |
| Hordeum vulgare | ALRNSGIGLILDIGNDQLANIAASTSNAASWVQNNVRPYY |
| SEQ ID NO: 731 | PAVNIKYIAAGNEVQGGATQSILPAMRNLNAALSAAGLGA |
| | IKVSTSIRFDEVANSFPPSAGVFKNAYMTDVARLLASTGAP |
| | LLANVYPYFAYRDNPGSISLNYATFQPGTTVRDQNNGLTY |
| | TSLFDAMVDAVYAALEKAGAPAVKVVVSESGWPSAGGF |
| | AASAGNARTYNQGLINHVGGGTPKKREALETYIFAMFNE |
| | NQKTGDATERSFGLFNPDKSPAYNIQF |
| β-1,3-D-glucanase | AETAGTTITSMSYFSTADGPIITKSGVGQASYGFVMPIFNG |
| Paenibacillus spp. | GSATWNDVAQDLGVKVKVNGSWVDIDSVSSFVYNQNWG |
| SEQ ID NO: 732 | HWNDGGFTGYWFTLSATTEIQLYSKANEVTLEYSLVFQNI |
| | NKTTITAMTPTQGPQITAGFTGGAGFTYPIFNHDPAITYAA |
| | VADDLKVYVKPVNSSQWIDIDNNAASGWIYDQNFGQFTD |
| | GGGGYWFNVTESINVKLESKTSSTNIVYTISFNEPVRNSYV |
| | LTPYEGTTFTADASGAIGIPLPKIDGGAPIGTELGNFVYQINI |
| | NGQWVDLDNSSQSGFVYSANGYNNMSAANQWGYWADH |
| | IYGLWFQPIQVDMQIRIGYPLNGQAGGSVGSNFVNYTLIG |
| | NPDAPRPDVNDQEDIPIGTPNDSAIEGMNLIWQDEFNGTAL |
| | DQSKWNYETGYYLNDDPNTWGWGNSELQHYTDRAQNV |
| | FVQDGKLNIKALNEPKSFPQDPSRYAQYSSGKINTKDHFSL |
| | KYGRVDFRAKLPTGNGIWPALWMLPQDNVYGTWASSGEI |
| | DVMEAKGRLPGSTSGAVHFGGQWPTNRYLSGEYHFPEGQ |
| | TFANDYHVYSVVWEEDNIKWYVDGKFFFKVTRDQWYSA |
| | AAPNNPNAPFDQPFYLIMNLAIGGTFDGGRTPDPSDIPATM |
| | QVDYVRVYKEGEGGGQNPGNVPVTGVTVNPTTAQVEVG |
| | QSVQLNASVAPSNATNKQVTWSVSGSSIASVSPNGLVTGL |
| | AQGTTTVTATTADGNKAASATITVAPAPSTVIVIGDEVKG |
| | LKKIGDDLLFYVNGATFADLHYKVNNGGQLNVAMAPTG |
| | NGNYTYPVHNLKHGDTVEYFFTYNPGQGALDTPWQTYV |
| | HGVTQGTPE |
| β-1,3-D-glucanase | AGTTVTSMEYFSPADGPVISKSGVGKASYGFVMPKFNGGS |
| (Bacillus circulans strain | ATWNDVYSDVGVNVKVGNNWVDIDQAGGYIYNQNWGH |
| WL-12) | WSDGGFNGYWFTLSATTEIQLYSKANGVKLEYQLVFQNI |
| SEQ ID NO: 733 | NKTTITAMNPTQGPQITASFTGGAGFTYPTFNNDSAVTYE |
| | AVADDLKVYVKPVNSSSWIDIDNNAASGWIYDHNFGQFT |
| | DGGGGYWFNVTESINVKLESKTSSANLVYTITFNEPTRNS |
| | YVITPYEGTTFTADANGSIGIPLPKIDGGAPIAKELGNFVYQ |
| | ININGQWVDLSNSSQSKFAYSANGYNNMSDANQWGYWA |
| | DYIYGLWFQPIQENMQIRIGYPLNGQAGGNIGNNFVNYTFI |
| | GNPNAPRPDVSDQEDISIGTPTDPAIAGMNLIWQDEFNGTT |
| | LDTSKWNYETGYYLNNDPATWGWGNAELQHYTNSTQNV |
| | YVQDGKLNIKAMNDSKSFPQDPNRYAQYSSGKINTKDKL |
| | SLKYGRVDFRAKLPTGDGVWPALWMLPKDSVYGTWAAS |
| | GEIDVMEARGRLPGSVSGTIHFGGQWPVNQSSGGDYHFPE |
| | GQTFANDYHVYSVVWEEDNIKWYVDGKFFYKVTNQQW |
| | YSTAAPNNPNAPFDEPFYLIMNLAVGGNFDGGRTPNASDI |
| | PATMQVDYVRVYKEQ |
| Amylase (amyE) | ETANKSNELTAPSIKSGTILHAWNWSFNTLKHNMKDIHDA |
| Bacillus subtilis 168 | GYTAIQTSPINQVKEGNQGDKSMSNWYWLYQPTSYQIGN |
| SEQ ID NO: 734 | RYLGTEQEFKEMCAAAEEYGIKVIVDAVINHTTSDYAAIS |
| | NEVKSIPNWTHGNTQIKNWSDRWDVTQNSLLGLYDWNT |
| | QNTQVQSYLKRFLDRALNDGADGFRFDAAKHIELPDDGS |
| | YGSQFWPNITNTSAEFQYGEILQDSASRDAAYANYMDVT |
| | ASNYGHSIRSALKNRNLGVSNISHYASDVSADKLVTWVES |
| | HDTYANDDEESTWMSDDDIRLGWAVIASRSGSTPLFFSRP |
| | EGGGNGVRFPGKSQIGDRGSALFEDQAITAVNRFHNVM |
| | AGQPEELSNPNGNNQIFMNQRGSHGVVLANAGSSSVSINT |
| | ATKLPDGRYDNKAGAGSFQVNDGKLTGTINARSVAVLYP |
| | DDIAKAPHVFLENYKTGVTHSFNDQLTITLRADANTTKAV |
| | YQINNGPETAFKDGDQFTIGKGDPFGKTYTIMLKGTNSDG |
| | VTRTEKYSFVKRDPASAKTIGYQNPNHWSQVNAYIYKHD |
| | GSRVIELTGSWPGKPMTKNADGIYTLTLPADTDTTNAKVI |
| | FNNGSAQVPGQNQPGFDYVLNGLYNDSGLSGSLPH |
| Amylase | MKQQKRLYARLLPLLFALIFLLPHSATAAANLNGTLMQYF |
| Bacillus licheniformis | EWYMPNDGQHWKRLQNDSAYLAEHGITAVWIPPAYKGT |
| SEQ ID NO: 735 | SQDDVGYGAYDLYDLGEFHQKGTVRTKYGTKGELQSAIN |
| | SLHSRGINVYGDVVINHKGGADATEDVTAVEVDPADRNR |
| | VTSGEQRIKAWTHFQFPGRGSTYSDFKWHWYHFDGTDW |
| | DESRKLNRIYKFQGKAWDWEVSNENGNYDYLMYADIDY |
| | DHPDVTAEIKRWGTWYANELQLDGFRLDAVKHIKFSFLR |
| | DWVNHVREKTGKEMFTVAEYWQNDLGALENYLNKTNF |
| | NHSVFDVPLHYQFHAASTQGGGYDMRKLLNGTVVSKHP |
| | VKAVTFVDNHDTQPGQSLESTVQTWFKPLAYAFILTREAG |
| | YPQIFYGDMYGTKGASQREIPALKHKIEPILKARIQYAYGA |
| | QHDYFDHHDIVGWTREGDSSVANSGLAALITDGPGGTKR |
| | MYVGRQNAGETWHDITGNRSDSVVINAEGWGEFHVNGG |
| | SVSIYVQR |
| β-1,3-D-glucanase | AGWAAPAASSGAGAIQASSEEPGISADQAGAGAAFKDIQG |
| (LamA1) | TWASRQVGKWAGLGLVNGSGGQFRPGDTVSRAEFAKLV |
| Paenibacillus sp. | NALFGFTAKTGGTLGDVAPGKWYAEQVAIALQAGYMEG |
| CCRC17245 | YPGGLFKPEAAVTRQEAAKIAALLFPLATADSAAVLSGFK |
| SEQ ID NO: 767 | DRTAIGGFAVQPLADLVSAGALKGFADGTLRPQQPLTRAE |
| | AVVLLDRLAGEIIRQPGSYDGVKSDSGLLIASADTILKQAE |
| | VKGNVLITAGVGEGEVTLDGLSADGTLYVNGGGSHSVHL |
| | RNAKVGKVVVNKSGGPVRVVLEGSSKVGEMSLETGAVV |
| | EVGEQAEVASLQVEQSAGGTELNVKGTVGELQTQASGVT |
| | LNGETFEQGKVLEVQQGKAADKTEPQNGNAPAGGTSGGG |
| | AASPGNGGGSGGGGNGGGGTAGENLAAPVLTPDPVNNVL |
| | GRDVALTFADNPAWRNAISEITLNGRKLTLTADYLLSAGS |
| | LTLKASVFAETGDHTLIIKAAGYTDVSVTQPMGKWELVW |
| | GDEFDGSGTHVDANGVNLDKWGYQNGTGAEYGLDGWG |
| | NNEQQYYTKDNLKVQDGKLTITAKKQPLGGKPYTSGRLW |
| | TSPTFTKQYGRFEASIKLPEGEGLWPAFWMMPKDSKYGV |
| | WASSGELDIMEVRGRLPEESSGTIHYGKPWPNNKSTGTDY |
| | HFPAGQSISSGFHTYAVEWEPGEIRWYVDGNLFQKVDEW |
| | SSEGAGQPDKYAFPAPFDQPFYIILNLAVGGNFDGNRLPPD |
| | SKLPAEMQVDYVRAYELDGKPYKTPVEPVLAKEPIPAEAR |
| | QPVDGSYIADSNFEQGLTDIPVSSQPLSADKWNFLHTPDY |
| | GGAGSASIEQIENRNFAKIVPTSAGNQNYSLQMIQYAPLVR |
| | GHVYKLSFDAKSDAERSIAVKMGGDGDNGWAAYSDNFD |
| | VKLQASLQHYEYRFVMGAQTDLTARLEFNAGLNTHPVWI |
| | GHVRLEETDQVTDPDGAKTPLEDGNHIYNGTFDLGTMDR |
| | MKYWHFVTEAPDGGADASASVDPDARELAVDIRSGGSHP |
| | QAVRLLQKGINLLQNDTYELTFEAKAGSPRSIGVTLLSKD |
| | GSTIYGKAEGLAVGTTAEQQTVTFTMPVQVSDPEGQLVFE |
| | LGGGQAGKAALTLDNIRLIRTTNNNVDYSKVSLYPLVNGD |
| | FSAGLSGWEPFTQGAAANFSAADGIAKVSVSNVGTEAWNI |
| | MFNQSNLNLTKGFTYVLAFDAKSSAARDTEVTLEDAAYN |
| | RRFDSGFISLGTDWQHYEYTVKAAADDNVALKLLLGKTP |
| | QAPNGAHDVSFRNVVLEVKDAPLQRPPALAADATDNRYG |
| | QPVQIGFKDNEAWRTAISSILVNDRVLEAGAYEIQPGALIL |
| | LAPSFSSEGTYRITVKAAGYADTSVTQVLIAGDGNLLVNG |
| | GFDQEKTAWELWVANEGDTTFDVKDGAAELNIHYYGGL |
| | DPQWGVPFSWYTQLMQSGVKVEAGKTYELSFRAWSSVD |
| | RPILVELTGYNNNQQLPFSITGDSQEVYTAVLKPSANAVFT |
| | LKYLLGNVITDGLTTPDAEHQLHLDDIKLTEVKGGPQLTA |
| | DTTENQAGHEIELTFPDDPDWRGAISGVLINGTAAGMDKV |
| | AAGPGSLKLEASLFPSPGSYTISILAQGYAGNTVSQLILSAS |
| | PNVALGKTATASTSVQSASGAVDGNANTRWESDFNDPQ |
| | WLSVDLGGLYRIDSVLLNWEGAYGKTYQVQISQAEQPGE |
| | NDWTDWYTEAAGNGGQDLVFAAPAEARHVRILGTARAT |
| | QYGYSLWEMEVYGTPAEDQTAAGEDVNP |
| β-1,3-D-glucanase - | TQPMGKWELVWGDEFDGSGTHVDANGVNLDKWGYQNG |
| functional domain | TGAEYGLDGWGNNEQQYYTKDNLKVQDGKLTITAKKQP |
| (LamA1) | LGGKPYTSGRLWTSPTFTKQYGRFEASIKLPEGEGLWPAF |
| Paenibacillus sp. | WMMPKDSKYGVWASSGELDIMEVRGRLPEESSGTIHYGK |
| CCRC17245 | PWPNNKSTGTDYHFPAGQSISSGFHTYAVEWEPGEIRWYV |
| SEQ ID NO: 768 | DGNLFQKVDEWSSEGAGQPDKYAFPAPFDQPFYIILNLAV |
| | GGNFDGNRLPPDSKLPAEMQVDYVRAYELDGKPYKTPVE |
| | PVLAKEPIPAEA |
| β-1,3-D-glucanase | QIGVCYGMLGDTLPSPSDVVALYKQQNIQRMRLYGPDPG |
| (AtPr2) | ALAALRGSDIELILDVPSSDLERLASSQTEADKWVQENVQ |
| Arabidopsis thaliana | SYRDGVRFRYINVGNEVKPSVGGFLLQAMQNIENAVSGA |
| SEQ ID NO: 769 | GLEVKVSTAIATDTTTDTSPPSQGRFRDEYKSFLEPVIGFLA |
| | SKQSPLLVNLYPYFSYMGDTANIHLDYALFTAQSTVDNDP |
| | GYSYQNLFDANLDSVYAALEKSGGGSLEIVVSETGWPTEG |
| | AVGTSVENAKTYVNNLIQHVKNGSPRRPGKAIETYIFAMF |
| | DENKKEPTYEKFWGLFHPDRQSKYEVNFN |
| β-1,3-D-glucanase | QIGVCYGMKAKILPSKRDVVALYNQNNIRRMRLYDPNIEA |
| (CsPr2) | LEALRGSNIEVMLGLPNENLQRIASNQAEANTWVQNNVR |
| Citrus sinensis | NFANNVKFKYIAVGNEAKPGDNFAQYLVPAMRNIQNAIN |
| SEQ ID NO: 770 | GAGLGNQIKVSTAIETGALGESFPPSRGSFKQDYRPILDPLI |
| | RFLNENRSPLLLNLYPYFAIAGNRQISLDYALFRSQQTVVS |
| | DGSLSYRSLFDAILDAVYAALEKTGGGSLDIVISESGWPTA |
| | GGDGALTNVDNARTYNNNLIQHVKRGSPKRPGRPIETYIF |
| | AMFDENGKMGPEIERHWGLFAPNRQPKYQINFN |
| β-1,3-D-glucanase | SAPAPPSGWSQVFLDDFDGAAGSSVNTANWQFDTGTSYP |
| (Curd) | GGAGNWGTGEVESMTSSTSNVSLDGNGDLLITPRRDASG |
| Streptomyces sioyaensis | NWTSGRIETTRTDFQPPAGGKLRVEARLQMPNVTGDAAA |
| SEQ ID NO: 771 | GYWPAFWMLGAPFRGNYQNWPGVGELDIMENVQGLNKT |
| | WATMHCGTSPGGPCNETSGIGNLTACPNTTCHSGLHTYT |
| | MEWDRSVSPEAIRFSVDGVTYQTVTANHMDAVTWTNAT |
| | NHGFFVILNVAMGGGFPGAFGGGPTGATEPGHPMVVDYV |
| | QVLQSSGGGGGGGGGTTPPPTGDRDAYGQIQAESYDGQS |
| | GVATETTTDTGGGQDMGYLANGDWALYKGVNFGSTPAT |
| | QFYGRVASGAGGGVSGLVEVRLDSRTNAPIGSFAVGDTG |
| | GWQSWRTVPANIGSVTGTHDVYLTFSSGQPADFVNVNWF |
| | DFGH |
| β-1,3-D-glucanase | APGDLLWSDEFDGAAGSAPNPAVWNHETGAHGWGNAEL |
| (DK-1) | QNYTASRANSALDGQGNLVITARREGDGSYTSARMTTQG |
| Cellulosimicrobium | KYQPQYGRIEARIQIPRGQGIWPAFWMLGGSFPGTPWPSS |
| cellulans strain .DK-1 | GEIDIMENVGFEPHRVHGTVHGPGYSGGSGITGMYQHPQG |
| SEQ ID NO: 772 | WSFADTFHTFAVDWKPGEITWFVDGQQFHRVTRASVGAN |
| | AWVFDQPFFLILNVAVGGQWPGYPDGTTQLPQQMKVDY |
| | VRVYDNGSGSSNPGNPGTGLPTGTGAVRAANGMCIDVPW |
| | ADPTDGNPVQIVTCSGNAAQTWTRGSDGTVRALGKCLDV |
| | RDGSTTRGAAVQVWTCNGTGAQKWAYDAGSKALRNPQS |
| | GLCLDATGGAPLHDGQRLQTWTCNGTTAQQWTL |
| β-1,3-D-glucanase | ATTPLAAAPVAAGNWGDDFDGPAGAAVDPAKWTLETGG |
| (QLK1) | SGNGNHELQYYTAGAANAALDGQGHLVITAKRNTDPGLS |
| Kitasatospora phosalacinea | CWYGTCQYTSARLNTSRTFTQAYGHFESRIKIPRGQGIWP |
| strain SYBCQL | AFWMLGNDLGTAGWPNSGEIDVMENIGREPGTVHGTIHG |
| SEQ ID NO: 773 | PGYSGAGGIGAPYSLPAGQSFADAFHTFAVDWSPTAITWS |
| | VDGTAYQTRTPADLGGNRWVFDHPFFVILNLAVGGDWPG |
| | SPDGSSTYPQTMTVDYVHTTTWGGSTGGSYTGQITGPGG |
| | MCMDVAGASSADSTPIQLHNCTGNAAQQWTVGADGTVR |
| | ALGKCLDVAAASHNDGAAIQLYTCNGTSAQQWTHRSGN |
| | DLLNPGSGKCLDSPNGSSADGTHLQLWTCNGTGAQKWTL |
| | G |
| β-1,3-D-glucanase | RTAAPEQARTAAGAAAAVSTFSDTFDGPAGAAVDSSKWT |
| (17-W) | LETGDNVNNHERQYYTSGTKNAALDGQGHLVITARKENP |
| Streptomyces sp. SYBC17 | AGYQCWYGSCQYTSARLNTAGKFNAQYGHVEARMKIPR |
| SEQ ID NO: 774 | GQGMWPAFWMLGTPVNWPDSGEIDVMENVGFEPSTVHG |
| | TIHGPGYSGSGGIGAAYSLPNGQAFADAFHTFAVDWAPDS |
| | ITWSVDGNVYQRRTPADLGGKSWVFNKPFFLILNLAVGG |
| | YWPGDPDGSTQFPQTLVVDSVSVTTSGGGAGVPIRGLAGK |
| | CVDVAGANSANGTPVQLYDCNGTGAQAWTAGSDGTLRA |
| | LGKCLDVSGGGTADGTPVQLWDCNGSPAQQWALPAARD |
| | IVNPQANKCLDVTGNNAANGTRLQIWTCTGGANQKWTV |
| | G |
| β-1,3-D-glucanase | AGPAGETAGRTVQKAAQGAEAAPAAVLFEENFDGPAGSA |
| (Bgls27) | VDSRRWQLETGDNSGNNHERQYYTPGNANAALDGNGNL |
| Streptomyces sp. S27 | VITARKENPGNYQCWYGRCEYTSARMNTAGKFTTTYGHI |
| SEQ ID NO: 775 | EARMKLPRGQGMWPAFWMLGHDIGSVGWTNSGEIDIME |
| | NVGYEPGTVHGTLHGPGYSGGEGIGAGYTLPGGRAFADD |
| | FHTFAVDWSPNSITWSVDGQVYQRRTPADLGGDRWVFDK |
| | PFFLILNLAVGGDWPGLPDSSTVFPQKLVVDYVRVTSGGD |
| | SGGGGGGRTGTITGLAGKCLDVAWADTANGTPVQIHDCN |
| | GNAAQQWTVGTDGTIRALGKCLDVSGAGKADGTPVQIW |
| | DCNGTAAQQWVVTGARDIVNPNADKCLDVRDNNSANGT |
| | KTQIWTCSGTANQKWNTP |
| β-1,3-D-glucanase | APNWNLVWSDEFNGTSLNRANWTPEIGTGSGGWGNNEL |
| (BglM) | QYYTDRAQNVQVTGGNLVITAQKESYGGMNYTSARIKTQ |
| Paenibacillus sp IAM1165 | DLKSFTYGKVEARIKLPSGQGLWPAFWMLGSNISSVGWP |
| SEQ ID NO: 776 | KSGEIDIMERVNNNPYVNGTVHWDAGGHADFGRVSGNL |
| | DFSQFHVYSIEWDSKYIRWFVDGQQFNEFYIENGTGNTEE |
| | FQRPFFILLNLAVGGNWPGSPNNSTPFPSQMLVDYVRVYQ |
| | DTGASNVISDGIYTIASKASGKVMDVVDVSTARGAKIQQW |
| | TNYVANNQRFRVESTGDGYYKLTAVHSGKVLDVPSSSTS |
| | TGVQLQQWDDNGSNAQRWKIVDVGGGYYKLVSKVSGLA |
| | VDVASASTADGAVVQQWTDNGTDAQKWLFTKIN |
| Endochitinase | DSPKQSQKIVGYFPSWGVYGRNYQVADIDASKLTHLNYA |
| (ChiC) | FADICWKGKHGNPSTHPDNPNKQTWNCKESGVPLQNKEV |
| Bacillus thuringiensis (E0) | PNGTLVLGEPWADVTKSYPGSGTTWEDCDKYARCGNFGE |
| SEQ ID NO: 777 | LKRLKAKYPHLKTIISVGGWTWSNRFSDMAADEKTRKVF |
| | AESTVAFLRAYGFDGVDLDWEYPGVETIPGGSYRPEDKQ |
| | NFTLLLQDVRNALNKAGAEDGKQYLLTIASGASQRYADH |
| | TELKKISQILDWINIMTYDFHGGWEATSNHNAALYKDPND |
| | PAANTNFYVDGAIDVYTNEGVPVDKLVLGVPFYGRGWKS |
| | CGKENNGQYQPCKPGSDGKLASKGTWDDYSTGDTGVYD |
| | YGDLTANYVNKNGFVRYWNDTAKVPYLYNATTGTFISYD |
| | DNESMKYKTDYIKTKGLSGAMFWELSGDCRTSPKYSCSG |
| | PKLLDTLVKELLGGPINQKDTEPPTNVKNIIVTNKTSSSVQ |
| | LSWTASTDNVGVTEYEITAGEEKWSATTNSITIKNLKPNTE |
| | YTFSVIAKDASGNKSHPTALTVKTDEANTTPPDGNGTATF |
| | SVTSNWGSGYNFSIIIKNNGTIPIKNWKLEFDYSGNLTQVW |
| | DSKISSKTNNHYVITNAGWNGEIPPGGSITIGGAGTGNPAE |
| | LLNAVISEN |
| Chitinase | MSTRKAVIGYYFIPTNQINNYTETDTSVVPFPVSNITPAKA |
| (ChiB) | KQLTHINFSFLDINSNLECAWDPATNDAKARDVVNRLTAL |
| Serratia marcescens | KAHNPSLRIMFSIGGWYYSNDLGVSHANYVNAVKTPASR |
| SEQ ID NO : 778 | AKFAQSCVRIMKDYGFDGVDIDWEYPQAAEVDGFTAALQ |
| | EIRTLLNQQTVADGRQALPYQLTIAGAGGAFFLSRYYSKL |
| | AQIVAPLDYINLMTYDLAGPWEKVTNHQAALFGDAAGPT |
| | FYNALREANLGWSWEELTRAFPSPFSLTVDAAVQQHLMM |
| | EGVPSAKIVMGVPFYGRAFKGVSGGNGGQYSSHSTPGEDP |
| | YPSTDYWLVGCEECVRDKDPRIASYRQLEQMLQGNYGYQ |
| | RLWNDKTKTPYLYHAQNGLFVTYDDAESFKYKAKYIKQQ |
| | QLGGVMFWHLGQDNRNGDLLAALDRYFNAADYDDSQL |
| | DMGTGLRYTGVGPGNLPIMTAPAYVPGTTYAQGALVSYQ |
| | GYVWQTKWGYITSAPGSDSAWLKVGRVA |

The native amino acid sequence of the glucanase of SEQ ID NO: 767 includes the signal peptide MTLSSGKSNRFRRRFAAVLFGTVLLAGQIPA (SEQ ID NO: 779) at the amino-terminus of the sequence, immediately preceding the first amino acid of SEQ ID NO: 767. This signal peptide is not included in SEQ ID NO: 767. However, the signal peptide of SEQ ID NO: 779, or another signal peptide, can optionally be included at the amino terminus of the glucanase of SEQ ID NO: 767, at the amino terminus of the truncated glucanase of SEQ ID NO: 768, or at the amino-terminus of any of the other peptides described herein.

The native amino acid sequence of the glucanase of SEQ ID NO: 769 includes the signal peptide MSESRSLASPPMLMILLSLVIASFFNHTAG (SEQ ID NO: 780) at the amino-terminus of the sequence, immediately preceding the first amino acid of SEQ ID NO: 769. This signal peptide is not included in SEQ ID NO: 769. However, the signal peptide of SEQ ID NO: 780, or another signal peptide, can optionally be included at the amino terminus of the glucanase of SEQ ID NO: 769, or at the amino-terminus of any of the other peptides described herein.

The native amino acid sequence of the glucanase of SEQ ID NO: 770 includes the signal peptide MAKFFSSPNTSSTAPVVLFVVGLLMATLHTASA (SEQ ID NO: 781) at the amino-terminus of the sequence, immediately preceding the first amino acid of SEQ ID NO: 770. This signal peptide is not included in SEQ ID NO: 770. However, the signal peptide of SEQ ID NO: 781, or another signal peptide, can optionally be included at the amino terminus of the glucanase of SEQ ID NO: 770, or at the amino-terminus of any of the other peptides described herein.

The native amino acid sequence of the glucanase of SEQ ID NO: 771 includes the signal peptide MSDSSGTPRPRSHSRPRSRSVRRALMAAVATFGLAAAVATAATGPADA (SEQ ID NO: 782) at the amino-terminus of the sequence, immediately preceding the first amino acid of SEQ ID NO: 771. This signal peptide is not included in SEQ ID NO: 771. However, the signal peptide of SEQ ID NO: 782, or another signal peptide, can optionally be included at the amino terminus of the glucanase of SEQ ID NO: 771, or at the amino-terminus of any of the other peptides described herein.

The native amino acid sequence of the glucanase of SEQ ID NO: 772 includes the signal peptide MDLARHRSLTPPTTPPGTSVGPRPRARRRLAGALVAALTAAAAALAVTV PATSAAA (SEQ ID NO: 783) at the amino-terminus of the sequence, immediately preceding the first amino acid of SEQ ID NO: 772. This signal peptide is not included in SEQ ID NO: 772. However, the signal peptide of SEQ ID NO: 783, or another signal peptide, can optionally be included at the amino terminus of the glucanase of SEQ ID NO: 772, or at the amino-terminus of any of the other peptides described herein.

The native amino acid sequence of the glucanase of SEQ ID NO: 773 includes the signal peptide MAAAPRTRRWSLGGFVLLVATALVAAAPFGSAPTGSA (SEQ ID NO: 784) at the amino-terminus of the sequence, immediately preceding the first amino acid of SEQ ID NO: 773. This signal peptide is not included in SEQ ID NO: 773. However, the signal peptide of SEQ ID NO: 784, or another signal peptide, can optionally be included at the amino terminus of the glucanase of SEQ ID NO: 773, or at the amino-terminus of any of the other peptides described herein.

The native amino acid sequence of the glucanase of SEQ ID NO: 774 includes the signal peptide MASPRLLRRCLFAALSAALVGSVAVGPAQA (SEQ ID NO: 785) at the amino-terminus of the sequence, immediately preceding the first amino acid of SEQ ID NO: 774. This signal peptide is not included in SEQ ID NO: 774. However, the signal peptide of SEQ ID NO: 785, or another signal peptide, can optionally be included at the amino terminus of the glucanase of SEQ ID NO: 774, or at the amino-terminus of any of the other peptides described herein.

The native amino acid sequence of the glucanase of SEQ ID NO: 775 includes the signal peptide MVMHPTTPHTPHDPPRGKPARRRRSRRWASAATLLTLAVTMAVTGTAA (SEQ ID NO: 786) at the amino-terminus of the sequence, immediately preceding the first amino acid of SEQ ID NO: 775. This signal peptide is not included in SEQ ID NO: 775. However, the signal peptide of SEQ ID NO: 786, or another signal peptide, can optionally be included at the amino terminus of the glucanase of SEQ ID NO: 775, or at the amino-terminus of any of the other peptides described herein.

The native amino acid sequence of the glucanase of SEQ ID NO: 776 includes the signal peptide MMLRKGICVVILFSLLVVLLPVNKTNA (SEQ ID NO: 787) at the amino-terminus of the sequence, immediately preceding the first amino acid of SEQ ID NO: 776. This signal peptide is not included in SEQ ID NO: 776. However, the signal peptide of SEQ ID NO: 787, or another signal peptide, can optionally be included at the amino terminus of the glucanase of SEQ ID NO: 776, or at the amino-terminus of any of the other peptides described herein.

### Isolated Polypeptides—Glucanases/Amylases and Chitinases

The glucanases, amylases and chitinases described in Table 19 can also be provided as isolated polypeptides. Accordingly, an isolated polypeptide is provided wherein the polypeptide has an amino acid sequence comprising or consisting of any one of SEQ ID NOs: 732, 735, and 767-778.

The isolated polypeptide can have an amino acid sequence comprising or consisting of any one of SEQ ID NOs: 732, 767-776 and 778.

The isolated polypeptide can have an amino acid sequence comprising or consisting of any one of SEQ ID NOs: 767-769, 771-773, 775, and 778.

The isolated polypeptide can have an amino acid sequence comprising or consisting of any one of SEQ ID NOs: 767-769, 772-773, 775, and 778.

The isolated polypeptide can have an amino acid sequence comprising or consisting of SEQ ID NO: 772.

### Additional Modifications

Any bioactive priming polypeptide, whether naturally occurring or non-natural and whether provided as an isolated polypeptide or in a composition, can be further modified via chemical modification to increase performance as well as stability of the polypeptides. Such bioactive priming polypeptides include flagellin polypeptides, retro inverso polypeptides, thionin polypeptides, RHPP polypeptides, serine protease polypeptides, ACC deaminase polypeptides, glucanase polypeptides, chitinase polypeptides, and amylase polypeptides. Specific sequences that can be chemically modified include SEQ ID NOs: 1-610, 620-719, 721-735, and 745-778. Chemically modified sequences can be provided in the compositions described herein. Further, when the chemically modified sequence comprises or consists of any one of SEQ ID NOs 732, 735 and 745-778, the chemically modified polypeptide can be provided as an isolated polypeptide.

These bioactive priming polypeptides can also be conjugated to other moieties, including a plant binding domain and a polypeptide, and other carriers such as oils, plastics, beads, ceramic, soil, fertilizers, pellets, and most structural materials.

In addition, polypeptides can be chemically synthesized with D-amino acids, β2-amino acids, β3-amino acids, homo amino acids, gamma amino acids, peptoids, N-methyl amino acids, and other non-natural amino acid mimics and derivatives.

The polypeptides can be modified by either natural processes, such as posttranslational processing, or by chemical modification techniques that are well known in the art. Modifications can occur anywhere in a polypeptide, including the polypeptide backbone, the amino acid side-chains and the amino or carboxyl termini. The same type of modification can be present in the same or varying degrees at several sites in a polypeptide. Also, a polypeptide can contain many types of modifications.

Peptides can be branched, for example, as a result of ubiquitination, and they can be cyclic, with or without branching. Cyclic, branched, and branched cyclic polypeptides can result from posttranslational natural processes or may be made by synthetic methods.

Modifications include acetylation, acid addition, acylation, ADP-ribosylation, aldehyde addition, alkylamide addition, amidation, amination, biotinylation, carbamate addition, chloromethyl ketone addition, covalent attachment of a nucleotide or nucleotide derivative, cross-linking, cyclization, disulfide bond formation, demethylation, ester addition, formation of covalent cross-links, formation of cysteine-cysteine disulfide bonds, formation of pyroglutamate, formylation, gamma-carboxylation, glycosylation, GPI anchor formation, hydrazide addition, hydroxyamic acid addition, hydroxylation, iodination, lipid addition, methylation, myristoylation, oxidation, PEGylation, proteolytic processing, phosphorylation, prenylation, palmitoylation, addition of a purification tag, pyroglutamyl addition, racemization, selenoylation, sulfonamide addition, sulfation, transfer-RNA mediated addition of amino acids to proteins such as arginylation, ubiquitination, and urea addition. (see, e.g., Creighton et al. (1993) Proteins—Structure and Molecular Properties, 2nd Ed., T. E. Creighton, W. H. Freeman and Company, New York; Johnson, ed. (1983) Posttranslational Covalent Modification Of Proteins, Academic Press, New York; Seifter et al. (1990) Meth. Enzymol., 182: 626-646; Rattan et. al. (1992) Ann. N.Y. Acad. Sci., 663: 48-62; and the like).

Using known methods of protein engineering and recombinant DNA technology, variants can be generated to improve or alter the characteristics of the polypeptides described herein. Such variants include deletions, insertions, inversions, repeats, duplications, extensions, and substitutions (e.g., conservative substitutions) selected according to general rules well known in the art so as have little effect on activity.

The polypeptide can comprise an amino acid sequence having at least 70% identity to any one of SEQ ID NOs. 1-735, 745-787, and 794-797 wherein the polypeptide has bioactive priming activity.

The polypeptide can comprise an amino acid sequence having at least 75% identity to any one of SEQ ID NOs. 1-735, 745-787, and 794-797 wherein the polypeptide has bioactive priming activity.

The polypeptide can comprise an amino acid sequence having at least 80% identity to any one of SEQ ID NOs. 1-735, 745-787, and 794-797 wherein the polypeptide has bioactive priming activity.

The polypeptide can comprise an amino acid sequence having at least 85% identity to any one of SEQ ID NOs. 1-735, 745-787, and 794-797 wherein the polypeptide has bioactive priming activity.

The polypeptide can comprise an amino acid sequence having at least 90% identity to any one of SEQ ID NOs. 1-735, 745-787, and 794-797 wherein the polypeptide has bioactive priming activity.

The polypeptide can comprise an amino acid sequence having at least 95% identity to any one of SEQ ID NOs. 1-735, 745-787, and 794-797 wherein the polypeptide has bioactive priming activity.

The polypeptide can comprise an amino acid sequence having at least 98% identity to any one of SEQ ID NOs. 1-735, 745-787, and 794-797 wherein the polypeptide has bioactive priming activity.

The polypeptide can comprise an amino acid sequence having at least 99% identity to any one of SEQ ID NOs. 1-735, 745-787, and 794-797 wherein the polypeptide has bioactive priming activity.

b. Preparation of Bioactive Priming Polypeptides

Methods and approaches are provided for cloning, genetically modifying and expressing the bioactive priming polypeptides (for example, flagellins) and the bioactive priming polypeptides (for example, Bt.4Q7Flg22) using those methods well understood and commonly used by one of ordinary skill in the art. The methods described herein can be used with any of the bioactive priming polypeptides as described herein and therefore include any of the flagellins, flagellin-associated polypeptides, thionins, RHPP, serine proteases, ACC deaminases, glucanases and/or any combinations thereof.

Bioactive priming polypeptides can be provided as part of a fusion protein, as a free polypeptide, immobilized on the surface of a particle, or impregnated on or into a matrix. Several expression systems can be used for the production of free polypeptide.

The flagellin-derived full-coding, partial coding (flagellin polypeptides) and flagellin-associated polypeptides can be overexpressed in Bacillus strain, for example, Bacillus thuringiensis strain BT013A, in Bacillus cereus or in Bacillus subtilis. The flagellins and flagellin-derived polypeptides are cloned using an appropriate expression vector to allow for the abundant production of the polypeptide. However, when an expression system such as a Bacillus strain is used, preferably, the peptides are not bound to an exosporium of a Bacillus cereus family member or an intact Bacillus cereus family member spore (i.e., the polypeptides are provided as “free polypeptides.”

For example, in order to facilitate cloning of the target nucleotides that encode the bioactive priming polypeptide(s) as described herein, an E. coli compatible shuttle vector pSUPER was constructed by fusing the pBC plasmid backbone described above with the E. coli pUC57 cloning vector at compatible BamHI restriction endonuclease sites. The resulting, pSUPER vector carries dual selection markers (ampicillin selection in E. coli and tetracycline selection in Bacillus spp). Cloning was performed by PCR amplification of target nucleotides with specific primers synthesized with 15 bp overlapping the pSUPER insertion site. Specific gene encoding polypeptides were fused to the pSUPER vector with In-Fusion HD Cloning Kit (Clontech). Sequence verified pSUPER constructs were amplified using the pBC suitable backbone Reverse and Forward primers. The resulting PCR products were self-ligated to generate the pBC plasmid that was used to transform the donor Bacillus spp. strain. The final construct was verified to be completely intrageneric by Sanger sequencing.

The bioactive priming polypeptides/peptides as described herein are produced in large amounts for field and grower applications by using a free expression system that can utilize a Bacillus subtilis and/or Bacillus thuringiensis strain as the designated heterologous expression strain. The base expression plasmid designated pFEe4B consists of an E. coli section (=e) and a Bacillus section (=pFE). The e section was derived from pUC19 and enables selection and amplification of the vector in E. coli for cloning purposes. It comprises the beta-lactamase gene (bla) conferring resistance to beta-lactam antibiotics such as ampicillin and other penicillin derivatives, as well as an E. coli origin of replication allowing vector multiplication. The pFE section provides selection and plasmid amplification in Bacillus spp. and drives expression of the heterologous polypeptide/peptide of interest. As such it contains a gene conferring resistance to tetracycline (tetL), as well as the gene for a replication protein (repU) responsible for amplifying the plasmid in Bacillus spp., both of which were derived from the native Bacillus cereus plasmid pBC16. The expression cassette of pFEe4B contains a secretion signal (amyQ, SEQ ID NO: 736, Table 20), a cloning site and a terminator (rspD), the former resulting in secretion of the expressed protein/peptide from the host strain cells into the surrounding medium, and the latter preventing transcription beyond the open reading frame of interest. Expression in pFEe4B is driven by a modified autoinducible promoter, which initiates expression once the culture reaches a sufficient optical density. In the pFEe4b expression system, expression is controlled by an IPTG-inducible promoter sequence from Bacillus subtilis. This promoter consists of a modified constitutive promoter combined with the E. coli lac repressor (lacI) and a ribosome binding site. Thus, expression from pFEe4B-encoded polypeptides/peptides depends on the presence of suitable induction agents such as isopropyl beta-D-1-thiogalactopyranoside (IPTG). However other pFe systems useful for expression of the polypeptides as described herein do not rely on such induction systems for their expression. The pFEe4 plasmid further harbors the E. coli lad gene under control of the Bacillus licheniformis penicillinase promoter to prevent expression of polypeptide/peptide as described herein in absence of any induction agent.

Other commercially available expression vectors, for example, any of those derived from Bacillus subtilis, can also be useful. Other expression vectors were selected for producing the recombinant bioactive priming polypeptides due to the following desired criteria: the recombinant microorganism is non-pathogenic and is considered as generally regarded as safe (GRAS) organisms, it has no significant bias in codon usage and it is capable of secreting extracellular proteins directly into the culture medium providing for a cell free version(s) of the bioactive priming polypeptides.

One exemplary system of producing Bt.4QFlg22, Bt.Flg22Syn01, and thionins using fermentation is provided. The polypeptides can be provided in a confirmation to stabilize the polypeptide and enhance activity for an alternative production method, namely bacterial fermentation. The polypeptide (e.g., a polypeptide having an amino acid sequence comprising any one of SEQ ID NOs: 226, 571, or 620) can be combined with an amyQ secretion signal from Bacillus amyloliquefaciens alpha-amylase) fused to glutathione S-transferase (GST, Schistosoma japonicum) and an enterokinase cleavage tag sequence as described in Table 20.

TABLE 20
Sequences useful for increasing stability of an expressed flagellin or flagellin-
associated polypeptide.

| | |
|---|---|
| amyQ secretion signal | MIQKRKRTVSFRLVLMCTLLEVSLPITKTSA |
| (Bacillus amyloliquefaciens) | |
| SEQ ID NO: 736 | |
| GST | MSPILGYWKIKGLVQPTRLLLEYLEEKYEEHLYERDEGDKWRN |
| (Schistosoma japonicum) | KKFELGLEFPNLPYYIDGDVKLTQSMAIIRYIADKHNMLGGCPK |
| SEQ ID NO: 737 | ERAEISMLEGAVLDIRYGVSRIAYSKDFETLKVDFLSKLPEMLK |
| | MFEDRLCHKTYLNGDHVTHPDFMLYDALDVVLYMDPMCLDA |
| | FPKLVCFKKRIEAIPQIDKYLKSSKYIAWPLQGWQATEGGGDHP |
| | PK |
| linker | GGGGGGS |
| SEQ ID NO: 738 | |
| Enterokinase cleavage tag | DDDDK |
| (Consensus cleavage target | |
| for bovine Enterokinase, | |
| light chain protease) | |
| SEQ ID NO: 739 | |

The sequences in Table 20 can be cloned alongside the sequence of interest (e.g., SEQ ID NO: 226, 571, or 620) into a standard cloning vector containing an ampicillin selection marker and either a chloramphenicol (Cm) or Tetracycline (Tet) selection marker that can replicate in E. coli and then be transferred to Bacillus subtilis strain K08 for production purposes, according to standard methods in the art. The fermentation product will result in a fusion protein (e.g., a GST-Bt.4Q7Flg22 fusion protein) which can be applied to the plant or plant part as a fusion protein, or isolated and applied with the GST tag cleaved to result in a purified polypeptide.

Other expression systems common in the art can be utilized to express bioactive priming polypeptides in a similar manner.

The bioactive priming polypeptides as described herein can be produced and purified either by the use of a protein tag(s) using affinity purification or by using column protease cleavage methods which release the un-tagged polypeptide(s). Methods of using this approach to make free versions of the bioactive priming polypeptides are commonly known and understood by one of ordinary skill in the art.

Protein tags usually comprise a relatively small sequence of amino acids incorporated into a translated polypeptide, basically providing a molecular tether for the bioactive priming polypeptide of interest. They are commonly used to aid in the expression and purification of recombinant polypeptides. The glutathione S-transferase (GST) tag was selected for the purposes of affinity purification of the bioactive priming polypeptides as described. A GST tag can be fused to either the N- or C-terminus of a polypeptide. GST tags are frequently combined with other tags for dual-labeling. Tags for the bioactive priming polypeptides can be useful to affinity purify them. The tags can also be cleaved off of the bioactive priming polypeptides using specific proteases and column-specific protease cleavage methods to release the purified un-tagged bioactive priming polypeptide or full-length precursor protein of interest. These methods are also common and well known to one of ordinary skill in the art. Other tags that can be utilized are known in the art, and include polyhistidine (His) tags, FLAG tags, antibody epitopes, streptavidin/biotin, among other purification tools.

Protein tags can be provided within the plasmid to produce the polypeptide. Ideally, the plasmid comprises, alongside the sequence encoding the polypeptide of interest, a secretion signal (e.g., the amyE or amyQ secretion signal) to promote secretion, and a protein tag (e.g., glutathione S transferase) to enhance the stability of the polypeptide, thereby enhancing production and stability. In preferred cases, the protein tag (e.g., GST) is linked to the polypeptide using a linker sequence comprising a consensus cleavage sequence. This can allow the addition of a targeted kinase that can cleave the tag and release the purified, isolated polypeptide. A suitable consensus cleavage sequence can comprise an enterokinase cleavage sequence (DDDDK, SEQ ID NO: 739), which can be cleaved by simple application of a bovine enterokinase, for example.

Therefore, a method is provided for producing a polypeptide comprising producing a fusion protein comprising any polypeptide described herein and an Enterokinase (EK) cleavage site via fermentation, the EK cleavage site serving to enhance activity and stability of the polypeptide. The fusion protein encoded by the plasmid can further comprise a protein tag (e.g., a poly-histidine (His) tag, a FLAG tag, an antibody epitope, streptavidin/biotin, glutathione S-transferase (GST), or any combination thereof), wherein the enterokinase cleavage site comprises a linking region connecting the polypeptide and the protein tag. The fusion protein can also comprise a secretion signal. The secretion signal can comprise an amyE or amyQ secretion signal (e.g., SEQ ID NO: 736), or it can comprise any one of SEQ ID NOs 563-570 or 779-787 or 797 as described above or any other secretion sequences that are well known to those skilled in the art. The polypeptide comprising the enterokinase (EK) cleavage site can be more stable and produced in higher yields using fermentation than a polypeptide lacking the enterokinase (EK) cleavage site. When desired, an enterokinase (e.g., a bovine enterokinase) can be applied to the fusion protein to activate (e.g., isolate) the polypeptide of interest. The enterokinase can be applied on-site to enable maximum stability of the bioactive priming polypeptide prior to administration.

The bioactive priming polypeptides can be provided in a synthetic form using commercially available peptide synthesis technologies to produce high purity polypeptides. Synthetic production of the bioactive priming polypeptides utilizes solid-phase or solution-phase peptide synthesis methodologies that are well known to one of ordinary skill in the art. Chemical synthesis methodologies include: a stepwise assembly of peptides from amino acid precursors, whereby peptide elongation proceeds via cleavage of a reversible amino acid protecting group followed by a coupling reaction between amino acids. Solid phase peptide synthesis is used to add a covalent attachment step that links the nascent peptide chain to an insoluble polymeric support whereby the anchored peptide can be extended by a series of cycles. Polypeptides may be optionally assembled in smaller units or fragments, that are later conjugated to product the full-length polypeptide sequence. Polypeptide extension reactions are driven to completion and then the synthesized polypeptide is removed from the solid support by washing with a strong acid, followed by steps to produce a highly purified peptide, optionally to include precipitation, salt exchange, filtration and lyophilization Mass spectrometry, nitrogen content, amino acid composition, and high-pressure liquid chromotography analyses are performed after the completion of synthesis and purification for confirmation of molecular mass, polypeptide sequence and determination of purity.

Any of the bioactive priming polypeptides as described herein for flagellin-associated polypeptides (Tables 1-5), RHPP (Table 11-13), thionin and thionin-like polypeptides (Table 15), serine proteases (Table 17), ACC deaminase (Table 18), or glucanases, amylases, and chitinases (Table 19) can be provided in synthetic forms.

Additionally, such methods can be used for making and using conserved assistance sequences preferably named signature (SEQ ID NOs: 542-548), signal anchor sorting (SEQ ID NOs: 549-562) and secretion (SEQ ID NOs: 563-570) sequences.

Retro inverso can also be made synthetically or chemically manufactured. Synthetic polypeptides produced in the all-D confirmation are prepared by replacing all the L-amino acid residues with their D-enantiomers resulting in a reversed or retro-all-D-isomer Flg polypeptide. Solid phase synthesis is used to prepare the retro-inverso versions of the Flg polypeptide(s). After synthesis and purification of the retro-inverso polypeptide(s), the amino acid composition is confirmed using mass spectrometry of the Flg polypeptide(s). The purity of the retro-inverso polypeptide(s) is then confirmed at a level greater or equal to 95% using HPLC analysis. The retro-inverso versions of the Flg polypeptide(s) are further characterized using HPLC retention time, relative molecular mass and amino acid composition values (IC50 μM). Retro inverso production using recombinant DNA technology generally involves the use of non-ribosomal protein synthesis mechanisms.

Retro-inverso synthetic Flg bioactive priming polypeptides prepared by solid phase synthesis could be tested for their capacity to bind to the FLS2 or alternative FLS receptors, for example, FLS3 also found in plants. Competitive ELISA experiments or in vivo binding assays with labeled peptides (e.g. biotin, GST) could be used to confirm the binding affinities of retro inverso Flg-associated polypeptides to plant FLS receptors.

Recombinant Bacteria that Express Bioactive Priming Polypeptides

A recombinant microorganism that expresses or overexpresses a polypeptide is also provided. The polypeptide comprises the polypeptides as described above for the composition. For example, the polypeptide can comprise a flagellin or flagellin-associated polypeptide, a RHPP; a thionin or thionin-like polypeptide), a glucanase polypeptide, an amylase polypeptide, a chitinase polypeptide, a serine protease polypeptide, or an ACC deaminase polypeptide. For example, the polypeptide can comprise a flagellin or flagellin-associated polypeptide having an amino acid sequence comprising any one of SEQ ID NOs: 226, 1-225, 227-375, 526, 528, 530, 532, 534, 536, 538, 540, 541, or 571-603; or an RHPP having an amino acid sequence comprising any one of 604, 606-610 and 745-755; or a thionin or thionin-like polypeptide having an amino sequence comprising any one of SEQ ID NOs: 620-719; or a glucanase polypeptide having an amino acid sequence comprising any one of SEQ ID NOs: 731-733 and 767-776; or an amylase having an amino acid sequence comprising SEQ ID NO: 734 or SEQ ID NO: 735; or a chitinase having an amino acid sequence comprising SEQ ID NO: 777 or SEQ ID NO: 778; or a serine protease having an amino acid sequence comprising any one of SEQ ID NOs: 721, 722 and 794-796; or an ACC deaminase polypeptide having an amino acid sequence comprising any one of SEQ ID NOs: 723-730.

The polypeptide can be overexpressed by the microorganism.

The recombinant microorganism can comprise a microorganism that is capable of making recombinant bioactive priming polypeptides or their precursors in an effective manner. The preferred microorganism would be from the genus Bacillus, a bacterium of the genus Paenibacillus, a fungus of the genus Penicillium, a bacterium of the genus Glomus, a bacterium of the genus Pseudomonas, a bacterium of the genus Arthrobacter, a bacterium of the genus Paracoccus, a bacterium of the genus Rhizobium, a bacterium of the genus Bradyrhizobium, a bacterium of the genus Azosprillium, a bacterium of the genus Enterobacter, a bacterium of the genus Escherichia, or any combination thereof.

The recombinant microorganism can comprise a bacterium of the genus Bacillus, a bacterium of the genus Paenibacillus, or any combination thereof.

For example, the microorganism can comprise Bacillus mycoides, Bacillus pseudomycoides, Bacillus cereus, Bacillus thuringiensis, Bacillus megaterium, Bacillus subtilis, Bacillus firmus, Bacillus aryabhattai, Bacillus amyloliquefaciens, Bacillus licheniformis, Bacillus circulans, Bacillus flexus, Bacillus nealsonii, Bacillus pumulis, Paenibacillus genus bacterium or a combination thereof.

Methods and approaches are commonly used by one of ordinary skill in the art to determine and verify the genus and species of the bacteria. A common method provides chromosomal DNA isolated from the bacteria with PCR amplification of the 16s rRNA region using universal primers (ACTCCTACGGGAGGCAGCAGT, SEQ ID NO: 740) and (GGGTTGCGCTCGTTG/AC, SEQ ID NO: 741). The PCR amplicons are then purified and sequenced for correct identification of the appropriate bacterial strain, for example a specific strain in the genera of Bacillus.

Sample protocols are generally known to one in the art for the preparation of chromosomal DNA, transformation of the DNA of genes encoding the polypeptides using a plasmid, producing the polypeptides in a host bacterium, for example, a Bacillus strain.

The Bacillus strains provided can produce any bioactive priming polypeptide as described herein or a combination thereof. For example, the strain can comprise: (a) Bacillus aryabhattai CAP53 (NRRL No. B-50819),

(b) Bacillus aryabhattai CAP56 (NRRL No. B-50817),
(c) Bacillus flexus BT054 (NRRL No. B-50816),
(d) Paracoccus kondratievae NC35 (NRRL No. B-50820),
(e) Bacillus mycoides BT155 (NRRL No. B-50921),
(f) Bacillus nealsonii BOBA57 (NRRL No. NRRL B-50821),
(g) Bacillus mycoides EE118 (NRRL No. B-50918),
(h) Bacillus subtilis EE148 (NRRL No. B-50927),
(i) Bacillus mycoides EE141 (NRRL NO. B-50916),
(j) Bacillus mycoides BT46-3 (NRRL No. B-50922),
(k) Bacillus cereus family member EE128 (NRRL No. B-50917),
(l) Paenibacillus massiliensis BT23 (NRRL No. B-50923),
(m) Bacillus cereus family member EE349 (NRRL No. B-50928),
(n) Bacillus subtilis EE218 (NRRL No. B-50926),
(o) Bacillus megaterium EE281 (NRRL No. B-50925),
(p) Bacillus cereus family member EE-B00377 (NRRL B-67119);
(q) Bacillus pseudomycoides EE-B00366 (NRRL B-67120),
(r) Bacillus mycoides EE-B00363 (NRRL B-67121),
(s) Bacillus pumilus EE-B00143 (NRRL B-67123),
(t) Bacillus thuringiensis EE-B00184 (NRRL B-67122),
(u) Bacillus mycoides EE116 (NRRL No. B-50919),
(v) Bacillus cereus family member EE417 (NRRL No. B-50974),
(w) Bacillus subtilis EE442 (NRRL No. B-50975),
(x) Bacillus subtilis EE443 (NRRL No. B-50976),
(y) Bacillus cereus family member EE444 (NRRL No. B-50977),
(z) Bacillus subtilis EE405 (NRRL No. B-50978),
(aa) Bacillus cereus family member EE439 (NRRL No. B-50979),
(bb) Bacillus megaterium EE385 (NRRL No. B-50980),
(cc) Bacillus cereus family member EE387 (NRRL No. B-50981),
(dd) Bacillus circulans EE388 (NRRL No. B-50982),
(ee) Bacillus thuringiensis EE319 (NRRL No. B-50983),
(ff) Bacillus cereus family member EE377 (NRRL No. B-67119),
(gg) Bacillus mycoides EE363 (NRRL No. B-67121),
(hh) Bacillus pseudomycoides EE366 (NRRL No. B-67120);
(ii) Bacillus thuringiensis BT013A (NRRL No. B-50924);
or any combination thereof. Each of these strains has been deposited with the United States Department of Agriculture (USDA) Agricultural Research Service (ARS), having the address 1815 North University Street, Peoria, Ill. 61604 U.S.A., and are identified by the NRRL deposit numbers provided in parentheses. Strains (a)-(d) and (g) were deposited on Mar. 11, 2013. Strains (e), (g)-(o), (u), and (ii) were deposited on Mar. 10, 2014. Strains (v)-(hh) were deposited on Sep. 10, 2014. Strain (ee) was deposited on Sep. 17, 2014. Strains (p)-(t), (ff), (gg), and (hh) were deposited on Aug. 19, 2015. Bacillus thuringiensis BT013A is also known as Bacillus thuringiensis 4Q7.

The isolation and characterization of these strains are described in the Examples found within International Publication No: WO/2017/161091, incorporated herein by reference in its entirety. For ease of identification of the organism, International Publication No: WO/2017/161091 A1 also provides the partial 16S ribosomal RNA sequences for each of these strains in a sequence list and in Table 17.

Any of the recombinant microorganisms can be used to overexpress a bioactive priming polypeptide as described herein for a flagellin-associated polypeptide (Tables 1-5), an RHPP (Table 11-13), a thionin or thionin-like polypeptide (Table 15), a serine protease polypeptide (Table 17), an ACC deaminase polypeptide (Table 18) or a glucanase, amylase or chitinase polypeptide (Table 19).

The recombinant microorganism can comprise a mixture of two or more of any of the recombinant microorganisms described herein.

The recombinant microorganism can be inactivated. Inactivation results in microorganisms that are unable to reproduce. Inactivation of microorganisms can be advantageous, for example because it allows for delivery of the microorganism to a plant or a plant growth medium while reducing or eliminating any detrimental effects that the live microorganism may have on a plant or on the environment. The recombinant microorganism can be inactivated by any physical or chemical means, e.g., by heat treatment, gamma irradiation, x-ray irradiation, UV-A irradiation, UV-B irradiation, or treatment with a solvent such as glutaraldehyde, formaldehyde, hydrogen peroxide, acetic acid, bleach, chloroform, or phenol, or any combination thereof.

c. Inducers

The inducer compound can comprise an amino acid or isomer thereof, a substituted or unsubstituted benzoic acid or derivative or salt thereof, a dicarboxylic acid or derivative or salt thereof, a benzodiathiazole, a betaine, a proline, a bacteriocide, a callose synthase inhibitor, a succinate dehydrogenase inhibitor, or salt thereof, or any combination thereof.

Amino Acid.

The composition can comprise an amino acid. The amino acids that can be used in the compositions herein are preferably distinct from the amino acids that comprise the polypeptides. For example, the amino acid can be an isolated amino acid. Further, the amino acid can comprise any isomer or stereoisomer of any amino acid. For example, the amino acid can be a D or an L amino acid and could be an alpha or beta isomer of an amino acid. The amino acids may be a proteinogenic (e.g., canonical) or non-proteinogenic amino acid. Particularly suitable amino acids that can be used as inducer compounds include cysteine and β-amino butyric acid (BABA), discussed below.

β-amino butyric acid (BABA) is an isomer of the amino acid aminobutyric acid with the chemical formula C₄H₉NO₂. BABA is a non-proteinogenic amino acid and not found in native proteins. It can induce plant disease resistance and also improves resistance to abiotic stresses when applied to plants.

L-Cysteine.

Cysteine has traditionally been considered to be a hydrophilic amino acid, based largely on the chemical parallel between its sulfhydryl group and the hydroxyl groups in the side chains of other polar amino acids with the formula HO₂CCH(NH₂)CH₂SH. The thiol side chain in cysteine often participates in enzymatic reactions, as a nucleophile. The thiol is susceptible to oxidation to give the disulfide derivative cystine, which serves an important structural role in many proteins. Cysteine has traditionally been considered to be a hydrophilic amino acid, based largely on the chemical parallel between its sulfhydryl group and the hydroxyl groups in the side chains of other polar amino acids. However, cysteine is also considered a proteinogenic amino acid. Cysteine can be provided to treat HLB in the forms of L- or D-cysteine and in any form that is provided as a cysteine analog, acid or salt thereof. L-cysteine levels in the plant have a multi-pronged effect and modulate plant responses to stress, in part through the synthesis of sulfur containing antimicrobial proteins and maintenance of cellular redox state (Gotor et al., “Signaling in the plant cytosol: cysteine or sulfide?” Amino Acids: 47: 2155-2164, 2015).

The cysteine included in the compositions described herein can be any analog, acid or salt of cysteine. For example, the compositions can comprise a cysteine having the form of L-cysteine, D-cysteine, DL-cysteine, analogs of L-cysteine comprising: DL homocysteine, L-cysteine methyl ester, L-cysteine ethyl ester, N-carbamoyl cysteine, N-acetylcysteine, L-cysteine sodium salt, L-cysteine monosodium salt L-cysteine disodium salt, L-cysteine monohydrochloride, L-cysteine hydrochloride, L-cysteine ethyl ester hydrochloride, L-cysteine methyl ester hydrochloride, other selenocysteines, seleno-DL-cysteine, N-isobutyryl-L-cysteine, N-isobutyryl-L-cyteine or an acid of cysteine such as cysteine sulfinic acid.

Benzoic Acid.

The composition can comprise a substituted or unsubstituted benzoic acid. Preferably, the substituted benzoic acid comprises salicylic acid or any derivative, analog or salt thereof. For example, the composition can comprise salicylic acid. Another analog of salyclic acid that can be used in the composition is benzothiadiazole, discussed below.

Benzothiadiazole.

The composition can comprise a benzothiadiazole as the inducer compound. Preferably, the benzothiadiazole comprises Benzo (1,2,3)-thiadiazole-7-carbothioic acid-S-methyl ester (BTH; C₈H₆N₂OS₂) available commercially as Actigard 50WG fungicide (Syngenta). BTH induces systemic and/or host plant acquired resistance and exhibits a unique mode of action which mimics the natural systemic acquired resistance (SAR) response found in most plant species. BTH is a salicylic acid analog with increased stability that is used agriculturally as an activator of plant immune responses and is approved for application to citrus trees as root drench or irrigation treatment to prevent HLB. This BTH inducer compound is advantageously used in combinations with Flg22 peptides to prevent and reduce citrus disease.

Dicarboxylic Acid.

The composition can comprise a dicarboxylic acid. Preferably, the dicarboxylic acid comprises oxalic acid. Therefore, the composition can comprise oxalic acid.

Bacteriocide.

The composition can comprise a bacteriocide. The bacteriocide can comprise streptomycin, penicillins, tetracyclines, oxytetracycline, kasugamycin, ampicillin, copper oxide, copper hydroxide, copper sulfide, copper sulfate, fine particle coppers, oxolinic acid, chlorotetracycline, acetic acid, or any combination thereof. Preferably, the bacteriocide comprises oxytetracycline.

Callose Synthase Inhibitor.

The composition can comprise a callose synthase inhibitor. Callose is a multi-functional polysaccharide in the form of β-1,3-glucan and some β-1,6-glucan linkages that is produced by a family of callose synthase enzymes. Callose is deposited in the cell wall to regulate various developmental processes and plant responses to abiotic and biotic stress. For example, callose is deposited around the plasmodesmata that connects cells, thus regulating flow between cells. During phloem formation, the callose is degraded between the developing sieve tube elements, thus opening the connections and allowing for transport of carbohydrates, primarily sucrose, in the plant. Callose can also act as a physical barrier to infection and is deposited within the cell wall in response to fungal and bacterial infection. The synthesis and breakdown of callose must be tightly regulated by the plant. Thus, callose degradation is facilitated by a family plant β-1,3-endoglucanases that either hydrolyze or transfer glycosides. Bacteria also express β-1,3-endoglucanases for degradation of β1,3-glucans derived from fungal and plant cell walls. Mis-regulation of callose deposition may occur in response to CLas infection due to increased activity of callose synthase and/or decreased β-1,3-endoglucanase activity. Compositions comprising callose synthase inhibitors can help clear phloem blockages from callose build up and assist with recovery in plants infected with HLB or CLas. The callose synthase inhibitors can comprise 2-deoxy-D-glucose (2-DDG), 3-aminobenzamide, 3-methoxybenzamide or any combination thereof. Preferably, the callose synthase inhibitor comprises 2-deoxy-D-glucose (2-DDG). 2-DDG is a non-metabolizable glucose analogue. It is a known inhibitor of callose synthase and when used in the compositions and methods described herein can aid in the removal of callose build-up caused by infection of citrus with the CLas bacteria.

Succinate Dehydrogenase Inhibitor.

The composition can comprise a succinate dehydrogenase inhibitor. Succinate dehydrogenase is a mitochondrial metabolic enzyme complex and is integral for cell respiration. The succinate dehydrogenase inhibitors can be used as a fungicide (e.g., the composition can comprise a fungicide comprising a succinate dehydrogenase inhibitor). The succinate dehydrogenase inhibitor can comprise a phenyl-benzamide, phenyl-oxo-ethyl thiophene amide, pyridinyl-ethyl-benzamide, furan-carboxamide, oxathin-carboxamide, thiazole-carboxamide, pyrazole-4-carboxamide, N-cyclopropyl-N-benzyl-pyrazole-carboxamide, N-methoxy-(phenyl-ethyl)-pyrazole-carboxamide, pyridine-carboxamide, or pyrazine-carboxamide, pydiflumetofen, benodanil, flutolanil, mepronil, isofetamid, fluopyram, fenfuram, carboxin, oxycarboxin, thifluzamide, benzovindiflupyr, bixafen, fluindapyr, fluxapyroxad, furametpyr, inpyrfluxam, isopyrazam, penflufen, penthiopyrad, sedaxane, isoflucypram, pydiflumetofen, boscalid, or pyraziflumid or any combination, homolog, or analog thereof. For example, the succinate dehydrogenase inhibitor fungicide can comprise a phenyl-benzamide, phenyl-oxo-ethyl thiophene amide, pyridinyl-ethyl-benzamide, furan-carboxamide, oxathin-carboxamide, thiazole-carboxamide, pyrazole-4-carboxamide, N-cyclopropyl-N-benzyl-pyrazole-carboxamide, N-methoxy-(phenyl-ethyl)-pyrazole-carboxamide, pyridine-carboxamide, or pyrazine-carboxamide, pydiflumetofen, isofetamid, oxycarboxin, benzovindiflupyr, bixafen, fluindapyr, inpyrfluxam, isopyrazam, penthiopyrad, isoflucypram, pydiflumetofen, pyraziflumid or any combination thereof. For example, the succinate dehydrogenase inhibitor can comprise bixafen.

Betaine.

The composition can comprise a betaine. As used herein, “betaine” refers to any betaine, betaine homolog, or betaine analog. The betaine can comprise glycine betaine, glycine betaine aldehyde, β-alanine betaine, betaine hydrochloride, cetyl betaine, proline betaine, choline-O-sulfate betaine, cocaamidopropyl betaine, oleyl betaine, sulfobetaine, lauryl betaine, octyl betaine, caprylamidopropyl betaine, lauramidopropyl betaine, isostearamidopropyl betaine, or a combination, homolog, or analog of any thereof.

For example, the betaine can comprise glycine betaine, glycine betaine aldehyde, β-alanine betaine, betaine hydrochloride, cetyl betaine, choline-O-sulfate betaine, cocaamidopropyl betaine, oleyl betaine, sulfobetaine, lauryl betaine, octyl betaine, caprylamidopropyl betaine, lauramidopropyl betaine, isostearamidopropyl betaine, or a combination, homolog, or analog of any thereof.

For example, the betaine can comprise glycine betaine or betaine hydrochloride.

The betaine can be derived from a plant source such as wheat (e.g., wheat germ or wheat bran) or a plant of the genus Beta (e.g., Beta vulgaris (beet)).

The betaine homolog or analog can comprise ectoine, choline, phosphatidylcholine, acetylcholine, cytidine disphosphate choline, dimethylethanolamine, choline chloride, choline salicylate, glycerophosphocholine, phosphocholine, a sphingomyelin, choline bitartrate, propio betaine, deanol betaine, homodeanol betaine, homoglycerol betaine, diethanol homobetaine, triethanol homobetaine, or a combination of any thereof.

Proline.

The composition can comprise a proline. As used herein, “proline” refers to any proline, proline analog, or proline homolog. The proline can comprise L-proline, D-proline, hydroxyproline, hydroxyproline derivatives, proline betaine, or a combination, derivative, homolog, or analog of any thereof.

For example, the proline can comprise L-proline.

The proline homolog or analog can comprise α-methyl-L-proline, α-benzyl-Lproline, trans-4-hydroxy-L-proline, cis-4-hydroxy-L-proline, trans-3-hydroxy-L-proline, cis-3-hydroxy-L-proline, trans-4-amino-L-proline, 3,4-dehydro-α-proline, (2S)-aziridine-2-carboxylic acid, (2S)-azetidine-2-carboxylic acid, L-pipecolic acid, proline betaine, 4-oxo-L-proline, thiazolidine-2-carboxylic acid, (4R)-thiazolidine-4-carboxylic acid, or a combination of any thereof. Compositions comprising a proline are effective protein stabilizers and can help prevent protein unfolding during periods of stress, including biotic and abiotic.

Unless otherwise specified each inducer compound can comprise from about 0.000001 wt. % to about 95 wt. %, from about 0.000001 wt. % to about 10 wt. %, from about 0.001 wt. % to about 5 wt. %, or from about 0.001 wt. % to about 1 wt. % of the composition, according to the total weight of the composition.

### II. Specific Compositions in Embodiments

The compositions herein can comprise any of the bioactive priming polypeptides or polypeptides described herein. Further, the compositions can consist essentially of the bioactive priming polypeptides or polypeptides as described herein.

The composition can comprise at least one bioactive priming polypeptide.

The composition can comprise at least one flagellin or flagellin-associated polypeptide. An amino acid sequence of the flagellin or flagellin associated polypeptide can comprise any one of SEQ ID NOs: 226, 289, 290, 291, 293, 294, 295, 300, 437, 526, 532, 534, 536, 538, 540, 571-585, and 587-603. In some cases, the amino acid sequence of the flagellin or flagellin associated polypeptide comprises any one of SEQ ID NOs: 226, 293, 295, 300, 540, 571-579, and 589-590. For example, the composition can comprise a flagellin or flagellin-associated polypeptide having an amino acid sequence comprising SEQ ID NO: 226, 590 or 571. For example, the composition can comprise a flagellin or flagellin-associated polypeptide having an amino acid sequence comprising SEQ ID NO: 226 or 571. For example, the composition can comprise a flagellin or flagellin-associated polypeptide having an amino acid sequence comprising or consisting of SEQ ID NO: 226.

The composition can comprise at least one retro inverso flagellin or flagellin-associated polypeptide. The retro-inverso flagellin or flagellin associated polypeptide can comprise a retro-inverso Flg22 polypeptide, a retro-inverso FlgII-28 polypeptide and/or an Flg15 polypeptide.

The composition can comprise at least one retro inverso Flg22 polypeptide. An amino acid sequence of the retro inverso Flg22 polypeptide can comprise any one of SEQ ID NOs: 376-450, 527, 531, 533, 535, 537 and 539.

The composition can comprise at least one retro-inverso FlgII-28 polypeptide. An amino acid sequence of the retro-inverso FlgII-28 polypeptide can comprise any one of SEQ ID NOs: 451-525.

The composition can comprise at least one retro-inverso Flg15 polypeptide. An amino acid sequence of the retro-inverso Flg15 polypeptide can comprise SEQ ID NOs: 529.

The composition can comprise at least one RHPP. An amino acid sequence of the RHPP polypeptide can comprise any one of SEQ ID Nos: 604, 607, 608, and 745-755. For example, the composition can comprise an RHPP having an amino acid sequence comprising SEQ ID NO: 604.

The composition can comprise at least one retro-inverso RHPP polypeptide. An amino acid sequence of the retro-inverso RHPP polypeptide can comprise any one of SEQ ID NO: 605, 609, 610, and 756-766.

The composition can comprise at least one thionin or thionin-like polypeptide. An amino acid sequence of the thionin or thionin-like polypeptide can comprise any one of SEQ ID NOs: 620-719. For example, the composition can comprise a thionin or thionin-like polypeptide having an amino acid sequence comprising SEQ ID NO: 620. In some instances, the thionin or thionin-like polypeptide can be fused to a phloem targeting sequence to form a fused polypeptide. The phloem or phloem targeting sequence can comprise any one of SEQ ID NOs: 611-619 or any combination thereof. In some cases, the phloem or phloem targeting sequence comprises SEQ ID NO: 611. In some cases, the fusion polypeptide comprising a thionin or thionin-like polypeptide and a phloem or phloem targeting sequence can comprise SEQ ID NO: 720.

The composition can comprise at least one glucanase polypeptide. An amino acid sequence of the glucanase polypeptide can comprise any one of SEQ ID NOs: 731-735 and 767-776. For example, the composition can comprise a β-1,3-glucanase. An amino acid sequence of the β-1,3-glucanase can comprise SEQ ID NO: 772 or 732.

The composition can comprise at least one amylase. An amino acid sequence of the amylase polypeptide can comprise SEQ ID NO: 734 or SEQ ID NO: 735.

The composition can comprise at least one chitinase. An amino acid sequence of the chitinase polypeptide can comprise SEQ ID NO: 777 or SEQ ID NO: 778.

The composition can comprise at least one serine protease polypeptide. An amino acid sequence of the serine protease polypeptide can comprise any one of SEQ ID NOs: 721, 722 and 794-796. For example, the composition can comprise a serine protease polypeptide having an amino acid sequence comprising SEQ ID NO: 722 or 795. For example, the composition can comprise a serine protease polypeptide having an amino acid sequence comprising SEQ ID NO: 794 or 796.

The composition can comprise at least one ACC deaminase polypeptide. An amino acid sequence of the ACC deaminase polypeptide can comprise any one of SEQ ID NOs: 723-730. For example, the composition can comprise an ACC deaminase polypeptide having an amino acid sequence comprising SEQ ID NO: 730.

The composition can comprise at least two bioactive polypeptides.

The composition can comprise a flagellin or flagellin associated polypeptide and a thionin or thionin-like polypeptide. For example, the composition can comprise a flagellin or flagellin associated polypeptide having an amino acid sequence comprising SEQ ID NO: 571 or 226 and a thionin polypeptide having an amino acid sequence comprising SEQ ID NO: 620.

The composition can comprise a flagellin or flagellin associated polypeptide and an RHPP polypeptide. For example, the composition can comprise a flagellin or flagellin associated polypeptide having an amino acid sequence comprising SEQ ID NO: 571 or 226 and an RHPP polypeptide having an amino acid sequence comprising SEQ ID NO: 604.

The composition can comprise a flagellin or flagellin associated polypeptide and a serine protease. For example, the composition can comprise a flagellin or flagellin associated polypeptide having an amino acid sequence comprising SEQ ID NO: 571 and a serine protease having an amino acid sequence comprising SEQ ID NO: 722. As another example, the composition can comprise a flagellin or flagellin associated polypeptide having an amino acid sequence comprising SEQ ID NO: 226 and a serine protease having an amino acid sequence comprising SEQ ID NO: 794. For example, the composition can comprise a flagellin or flagellin associated polypeptide having an amino acid sequence comprising SEQ ID NO: 226 and a serine protease having an amino acid sequence comprising SEQ ID NO: 722. As another example, the composition can comprise a flagellin or flagellin associated polypeptide having an amino acid sequence comprising SEQ ID NO: 226 and a serine protease having an amino acid sequence comprising SEQ ID NO: 796. For example, the composition can comprise a flagellin or flagellin associated polypeptide having an amino acid sequence comprising SEQ ID NO: 226 and a serine protease having an amino acid sequence comprising SEQ ID NO: 795.

The composition composition can comprise a flagellin or flagellin associated polypeptide and a glucanase. For example, the composition can comprise a flagellin or flagellin associated polypeptide having an amino acid sequence comprising SEQ ID NO: 571 and a glucanase having an amino acid sequence comprising any one of SEQ ID NOs: 731-735. In some cases, the composition can comprise a flagellin or flagellin associated polypeptide having an amino acid sequence comprising SEQ ID NO: 571 and a glucanase having an amino acid sequence comprising any one of SEQ ID NOs: 731-733 or any one of SEQ ID NOs: 767-766. As another example, the composition can comprise a flagellin or flagellin associated polypeptide having an amino acid sequence comprising SEQ ID NO: 226 and a glucanase having an amino acid sequence comprising any one of SEQ ID NOs: 731-733 and an amylase polypeptide having an amino acid sequence comprising SEQ ID NO: 734 or SEQ ID NO: 735. In some cases, the composition can comprise a flagellin or flagellin associated polypeptide having an amino acid sequence comprising SEQ ID NO: 226 and a glucanase having an amino acid sequence comprising any one of SEQ ID NOs: 731-733 or any one of SEQ ID NOs: 767-766. In some compositions the amino acid sequence of the glucanase polypeptide can comprise SEQ ID NO: 772. In some compositions the amino acid sequence of the glucanase polypeptide (e.g., a β-1,3-glucanase) can comprise SEQ ID NO: 732.

The composition can comprise glucanase and an amylase. For example, the composition can comprise a glucanase polypeptide (e.g., a β-1,3-glucanase) having an amino acid sequence comprising SEQ ID NO: 731-733 and 767-766 and an amylase polypeptide having an amino acid sequence comprising SEQ ID NO: 734 or SEQ ID NO: 735. In some compositions the amino acid sequence of the glucanase polypeptide can comprise SEQ ID NO: 772. In some compositions the amino acid sequence of the glucanase polypeptide (e.g., the β-1,3-glucanase) can comprise SEQ ID NO: 732.

The composition can comprise glucanase and a chitinase. For example, the composition can comprise a glucanase polypeptide (e.g., a β-1,3-glucanase) having an amino acid sequence comprising SEQ ID NO: 731-733 and 767-766 and a chitinase polypeptide having an amino acid sequence comprising SEQ ID NO: 777 or SEQ ID NO: 778. In some compositions the amino acid sequence of the glucanase polypeptide (e.g., a β-1,3-glucanase) can comprise SEQ ID NO: 772. In some compositions the amino acid sequence of the glucanase polypeptide (e.g., the β-1,3-glucanase) can comprise SEQ ID NO: 732.

The composition can comprise a glucanase and a serine protease. For example, the composition can comprise a glucanase polypeptide (e.g., a β-1,3-glucanase) having an amino acid sequence comprising SEQ ID NO: 731-733 and 767-766 and a serine protease polypeptide having an amino acid sequence comprising SEQ ID NO: 721, SEQ ID NO: 722 or any one of SEQ ID NO: 794-796. In some compositions, the amino acid sequence of the glucanase polypeptide (e.g., a β-1,3-glucanase) can comprise SEQ ID NO: 772. In some compositions the amino acid sequence of the glucanase polypeptide (e.g., the β-1,3-glucanase) can comprise SEQ ID NO: 732.

Compositions described herein having a glucanase in combination with an amylase, chitinase, or serine protease can further comprise at least one flagellin or flagellin associated polypeptide.

For example, a composition can comprise at least one flagellin or flagellin associated polypeptide, a β-1,3-endoglucanase and an amylase. For instance, the composition can comprise a flagellin or flagellin associated polypeptide having an amino acid sequence comprising SEQ ID NO: 226 or 571, a β-1,3-endoglucanase having an amino acid sequence comprising any one of SEQ ID NO: 731-733 and 767-776, and an amylase having an amino acid sequence comprising SEQ ID NO: 734 or 735. In some compositions the amino acid sequence of the glucanase polypeptide (e.g., the β-1,3-glucanase) can comprise SEQ ID NO: 732 or 772.

Alternatively, the composition can comprise at least one flagellin or flagellin associated polypeptide, a β-1,3-endoglucanase and a chitinase. For instance, the composition can comprise a flagellin or flagellin associated polypeptide having an amino acid sequence comprising SEQ ID NO: 226 or 571, a β-1,3-endoglucanase having an amino acid sequence comprising any one of SEQ ID NO: 731-733 and 767-776, and a chitinase having an amino acid sequence comprising SEQ ID NO: 777 or SEQ ID NO: 778. In some compositions the amino acid sequence of the glucanase polypeptide (e.g., the β-1,3-glucanase) can comprise SEQ ID NO: 732 or 772.

Alternatively, the composition can comprise at least one flagellin or flagellin associated polypeptide, a β-1,3-endoglucanase and a serine protease. For instance, the composition can comprise a flagellin or flagellin associated polypeptide having an amino acid sequence comprising SEQ ID NO: 226 or 571, a β-1,3-endoglucanase having an amino acid sequence comprising any one of SEQ ID NO: 731-733 and 767-776, and a serine protease having an amino acid sequence comprising SEQ ID NO: 721, SEQ ID NO: 722, or any one of SEQ ID NOs: 794-796. In some compositions the amino acid sequence of the glucanase polypeptide (e.g., the β-1,3-glucanase) can comprise SEQ ID NO: 732 or 772.

The composition can comprise a flagellin or flagellin associated polypeptide and an amylase. For example, the composition can comprise a flagellin or flagellin associated polypeptide having an amino acid sequence comprising SEQ ID NO: 226 or 571 and an amylase having an amino acid sequence comprising SEQ ID NO: 734 or 735.

The composition can comprise a flagellin or flagellin associated polypeptide and a chitinase. For example, the composition can comprise a flagellin or flagellin associated polypeptide having an amino acid sequence comprising SEQ ID NO: 226 or 571 and a chitinase having an amino acid sequence comprising SEQ ID NO: 777 or 778.

The composition can comprise a flagellin or flagellin associated polypeptide and an ACC deaminase. For example, the composition can comprise a flagellin or flagellin associated polypeptide having an amino acid sequence comprising SEQ ID NO: 226 or 571 and an ACC deaminase having an amino acid sequence comprising SEQ ID NO: 730.

The composition can comprise a root hair promoting polypeptide (RHPP) or a retro inverso root hair promoting polypeptide (RI-RHPP) and a glucanase. For example, the composition can comprise an RHPP having an amino acid sequence comprising any one of SEQ ID NOs: 604, 607-608 and 745-756 or an RI-RHPP comprising any one of SEQ ID NOs: 605, 609-610 and 757-766 and a β-1,3-glucanase having an amino acid sequence comprising any one of SEQ ID NOs: 731-733 and 767-776.

The composition can comprise a root hair promoting polypeptide (RHPP) or a retro inverso root hair promoting polypeptide (RI-RHPP) and an ACC deaminase. For example, the composition can comprise an RHPP having an amino acid sequence comprising any one of SEQ ID NOs: 604, 607-608 and 745-756 or an RI-RHPP comprising any one of SEQ ID NOs: 605, 609-610 and 757-766 and an ACC deaminase having an amino acid sequence comprising any one of SEQ ID NOs: 723-730.

The composition can comprise a bioactive polypeptide and at least one inducer compound.

The composition can comprise a flagellin or flagellin associated polypeptide and a callose synthase inhibitor. For example, the composition can comprise a flagellin or flagellin associated polypeptide having an amino acid sequence comprising SEQ ID NO: 226 or 571 and a callose synthase inhibitor. The callose synthase inhibitor can comprise 2-DDG. Optionally, the composition can further comprise a bacteriocide (e.g., oxytetracycline).

The composition can comprise a flagellin or flagellin associated polypeptide and an amino acid. For example, the composition can comprise a flagellin or flagellin associated polypeptide having an amino acid sequence comprising SEQ ID NO: 226 or 571 and an amino acid. The amino acid can comprise L-cysteine or β-amino-butyric acid (BABA). Preferably, the amino acid comprises β-amino-butyric acid (BABA). Optionally, the composition can further comprise a bacteriocide (e.g., oxytetracycline).

The composition can comprise a flagellin or flagellin associated polypeptide and a substituted or unsubstituted benzoic acid. For example, the composition can comprise a flagellin or flagellin associated polypeptide having an amino acid sequence comprising SEQ ID NO: 226 or 571 and a substituted or unsubstituted benzoic acid. The substituted benzoic acid can comprise salicylic acid. Optionally, the composition can further comprise a bacteriocide (e.g., oxytetracycline).

The composition can comprise a flagellin or flagellin associated polypeptide and a benzothiadiazole. For example, the composition can comprise a flagellin or flagellin associated polypeptide having an amino acid sequence comprising SEQ ID NO: 226 or 571 and a benzothiadiazole. The benzothiadiazole can comprise benzo (1,2,3)-thiadiazole-7-carbothioic acid-S-methyl ester, available commercially as ACTIGARD 50WG fungicide. Optionally, the composition can further comprise a bacteriocide (e.g., oxytetracycline).

The composition can comprise a flagellin or flagellin associated polypeptide and a dicarboxylic acid. For example, the composition can comprise a flagellin or flagellin associated polypeptide having an amino acid sequence comprising SEQ ID NO: 226 or 571 and a dicarboxylic acid. The dicarboxylic acid can comprise oxalic acid. Optionally, the composition can further comprise a bacteriocide (e.g., oxytetracycline).

The composition can comprise a flagellin or flagellin associated polypeptide and a betaine. For example, the composition can comprise a flagellin or flagellin associated polypeptide having an amino acid sequence comprising SEQ ID NO: 226 or 571 and a betaine. The betaine can comprise betaine hydrochloride or glycine betaine. Optionally, the composition can further comprise a bacteriocide (e.g., oxytetracycline).

The composition can comprise a flagellin or flagellin associated polypeptide and a proline. For example, the composition can comprise a flagellin or flagellin associated polypeptide having an amino acid sequence comprising SEQ ID NO: 226 or 571 and a proline. The proline can comprise L-proline. Optionally, the composition can further comprise a bacteriocide (e.g., oxytetracycline).

The composition can comprise a flagellin or flagellin associated polypeptide and an herbicide. For example, the composition can comprise a flagellin or flagellin associated polypeptide having an amino acid sequence comprising SEQ ID NO: 226 or 571 and a herbicide. The herbicide can comprise lactofen. Optionally, the composition can further comprise a bacteriocide (e.g., oxytetracycline). The composition can comprise a flagellin or flagellin associated polypeptide and a bacteriocide. For example, the composition can comprise a flagellin or flagellin associated polypeptide having an amino acid sequence comprising SEQ ID NO: 226 or 571 and a bacteriocide. The bacteriocide can comprise oxytetracycline.

When a composition includes the retro-inverso form of a Flg bioactive priming polypeptide (for example, RI Bt.4Q7 Flg 22 (SEQ ID NO: 376), the polypeptide exhibits enhanced stability and less degradation over time providing for more activity at the plant cell membrane surface, which enhances the ability of the polypeptide to bind to the receptor and be taken into the plant. Retro inverso forms of such Flg-associated bioactive priming polypeptides are used to provide enhanced stability of the agriculturally applied formulation whereby the Flg polypeptide(s) exhibits enhanced protection from proteolytic cleavage, which contributes to an overall greater activity and shelf life of the composition.

When the polypeptide comprises a root hair promoting polypeptide (RHPP), the composition can further comprise a flagellin or flagellin associated polypeptide. The RHPP can comprise any one of SEQ ID NOs: 604, 607-608 and 745-755. For example, the RHPP can comprise SEQ ID NO: 604. The amino acid sequence of the flagellin or flagellin-associated polypeptide can comprise any one of SEQ ID NOs: 1-525, 532, 534, 536, 538, 540, 571-585, 587, and 590, or any combination thereof. For example, the flagellin or flagellin associated polypeptide can comprise any one of SEQ ID NO: 226 or 571. In some instances, the RHPP can comprise SEQ ID NO: 604 and the flagellin or flagellin associated polypeptide can comprise SEQ ID NO: 226. In other instances, the RHPP can comprise SEQ ID NO: 604 and the flagellin or flagellin associated polypeptide can comprise SEQ ID NO: 571.

The polypeptides can be formulated in combination with an assistance polypeptide. The signature (SEQ ID NOs: 542-548), signal anchor sorting (SEQ ID NOs: 549-562) and secretion (SEQ ID NOs: 563-570) polypeptides can be combined with the bioactive priming polypeptides as described for targeting the polypeptides/peptides (Tables 1-5) to the plant cell membrane surface for improved binding and activation of the Flg-associated receptors. This means for efficient delivery and binding of the polypeptide to a plant provides growth promoting benefits, as well as enhanced protection to the plant or plant part.

The composition can comprise a glucanase polypeptide, an amylase polypeptide, an amino acid and a callose synthase inhibitor. Preferably, the glucanase polypeptide comprises a β-1,3-endoglucanase. For example, the composition can comprise a β-1,3-endoglucanase having an amino acid sequence comprising any one of SEQ ID NOs: 731-733 or 767-766 and an amylase having an amino acid sequence comprising SEQ ID NO: 734 or 735. The amino acid can comprise L-cysteine. The callose synthase inhibitor can comprise 2-DDG. The composition can further comprise at least one flagellin or flagellin associated polypeptide. The flagellin or flagellin associated polypeptide can have an amino acid sequence comprising SEQ ID NO: 226 or 571.

The composition can comprise a glucanase polypeptide, a chitinase polypeptide, an amino acid and a callose synthase inhibitor. Preferably, the glucanase polypeptide comprises a β-1,3-endoglucanase. For example, the composition can comprise a β-1,3-endoglucanase having an amino acid sequence comprising any one of SEQ ID NOs: 731-733 or 767-766 and an chitinase having an amino acid sequence comprising SEQ ID NO: 777 or 778. The amino acid can comprise L-cysteine. The callose synthase inhibitor can comprise 2-DDG. The composition can further comprise at least one flagellin or flagellin associated polypeptide. The flagellin or flagellin associated polypeptide can having an amino acid sequence comprising SEQ ID NO: 226 or 571.

The composition can comprise an a root hair promoting polypeptide (RHPP) or a retro inverso root hair promoting polypeptide (RI-RHPP) and a betaine. For example, the composition can comprise an RHPP having an amino acid sequence comprising any one of SEQ ID NOs: 604, 607-608 and 745-756 or an RI-RHPP comprising any one of SEQ ID NOs: 605, 609-610 and 757-766 and a betaine. The betaine can comprise betaine hydrochloride or glycine betaine. Optionally, the composition can further comprise a bacteriocide (e.g., oxytetracycline).

The composition can comprise a root hair promoting polypeptide (RHPP) or a retro inverso root hair promoting polypeptide (RI-RHPP) and a proline. For example, the composition can comprise an RHPP having an amino acid sequence comprising any one of SEQ ID NOs: 604, 607-608 and 745-756 or an RI-RHPP comprising any one of SEQ ID NOs: 605, 609-610 and 757-766 and a proline. The proline can comprise L-proline. Optionally, the composition can further comprise a bacteriocide (e.g., oxytetracycline).

The composition can comprise at least two inducer compounds.

The composition can comprise a bacteriocide and at least one of: 2-deoxy-D-glucose, BABA, benzothiadiazole and cysteine. For example, the composition can comprise a bacteriocide (i.e., oxytetracycline) and 2-deoxy-D-glucose.

The composition can comprise (A) at least one polypeptide and an inducer compound or (B) at least two polypeptides, optionally, with an inducer compound; or (C) at least two inducer compounds wherein:

(a) the polypeptide or polypeptides of (A) or (B) comprise:
(i) a flagellin or flagellin-associated polypeptide and an amino acid sequence of the flagellin or flagellin-associated polypeptide comprises any one of SEQ ID NOs: 571, 1-375, 526, 528, 530, 532, 534, 536, 538, 540, 541, 572-585, 587, and 589-603; or
(ii) a retro inverso Flg22 polypeptide and an amino acid sequence of the retro inverso Flg22 polypeptide comprises any one of SEQ ID NOs: 376-450, 527, 531, 533, 535, 537 and 539; or
(iii) a retro inverso FlgII-28 polypeptide and an amino acid sequence of the retro inverso FlgII-28 polypeptide comprises any one of SEQ ID NOs: 451-525, or 588; or
(iv) a retro inverso Flg15 polypeptide and an amino acid sequence of the retro inverso Flg15 polypeptide comprises SEQ ID NOs: 529 or 586; or
(v) a root hair promoting polypeptide (RHPP) and an amino acid sequence of the RHPP comprises any one of SEQ ID Nos: 604, 607, 608, and 745-755; or
(vi) a retro inverso root hair promoting polypeptide (RI RHPP) and an amino acid sequence of the RI RHPP comprises any one of SEQ ID NO: 605, 609, 610 and 756-766; or
(vii) a thionin or thionin-like polypeptide and an amino acid sequence of the thionin or thionin-like polypeptide comprises any one of SEQ ID NOs: 620-719; or
(viii) a glucanase polypeptide and an amino acid sequence of the glucanase polypeptide comprises any one of SEQ ID NOs: 731-733 and 767-776; or
(ix) an amylase polypeptide an an amino acid sequence of the amylase polypeptide comprises SEQ ID NO: 734 or 735; or
(x) a chitinase polypeptide and an amino acid sequence of the chitinase polypeptide comprises SEQ ID NO: 777 or 778; or
(xi) a serine protease polypeptide and an amino acid sequence of the serine protease polypeptide comprises SEQ ID NO: 721, 722 or 794-796; or
(xii) an ACC deaminase polypeptide and an amino acid sequence of the ACC deaminase polypeptide comprises any one of SEQ ID NOs: 723-730; or
(xiii) any combination thereof;

The inducer compound can comprise a callose synthase inhibitor, beta amino butyric acid (BABA), a betaine, a proline, salicylic acid, oxalic acid, a benzothiazole or any combination thereof when the polypeptide of (A) comprises any polypeptide from groups (i)-(v) but not polypeptides selected from the groups (vi) to (x);

The inducer compound can comprise a callose synthase inhibitor, β-amino butyric acid (BABA), a betaine, a proline, salicylic acid, oxalic acid or any combination thereof when the polypeptide of (A) comprises any polypeptide from groups (i)-(v) but not polypeptides selected from the groups (vi) to (x).

The inducer compound can comprise a callose synthase inhibitor, β-amino butyric acid (BABA), salicylic acid, oxalic acid or any combination thereof when the polypeptide of (A) comprises any polypeptide from groups (i)-(v) but not polypeptides selected from the groups (vi) to (x).

The inducer compound can comprise a callose synthase inhibitor, β-amino butyric acid (BABA), or any combination thereof when the polypeptide of (A) comprises any polypeptide from groups (i)-(v) but not polypeptides selected from the groups (vi) to (x).

The inducer compound can comprise a betaine or a proline when the polypeptide of (A) comprises any polypeptide from groups (i)-(v) but not polypeptides selected from the groups (vi) to (x).

The inducer compound can comprise salicylic acid or oxalic acid when the polypeptide of (A) comprises any polypeptide from groups (i)-(v) but not polypeptides selected from the groups (vi) to (x).

The inducer compound can comprise a bacteriocide, an amino acid or isomer thereof, a callose synthase inhibitor, a substituted or unsubstituted benzoic acid or derivative thereof, a dicarboxylic acid or a derivative thereof, a betaine, a proline, a benzothiazole, or any combination thereof when the polypeptide of (A) comprises any polypeptide from groups (vi) to (x).

The inducer compound can comprise the inducer compound and the inducer compound comprises a callose synthase inhibitor, beta amino butyric acid (BABA), betaine, a proline, salicylic acid, oxalic acid, a benzothiazole or any combination thereof when the two or more polypeptides of (B) comprise polypeptides selected from groups (i)-(v) but not polypeptides selected from the groups (vi) to (x).

The inducer compound can comprise a callose synthase inhibitor, β-amino butyric acid (BABA), a betaine, a proline, salicylic acid, oxalic acid or any combination thereof when the two or more polypeptides of (B) comprise polypeptides selected from groups (i)-(v) but not polypeptides selected from the groups (vi) to (x).

The inducer compound can comprise a callose synthase inhibitor, β-amino butyric acid (BABA), salicylic acid, oxalic acid or any combination thereof when the two or more polypeptides of (B) comprise polypeptides selected from groups (i)-(v) but not polypeptides selected from the groups (vi) to (x).

The inducer compound can comprise a callose synthase inhibitor, β-amino butyric acid (BABA), or any combination thereof when the two or more polypeptides of (B) comprise polypeptides selected from groups (i)-(v) but not polypeptides selected from the groups (vi) to (x).

The inducer compound can comprise a betaine or a proline when the two or more polypeptides of (B) comprise polypeptides selected from groups (i)-(v) but not polypeptides selected from the groups (vi) to (x).

The inducer compound can comprise salicylic acid or oxalic acid when the two or more polypeptides of (B) comprise polypeptides selected from groups (i)-(v) but not polypeptides selected from the groups (vi) to (x).

The composition can comprise at least one polypeptide selected from groups (i) to (x) and at least one inducer compound comprising a succinate dehydrogenase inhibitor.

The inducer compound can comprise a bacteriocide and at least one of a callose synthase inhibitor, β amino butyric acid (BABA), a proline, a benziothiaozole, salicylic acid, oxalic acid, succinate dehydrogenase inhibitor, or a betaine. The succinate dehydrogenase inhibitor can be bixafen. The callose synthase inhibitor can be 2-DDG. The bacteriocide can be oxytetracycline.

The inducer compound can comprise a bacteriocide and at least one of a callose synthase inhibitor, β amino butyric acid (BABA), a proline, a betaine, salicylic acid, succinate dehydrogenase inhibitor, or oxalic acid. The succinate dehydrogenase inhibitor can be bixafen. The callose synthase inhibitor can be 2-DDG. The bacteriocide can be oxytetracycline.

The inducer compound can comprise a bacteriocide and at least one of a callose synthase inhibitor, β amino butyric acid (BABA), salicylic acid, succinate dehydrogenase inhibitor, or oxalic acid. The succinate dehydrogenase inhibitor can be bixafen. The callose synthase inhibitor can be 2-DDG. The bacteriocide can be oxytetracycline.

The inducer compound can comprise a bacteriocide and a callose synthase inhibitor or β amino butyric acid (BABA). The callose synthase inhibitor can be 2-DDG. The bacteriocide can be oxytetracycline.

The inducer compound can comprise a callose synthase inhibitor and at least one of a beta amino butyric acid (BABA), a bacteriocide, a proline, a benzothiazole, salicylic acid, oxalic acid, a succinate dehydrogenase inhibitor, or a betaine. The succinate dehydrogenase inhibitor can be bixafen. The callose synthase inhibitor can be 2-DDG. The bacteriocide can be oxytetracycline.

For example, the composition can comprise (a) a flagellin or flagellin associated polypeptide and L-cysteine; or (b) a flagellin or flagellin associated polypeptide and 2-deoxy-D-glucose; or (c) a flagellin or flagellin associated polypeptide and an ACC deaminase; or (d) a flagellin or flagellin associated polypeptide and salicylic acid; or (e) a flagellin or flagellin associated polypeptide and oxalic acid; or (f) a flagellin or flagellin associated polypeptide and a benzothiadiazole; or (g) a flagellin or flagellin associated polypeptide and BABA; or (h) a flagellin or flagellin associated polypeptide and a betaine; or (i) a flagellin or flagellin associated polypeptide and a proline; or (j) a flagellin or flagellin associated polypeptide and a serine protease; or (k) a flagellin or flagellin associated polypeptide and a thionin or thionin-like polypeptide; or (l) a flagellin or flagellin associated polypeptide and an amylase; or (m) a flagellin or flagellin associated polypeptide and a chitinase; or (n) a bacteriocide and at least one of: 2-deoxy-D-glucose, BABA, benzothiadiazole, or cysteine; or (o) a serine protease; or (p) a thionin or thionin-like polypeptide; or (q) a serine protease and a thionin or thionin-like polypeptide; or (r) a flagellin or flagellin associated polypeptide and a glucanase; or (s) a flagellin or flagellin associated polypeptide, a glucanase, an amylase; or (t) a flagellin or flagellin associated polypeptide, a glucanase, an amylase, 2-DDG; or (u) a flagellin or flagellin associated polypeptide, a glucanase, an amylase, 2-DDG, and cysteine; or (v) a glucanase, an amylase, 2-DDG, and cysteine; or (w) a glucanase and an amylase; or (x) a flagellin or flagellin associated polypeptide, a glucanase, a chitinase; or (y) a flagellin or flagellin associated polypeptide, a glucanase, a chitinase, 2-DDG; or (z) a flagellin or flagellin associated polypeptide, a glucanase, a chitinase, 2-DDG, and cysteine; or (aa) a glucanase, a chitinase, 2-DDG, and cysteine; or (bb) a glucanase and a chitinase; or (cc) a glucanase and a chitinase; or (dd) a flagellin or flagellin associated polypeptide, a glucanase, and a serine protease; or (ee) a glucanase and an RHPP polypeptide or retro inverso RHPP polypeptide; or (ff) an RHPP polypeptide or retro-inverso RHPP polypeptide and a betaine; or (gg) an RHPP peptide or retro-inverso RHPP polypeptide and a proline; or (hh) an RHPP polypeptide or RHPP retro-inverso polypeptide and an ACC deaminase.

Further, any composition (a)-(hh) can further comprise a bacteriocide. The bacteriocide can comprise oxytetracycline.

Further, any composition (a)-(hh) can further comprise a succinate dehydrogenase inhibitor. The succinate dehydrogenase inhibitor can comprise bixafen.

Another composition is provided, the composition comprising bixafen and a free polypeptide (i.e., not bound to an exosporium of a Bacillus cereus family member or an intact Bacillus cereus family member spore). The free polypeptide can comprise (i) a flagellin or flagellin-associated polypeptide; or (ii) a retro inverso flagellin or flagellin-associated polypeptide; or (iii) a root hair promoting polypeptide (RHPP); or (iv) a retro inverso root hair promoting polypeptide (RI RHPP); or (v) a thionin or thionin-like polypeptide; or (vi) a glucanase polypeptide; or (vii) a serine protease polypeptide; or (viii) an ACC deaminase (1-aminocyclopropane-1-carboxylate deaminase) polypeptide; or (ix) an amylase; or (x) a chitinase; or (xi) any combination thereof.

The composition can comprise a free polypeptide comprising a root hair promoting polypeptide (RHPP), a retro-inverso root hair promoting polypeptide (RI-RHPP), a chitinase, a flagellin or flagellin associated polypeptide, a glucanase, a serine protease or any combination thereof.

The composition can comprise a free polypeptide wherein an amino acid sequence of the free polypeptide can comprise any one of SEQ ID NOs: 604, 606, 607 and 745-755 (root hair promoting polypeptide, RHPP), any one of SEQ ID NOs: 605, and 756-766 (retro-inverso root hair promoting polypeptide, RI-RHPP), any one of SEQ ID NOs 226 and 571 (flagellin or flagellin associated polypeptide), any one of SEQ ID NOs: 731-733 and 767-778 (glucanase), any one of SEQ ID NOs: 777 and 778 (chitinases) or any one of SEQ ID NOs: 721, 722 and 794-796 (serine proteases).

The composition can comprise bixafen and a free polypeptide comprising a root hair promoting polypeptide and an amino acid sequence of the RHPP can comprise any one of SEQ ID NOs: 604, 606, 607 and 745-755. For example, the amino acid sequence of the RHPP can comprise SEQ ID NO: 604.

As described herein, the compositions can comprise (A) at least one bioactive polypeptide and an inducer compound, (B) at least two bioactive polypeptides, optionally with an inducer compound, or (C) two inducer compounds. Preferred formulations are provided, therefore, for compositions comprising (a) polypeptides alone, (b) polypeptide(s) and inducer compound(s) and (c) inducer compounds alone. As described in the following preferred formulations “polypeptides” include “free polypeptides” as described in some compositions herein. Likewise, and as defined above, “bixafen” can be considered an inducer compound.

When the compositions comprise two or more bioactive polypeptides, the composition can comprise from about 0.0000001 wt. % to about 95% of the polypeptide(s), from about 0.01 wt. % to about 5 wt. % of the polypeptide(s), or from 0.005 wt. % to about 1 wt. % of the polypeptide(s), or from 0.005 wt. % to about 0.1 wt. % of the polypeptide(s) based on the total weight of the composition.

When the composition comprises at least one bioactive priming polypeptide and at least one inducer compound, the composition can comprise from about 0.0000001 wt. % to about 95 wt. % of the polypeptide and from about 0.000001 wt. % to about 95 wt. % of the inducer compound, based on the total weight of the composition. Alternatively, the composition can comprise from about 0.0000005 wt. % to about 10 wt. % of the polypeptide(s) and from about 0.000001 wt. % to about 95 wt. % of the inducer compound based on the total weight of the composition. Alternatively, the composition can comprise from about 0.001 wt. % to about 5 wt. % of the polypeptide(s) and from about 0.000001 wt. % to about 95 wt. % of the inducer compound based on the total weight of the composition. Alternatively, the composition can comprise from about 0.005 wt. % to about 1 wt. % (e.g., from about 0.005 wt. % to about 0.1 wt. %) of the polypeptide(s) and from about 0.000001 wt. % to about 95 wt. % of the inducer compound based on the total weight of the composition. In some cases, particularly when the inducer compound comprises a bacteriocide, the composition of any of these formulations can comprise from about 0.001 wt. % to about 95 wt. % of the inducer compound based on the total weight of the composition.

When the composition comprises two or more inducer compounds, the composition can comprise from about 0.000001 wt. % to about 95 wt. % of a first inducer and from about 0.000001 wt. % to about 95% wt. % of the second inducer based on the total weight of the composition. Alternatively, the composition can comprise from about 0.000001 wt. % to about 95 wt. % of the first inducer and from about 0.001 wt. % of the second inducer based on the total weight of the composition. Alternatively, the composition can comprise from about 0.001 wt. % of the first inducer and from about 0.000001 wt. % to about 95 wt. % of the second inducer based on the total weight of the composition. Preferably, the inducer compound comprises from about 0.000001 wt. % to about 95 wt. % of the composition, based on the total weight of the composition, when it comprises a callose synthase inhibitor, an amino acid, salicylic acid, oxalic acid, a betaine, a proline, a benzothiadiazole, a succinate dehydrogenase inhibitor, or any combination thereof. Preferably, the inducer compound comprises from about 0.001 wt. % to about 95 wt. % of the composition, based on the total weight of the composition, when the inducer compound comprises a bactericide.

The composition can include either an agrochemical or a carrier which is associated with the polypeptide in nature.

The agrochemical can be non-naturally occurring in combination with the polypeptide.

The agrochemical can include, but is not limited to, a preservative, a buffering agent, a wetting agent, a surfactant, a coating agent, a monosaccharide, a polysaccharide, an abrading agent, a pesticide, an insecticide, an herbicide, a nematicide, a bacteriocide, a fungicide, a miticide, a fertilizer, a biostimulant, a colorant, a humectant, an osmoprotectant, an antibiotic, an amino acid, a biological control agent, an osmoprotectant, or a combination thereof.

When the composition includes an amino acid, the amino acid can be provided separately from the amino acids that comprise the polypeptide. For example, an isolated amino acid can be used. Suitable amino acids include any natural or unnatural amino acids. For example, the composition can comprise cysteine.

The agrochemical can comprise an acid such as an acid that is present from chemical synthesis of any polypeptide described herein. For example, hydrochloric acid, acetic acid, or trifluoroacetic acid can be present if the polypeptide is synthesized such as by fermentation.

When the agrochemical is an acid, it can comprise from about 0.001 to about 30 wt. %, from about 0.01 to about 20 wt. %, or from about 0.1 to about 5 wt. % of the total weight of the composition.

Unless otherwise specified, each agrochemical can comprise from about 0.01 to about 99 wt. %, from about 0.1 to about 70 wt. %, or from about 0.1 to about 60 wt. % of the total weight of the composition.

When the composition includes a preservative, the preservative can comprise those based on dichlorophene and benzylalcohol hemi formal (PROXEL from ICI or ACTICIDE RS from Thor Chemie and KATHON MK from Dow Chemical) and isothiazolinone derivatives such as alkylisothiazolinones and benzisothiazolinones (ACTICIDE MBS from Thor Chemie). As further examples, suitable preservatives include MIT (2-methyl-4-isothiazolin-3-one), BIT (1,2-benzisothiazolin-3-one, which can be obtained from Avecia, Inc. as PROXEL GXL as a solution in sodium hydroxide and dipropylene glycol), 5-chloro-2-(4-chlorobenzyl)-3(2H)-isothiazolone, 5-chloro-2-methyl-2H-isothiazol-3-one, 5-chloro-2-methyl-2H-isothiazol-3-one, 5-chloro-2-methyl-2H-isothiazol-3-one-hydrochloride, 4,5-dichloro-2-cyclohexyl-4-isothiazolin-3-one, 4,5-dichloro-2-octyl-2H-isothiazol-3-one, 2-methyl-2H-isothiazol-3-one, 2-methyl-2H-isothiazol-3-one-calcium chloride complex, 2-octyl-2H-isothiazol-3-one, benzyl alcohol hemiformal, or any combination thereof.

When the composition includes a buffering agent, the buffering agent can comprise potassium, phosphoric acid, a phosphate salt, citric acid, a citrate salt, a sulfate salt, MOPS, or HEPES. The buffering agent can stabilize the polypeptide in the composition.

When the composition includes a wetting agent, the wetting agent can comprise organosilicones, polyoxyethoxylates, polysorbates, polyethyleneglycol and derivatives thereof, ethoxylates, crop oils, and polysaccharides.

When the composition includes a surfactant, the surfactant can comprise a heavy petroleum oil, a heavy petroleum distillate, a polyol fatty acid ester, a polyethoxylated fatty acid ester, an aryl alkyl polyoxyethylene glycol, a polyoxyethylenepolyoxypropylene monobutyl ether, an alkyl amine acetate, an alkyl aryl sulfonate, a polyhydric alcohol, an alkyl phosphate, an alcohol ethoxylate, an alkylphenol ethoxylate, an alkyphenol ethoxylate, an alkoxylated polyol, an alky polyethoxy ether, an alkylpolyoxethylene glycerol, ethoxylated and soybean oil derivatives, an organosilicone-based surfactant or any combination thereof. Surfactants can be included in a range of compositions including those for foliar use.

When the composition includes a coating agent, the coating agent can comprise a tackifier, polymers, filling agents, or bulking agents.

The tackifier can include, but is not limited to, carboxymethylcellulose and natural and synthetic polymers in the form of powders, granules, or latexes, such as gum Arabic, chitin, polyvinyl alcohol and polyvinyl acetate, as well as natural phospholipids, such as cephalins and lecithins, and synthetic phospholipids. Tackifiers include those composed preferably of an adhesive polymer that can be natural or synthetic without phytotoxic effect on the seed to be coated. Additional tackifiers that can be included, either alone or in combination, include, for example, polyesters, polyether esters, polyanhydrides, polyester urethanes, polyester amides; polyvinyl acetates; polyvinyl acetate copolymers; polyvinyl alcohols and tylose; polyvinyl alcohol copolymers; polyvinylpyrolidones; polysaccharides, including starches, modified starches and starch derivatives, dextrins, maltodextrins, alginates, chitosanes and celluloses, cellulose esters, cellulose ethers and cellulose ether esters including ethylcelluloses, methylcelluloses, hydroxymethylcelluloses, hydroxypropylcelluloses and carboxymethylcellulose; fats; oils; proteins, including casein, gelatin and zeins; gum arabics; shellacs; vinylidene chloride and vinylidene chloride copolymers; lignosulfonates, in particular calcium lignosulfonates; polyacrylates, polymethacrylates and acrylic copolymers; polyvinylacrylates; polyethylene oxide; polybutenes, polyisobutenes, polystyrene, polybutadiene, polyethyleneamines, polyethylenamides; acrylamide polymers and copolymers; polyhydroxyethyl acrylate, methylacrylamide monomers; and polychloroprene, or any combination thereof. Tackifiers can be used in a range of compositions including those for seed treatment.

When the composition includes an abrading agent, the abrading agent can comprise talc, graphite, or a combination of both.

A humectant is a hygroscopic substance that assists with the retention of moisture. When the composition includes a humectant, the humectant can comprise: glycerol, glycerin, a glycerol derivative (e.g. glycerol monosterate, glycerol triacetate, triacetin, propylene glycol, hexylene glycol, or butylene glycol), triethylene glycol, tripolypropylene glycol, glyceryl triacetate, sucrose, tagatose, a sugar alcohol or a sugar polyol (e.g glycerol, sorbitol, xylitol, mannitol, or mantitol), a polymeric polyol (e.g. polydextrose, a collagen, an aloe or an aloe vera gel), or an alpha hydroxy acid (e.g. lactic acid, honey, molasses, quillaia, sodium hexametaphosphate, lithium chloride or urea). Synthetic humectants can also comprise: butylene glycol, and tremella extract.

When the composition includes a pesticide, the pesticide can comprise an insecticide, a herbicide, a fungicide, a bacteriocide, a nematicide, a miticide, or any combination thereof.

When the composition includes an insecticide, the insecticide can comprise clothianidin, imidacloprid, an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or any combination thereof. For example, the insecticide can comprise clothianidin or imidacloprid.

The agrochemical can comprise an herbicide. The herbicide can comprise 2,4-D, 2,4-DB, acetochlor, acifluorfen, alachlor, ametryn, atrazine, aminopyralid, benefin, bensulfuron, bensulfuron methyl bensulide, bentazon, bispyribac sodium, bromacil, bromoxynil, butylate, carfentrazone, chlorimuron, 2-chlorophenoxy acetic acid, chlorsulfuron, chlorimuron ethyl, clethodim, clomazone, clopyralid, cloransulam, CMPP-P-DMA, cycloate, DCPA, desmedipham, dicamba, dichlobenil, diclofop, 2,4-dichlorophenol, dichlorophenoxyacetic acid, dichlorprop, dichlorprop-P, diclosulam, diflufenzopyr, dimethenamid, dimethyl amine salt of 2,4-dichlorophenoxyacetic acid, diquat, diuron, DSMA, endothall, EPTC, ethalfluralin, ethofumesate, fenoxaprop, fluazifop-P, flucarbazone, flufenacet, flumetsulam, flumiclorac, flumioxazin, fluometuron, fluroxypyr, fluorxypyr 1-methyleptylester, fomesafen, fomesafen sodium salt, foramsulfuron, glufosinate, glufosinate-ammonium, glyphosate, halosulfuron, halosulfuron-methyl, hexazinone, 2-hydroxyphenoxy acetic acid, 4-hydroxyphenoxy acetic acid, imazamethabenz, imazamox, imazapic, imazaquin, imazethapyr, isoxaben, isoxaflutole, lactofen, linuron, mazapyr, MCPA, MCPB, mecoprop, mecoprop-P, mesotrione, metolachlor-s, metribuzin, metsulfuron, metsulfuron-methyl, molinate, MSMA, napropamide, naptalam, nicosulfuron, norflurazon, oryzalin, oxadiazon, oxyfluorfen, paraquat, pelargonic acid, pendimethalin, phenmedipham, picloram, primisulfuron, prodiamine, prometryn, pronamide, propanil, prosulfuron, pyrazon, pyrithiobac, pyroxasulfone, quinclorac, quizalofop, rimsulfuron, sethoxydim, siduron, simazine, sulfentrazone, sulfometuron, sulfosulfuron, tebuthiuron, terbacil, thiazopyr, thifensulfuron, thifensulfuron-methyl, thiobencarb, tralkoxydim, triallate, triasulfuron, tribenuron, tribernuron-methyl, triclopyr, trifluralin, triflusulfuron, or any combination thereof.

When the composition includes a nematicide, the nematicide can comprise Bacillus firmus, fluopyram, antibiotic nematicides such as abamectin; carbamate nematicides such as acetoprole, Bacillus chitonosporus, chloropicrin, benclothiaz, benomyl, Burholderia cepacia, carbofuran, carbosulfan, and cleothocard; dazomet, DBCP, DCIP, alanycarb, aldicarb, aldoxycarb, oxamyl, diamidafos, fenamiphos, fosthietan, phosphamidon, cadusafos, chlorpyrifos, diclofenthion, dimethoate, ethoprophos, fensulfothion, fostiazate, harpins, heterophos, imicyafos, isamidofos, isazofos, methomyl, mecarphon, Myrothecium verrucaria, Paecilomyces lilacinus, Pasteuria nishizawae (including spores thereof), phorate, phosphocarb, terbufos, thionazin, triazophos, tioxazafen, dazomet, 1,2-dicloropropane, 1,3-dichloropropene, furfural, iodomethane, metam, methyl bromide, methyl isothiocyanate, xylenol, pydiflumetofen, or any combination thereof. For example, the nematicide can comprise Bacillus firmus strain i-2580, Pasteuria nishizawae (including spores thereof), or fluopyram.

When the composition includes a bacteriocide, the bacteriocide can comprise streptomycin, penicillins, tetracyclines, oxytetracycline, kasugamycin, ampicillin, oxolinic acid, chlorotetracycline, copper oxide, copper hydroxide, copper sulfide, copper sulfate, fine particle coppers, or any combination thereof. For example, the bacteriocide can comprise oxytetracycline.

Biological control agents are broadly defined as microorganisms that can be used instead of synthetic pesticides or fertilizers. When the composition includes a biological control agent, the biological control agent can comprise Bacillus thuringiensis, Bacillus megaterium, Bacillus mycoides isolate J, Bacillus methylotrophicus, Bacillus vallismortis, Chromobacterium subtsugae, Delftia acidovorans, Streptomyces lydicus, Streptomyces colombiensis, Streptomyces galbus K61, Penicillium bilaii, Banda de Lupinus albus doce (BLAD), an Aureobasidium pullalans strain, a lipopeptide-producing Bacillus subtilis strain, a lipopeptide-producing Bacillus amyloliquefaciens strain, a Bacillus firmus strain or a Bacillus pumilus strain. As another example, when the composition includes a biological control agent, the biological control agent can comprise Bacillus subtilis strain QST713, Bacillus pumulis strain QST 2808, Aureobasidium pullalans strain DMS 14940 Aureobasidium pulladans strain 14941, Penicillium bilaii, Banda de Lupinus albus doce (BLAD), and/or an Aureobasidium pullalans strain.

When the composition comprises an osmoprotectant the osmoprotectant can comprise a betaine or a proline. The betaine can comprise betaine hydrochloride or glycine betaine. The proline can comprise L-proline.

The agrochemical can include a fungicide. The fungicide can comprise a strobilurin fungicide, a triazole fungicide, a succinate dehydrogenase inhibitor fungicide, a laminarin, a pheylamide, a methyl benzimidazole carbamate, a anilino-pyrimidine, a phenylpyrrole, a dicarboximide, a carbamate, a piperidinyl-thiazole-isoxazoline, a demethylation inhibitor, a phosphonate, an inorganic copper, an inorganic sulfur, a thiocarbamate, a dithiocarbamate, a phthalimide, a chloronitrile, or a sulfamide.

The fungicide can comprise a harpin or harpin-like polypeptide. Harpin and harpin-like polypeptides are described in U.S Patent Publication No. 2019/0023750, hereby incorporated by reference in its entirety. The harpin or harpin-like polypeptides can be derived from Xanthomonas species or diverse bacteria genera including Pantoea sesami, Erwinia gerudensis, Pantoea sesami, or Erwinia gerudensis. Additional harpin-like fungicide polypeptides can be derived from the full length HpaG-like protein from Xanthamonas citri. Representative harpin-like polypeptides that can be incorporated into the compositions herein as a fungicide are described in Table 21, below. The harpins can advantageously be injected into a plant (i.e., into a tree trunk) to generate an immune response in combination with the bioactive polypeptides and inducers described herein.

TABLE 21
Fungicidal Harpin and Harpin-Like Polypeptides

| SEQ ID NO: | Peptide Sequence Amino Acid |
|---|---|
| Harpin-like (HpaG-like) | NQGISEKQLDQLLTQLIMALLQQ |
| SEQ ID NO: 788 | |
| Xanthomonas species | |
| MW 2626.35 Da | |
| Harpin-like (HpaG-like) | LDQLLTQLIMAL |
| SEQ ID NO: 789 | |
| Xanthomonas species | |
| MW 2626.35 Da | |
| Harpin-like (HpaG-like) | SEKQLDQLLTQLIMALLQQ |
| SEQ ID NO: 790 | |
| Xanthomonas species | |
| MW 2626.35 Da | |
| HpaG-Like Protein | MMNSLNTQLGANSSFFQVDPSQNTQSGSNQGN |
| SEQ ID NO: 791 | QGISEKQLDQLLTQLIMALLQQSNNAEQGQGQG |
| Xanthamonas citri | QGGDSGGQGGNRQQAGQSNGSPSQYTQMLMNI |
| | VGDILQAQNGGGFGGGFGGGFGGGLGTSLGTSL |
| | GTSLASDTGSMQ |
| HpaG Homolog Active Fraction | QLEQLMTQLRARLCRLMAM |
| SEQ ID NO: 792 | |
| Pantoea sesami | |
| HpaG Homolog Active Fraction | QLEQLMTQLRARLKRLMAM |
| SEQ ID NO: 793 | |
| Erwinia gerudensis | |

The fungicide can comprise acibenzolar-S-methyl, aldimorph, aluminum-tris, ametocradin, ampropylfos, ampropylfos potassium, andoprim, anilazine, azaconazole, azoxystrobin, benalaxyl, benodanil, benomyl, benzamacril, benzamacryl-isobutyl, benzovindflupyr, bialaphos, binapacryl, biphenyl, bitertanol, bixafen, blasticidin-S, boscalid, bromuconazole, bupirimate, buthiobate, calcium polysulphide, capsimycin, captafol, captan, carbendazim, carvon, quinomethionate, chlobenthiazone, chlorantraniliprole, chlorfenazole, chloroneb, chloropicrin, chlorothalonil, chlozolinate, clozylacon, cresilym methyl, cufraneb, cymoxanil, cyproconazole, cyprodinil, cyprofuram, debacarb, dichlorophen, diclobutrazole, diclofluanid, diclomezine, dicloran, diethofencarb, dimethirimol, dimethomorph, dimoxystrobin, diniconazole, diniconazole-M, dinocap, diphenylamine, dipyrithione, ditalimfos, dithianon, dodemorph, dodine, drazoxolon, edifenphos, epoxiconazole, etaconazole, ethirimol, etridiazole, famoxadone, fenamidone, fenapanil, fenarimol, fenbuconazole, fenfuram, fenitropan, fenpiclonil, fenpropidin, fenpropimorph, fentin acetate, fentin hydroxide, ferbam, ferimzone, fluazinam, fludioxonil, flumetover, fluopyram, fluoromide, fluoxastrobin fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole, imazalil, imibenconazole, iminoctadine, iminoctadine albesilate, iminoctadine triacetate, iodocarb, iprobenfos (IBP), iprodione, irumamycin, isoprothiolane, isovaledione, kasugamycin, kresoxim-methyl, copper preparations, such as: copper hydroxide, copper naphthenate, copper oxychloride, copper sulphate, copper oxide, oxine-copper and Bordeaux mixture, mancopper, mancozeb, mandipropamid, maneb, meferimzone, mefentrifuconazole, mepanipyrim, mepronil, metalaxyl, metconazole, metalzxyl, methasulfocarb, methfuroxam, metiram, metomeclam, metominostrobin, metrafenone, metsulfovax, mildiomycin, myclobutanil, myclozolin, nickel dimethyldithiocarbamate, nitrothal-isopropyl, nuarimol, ofurace, oxadixyl, oxamocarb, oxathiapiprolin, oxolinic acid, oxycarboxim, oxyfenthiin, paclobutrazole, pefurazoate, penconazole, pencycuron, penthiopyrad, phosdiphen, picoxystrobin, pimaricin, piperalin, polyoxin, polyoxorim, potassium phosphite, probenazole, prochloraz, procymidone, propamocarb, propanosine-sodium, propiconazole, propineb, prothiocinazole, pydiflumetofen, pyrazophos, pyrifenox, pyrimethanil, pyroquilon, pyroxyfur, quinconazole, quinoxyfen, quintozene (PCNB), a strobilurin, sulphur and sulphur preparations, tebuconazole, tecloftalam, tecnazene, tetcyclasis, tetraconazole, thiabendazole, thicyofen, thifluzamide, thiophanate-methyl, tioxymid, tolclofos-methyl, tolylfluanid, triadimefon, triadimenol, triazbutil, a triazole, triazoxide, trichlamide, tricyclazole, triclopyr, tridemorph, trifloxystrobin, triflumizole, triforine, uniconazole, validamycin A, vinclozolin, viniconazole, zarilamide, zineb, ziram and also Dagger G, OK-8705, OK-8801, a-(1,1-dimethylethyl)-(3-(2-phenoxyethyl)-1H-1,2,4-triazole-1-ethanol, a-(2,4-dichlorophenyl)-[3-fluoro-3-propyl-1H-1,2,4-triazole-1-ethanol, a-(2,4-dichlorophenyl)-[3-methoxy-α-methyl-1H-1,2,4-triazole-1-ethanol, a-(5-methyl-1,3-dioxan-5-yl)-[3-[[4-(trifluoromethyl)-phenyl]-methylene]-1H-1,2,4-triazole-1-ethanol, (5RS,6RS)-6-hydroxy-2,2,7,7-tetramethyl-5-(1H-1,2,4-triazol-1-yl)-3-octanone, (E)-a-(methoxyimino)-N-methyl-2-phenoxy-phenylacetamide, 1-isopropyl {2-methyl-1-[[[1-(4-methylphenyl)-ethyl]-amino]-carbonyl]-propyl}carbamate, 1-(2,4-dichlorophenyl)-2-(1H-1,2,4-triazol-1-yl)-ethanone-O-(phenyl methyl)-oxime, 1-(2-methyl-1-naphthalenyl)-1H-pyrrole-2,5-dione, 1-(3,5-dichlorophenyl)-3-(2-propenyl)-2,5-pyrrolidindione, 1-[(diiodomethyl)-sulphonyl]-4-methyl-benzene, 1-[[2-(2,4-dichlorophenyl)-1,3-dioxolan-2-yl]-methyl]-1H-imidazole, 1-[[2-(4-chlorophenyl)-3-phenyloxiranyl]-methyl]-1H-1,2,4-triazole, 1-[1-[2-[(2,4-dichlorophenyl)-methoxy]-phenyl]-ethenyl]-1H-imidazole, 1-methyl-5-nonyl-2-(phenylmethyl)-3-pyrrolidinole, 2′,6′-dibromo-2-methyl-4′-trifluoromethoxy-4′-trifluoro-methyl-1,3-thiazole-carboxanilide, 2,2-dichloro-N-[1-(4-chlorophenyl)-ethyl]-1-ethyl-3-methyl-cyclopropanecarboxamide, 2,6-dichloro-5-(methylthio)-4-pyrimidinyl-thiocyanate, 2,6-dichloro-N-(4-trifluoromethylbenzyl)-benzamide, 2,6-dichloro-N-[[4-(trifluoromethyl)-phenyl]-methyl]-benzamide, 2-(2,3,3-triiodo-2-propenyl)-2H-tetrazole, 2-[(1-methylethyl)-sulphonyl]-5-(trichloromethyl)-1,3,4-thiadiazole, 2-[[6-deoxy-4-O-(4-O-methyl-(3-D-glycopyranosyl)-a-D-glucopyranos yl]-amino]-4-methoxy-1H-pyrrolo [2,3-d]pyrimidine-5-carbonitrile, 2-aminobutane, 2-bromo-2-(bromomethyl)-pentanedinitrile, 2-chloro-N-(2,3-dihydro-1,1,3-trimethyl-1H-inden-4-yl)-3-pyridinecarboxamide, 2-chloro-N-(2,6-dimethylphenyl)-N-(isothiocyanatomethyl)-acetamide, 2-phenylphenol (OPP), 3,4-dichloro-1-[4-(difluoromethoxy)-phenyl]-pyrrole-2,5-dione, 3,5-dichloro-N-[cyano[(1-methyl-2-propynyl)-oxy]-methyl]-benzamide, 3-(1,1-dimethylpropyl-1-oxo-1H-indene-2-carbonitrile, 3-[2-(4-chlorophenyl)-5-ethoxy-3-isoxazolidinyl]-pyridine, 4-chloro-2-cyano-N,N-dimethyl-5-(4-methylphenyl)-1H-imidazole-1-sulphonamide, 4-methyl-tetrazolo[1,5-a]quinazolin-5(4H)-one, 8-(1,1-dimethylethyl)-N-ethyl-N-propyl-1,4-dioxaspiro[4,5]decane-2-methanamine, 8-hydroxyquinoline sulphate, 9H-xanthene-2-[(phenylamino)-carbonyl]-9-carboxylic hydrazide, bis-(1-methylethyl)-3-methyl-4-[(3-methylbenzoyl)-oxy]-2,5-thiophenedicarboxylate, cis-1-(4-chlorophenyl)-2-(1H-1,2,4-triazol-1-yl)-cycloheptanol, cis-4-[3-[4-(1,1-dimethylpropyl)-phenyl-2-methylpropyl]-2,6-dimethyl-morpholine hydrochloride, ethyl [(4-chlorophenyl)-azo]-cyanoacetate, potassium bicarbonate, methanetetrathiol-sodium salt, methyl 1-(2,3-dihydro-2,2-dimethyl-inden-1-yl)-1H-imidazole-5-carboxylate, methyl N-(2,6-dimethylphenyl)-N-(5-isoxazolylcarbonyl)-DL-alaninate, methyl N-(chloroacetyl)-N-(2,6-dimethylphenyl)-DL-alaninate, N-(2,3-dichloro-4-hydroxyphenyl)-1-methyl-cyclohexanecarboxamide, N-(2,6-dimethyl phenyl)-2-methoxy-N-(tetra hydro-2-oxo-3-furanyl)-acetamide, N-(2,6-dimethyl phenyl)-2-methoxy-N-(tetrahydro-2-oxo-3-thienyl)-acetamide, N-(2-chloro-4-nitrophenyl)-4-methyl-3-nitro-benzenesulphonamide, N-(4-cyclohexylphenyl)-1,4,5,6-tetrahydro-2-pyrimidinamine, N-(4-hexylphenyl)-1,4,5,6-tetrahydro-2-pyrimidinamine, N-(5-chloro-2-methylphenyl)-2-methoxy-N-(2-oxo-3-oxazolidinyl)-acetamide, N-(6-methoxy)-3-pyridinyl)-cyclopropanecarboxamide, N-[2,2,2-trichloro-1-[(chloroacetyl)-amino]-ethyl]-benzamide, N-[3-chloro-4,5-bis(2-propinyloxy)-phenyl]-N′-methoxy-methanimidamide, N-formyl-N-hydroxy-DL-alanine-sodium salt, 0,0-diethyl [2-(dipropylamino)-2-oxoethyl]-ethylphosphoramidothioate, O-methyl S-phenyl phenylpropylphosphoramidothioate, S-methyl 1,2,3-benzothiadiazole-7-carbothioate, and spiro[2H]-1-benzopyrane-2,1′(3′H)-isobenzofuran]-3′-one, N-trichloromethyl)thio-4-cyclohexane-1,2-dicarboximide, tetramethylthioperoxydicarbonic diamide, methyl N-(2,6-dimethylphenyl)-N-(methoxyacetyl)-DL-alaninate, 4-(2,2-difluoro-1,3-benzodioxol-4-yl)-1-H-pyrrol-3-carbonitril, or any combination thereof.

When the polypeptides are formulated or applied in combination with commercially available fungicides, the compositions can provide an extra layer of protection for enhancing disease prevention or spread in a plant. The combination of the polypeptides with a fungicide (e.g., a fungicide from the succinate dehydrogenase class) can protect a plant against a primary or secondary fungal infection which may occur if the plant has become compromised or weakened due to exposure to abiotic stress or disease.

The strobilurin fungicide can comprise a Strobilurin A, a Strobilurin B, a Strobilurin C, a Strobilurin D, a Strobilurin E, a Strobilurin F, a Strobilurin G, a Strobilurin H, an Azoxystrobin, a Trifloxystrobin, a Kresoxim methyl, a Fluoxastrobin, Picoxystrobin, or any combination thereof.

The strobilurin fungicide can comprise a non-naturally occurring strobilurin fungicide such as an Azoxystrobin, a Trifloxystrobin, a Kresoxim methyl, a Fluoxastrobin, or any combination thereof. For example, the strobilurin fungicide can comprise a Trifloxystrobin, Fluoxastrobin or Picoxystrobin. Strobilurin fungicides are used to control a range of fungal diseases, including water molds, downy mildews, powdery mildews, leaf spotting and blighting fungi, fruit rotters, and rusts. They are useful for treating a variety of crops, including cereals, field crops, fruits, tree nuts, vegetables, turfgrasses, and ornamentals.

The triazole fungicide can comprise prothioconazole, imidazole, imidazil, prochloraz, propiconazole, triflumizole, diniconazole, flusilazole, penconazole, hexaconazole, cyproconazole, myclobutanil, tebuconazole, difenoconazole, tetraconazole, fenbuconazole, epoxiconazole, metconazole, fluquinconazole, triticonazole, or any combination thereof.

The succinate dehydrogenase inhibitor fungicide can comprise a phenyl-benzamide, phenyl-oxo-ethyl thiophene amide, pyridinyl-ethyl-benzamide, furan-carboxamide, oxathin-carboxamide, thiazole-carboxamide, pyrazole-4-carboxamide, N-cyclopropyl-N-benzyl-pyrazole-carboxamide, N-methoxy-(phenyl-ethyl)-pyrazole-carboxamide, pyridine-carboxamide, or pyrazine-carboxamide, pydiflumetofen, benodanil, flutolanil, mepronil, isofetamid, fluopyram, fenfuram, carboxin, oxycarboxin, thifluzamide, benzovindiflupyr, bixafen, fluindapyr, fluxapyroxad, furametpyr, inpyrfluxam, isopyrazam, penflufen, penthiopyrad, sedaxane, isoflucypram, pydiflumetofen, boscalid, pyraziflumid or any combination thereof.

For example, the succinate dehydrogenase inhibitor fungicide can comprise a phenyl-benzamide, phenyl-oxo-ethyl thiophene amide, pyridinyl-ethyl-benzamide, furan-carboxamide, oxathin-carboxamide, thiazole-carboxamide, pyrazole-4-carboxamide, N-cyclopropyl-N-benzyl-pyrazole-carboxamide, N-methoxy-(phenyl-ethyl)-pyrazole-carboxamide, pyridine-carboxamide, or pyrazine-carboxamide, pydiflumetofen, isofetamid, oxycarboxin, benzovindiflupyr, bixafen, fluindapyr, inpyrfluxam, isopyrazam, penthiopyrad, isoflucypram, pydiflumetofen, pyraziflumid or any combination thereof.

For example, the succinate dehydrogenase inhibitor can comprise bixafen.

The composition can comprise a root hair promoting polypeptide or a retro inverso root hair promoting polypeptide and a succinate dehydrogenase inhibitor. For example, the composition can comprise a root hair promoting polypeptide or a retro inverso root hair promoting polypeptide having an amino acid sequence comprising any one of SEQ ID NOs: 745-766 and a succinate dehydrogenase inhibitor. The succinate dehydrogenase inhibitor can comprise bixafen. The root hair promoting polypeptide or the retro inverso root hair promoting polypeptide can comprise a free polypeptide.

The composition can comprise flagellin or flagellin associated polypeptide or a retro inverso flagellin or flagellin-associated polypeptide and a succinate dehydrogenase inhibitor. For example, the composition can comprise a flagellin or flagellin associated polypeptide having an amino acid sequence comprising any one of SEQ ID NOs: 226, 289, 290, 291, 293, 294, 295, 300, 437, 526, 532, 534, 536, 538, 540, 571-585, and 587-603 or a retro inverso flagellin or flagellin associated polypeptide having an amino acid sequence comprising any one of SEQ ID NOs: 376-525, 527, 529, 531, 533, 535, 537, 539, or 588, or 586 and a succinate dehydrogenase inhibitor. The succinate dehydrogenase inhibitor can comprise bixafen. The serine protease can comprise a free polypeptide.

The composition can comprise a glucanase and a succinate dehydrogenase inhibitor. For example, the composition can comprise glucanase having an amino acid sequence comprising any one of SEQ ID NOs: 731-733 and 767-776 and a succinate dehydrogenase inhibitor. The succinate dehydrogenase inhibitor can comprise bixafen. The glucanase can comprise a free polypeptide.

The composition can comprise a chitinase and a succinate dehydrogenase inhibitor. For example, the composition can comprise a chitinase having an amino acid sequence comprising SEQ ID NO: 777 or 778 and a succinate dehydrogenase inhibitor. The succinate dehydrogenase inhibitor can comprise bixafen. The chitinase can comprise a free polypeptide.

The composition can comprise a serine protease and a succinate dehydrogenase inhibitor. For example, the composition can comprise a serine protease having an amino acid sequence comprising any one of SEQ ID NOs: 721, 722, and 794-796 and a succinate dehydrogenase inhibitor. The succinate dehydrogenase inhibitor can comprise bixafen. The serine protease can comprise a free polypeptide.

The composition can comprise a thionin and a succinate dehydrogenase inhibitor. For example, the composition can comprise a thionin having an amino acid sequence comprising any one of SEQ ID NOs: 620-719 and a succinate dehydrogenase inhibitor. The succinate dehydrogenase inhibitor can comprise bixafen. The thionin or thionin-like polypeptide can comprise a free polypeptide.

The composition can comprise an ACC deaminase polypeptide and a succinate dehydrogenase inhibitor. For example, the composition can comprise an ACC deaminase polypeptide having an amino acid sequence comprising any one of SEQ ID NOs: 723-730 and a succinate dehydrogenase inhibitor. The succinate dehydrogenase inhibitor can comprise bixafen. The ACC deaminase can comprise a free polypeptide.

The bioactive priming polypeptides can be delivered in combination with strobilurins and triazole fungicides, especially fluoxastrobin or trifloxystrobin in combination with prothioconazole. As an additional example, the bioactive priming polypeptides can be delivered in combination with a succinate dehydrogenase inhibitor fungicide (e.g., bixafen).

In addition, the fungicide can comprise azoxystrobin, carboxin, difenoconazole, fludioxonil, fluxapyroxad, ipconazole, mefenoxam, pyraclostrobin, silthiofam, sedaxane, thiram, triticonazole or any combination thereof.

In addition to foliar applied fungicides as described herein, the bioactive priming polypeptides can be provided in combination with a fungicide, an insecticide, a nematicide, a bacteriocide, and a miticide or any agrochemical which is a biological agent.

The agrochemical can include a fertilizer. The fertilizer can comprise ammonium sulfate, ammonium nitrate, ammonium sulfate nitrate, ammonium chloride, ammonium bisulfate, ammonium polysulfide, ammonium thiosulfate, aqueous ammonia, anhydrous ammonia, ammonium polyphosphate, aluminum sulfate, calcium nitrate, calcium ammonium nitrate, calcium sulfate, calcined magnesite, calcitic limestone, calcium oxide, calcium nitrate, dolomitic limestone, hydrated lime, calcium carbonate, diammonium phosphate, monoammonium phosphate, magnesium nitrate, magnesium sulfate, potassium acetate, potassium nitrate, potassium chloride, potassium magnesium sulfate, potassium phosphate, tribasic potassium phosphate, potassium sulfate, sodium nitrates, magnesian limestone, magnesia, urea, urea-formaldehydes, urea ammonium nitrate, sulfur-coated urea, polymer-coated urea, isobutylidene diurea, K₂SO4-Mg₂SO₄, kainite, sylvinite, kieserite, Epsom salts, elemental sulfur, marl, ground oyster shells, fish meal, oil cakes, fish manure, blood meal, rock phosphate, super phosphates, slag, bone meal, wood ash, manure, bat guano, peat moss, compost, green sand, cottonseed meal, feather meal, crab meal, fish emulsion, humic acid, or any combination thereof.

The fertilizer can comprise a liquid fertilizer or a dry fertilizer.

The agrochemical can comprise a micronutrient fertilizer material, the micronutrient fertilizer material comprising boric acid, a borate, a boron frit, copper sulfate, a copper frit, a copper chelate, a sodium tetraborate decahydrate, an iron sulfate, an iron oxide, iron ammonium sulfate, an iron frit, an iron chelate, a manganese sulfate, a manganese oxide, a manganese chelate, a manganese chloride, a manganese frit, a sodium molybdate, molybdic acid, a zinc sulfate, a zinc oxide, a zinc carbonate, a zinc frit, zinc phosphate, a zinc chelate, or any combination thereof.

The agrochemical can comprise an insecticide, the insecticide comprising an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or any combination thereof.

When the composition includes a biostimulant, the biostimulant can comprise a seaweed extract, an elicitor, a polysaccharide, a monosaccharide, a protein extract, a soybean extract, a humic acid, a plant hormone, a plant growth regulator, or any combination thereof.

A variety of colorants can be employed, including organic chromophores classified as nitroso, nitro, azo, including monoazo, bisazo, and polyazo, diphenylmethane, triarylmethane, xanthene, methane, acridine, thiazole, thiazine, indamine, indophenol, azine, oxazine, anthraquinone, phthalocyanine, or any combination thereof.

The composition can further comprise a carrier.

The carrier of the composition can include, but is not limited to, water, peat, wheat, bran, vermiculite, clay, pasteurized soil, calcium carbonate, calcium bicarbonate, dolomite, gypsum, bentonite, a clay, a rock phosphate, a phosphorous compound, titanium dioxide, humus, talc, alginate, activated charcoal, or a combination thereof.

The composition can be in the form of an aqueous solution, a slurry or dispersion, an emulsion, a solid such as a powder or granule, or any other desirable form for applying the composition to a plant or plant part.

The composition can comprise a majority of the bioactive priming polypeptides and/or inducer compounds with the remainder of the composition being agrochemicals or carriers. More specifically, the composition can comprise from about 0.00001% to about 95% of the polypeptides, from about 0.1 to about 80 wt. % of the agrochemicals, and from about 5 to about 50 wt. % carrier based on the total weight of the composition. Alternatively, the composition can comprise from about 0.01 to about 5 wt. % of the polypeptides, from about 0.2 to about 70 wt. % of the agrochemicals, and from about 10 to about 30 wt. % carrier based on the total weight of the composition, or the composition can comprise from about 0.05 wt. % to about 1 wt. % of the polypeptides, from about 30 to about 60 wt. % of the agrochemicals, and from about 40 to about 69 wt. % carrier based on the total weight of the composition. Alternatively, the composition can comprise any detectable amount of the polypeptides, and from about 0.1 to about 80 wt. % of the agrochemicals and from about 5 to about 50 wt. % of the carrier, based on the total weight of the composition.

The composition can comprise a majority of agrochemicals or carriers with the remainder being polypeptides and/or inducer compounds. More specifically, the composition can comprise 0.0000005 wt. % to about 10 wt. % of the polypeptide(s), from about 0.01% to about 99 wt. % of the agrochemical distinct from the inducer compound, and from about 1 to about 99.99 wt % carrier, based on the total weight of the composition. Alternatively, the composition can comprise from about 0.001% to about 5% of the polypeptide(s), from about 0.1% to about 70 wt. % of the agrochemical, and from about 25 to about 99.9 wt % carrier based on the total weight of the composition. Even more specifically, the composition can comprise from about 0.005% to about 0.1% of the polypeptide(s), from about 0.1% to about 60 wt. % of the agrochemical, and from about 40 to about 99.8 wt. % carrier based on the total weight of the composition. Alternatively, the composition can comprise any detectable amount of the polypeptides, any detectable amount of the inducer compound, and from about 1 to 99.99 wt. % of the carrier based on the total weight of the composition. In any of these compositions, the composition can comprise from about 0.0000001 wt. % to about 95 wt. % of the inducer compound based on the total weight of the composition. For example, the composition can comprise from about 0.001 wt. % to about 95 wt. % based on the total weight of the composition.

Even more specifically, the composition can comprise from about 0.00001% to about 95% of the polypeptides, from about 0.000001 wt. % to about 95 wt. % of the inducer compound, from about 0.1 to about 80 wt. % of the agrochemicals, and from about 5 to about 50 wt. % carrier based on the total weight of the composition. Alternatively, the composition can comprise from about 0.01 to about 5 wt. % of the polypeptides, from about 0.000001 wt. % to about 95 wt. % of the inducer compound, from about 0.2 to about 70 wt. % of the agrochemicals, and from about 10 to about 30 wt. % carrier based on the total weight of the composition, or the composition can comprise from about 0.05 wt. % to about 1 wt. % of the polypeptides, from about 0.000001 wt. % to about 95 wt. % of the inducer compound, from about 30 to about 60 wt. % of the agrochemicals, and from about 40 to about 69 wt. % carrier based on the total weight of the composition. Alternatively, the composition can comprise any detectable amount of the polypeptides, and any detectable amount of the inducer compound and from about 0.1 to about 80 wt. % of the agrochemicals and from about 5 to about 50 wt. % of the carrier, based on the total weight of the composition

When the composition comprises two or more inducer compounds, the composition can comprise from about 0.000001 wt. % to about 95 wt. % of a first inducer and from about 0.000001 wt. % to about 95% wt. % of the second inducer, from about 0.1 to about 80 wt. % of the agrochemicals, and from about 5 to about 50 wt. % carrier based on the total weight of the composition. Alternatively, the composition can comprise from about 0.000001 wt. % to about 95 wt. % of the first inducer and from about 0.001 wt. % of the second inducer, from about 0.1 to about 80 wt. % of the agrochemicals, and from about 5 to about 50 wt. % carrier based on the total weight of the composition. Alternatively, the composition can comprise from about 0.001 wt. % of the first inducer and from about 0.000001 wt. % of the second inducer, from about 0.1 to about 80 wt. % of the agrochemicals, and from about 5 to about 99 wt. % carrier based on the total weight of the composition.

The inducer compound can comprise from about 0.000001 wt. % to about 95 wt. % of the composition based on the total weight of the composition. Preferably, the inducer compound comprises from about 0.000001 wt. % to about 95 wt. % of the composition based on the total weight of the composition when the inducer compound comprises a callose synthase inhibitor, an amino acid, salicylic acid, oxalic acid, a betaine, a proline, a benzothiadiazole or any combination thereof. The inducer compound can comprise from about 0.001 wt. % to about 95 wt. % based on the total weight of the composition. Preferably, the inducer compound comprises from about 0.001 wt. % to about 95 wt. % based on the total weight of the composition when the inducer compound comprises a bactericide.

Bioactive priming polypeptides such as the flagellin and flagellin-associated polypeptides, thionin (defensin family), or other growth promoting or altering bioactive priming polypeptides such RHPP, serine proteases, glucanases, amylases, chitinases, or ACC deaminases can be provided as compositions that can either be exogenously and/or endogenously applied to a plant or a plant part and provide enhanced plant growth, productivity and enhanced health of that plant or plant part as described in more detail below.

The bioactive priming polypeptides can be added separately (in individual compositions) or in combination as a composition that are useful as applications to provide a benefit to plants and/or plant parts.

In combination, the polypeptides can be formulated and delivered in a purified polypeptide form either as a genetic fusion on the same recombinant vector, or separately using different recombinant vectors.

The bioactive priming polypeptides can also be created and delivered to a plant or plant part as polypeptides from multiple actives in a fusion protein. Examples of this include delivery of multiple flagellin associated polypeptides produced in series with protease cleavage sites between each polypeptide as is within the skill of one of ordinary skill in the art. Such fusion proteins can include any combination of the bioactive priming polypeptides as described herein, including bioactive priming polypeptides from different classes, such as combinations of flagellin associated polypeptides with RHPP. Bioactive priming polypeptides can also be utilized as protein fusions to plant binding domains, which can direct the polypeptides to distinct locations within the plant where they are most desired or needed for their activities to be beneficial.

Additionally, the polypeptides can be added to formulations provided in a synthetic compound form.

The flagellin and flagellin-associated bioactive priming polypeptides as described herein can be provided individually or in combination containing at least two multiple bioactive priming polypeptides to provide a composition that meets the specific needs of a plant over a wide range of desired host responses and cropping systems.

### IV(A) Isolated Polypeptides

An isolated polypeptide (peptide) is also provided. The isolated polypeptide can enable bioactive priming of a plant or a plant part to increase growth, yield, health, longevity, productivity, and/or vigor of a plant or a plant part and/or decrease abiotic stress in the plant or the plant part and/or protect the plant or the plant part from disease, insects, and/or nematodes, and/or increase the innate immune response of the plant or the plant part and/or change plant architecture. The isolated polypeptide can comprise any of the polypeptides described above in connection with the compositions described herein.

Further, the isolated polypeptide can comprise a Root Hair Promoting polypeptide (RHPP). The RHPP can comprise or consist of any one of SEQ IDs NOs 745-755 (Tables 11 an 12, above). The RHPP can also comprise or consist of a polypeptide having greater than 70% sequence identity, greater than 75% sequence identity, greater than 80% sequence identity, greater than 85% sequence identity, greater than about 90%, greater than about 91%, greater than about 92%, greater than about 93%, greater than about 94%, greater than about 95%, greater than about 96%, greater than about 97%, greater than about 98%, or greater than about 99% sequence identity to any one of SEQ ID NOs: 745-755.

The isolated polypeptide can comprise a retro-inverso Root Hair Promoting polypeptide (RI-RHPP). The RI-RHPP can comprise or consist of any one of SEQ ID NOs: 756-766 (Table 13). The RI-RHPP can also comprise or consist of a polypeptide having greater than 70% sequence identity, greater than 75% sequence identity, greater than 80% sequence identity, greater than 85% sequence identity, greater than about 90%, greater than about 91%, greater than about 92%, greater than about 93%, greater than about 94%, greater than about 95%, greater than about 96%, greater than about 97%, greater than about 98%, or greater than about 99% sequence identity to any one of SEQ ID NOs: 756-766.

The isolated polypeptide can comprise a glucanase, an amylase or a chitinase. For example, the isolated polypeptide can comprise a β-1,3-glucanase, an amylase, or a chitinase. The β-1,3-glucanase can comprise or consist of any one of SEQ ID NOs: 732, 735, and 767-776 (Table 19). The amylase can comprise or consist of any one of SEQ ID NOs: 734 and 735 (Table 19). The chitinase can comprise or consist of SEQ ID NO: 777 or 778 (Table 19). Further, the glucanase, amylase, or chitinase can comprise or consist of a polypeptide having greater than 70% sequence identity, greater than 75% sequence identity, greater than 80% sequence identity, greater than 85% sequence identity, greater than about 90%, greater than about 91%, greater than about 92%, greater than about 93%, greater than about 94%, greater than about 95%, greater than about 96%, greater than about 97%, greater than about 98%, or greater than about 99% sequence identity to any one of SEQ ID NOs: 732, 735, and 767-778.

Accordingly, the isolated polypeptide can comprise the amino acid sequence of any one of SEQ ID NOs: 732, 735, 746-755 and 757-778; or the polypeptide consists of the amino acid sequence of any one of SEQ ID NOs: 732, 735, 745-778.

The amino acid sequence of the isolated polypeptide can comprise any one of SEQ ID NOs: 747, 758, 767-769, 771, 772, 773, 775, and 778, or the amino acid sequence of the polypeptide consists of any one of SEQ ID NOs: 747, 758, 767-769, 771, 772, 773, 775, and 778.

The amino acid sequence of the isolated polypeptide can comprise any one of SEQ ID NOs: 732, 746-750, 757-761, 767-776, and 778, or the amino acid sequence of the polypeptide consists of any one of SEQ ID NOs: 732, 746-750, 757-761, 767-776, and 778.

The amino acid sequence of the isolated polypeptide can comprise any one of SEQ ID NOs: 732, 746-755, 757-776, and 778, or the amino acid sequence of the polypeptide consists of any one of SEQ ID NOs: 732, 746-755, 757-776, and 778.

The amino acid sequence of the isolated polypeptide can comprise any one of SEQ ID NOs: 732, 735, 746-755, 757-778, or the amino acid sequence of the polypeptide consists of any one of SEQ ID NOs: 732, 735, 746-755, 757-778.

### IV. Applications

The agricultural composition, isolated polypeptides, and methods described herein can be used with any species of plant and/or the seeds thereof. The compositions and methods are typically used with seeds that are agronomically important.

The seed can be a transgenic seed from which a transgenic plant can grow that incorporates a transgenic event that confers, for example, tolerance to a particular herbicide or combination of herbicides, increased disease resistance, enhanced tolerance to insects, drought, stress and/or enhanced yield.

The seed can comprise a breeding trait, including for example, a disease tolerant breeding trait.

In some instances, the seed includes at least one transgenic trait and at least one breeding trait.

The bioactive priming polypeptide compositions and methods for applying the polypeptides can be used for the treatment of any suitable seed type, including, but not limited to, row crops and vegetables. For example, one or more plants or plant parts or the seeds of one or more plants can comprise abaca (manila hemp) (Musa textilis), alfalfa for fodder (Medicago sativa), alfalfa for seed (Medicago sativa), almond (Prunus dulcis), anise seeds (Pimpinella anisum), apple (Malus sylvestris), apricot (Prunus armeniaca), areca (betel nut) (Areca catechu), arracha (Arracacia xanthorrhiza), arrowroot (Maranta arundinacea), artichoke (Cynara scolymus), asparagus (Asparagus officinalis), avocado (Persea americana), bajra (pearl millet) (Pennisetum americanum), bambara groundnut (Vigna subterranea), banana (Musa paradisiaca), barley (Hordeum vulgare), beans, dry, edible, for grains (Phaseolus vulgaris), beans, harvested green (Phaseolus and Vigna spp.), beet, fodder (mangel) (Beta vulgaris), beet, red (Beta vulgaris), beet, sugar (Beta vulgaris), beet, sugar for fodder (Beta vulgaris), beet, sugar for seeds (Beta vulgaris), bergamot (Citrus bergamia), betel nut (Areca catechu), black pepper (Piper nigrum), black wattle (Acacia mearnsii), blackberries of various species (Rubus spp.), blueberry (Vaccinium spp.), Brazil nut (Bertholletia excelsa), breadfruit (Artocarpus altilis), broad bean, dry (Vicia faba), broad bean, harvested green (Vicia faba), broccoli (Brassica oleracea var. botrytis), broom millet (Sorghum bicolor), broom sorghum (Sorghum bicolor), Brussels sprouts (Brassica oleracea var. gemmifera), buckwheat (Fagopyrum esculentum), cabbage, red, white, Savoy (Brassica oleracea var. capitata), cabbage, Chinese (Brassica chinensis), cabbage, for fodder (Brassica spp.), cacao (cocoa) (Theobroma cacao), cantaloupe (Cucumis melo), caraway seeds (Carum carvi), cardamom (Elettaria cardamomum), cardoon (Cynara cardunculus), carob (Ceratonia siliqua), carrot, edible (Daucus carota spp. sativa), carrot, for fodder (Daucus carota sativa), cashew nuts (Anacardium occidentale), cassava (manioc) (Manihot esculenta), castor bean (Ricinus communis), cauliflower (Brassica oleracea var. botrytis), celeriac (Apium graveolens var. rapaceum), celery (Apium graveolens), chayote (Sechium edule), cherry, all varieties (Prunus spp.), chestnut (Castanea sativa), chickpea (gram pea) (Cicer arietinum), chicory (Cichorium intybus), chicory for greens (Cichorium intybus), chili, dry (all varieties) (Capsicum spp. (annuum)), chili, fresh (all varieties) (Capsicum spp. (annuum)), cinnamon (Cinnamomum verum), citrus (all varieties, family Rutaceae), citron (Citrus medica), citronella (Cymbopogon citrates; Cymbopogon nardus), clementine (Citrus reticulata), clove (Eugenia aromatica; Syzygium aromaticum), clover for fodder (all varieties) (Trifolium spp.), clover for seed (all varieties) (Trifolium spp.), cocoa (cacao) (Theobroma cacao), coconut (Cocos nucifera), cocoyam (Colocasia esculenta), coffee (Coffea spp.), cola nut, all varieties (Cola acuminata), colza (rapeseed) (Brassica napus), corn (maize), for cereals (Zea mays), corn (maize), for silage (Zea mays), corn (maize), for vegetable (Zea mays), corn for salad (Valerianella locusta), cotton, all varieties (Gossypium spp.), cottonseed, all varieties (Gossypium spp.), cowpea, for grain (Vigna unguiculata), cowpea, harvested green (Vigna unguiculata), cranberry (Vaccinium spp.), cress (Lepidium sativum), cucumber (Cucumis sativus), currants, all varieties (Ribes spp.), custard apple (Annona reticulate), dasheen (Colocasia esculenta), dates (Phoenix dactylifera), drumstick tree (Moringa oleifera), durra (sorghum) (Sorghum bicolour), durum wheat (Triticum durum), earth pea (Vigna subterranea), edo (eddoe) (Xanthosoma spp.; Colocasia spp.), eggplant (Solanum melongena), endive (Cichorium endivia), fennel (Foeniculum vulgare), fenugreek (Trigonella foenum-graecum), fig (Ficus carica), filbert (hazelnut) (Corylus avellana), fique (Furcraea macrophylla), flax for fiber (Linum usitatissimum), flax for oil seed (linseed) (Linum usitatissimum), formio (New Zealand flax) (Phormium tenax), garlic, dry (Allium sativum), garlic, green (Allium sativum), geranium (Pelargonium spp.; Geranium spp.), ginger (Zingiber officinale), gooseberry, all varieties (Ribes spp.), gourd (Lagenaria spp; Cucurbita spp.), gram pea (chickpea) (Cicer arietinum), grapes for raisins (Vitis vinifera), grapes for table use (Vitis vinifera), grapes for wine (Vitis vinifera), grapefruit (Citrus paradisi), grass esparto (Lygeum spartum), grass, orchard (Dactylis glomerata), grass, Sudan (Sorghum bicolor var. sudanense), groundnut (peanut) (Arachis hypogaea), guava (Psidium guajava), guinea corn (sorghum) (Sorghum bicolor), hazelnut (filbert) (Corylus avellana), hemp fiber (Cannabis sativa spp. indica), hemp, manila (abaca) (Musa textilis), hemp, sun (Crotalaria juncea), hempseed (marijuana) (Cannabis sativa), henequen (Agave fourcroydes), henna (Lawsonia inermis), hop (Humulus lupulus), horse bean (Vicia faba), horseradish (Armoracia rusticana), hybrid maize (Zea mays), indigo (Indigofera tinctoria), jasmine (Jasminum spp.), Jerusalem artichoke (Helianthus tuberosus), jowar (sorghum) (Sorghum bicolor), jute (Corchorus spp.), kale (Brassica oleracea var. acephala), kapok (Ceiba pentandra), kenaf (Hibiscus cannabinus), kiwi or kiwifruit (Actinidia deliciosa), kohlrabi (Brassica oleracea var. gongylodes), lavender (Lavandula spp.), leek (Allium ampeloprasum; Allium porrum), lemon (Citrus limon), lemongrass (Cymbopogon citratus), lentil (Lens culinaris), lespedeza, all varieties (Lespedeza spp.), lettuce (Lactuca sativa var. capitata), lime, sour (Citrus aurantifolia), lime, sweet (Citrus limetta), linseed (flax for oil seed) (Linum usitatissimum), licorice (Glycyrrhiza glabra), litchi (Litchi chinensis), loquat (Eriobotrya japonica), lupine, all varieties (Lupinus spp.), Macadamia (Queensland nut) (Macadamia spp. ternifolia), mace (Myristica fragrans), maguey (Agave atrovirens), maize (corn) (Zea mays), maize (corn) for silage (Zea mays), maize (hybrid) (Zea mays), maize, ordinary (Zea mays), mandarin (Citrus reticulata), mangel (fodder beet) (Beta vulgaris), mango (Mangifera indica), manioc (cassava) (Manihot esculenta), maslin (mixed cereals) (mixture of Triticum spp. and Secale cereale), medlar (Mespilus germanica), melon, except watermelon (Cucumis melo), millet broom (Sorghum bicolor), millet, bajra (Pennisetum americanum), bulrush (Pennisetum americanum), millet, finger (Eleusine coracana), millet (Urochola ramosa), foxtail (Setaria italica), millet, Japanese (Echinochloa esculenta), millet, pearl (bajra, bulrush) (Pennisetum americanum), millet, proso (Panicum miliaceum), mint, all varieties (Mentha spp.), mulberry for fruit, all varieties (Morus spp.), mulberry for silkworms (Morus alba), mushrooms (Agaricus spp.; Pleurotus spp.; Volvariella), mustard (Brassica nigra; Sinapis alba), nectarine (Prunus persica var. nectarina), New Zealand flax (formio) (Phormium tenax), Niger seed (Guizotia abyssinica), nutmeg (Myristica fragrans), oats, for fodder (Avena spp.), oil palm (Elaeis guineensis), okra (Abelmoschus esculentus), olive (Olea europaea), onion seed (Allium cepa), onion, dry (Allium cepa), onion, green (Allium cepa), opium (Papaver somniferum), orange (Citrus sinensis), orange, bitter (Citrus aurantium), ornamental plants (various), palm palmyra (Borassus flabellifer), palm (Arecaceae), palm kernel oil (Elaeis guineensis), palm, oil (Elaeis guineensis), palm, sago (Metroxylon sagu), papaya (pawpaw) (Carica papaya), parsnip (Pastinaca sativa), pea, edible dry, for grain (Pisum sativum), pea, harvested green (Pisum sativum), peach (Prunus persica), peanut (groundnut) (Arachis hypogaea), pear (Pyrus communis), pecan nut (Carya illinoensis), pepper, black (Piper nigrum), pepper, dry (Capsicum spp.), persimmon (Diospyros kaki; Diospyros virginiana), pigeon pea (Cajanus cajan), pineapple (Ananas comosus), pistachio nut (Pistacia vera), plantain (Musa sapientum), plum (Prunus domestica), pomegranate (Punica granatum), pomelo (Citrus grandis), poppy seed (Papaver somniferum), potato (Solamum tuberosum), potato, sweet (Ipomoea batatas), prune (Prunus domestica), pumpkin, edible (Cucurbita spp.), pumpkin, for fodder (Cucurbita spp.), pyrethum (Chrysanthemum cinerariaefolium), quebracho (Aspidosperma spp.), Queensland nut (Macadamia spp. ternifolia), quince (Cydonia oblonga), quinine (Cinchona spp.), quinoa (Chenopodium quinoa), ramie (Boehmeria nivea), rapeseed (colza) (Brassica napus), raspberry, all varieties (Rubus spp.), red beet (Beta vulgaris), redtop (Agrostis spp.), rhea (Boehmeria nivea), rhubarb (Rheum spp.), rice (Oryza sativa; Oryza glaberrima), rose (Rose spp.), rubber (Hevea brasiliensis), rutabaga (swede) (Brassica napus var. napobrassica), rye (Secale cereale), ryegrass seed (Lolium spp.), safflower (Carthamus tinctorius), sainfoin (Onobrychis viciifolia), salsify (Tragopogon porrifolius), sapodilla (Achras sapota), satsuma (mandarin/tangerine) (Citrus reticulata), scorzonera (black salsify) (Scorzonera hispanica), sesame (Sesamum indicum), shea butter (nut) (Vitellaria paradoxa), sisal (Agave sisalana), sorghum (Sorghum bicolor), sorghum, broom (Sorghum bicolor), sorghum, durra (Sorghum bicolor), sorghum, guinea corn (Sorghum bicolor), sorghum, jowar (Sorghum bicolor), sorghum, sweet (Sorghum bicolor), soybean (Glycine max), soybean hay (Glycine max), spelt wheat (Triticum spelta), spinach (Spinacia oleracea), squash (Cucurbita spp.), strawberry (Fragaria spp.), sugar beet (Beta vulgaris), sugar beet for fodder (Beta vulgaris), sugar beet for seed (Beta vulgaris), sugarcane for fodder (Saccharum officinarum), sugarcane for sugar or alcohol (Saccharum officinarum), sugarcane for thatching (Saccharum officinarum), sunflower for fodder (Helianthus annuus), sunflower for oil seed (Helianthus annuus), sunhemp (Crotalaria juncea), swede (Brassica napus var. napobrassica), swede for fodder (Brassica napus var. napobrassica), sweet corn (Zea mays), sweet lime (Citrus limetta), sweet pepper (Capsicum annuum), sweet potato (Lopmoea batatas), sweet sorghum (Sorghum bicolor), tangerine (Citrus reticulata), tannia (Xanthosoma sagittifolium), tapioca (cassava) (Manihot esculenta), taro (Colocasia esculenta), tea (Camellia sinensis), teff (Eragrostis abyssinica), timothy (Phleum pratense), tobacco (Nicotiana tabacum), tomato (Lycopersicon esculentum), trefoil (Lotus spp.), triticale, for fodder (hybrid of Triticum aestivum and Secale cereale), tung tree (Aleurites spp.; Fordii), turnip, edible (Brassica rapa), turnip, for fodder (Brassica rapa), urena (Congo jute) (Urena lobata), vanilla (Vanilla planifolia), vetch, for grain (Vicia sativa), walnut (Juglans spp., especially Juglans regia), watermelon (Citrullus lanatus), wheat (Triticum aestivum), yam (Dioscorea spp.), or yerba mate (Ilex paraguariensis).

The isolated polypeptides and compositions disclosed herein can also be applied to turf grass, ornamental grass, flowers, ornamentals, trees, horticultural plants, and shrubs.

The isolated polypeptides and compositions comprising the bioactive priming polypeptides are also suitable for use in the nursery, lawn and garden, floriculture, horticulture or the cut flower industry and provide benefits for enhanced plant productivity, protection health, vigor and longevity. For example, they can be applied to perennials, annuals, forced bulbs, or pseudo bulbs, herbs, groundcovers, trees, shrubs, ornamentals (e.g., orchids, etc.), tropical plants, vines and nursery stock.

The isolated polypeptides and compositions comprising the bioactive priming polypeptides and/or inducer compounds described herein are suitable for treating plants, plant parts and plant propagation material(s), for example, any plant or plant part, such as seeds, roots, stems, vascular system (e.g., phloem and xylem), floral organs, root stocks, scions, bulb, pseudobulbs, rhizomes, tubers, etc. The compositions comprising the bioactive priming polypeptides and/or inducer compounds described herein can be applied to the soil surrounding the plant (e.g., in furrow).

The isolated polypeptides and bioactive priming compositions can be applied as seed treatments to treat for a number of pests, diseases, nutrient deficiencies while enhancing plant growth and productivity.

Seed coating or dressing compositions can be, for example, a liquid carrier composition, a slurry composition, or a powder composition applied with conventional additives that are provided to make the seed treatment have sticky qualities to stick to and coat the seeds. Suitable additives for a seed composition comprise: talcs, graphites, gums, stabilizing polymers, coating polymers, finishing polymers, slip agents for seed flow and plantability, cosmetic agents and cellulosic materials such as carboxymethyl cellulose and the like. The bioactive priming polypeptide seed treatments can further comprise colorant agents and other such additives.

The bioactive priming compositions can be applied individually as seed treatments or in combination with other additives such as fungicides, insecticides, inoculants, plant growth regulators, plant growth promoting microbes, fertilizers and fertilizer enhancers, seed nutrients, biological control agents, herbicidal antidotes and seedling disease treatments and with other conventional seed treatments.

The seed treatment composition as described herein can be applied to seeds in a suitable carrier such as water or a powder that is not harmful to the seeds or the environment. The seeds are then planted in conventional fashion.

Preferred seed treatments such as Bt.4Q7Flg22 (SEQ ID NO: 226 or SEQ ID NO: 571), Ec.Flg22 (SEQ ID NO: 526) and Gm.RHPP (SEQ ID NO: 604) are useful to enhance seedling development, decrease the time for germination, increase the number of seeds that germinate, and enhance seedling survivability. In addition, the seed treatment compositions enhance seed protection from microbial-based diseases which are known to contact the seed or the soil surrounding the seed and spread during early seedling establishment.

The seed treatment composition can comprise a polypeptide and/or an inducer compound as described herein and a fungicide, an insecticide, a nematocide, a biological control agent, a biostimulant, a microbe, or any combination thereof

The seed treatment composition can comprise a polypeptide and/or an inducer compound as described herein and clothianidin, Bacillus firmus, metalaxyl, or any combination thereof.

The seed treatment composition can comprise a polypeptide and/or an inducer compound as described herein, clothianidin and fluopyram.

The seed treatment can comprise a polypeptide and/or an inducer compound as described herein, metalaxyl and fluopyram.

The bioactive priming compositions described herein can be applied directly to the seed as a solution or in combination with other commercially available additives. Solutions containing the water-soluble polypeptide can be sprayed or otherwise applied to the seed as a seed slurry or a seed soak. Solids or dry materials containing soluble bioactive priming polypeptides are also useful to promote effective seedling germination, growth and protection during early seedling establishment.

The bioactive priming compositions described herein can be formulated with a solubilizing carrier such as water, buffer (e.g., citrate or phosphate buffer) and other treating agents (e.g., alcohol, other solvents) or any solubilizing agent. In addition, small amounts of drying agent enhancers, such as lower alcohols, etc. can be utilized in the composition. Surfactants, emulsifiers and preservatives can also be added at small (0.5% v/v or less) levels in order to enhance the stability of the seed coating product.

Seed treatments containing the bioactive priming compositions herein can be applied using any commercially available seed treatment machinery or can also be applied using any acceptable non-commercial method(s) such as the use of syringes or any other seed treatment device. General seed treatments coating procedures using bioactive priming polypeptides can be performed using a Wintersteiger HEGE 11 (Wintersteiger AG, Austria, Germany) and applied to the seed of major crops, namely corn, soybean, wheat, rice and various vegetables. The capacity of this seed treatment machinery can accommodate a large number of different seed types, sizes and amounts of seed (20-3000 grams). The seed is loaded into bowls of the seed treater machinery. The bowl selection depends on the treatment seed amount required and the size of the bowl selected: large 14.5 L bowl (500-3000 g seed per coating); medium 7 L bowl (80-800 g seed per coating); and small 1 L bowl (20-100 g seed per coating). Other larger seed treatment systems are also available.

The seed is distributed toward the radial peripheries of the rotatable bowls via an application of centrifugal force with the centrifugal coating device. The spinning disc located at the bottom of the bowl distributes the seed treatment evenly over the seed. At this point, the spin cycle is started which causes the seeds to revolve around the bowl center in a circle to evenly coat the seeds. The process of seed treatment coating is initiated after the seed is evenly dispersed around the spreader. Seed treatment sample material (such as a powdered, semi-liquid, liquid or a slurry) can be applied onto the rotatable disk as the disks are spinning within the rotatable bowls used to distribute the seed treatment evenly to provide a uniform coat and dress the surface of the seed.

A constant air flow delivered using compressed air (2-6 bars) can be provided during seed coating to assist with uniformly coating the seeds in the bowl. The amount of time for the coating of the seed depends on the amount of the seed, the viscosity of the seed treatment and the type of the seed used in the treatment. A seed treatment calculator is used to adjust for all volumes, for most major and commercially grown crops and the type of seed treatment being applied.

The seeds can be coated using a variety of methods including, but not limited to, pouring or pumping, drizzling or spraying an aqueous solution containing the bioactive priming polypeptides on or over a seed, spraying or applying onto a layer of seeds either with the use or without the use of a conveyor system. Suitable mixing devices include tumblers, mixing basins or drums, or other fluid applicating devices that include basins or drums used to contain the seed while coating.

After the seed has been treated and dried, the seeds are distributed into a larger storage container(s). Seeds are either air dried or dried with a continuous air stream that passes over the seeds. Seeds are then transferred into a separate container or bag for shipment, transfer or storage.

The bioactive priming compositions or isolated polypeptides can further be provided for delivery to a plant surface or plant plasma membrane as a foliar spray or a seed treatment to an area surrounding a plant or a plant part.

The bioactive priming compositions or isolated polypeptides can also be provided as a seed treatment application or on a matrix such as immobilized or impregnated on a particle, or a granule such as used in a broadcast treatment.

The bioactive priming compositions or isolated polypeptides as described herein can be applied to plants and plant parts using an exogenous application as a spray, soil treatment, in furrow, seed treatment, dip or wash or as an endogenous application as an injection, inoculation, irrigation, infiltration, etc.

The isolated polypeptides or compositions comprising polypeptides and/or an inducer compound can be applied directly to a plant or to the area surrounding a plant or plant part.

They can also be provided on a matrix material which is then provided to a plant or plant part.

The compositions containing the flagellin-associated bioactive priming polypeptides and/or inducer compounds can also be provided for direct delivery into a plant, plant tissues or a plant cell by various delivery methods, for example, injection, inoculation or infiltration (for example, infiltration into the stomata on the leaf). These polypeptides can also be provided in a manner where they can move systemically through a plant and influence signaling cascades in the plant that subsequently produce beneficial and productive outcomes to the plant or plant part.

The isolated polypeptides or bioactive priming composition described herein, can be provided for direct delivery into a plant, plant tissues, or a plant cell by an endogenous application. For example, the isolated polypeptides and/or bioactive priming compositions can be directly injected into a plant. Preferably, the injection allows direct delivery of the isolated polypeptide or the composition into the vascular system of the plant (e.g., into the phloem or xylem). In some cases, the isolated polypeptide delivered by direct injection into the vascular system of the plant comprises a glucanase (e.g., a β-1,3-glucanase).

Retro-inverso Flg bioactive priming polypeptides as described in Table 4 or Table 5 can be applied individually or in combination with any other flagellin, flagellin-associated or other bioactive priming polypeptide sequences as described herein. Combinations of such RI flagellin and flagellin-associated bioactive priming polypeptides are useful as plant protectants as well as plant growth promoting enhancers.

The signature (SEQ ID NO: 542-548; Table 6), signal anchor sorting (SEQ ID NO: 549-562, Table 7) and secretion assistance polypeptides (SEQ ID NOs 563-570; Table 8) can be used in combination with any of the flagellin coding (Table 1), N and/or C-terminal conserved sequences from Bacillus-derived flagellins (Table 2), flagellin-associated polypeptides: Flg22 and FlgII-28 (Table 3), the retro inverso forms of Flg22 and FlgII-28 (Table 4) or any of the other Flgs (Table 5) as described herein.

For example, any of the Flg-associated bioactive priming polypeptides or combinations (compositions) thereof can be provided in individual formulations and applied either simultaneously, sequentially in separate formulations or provided as fusion protein(s) that contain the assistance sequences as described in Tables 6-8 and applied directly or separately to a plant or plant part.

Compositions comprising a flagellin or flagellin-associated polypeptide and an inducer compound can be used to improved disease symptoms or pathogen titer in a plant (particularly in citrus plants). For example, compositions described herein can prevent or reduce callose deposition in or around phloem plasmodesmata that occurs in CLas infeted citrus plants. They can also decrease fruit drop caused by such infections.

Compositions comprising the flagellin or flagellin-associated polypeptides and an inducer compound described herein are also efficient at improving juice quality and/or quantity from citrus plants.

Compositions described herein can also be used to improve yield in a row crop (e.g., soybeans or corn).

Compositions comprising at least one proline, betaine or an ACC deaminase can also beneficially reduce abiotic stress in a plant or plant part.

Compositions comprising a glucanase and a serine protease can also beneficially reduce mold and/or prevent fungal spore germination on a fruit. In some cases, compositions comprising a glucanase and a serine protease can be applied as a fruit wash to the exterior of a fruit.

Compositions comprising oxytetracycline and 2-deoxy-D-glucose can beneficially increase fruit yield, fruit size and juice quality.

Compositions comprising a flagellin or flagellin associated polypeptide (e.g., SEQ ID NO: 226) and an ACC deaminase can beneficially increase yield of a crop.

Compositions comprising a β-1,3-glucanase can beneficially improve health and vigor of a plant (i.e., a citrus tree), particularly when injected into the trunk of the tree. The β-1,3-glucanase can be injected as an isolated polypeptide or as a composition described herein.

### V. Methods of Use

Methods are provided for increasing growth, yield, health, longevity, productivity, and/or vigor of a plant or a plant part and/or protecting the plant or the plant part from disease, and/or increasing the innate immune response of the plant or the plant part. The method can comprise applying the compositions as described herein to a plant, a plant part, or a plant growth medium from which the plant will be grown or a rhizosphere in an area surrounding the plant or the plant part to increase growth, yield, health, longevity, productivity, and/or vigor of the plant or the plant part and/or protect the plant or the plant part from disease, and/or increase the innate immune response of the plant or the plant part.

Another method is provided for increasing juice content and/or improving juice, sugar or acid content and/or improving a Brix:acid ratio of juice obtained from a plant, the method comprising applying any composition described herein to the plant or plant part, or plant growth medium in which the plant will be grown, or a rhizosphere in an area surrounding the plant or plant part to increase juice content and/or improve juice, sugar or acid content or improve a brix:acid ratio of juice obtained from the plant or plant part.

Methods using the bioactive priming compositions are also provided to increase the overall plant productivity in a field, orchard, planting bed, nursery, timberland, farm, lawn, garden, garden center or acreage. Applications and methods using the bioactive priming compositions are also useful for increasing plant growth, health and productivity in diverse crops (monocots and dicots), for example, corn, wheat, rice, sugarcane, soybean, sorghum, potatoes and a variety of vegetables.

A “bioactive priming” approach is also provided by direct application of the compositions, which can be applied either exogenously to a plant cell surface or endogenously to the interior of a plant and/or a plant cell. The compositions are provided for delivery to the plant surface or plasma cell membrane or to the interior of a plant, plant tissue or cell and are useful for regulating developmental processes that result in enhanced growth phenotypes such as increases in overall biomass, vegetative growth, seed fill, seed size, and number of seed that contribute to increases in the total yield of crop plants.

Application of the retro-inverso Flg polypeptides provided in agricultural formulations can result in enhanced plant protection from diseases and abiotic stresses while synergistically enhancing growth, productivity and yield while maintaining increased plant health with enhanced plant performance for longer periods of time.

Selection of the native L (Table 3) or the retro-inverso D (Table 4) forms of the Flg-associated polypeptides can depend on the environment, the plant/crop, or the combination of plant/crop and environment. In addition, the timing of the treatment application (for example, a foliar spray application) during the growing season are all relevant considerations. The retro inverso Flg bioactive priming polypeptides have enhanced binding affinity to cell surface membranes. Due to these features, the RI forms of the Flg bioactive priming polypeptides can be used to improve abiotic stress tolerance in a plant or plant part.

Additionally, the retro inverso forms of RI Ec.Flg22 and RI Bt.4Q7Flg22 can be useful to stimulate the closure of stomata under conditions of drought and heat stress and improve yields under those conditions. Control of stomatal closure using Flg-associated bioactive priming polypeptide applied to a plant during periods of environmental stress can assist in the regulation of water loss and stabilize turgor pressure in a plant when environmental conditions are unfavorable.

In the methods for increasing growth, yield, health, longevity, productivity, and/or vigor of a plant or a plant part and/or protecting the plant or the plant part from disease, and/or increasing the innate immune response of the plant or the plant part, the composition preferably comprises (A) at least one bioactive priming polypeptide and an inducer compound or (B) at least two bioactive priming polypeptides, optionally, with an inducer compound wherein: the polypeptide or polypeptides of (A) or (B) comprise: (i) a flagellin or flagellin-associated polypeptide; or (ii) a retro inverso flagellin or flagellin-associated polypeptide (iii) a root hair promoting polypeptide (RHPP); or (iv) a retro inverso root hair promoting polypeptide (RI RHPP); or (v) a thionin or thionin-like polypeptide; or (vi) a glucanase polypeptide; or (vii) a serine protease polypeptide; or (viii) an ACC deaminase (1-aminocyclopropane-1-carboxylate deaminase) polypeptide; or (ix) an amylase; or (x) a chitinase; or (xi) any combination thereof, with the provisos that:
(1) the inducer compound comprises a callose synthase inhibitor, β amino butyric acid (BABA), a betaine, a proline, a benzothiazole, salicylic acid, oxalic acid, a succinate dehydrogenase inhibitor, or any combination thereof when the polypeptide of (A) comprises any polypeptide from groups (i)-(v) but not polypeptides selected from the groups (vi) to (x); and
(2) the inducer compound comprises a bacteriocide, an amino acid or isomer thereof, a callose synthase inhibitor, a substituted or unsubstituted benzoic acid or derivative thereof, a dicarboxylic acid or a derivative thereof, a betaine, a succinate dehydrogenase inhibitor, or any combination thereof when the polypeptide of (A) comprises any polypeptide from groups (vi) to (x); and
(3) the composition comprises the inducer compound and the inducer compound comprises a callose synthase inhibitor, beta amino butyric acid (BABA), a betaine, a proline, a benzothiazole, salicylic acid, oxalic acid, a succinate dehydrogenase inhibitor, or any combination thereof when the two or more polypeptides of (B) comprise polypeptides selected from groups (i)-(v) but not polypeptides selected from the groups (vi) to (x).

In the methods for increasing growth, yield, health, longevity, productivity, and/or vigor of a plant or a plant part and/or protecting the plant or the plant part from disease, and/or increasing the innate immune response of the plant or the plant part, the composition can comprise an inducer compound comprising a callose synthase inhibitor, β-amino butyric acid (BABA), a betaine, a proline, salicylic acid, oxalic acid, a succinate dehydrogenase inhibitor, or any combination thereof when the polypeptide of (A) comprises any polypeptide from groups (i)-(v) but not polypeptides selected from the groups (vi) to (x).

In the methods for increasing growth, yield, health, longevity, productivity, and/or vigor of a plant or a plant part and/or protecting the plant or the plant part from disease, and/or increasing the innate immune response of the plant or the plant part, the composition can comprise an inducer compound comprising a callose synthase inhibitor, β-amino butyric acid (BABA), salicylic acid, oxalic acid, a succinate dehydrogenase inhibitor, or any combination thereof when the polypeptide of (A) comprises any polypeptide from groups (i)-(v) but not polypeptides selected from the groups (vi) to (x).

In the methods for increasing growth, yield, health, longevity, productivity, and/or vigor of a plant or a plant part and/or protecting the plant or the plant part from disease, and/or increasing the innate immune response of the plant or the plant part, the composition can comprise an inducer compound comprising a callose synthase inhibitor, β-amino butyric acid (BABA), a succinate dehydrogenase inhibitor, or any combination thereof when the polypeptide of (A) comprises any polypeptide from groups (i)-(v) but not polypeptides selected from the groups (vi) to (x).

In the methods for increasing growth, yield, health, longevity, productivity, and/or vigor of a plant or a plant part and/or protecting the plant or the plant part from disease, and/or increasing the innate immune response of the plant or the plant part, the composition can comprise an inducer compound comprising a betaine or a proline when the polypeptide of (A) comprises any polypeptide from groups (i)-(v) but not polypeptides selected from the groups (vi) to (x).

In the methods for increasing growth, yield, health, longevity, productivity, and/or vigor of a plant or a plant part and/or protecting the plant or the plant part from disease, and/or increasing the innate immune response of the plant or the plant part, the composition can comprise an inducer compound comprising salicylic acid or oxalic acid when the polypeptide of (A) comprises any polypeptide from groups (i)-(v) but not polypeptides selected from the groups (vi) to (x).

Alternatively, in the methods for increasing growth, yield, health, longevity, productivity, and/or vigor of a plant or a plant part and/or protecting the plant or the plant part from disease, and/or increasing the innate immune response of the plant or the plant part, the composition can comprise an inducer compound comprising a callose synthase inhibitor, β-amino butyric acid (BABA), a betaine, a proline, salicylic acid, oxalic acid, a succinate dehydrogenase inhibitor, or any combination thereof when the two or more polypeptides of (B) comprise polypeptides selected from groups (i)-(v) but not polypeptides selected from the groups (vi) to (x).

Alternatively, in the methods for increasing growth, yield, health, longevity, productivity, and/or vigor of a plant or a plant part and/or protecting the plant or the plant part from disease, and/or increasing the innate immune response of the plant or the plant part, the composition can comprise an inducer compound comprising a callose synthase inhibitor, β-amino butyric acid (BABA), salicylic acid, oxalic acid, a succinate dehydrogenase inhibitor, or any combination thereof when the two or more polypeptides of (B) comprise polypeptides selected from groups (i)-(v) but not polypeptides selected from the groups (vi) to (x).

Alternatively, in the methods for increasing growth, yield, health, longevity, productivity, and/or vigor of a plant or a plant part and/or protecting the plant or the plant part from disease, and/or increasing the innate immune response of the plant or the plant part, the composition can comprise an inducer compound comprising a callose synthase inhibitor, β-amino butyric acid (BABA), or any combination thereof when the two or more polypeptides of (B) comprise polypeptides selected from groups (i)-(v) but not polypeptides selected from the groups (vi) to (x).

Alternatively, in the methods for increasing growth, yield, health, longevity, productivity, and/or vigor of a plant or a plant part and/or protecting the plant or the plant part from disease, and/or increasing the innate immune response of the plant or the plant part, the composition can comprise an inducer compound comprising a betaine or a proline when the two or more polypeptides of (B) comprise polypeptides selected from groups (i)-(v) but not polypeptides selected from the groups (vi) to (x).

Alternatively, in the methods for increasing growth, yield, health, longevity, productivity, and/or vigor of a plant or a plant part and/or protecting the plant or the plant part from disease, and/or increasing the innate immune response of the plant or the plant part, the composition can comprise an inducer compound comprising salicylic acid or oxalic acid when the two or more polypeptides of (B) comprise polypeptides selected from groups (i)-(v) but not polypeptides selected from the groups (vi) to (x).

Alternatively in the methods for increasing growth, yield, health, longevity, productivity, and/or vigor of a plant or a plant part and/or protecting the plant or the plant part from disease, and/or increasing the innate immune response of the plant or the plant part, the composition can comprise a polypeptide selected from groups (i) to (x) and at least one inducer compound comprising a succinate dehydrogenase inhibitor.

Alternatively in the methods for increasing growth, yield, health, longevity, productivity, and/or vigor of a plant or a plant part and/or protecting the plant or the plant part from disease, and/or increasing the innate immune response of the plant or the plant part, the composition can comprise a callose synthase inhibitor and at least one inducer compound comprising a bacteriocide, an amino acid, substituted or unsubstituted benzoic acid or derivative or salt thereof, a dicarboxylic acid or derivative or salt thereof, a betaine, a proline, a benzothiadiazole, a succinate dehydrogenase inhibitor or any combination thereof. Preferably, the callose synthase inhibitor is 2-DDG. The bacteriocide can be oxytetracycline. The substituted benzoic acid can be salicylic acid. The dicarboxylic acid can be oxalic acid.

Alternatively, in the methods for increasing growth, yield, health, longevity, productivity, and/or vigor of a plant or a plant part and/or protecting the plant or the plant part from disease, and/or increasing the innate immune response of the plant or the plant part, the composition can comprise a bacteriocide and at least one inducer compound comprising of a β-amino butyric acid (BABA), a betaine, a proline, a benzothiadiazole, salicylic acid, oxalic acid, a succinate dehydrogenase inhibitor or any combination thereof. The bacteriocide can be oxytetracycline.

Alternatively, in the methods for increasing growth, yield, health, longevity, productivity, and/or vigor of a plant or a plant part and/or protecting the plant or the plant part from disease, and/or increasing the innate immune response of the plant or the plant part, the composition can comprise a bacteriocide and at least one of a callose synthase inhibitor, β amino butyric acid (BABA), a proline, a betaine, salicylic acid, a succinate dehydrogenase inhibitor, or oxalic acid. The callose synthase inhibitor can be 2-DDG. The bacteriocide can be oxytetracycline.

Alternatively, in the methods for increasing growth, yield, health, longevity, productivity, and/or vigor of a plant or a plant part and/or protecting the plant or the plant part from disease, and/or increasing the innate immune response of the plant or the plant part, the composition can comprise a bacteriocide and at least one of a callose synthase inhibitor, β amino butyric acid (BABA), salicylic acid, a succinate dehydrogenase or oxalic acid. The callose synthase inhibitor can be 2-DDG. The bacteriocide can be oxytetracycline. The succinate dehydrogenase can be bixafen.

Alternatively, in the methods for increasing growth, yield, health, longevity, productivity, and/or vigor of a plant or a plant part and/or protecting the plant or the plant part from disease, and/or increasing the innate immune response of the plant or the plant part, the composition can comprise a bacteriocide and a callose synthase inhibitor or β amino butyric acid (BABA). The callose synthase inhibitor can be 2-DDG. The bacteriocide can be oxytetracycline.

Alternatively, in the methods for increasing growth, yield, health, longevity, productivity, and/or vigor of a plant or plant part and/or protecting the plant or the plant part from disease and/or increasing the innate immune response of the plant or plant part, the method can comprise applying an isolated polypeptide to the plant or plant part. The isolated polypeptide can comprise a β-1,3-glucanase. Preferably, when the isolated polypeptide comprises a β-1,3-glucanase, the peptide is injected into a trunk of a plant. The isolated polypeptide can comprise an RHPP, a retro-inverso RHPP, a glucanase, a chitinase and/or an amylase as described herein. For example, the isolated polypeptide can comprise an RHPP having an amino acid sequence comprising or consisting of any one of SEQ ID NOs: 745-755, or a retro-inverso RHPP having an amino acid sequence comprising or consisting of any one of SEQ ID NOs: 756-766, a β-1,3-glucanase having an amino acid sequence comprising any one of SEQ ID NOs: 732 and 767-776, a chitinase having an amino acid sequence comprising any one of SEQ ID NOs: 777 and 778 or an amylase having an amino acid sequence comprising or consisting of SEQ ID NO: 735.

In the methods for increasing juice content and/or improving a Brix:acid ratio of juice obtained from a plant the composition can comprise (A) at least one polypeptide and an inducer compound; (B) at least two polypeptides, optionally, with an inducer compound; or (C) a callose synthase inhibitor and at least one of an inducer compound comprising a bacteriocide, an amino acid or isomer thereof, a substituted or unsubstituted benzoic acid or derivative or salt thereof, a dicarboxylic acid or derivative or salt thereof, a benzothiadiazole, a betaine, a proline or any combination thereof; or (D) a bacteriocide and at least one of an inducer compound selected from an amino acid or isomer thereof, a callose synthase inhibitor, a substituted or unsubstituted benzoic acid or derivative thereof, a dicarboxylic acid or derivative thereof, a betaine, a proline, a benzothiadiazole or any combination thereof; wherein: the polypeptide or polypeptides of (A) or (B) comprise: (i) a flagellin or flagellin-associated polypeptide; or (ii) a retro inverso flagellin or flagellin-associated polypeptide (iii) a root hair promoting polypeptide (RHPP); or (iv) a retro inverso root hair promoting polypeptide (RI RHPP); or (v) a thionin or thionin-like polypeptide; or (vi) a glucanase polypeptide; or (vii) a serine protease polypeptide; or (viii) an ACC deaminase (1-aminocyclopropane-1-carboxylate deaminase) polypeptide; or (ix) an amylase; or (x) a chitinase; or (xi) any combination thereof.

In addition, in the methods for increasing juice content and/or improving a Brix:acid ratio of juice obtained from a plant, the method can comprise applying an isolated polypeptide to the plant or plant part. The isolated polypeptide can comprise a β-1,3-glucanase. Preferably, when the isolated polypeptide comprises a β-1,3-glucanase, the peptide is injected into a trunk of a plant. The isolated polypeptide can comprise an RHPP, a retro-inverso RHPP, a glucanase, a chitinase and/or an amylase as described herein. For example, the isolated polypeptide can comprise an RHPP having an amino acid sequence comprising any one of SEQ ID NOs: 745-755, or a retro-inverso RHPP having an amino acid sequence comprising or consisting of any one of SEQ ID NOs: 756-766, a β-1,3-glucanase having an amino acid sequence comprising any one of SEQ ID NOs: 732 and 767-776, a chitinase having an amino acid sequence comprising any one of SEQ ID NOs: 777 and 778 or an amylase having an amino acid sequence comprising or consisting of SEQ ID NO: 735.

Methods for Increasing Growth, Vigor of a Plant and/or Protecting the Plant from a Disease:

In the methods for increasing growth, yield, health, longevity, productivity, and/or vigor of a plant or a plant part and/or protecting the plant or the plant part from disease, and/or increasing the innate immune response of the plant or the plant part the following isolated polypeptides or compositions can be used.

Any isolated polypeptide described herein (e.g., an isolated RHPP or RI-RHPP or an isolated glucanase, amylase and/or chitinase) can be used in this method. For example, an isolated RHPP or RI-RHPP having an amino acid sequence comprising or consisting of any one of SEQ ID NOs: 745-766 can be used in this method. Alternatively, an isolated glucanase, amylase, or chitinase having an amino acid sequence comprising or consisting of any one of SEQ ID NOs: 732 and 767-778 can be used in this method. For example, the isolated polypeptide can comprise a glucanase having an amino acid sequence comprising or consisting of any one of SEQ ID NO: 732 and 767-776. As another example, the isolated polypeptide can comprise an amylase having an amino acid sequence comprising or consisting of SEQ ID NO: 735. As another example, the isolated polypeptide can comprise a chitinase comprising or consisting of SEQ ID NO: 777 or SEQ ID NO: 778. In some instances, a β-1,3-glucanase (including β-1,3-glucanases not specifically identified herein) can be used in this method. Preferably, the β-1,3-glucanase is applied endogenously (e.g., injected into) the plant.

A composition comprising bixafen and a free polypeptide (i.e., not bound to an exosporium of a Bacillus cereus family member or an intact Bacillus cereus family member spore) can be used. The free polypeptide can comprise (i) a flagellin or flagellin-associated polypeptide; or (ii) a retro inverso flagellin or flagellin-associated polypeptide; or (iii) a root hair promoting polypeptide (RHPP); or (iv) a retro inverso root hair promoting polypeptide (RI RHPP); or (v) a thionin or thionin-like polypeptide; or (vi) a glucanase polypeptide; or (vii) a serine protease polypeptide; or (viii) an ACC deaminase (1-aminocyclopropane-1-carboxylate deaminase) polypeptide; or (ix) an amylase; or (x) a chitinase; or (xi) any combination thereof.

The composition can comprise a free polypeptide comprising a root hair promoting polypeptide (RHPP), a retro-inverso root hair promoting polypeptide (RI-RHPP), a chitinase, a flagellin or flagellin associated polypeptide, a glucanase, a serine protease or any combination thereof.

The composition can comprise a free polypeptide wherein an amino acid sequence of the free polypeptide can comprise any one of SEQ ID NOs: 604, 606, 607 and 745-755 (root hair promoting polypeptide, RHPP), any one of SEQ ID NOs: 605, and 756-766 (retro-inverso root hair promoting polypeptide, RI-RHPP), any one of SEQ ID NOs 226 and 571 (flagellin or flagellin associated polypeptide), any one of SEQ ID NOs: 731-733 and 767-778 (glucanase), any one of SEQ ID NOs: 777 and 778 (chitinases) or any one of SEQ ID NOs: 721, 722 and 794-796 (serine proteases).

The composition can comprise bixafen and a free polypeptide comprising a root hair promoting polypeptide and an amino acid sequence of the RHPP can comprise any one of SEQ ID NOs: 604, 606, 607 and 745-755. For example, the amino acid sequence of the RHPP can comprise SEQ ID NO: 604.

The composition can comprise at least one bioactive priming polypeptide.

The composition can comprise at least one flagellin or flagellin-associated polypeptide of (i). An amino acid sequence of the flagellin or flagellin associated polypeptide can comprise any one of SEQ ID NOs: 226, 289, 290, 291, 293, 294, 295, 300, 437, 532, 534, 536, 538, 540, 571-58, and 589-603. In some cases, the amino acid sequence of the flagellin or flagellin associated polypeptide comprises any one of SEQ ID NOs: 226, 293, 295, 300, 540, 571-579, and 589-590. For example, the composition can comprise a flagellin or flagellin-associated polypeptide having an amino acid sequence comprising SEQ ID NO: 226 or 571. For example, the composition can comprise a flagellin or flagellin-associated polypeptide having an amino acid sequence comprising SEQ ID NO: 226.

The composition can comprise at least one retro inverso flagellin or flagellin-associated polypeptide. The retro-inverso flagellin or flagellin associated polypeptide can comprise a retro-inverso Flg22 polypeptide, a retro-inverso FlgII-28 polypeptide and/or an Flg15 polypeptide.

The composition can comprise at least one retro inverso Flg22 polypeptide. An amino acid sequence of the retro inverso Flg22 polypeptide can comprise any one of SEQ ID NOs: 376-450, 527, 531, 533, 535, 537 and 539.

The composition can comprise at least one retro-inverso FlgII-28 polypeptide. An amino acid sequence of the retro-inverso FlgII-28 polypeptide can comprise any one of SEQ ID NOs: 451-525, or 588.

The composition can comprise at least one retro-inverso Flg15 polypeptide. An amino acid sequence of the retro-inverso Flg15 polypeptide can comprise SEQ ID NOs: 529 or 586.

The composition can comprise at least one RHPP. An amino acid sequence of the RHPP polypeptide can comprise any one of SEQ ID Nos: 604, 607, 608 and 745-755. For example, the composition can comprise an RHPP having an amino acid sequence comprising SEQ ID NO: 604.

The composition can comprise at least one retro-inverso RHPP polypeptide. An amino acid sequence of the retro-inverso RHPP polypeptide can comprise any one of SEQ ID NO: 605, 609, 610 and 756-766.

The composition can comprise at least one thionin or thionin-like polypeptide. An amino acid sequence of the thionin or thionin-like polypeptide can comprise any one of SEQ ID NOs: 620-719. For example, the composition can comprise a thionin or thionin-like polypeptide having an amino acid sequence comprising SEQ ID NO: 620. In some instances, the thionin or thionin-like polypeptide can be fused to a phloem targeting sequence to form a fused polypeptide. The phloem or phloem targeting sequence can comprise any one of SEQ ID NOs: 611-619 or any combination thereof. In some cases, the phloem or phloem targeting sequence comprises SEQ ID NO: 611. In some cases, the fusion polypeptide comprising a thionin or thionin-like polypeptide and a phloem or phloem targeting sequence can comprise SEQ ID NO: 720.

The composition can comprise at least one glucanase polypeptide. An amino acid sequence of the glucanase polypeptide can comprise any one of SEQ ID NOs: 731-733 and 767-776. The composition can comprise at least one amylase polypeptide. An amino acid sequence of the amylase polypeptide can comprise SEQ ID NO: 734 or SEQ ID NO: 735. The composition can comprise at least one chitinase polypeptide. An amino acid sequence of the chitinase polypeptide can comprise SEQ ID NO: 777 or SEQ ID NO: 778. In some instances, the composition can comprise two or more glucanases (e.g., β-1,3-glucanases), amylases or chitinases. For example, the composition can comprise a glucanase polypeptide (e.g., a 13-1,3-glucanase) having an amino acid sequence comprising SEQ ID NO: 731-733 and 767-776 and an amylase having an amino acid sequence comprising SEQ ID NO: 734 or SEQ ID NO: 735. Alternatively, the composition can comprise a glucanase (e.g., a β-1,3-glucanase) and a chitinase having an amino acid sequence comprising SEQ ID NO: 777 or SEQ ID NO: 778. Preferably, the composition can comprise a β-1,3-glucanase having an amino acid sequence comprising SEQ ID NO: 772.

The composition can comprise at least one serine protease polypeptide. An amino acid sequence of the serine protease polypeptide can comprise any one of SEQ ID NOs: 721, 722 and 794-796. For example, the composition can comprise a serine protease polypeptide having an amino acid sequence comprising SEQ ID NO: 722 or 795. For example, the composition can comprise a serine protease polypeptide having an amino acid sequence comprising SEQ ID NO: 794 or 796.

The composition can comprise at least one ACC deaminase polypeptide. An amino acid sequence of the ACC deaminase polypeptide can comprise any one of SEQ ID NOs: 723-730. For example, the composition can comprise an ACC deaminase polypeptide having an amino acid sequence comprising SEQ ID NO: 730.

The composition can comprise at least two bioactive polypeptides.

The composition can comprise a flagellin or flagellin associated polypeptide and a thionin or thionin-like polypeptide. For example, the composition can comprise a flagellin or flagellin associated polypeptide having an amino acid sequence comprising SEQ ID NO: 571 and a thionin polypeptide having an amino acid sequence comprising SEQ ID NO: 620.

The composition can comprise a flagellin or flagellin associated polypeptide and an RHPP polypeptide. For example, the composition can comprise a flagellin or flagellin associated polypeptide having an amino acid sequence comprising SEQ ID NO: 571 and an RHPP polypeptide having an amino acid sequence comprising SEQ ID NO: 604.

The composition can comprise a flagellin or flagellin associated polypeptide and a serine protease. For example, the composition can comprise a flagellin or flagellin associated polypeptide having an amino acid sequence comprising SEQ ID NO: 226 or 571 and a serine protease having an amino acid sequence comprising SEQ ID NO: 721, 722 and 794-796.

The composition can comprise a flagellin or flagellin associated polypeptide and a glucanase. For example, the composition can comprise a flagellin or flagellin associated polypeptide having an amino acid sequence comprising SEQ ID NO: 226 or 571 and a glucanase having an amino acid sequence comprising any one of SEQ ID NOs: 731-733 and 767-776. In some cases, the composition can comprise a flagellin or flagellin associated polypeptide having an amino acid sequence comprising SEQ ID NO: 226 or 571 and a glucanase having an amino acid sequence comprising any one of SEQ ID NOs: 732 or 772.

The composition can comprise a flagellin or flagellin associated polypeptide and an amylase. For example, the composition can comprise a flagellin or flagellin associated polypeptide having an amino acid sequence comprising SEQ ID NO: 226 or 571 and an amylase having an amino acid sequence comprising SEQ ID NO: 734 or 735.

The composition can comprise a flagellin or flagellin associated polypeptide and a chitinase. For example, the composition can comprise a flagellin or flagellin associated polypeptide having an amino acid sequence comprising SEQ ID NO: 226 or 571 and a chitinase having an amino acid sequence comprising SEQ ID NO: 777 or 778.

The composition can comprise a glucanase and an amylase, or a glucanase and a chitinase. For example, the composition can comprise a β-1,3-endoglucanase and an amylase. For example, the composition can comprise a β-1,3-endoglucanase having an amino acid sequence comprising any one of SEQ ID NO: 731-733 and 767-776 and an amylase having an amino acid sequence comprising SEQ ID NO: 734 or SEQ ID NO: 735. As an additional example, the composition can comprise a β-1,3-endoglucanase and a chitinase. For example, the composition can comprise a β-1,3-endoglucanase having an amino acid sequence comprising any one of SEQ ID NO: 731-733 and 767-776 and a chitinase having an amino acid sequence comprising SEQ ID NO: 777 or SEQ ID NO: 778.

Compositions described herein having a glucanase in combination with an amylase or a chitinase can further comprise at least one flagellin or flagellin associated polypeptide. For example, a composition can comprise at least one flagellin or flagellin associated polypeptide, a β-1,3-endoglucanase and an amylase. For instance, the composition can comprise a flagellin or flagellin associated polypeptide having an amino acid sequence comprising SEQ ID NO: 226 or 571, a β-1,3-endoglucanse having an amino acid sequence comprising any one of SEQ ID NO: 731-733 and 767-776 and an amylase having an amino acid sequence comprising SEQ ID NO: 734 or 735. As an additional example, a composition can comprise at least one flagellin or flagellin associated polypeptide, a β-1,3-endoglucanase and a chitinase. For instance, the composition can comprise a flagellin or flagellin associated polypeptide having an amino acid sequence comprising SEQ ID NO: 226 or 571, a β-1,3-endoglucanse having an amino acid sequence comprising any one of SEQ ID NO: 731-733 and 767-776 and a chitinase having an amino acid sequence comprising SEQ ID NO: 777 or SEQ ID NO: 778

The composition can comprise a flagellin or flagellin associated polypeptide and an ACC deaminase. For example, the composition can comprise a flagellin or flagellin associated polypeptide having an amino acid sequence comprising SEQ ID NO: 226 or 571 and an ACC deaminase having an amino acid sequence comprising SEQ ID NO: 730.

The composition can comprise a root hair promoting polypeptide (RHPP) or a retro-inverso root hair promoting polypeptide (RI-RHPP) and a glucanase. For example, the composition can comprise a RHPP having an amino acid sequence comprising any one of SEQ ID NOs: 745-756 or a RI-RHPP comprising any one of SEQ ID NOs: 757-766 and a β-1,3-glucanase having an amino acid sequence comprising any one of SEQ ID NOs: 731-733 and 767-776.

The composition can comprise a root hair promoting polypeptide (RHPP) or a retro-inverso root hair promoting polypeptide (RI-RHPP) and an ACC deaminase. For example, the composition can comprise an RHPP having an amino acid sequence comprising any one of SEQ ID NOs: 745-756 or an RI-RHPP comprising any one of SEQ ID NOs: 757-766 and an ACC deaminase having an amino acid sequence comprising any one of SEQ ID NOs: 723-730.

The composition can comprise a glucanase and a serine protease. For example, the composition can comprise a glucanase (e.g., a β-1,3-glucanase) having an amino acid sequence comprising any one of SEQ ID NOs: 731-733 and 767-776 and a serine protease having an amino acid sequence comprising SEQ ID NO: 721, 722 and 794-796. For example, the composition can comprise a glucanase having an amino acid sequence comprising SEQ ID NO: 772 or 732 and a serine protease having an amino acid sequence comprising any one of SEQ ID NOs: 722 and 794-796. The composition can comprise a bioactive polypeptide and at least one inducer compound.

The composition can comprise a flagellin or flagellin associated polypeptide and a callose synthase inhibitor. For example, the composition can comprise a flagellin or flagellin associated polypeptide having an amino acid sequence comprising SEQ ID NO: 226 or 571 and a callose synthase inhibitor. The callose synthase inhibitor can comprise 2-deoxy-D-glucose (2-DDG). Optionally, the composition can further comprise a bacteriocide (e.g., oxytetracycline).

The composition can comprise a flagellin or flagellin associated polypeptide and an amino acid. For example, the composition can comprise a flagellin or flagellin associated polypeptide having an amino acid sequence comprising SEQ ID NO: 226 or 571 and an amino acid. The amino acid can comprise L-cysteine or β-amino-butyric acid (BABA). Preferably, the amino acid comprises β-amino-butyric acid (BABA). Optionally, the composition can further comprise a bacteriocide (e.g., oxytetracycline).

The composition can comprise a flagellin or flagellin associated polypeptide and a substituted or unsubstituted benzoic acid. For example, the composition can comprise a flagellin or flagellin associated polypeptide having an amino acid sequence comprising SEQ ID NO: 226 or 571 and a substituted or unsubstituted benzoic acid. The substituted benzoic acid can comprise salicylic acid. Optionally, the composition can further comprise a bacteriocide (e.g., oxytetracycline).

The composition can comprise a flagellin or flagellin associated polypeptide and a benzothiadiazole. For example, the composition can comprise a flagellin or flagellin associated polypeptide having an amino acid sequence comprising SEQ ID NO: 226 or 571 and a benzothiadiazole. The benzothiadiazole can comprise benzo (1,2,3)-thiadiazole-7-carbothioic acid-S-methyl ester, available commercially as ACTIGARD 50WG fungicide. Optionally, the composition can further comprise a bacteriocide (e.g., oxytetracycline).

The composition can comprise a flagellin or flagellin associated polypeptide and a dicarboxylic acid. For example, the composition can comprise a flagellin or flagellin associated polypeptide having an amino acid sequence comprising SEQ ID NO: 226 or 571 and a dicarboxylic acid. The dicarboxylic acid can comprise oxalic acid. Optionally, the composition can further comprise a bacteriocide (e.g., oxytetracycline).

The composition can comprise a flagellin or flagellin associated polypeptide and a betaine. For example, the composition can comprise a flagellin or flagellin associated polypeptide having an amino acid sequence comprising SEQ ID NO: 226 or 571 and a betaine. The betaine can comprise betaine-hydrochloride or glycine betaine. Optionally, the composition can further comprise a bacteriocide (e.g., oxytetracycline).

The composition can comprise a flagellin or flagellin associated polypeptide and a proline. For example, the composition can comprise a flagellin or flagellin associated polypeptide having an amino acid sequence comprising SEQ ID NO: 226 or 571 and a proline. The proline can comprise L-proline. Optionally, the composition can further comprise a bacteriocide (e.g., oxytetracycline).

The composition can comprise a flagellin or flagellin associated polypeptide and an herbicide. For example, the composition can comprise a flagellin or flagellin associated polypeptide having an amino acid sequence comprising SEQ ID NO: 226 or 571 and a herbicide. The herbicide can comprise lactofen. Optionally, the composition can further comprise a bacteriocide (e.g., oxytetracycline).

The composition can comprise a flagellin or flagellin associated polypeptide and a succinate dehydrogenase inhibitor. For example, the composition can comprise a flagellin or flagellin associated polypeptide having an amino acid sequence comprising SEQ ID NO: 226 or 571 and a succinate dehydrogenase inhibitor. The succinate dehydrogenase inhibitor can comprise bixafen.

The composition can comprise a flagellin or flagellin associated polypeptide and a bacteriocide. For example, the composition can comprise a flagellin or flagellin associated polypeptide having an amino acid sequence comprising SEQ ID NO: 226 or 571 and a bacteriocide. The bacteriocide can comprise oxytetracycline.

The composition can comprise a root hair promoting polypeptide or a retro inverso root hair promoting polypeptide and a succinate dehydrogenase inhibitor. For example, the composition can comprise a root hair promoting polypeptide or a retro inverso root hair promoting polypeptide having an amino acid sequence comprising any one of SEQ ID NOs: 745-766 and a succinate dehydrogenase inhibitor. The succinate dehydrogenase inhibitor can comprise bixafen. The root hair promoting polypeptide or the retro inverso root hair promoting polypeptide can comprise a free polypeptide.

The composition can comprise flagellin or flagellin associated polypeptide or a retro inverso flagellin or flagellin-associated polypeptide and a succinate dehydrogenase inhibitor. For example, the composition can comprise a flagellin or flagellin associated polypeptide having an amino acid sequence comprising any one of SEQ ID NOs: 226, 289, 290, 291, 293, 294, 295, 300, 437, 526, 532, 534, 536, 538, 540, 571-585, and 587-603 or a retro inverso flagellin or flagellin associated polypeptide having an amino acid sequence comprising any one of SEQ ID NOs: 376-525, 527, 529, 531, 533, 535, 537, 539, or 588, or 586 and a succinate dehydrogenase inhibitor. The succinate dehydrogenase inhibitor can comprise bixafen. The serine protease can comprise a free polypeptide.

The composition can comprise a glucanase and a succinate dehydrogenase inhibitor. For example, the composition can comprise glucanase having an amino acid sequence comprising any one of SEQ ID NOs: 731-733 and 767-776 and a succinate dehydrogenase inhibitor. The succinate dehydrogenase inhibitor can comprise bixafen. The glucanase can comprise a free polypeptide.

The composition can comprise a chitinase and a succinate dehydrogenase inhibitor. For example, the composition can comprise a chitinase having an amino acid sequence comprising SEQ ID NO: 777 or 778 and a succinate dehydrogenase inhibitor. The succinate dehydrogenase inhibitor can comprise bixafen. The chitinase can comprise a free polypeptide.

The composition can comprise a serine protease and a succinate dehydrogenase inhibitor. For example, the composition can comprise a serine protease having an amino acid sequence comprising any one of SEQ ID NOs: 721, 722, and 794-796 and a succinate dehydrogenase inhibitor. The succinate dehydrogenase inhibitor can comprise bixafen. The serine protease can comprise a free polypeptide.

The composition can comprise a thionin and a succinate dehydrogenase inhibitor. For example, the composition can comprise a thionin having an amino acid sequence comprising any one of SEQ ID NOs: 620-719 and a succinate dehydrogenase inhibitor. The succinate dehydrogenase inhibitor can comprise bixafen. The thionin or thionin-like polypeptide can comprise a free polypeptide.

The composition can comprise an ACC deaminase polypeptide and a succinate dehydrogenase inhibitor. For example, the composition can comprise an ACC deaminase polypeptide having an amino acid sequence comprising any one of SEQ ID NOs: 723-730 and a succinate dehydrogenase inhibitor. The succinate dehydrogenase inhibitor can comprise bixafen. The ACC deaminase can comprise a free polypeptide.

The composition can comprise an amylase and a succinate dehydrogenase inhibitor. For example, the composition can comprise an amylase having an amino acid sequence comprising any one of SEQ ID NOs 734 and 735 and a succinate dehydrogenase inhibitor. The succinate dehydrogenase inhibitor can comprise bixafen. The amylase can comprise a free polypeptide.

The composition can comprise a root hair promoting polypeptide or a retro inverso root hair promoting polypeptide and a betaine. For example, the composition can comprise an root hair promoting polypeptide (RHPP) having an amino acid sequence comprising any one of SEQ ID NOs: 604, 607, 608, and 745-756 or a retro inverso root hair promoting polypeptide (RI-RHPP) comprising any one of SEQ ID NOs: 605, 757-766 and a betaine. The betaine can comprise betaine hydrochloride or glycine betaine. Optionally, the composition can further comprise a bacteriocide (e.g., oxytetracycline).

The composition can comprise a root hair promoting polypeptide or a retro inverso root hair promoting polypeptide and a proline. For example, the composition can comprise a root hair promoting polypeptide (RHPP) having an amino acid sequence comprising any one of SEQ ID NOs: 604, 607, 608, and 745-756 or a retro inverso root hair promoting polypeptide (RI-RHPP) comprising any one of SEQ ID NOs: 605, and 757-766 and a proline. The proline can comprise L-proline. Optionally, the composition can further comprise a bacteriocide (e.g., oxytetracycline).

Any composition particularly described herein is effective at treating citrus plants or plant parts and citrus diseases. They can also be employed as in furrow or foliar treatments to improve yield of a tree. They can also be employed as in furrow or foliar treatments to increase crop yield (e.g., in row crops).

For instance, methods of treating a plant disease in a plant in need thereof can comprise administering to the plant by trunk injection, a foliar spray, a soil drench or a seed treatment application, a composition which comprises a flagellin or flagellin associated polypeptide and at least one inducer compound comprising β-aminobutyric acid (BABA) or a salt thereof, 2-deoxy-D-glucose (2-DDG) or a salt thereof, salicylic acid (SA) or a salt thereof; and oxalic acid (OA) or a salt thereof, L-cysteine and an analog of L-cysteine and an acid or a salt thereof, an antimicrobial protein comprising a thionin or a thionin-like peptide or any combination thereof. Optionally, the composition can further comprise a bacteriocide (e.g., oxytetracycline). The flagellin or flagellin associated polypeptide can be a Flg22 polypeptide (e.g., a polypeptide having an amino acid sequence comprising SEQ ID NO: 226 or 571).

The disease can comprise Asian citrus greening, Huanglonging (HLB) disease, Asian soybean rust, Sclerotinia stem rot (or white mold), Pseudomonas leaf spot, or Cercospora leaf blight.

In the methods, the composition can be applied just prior to floral formation or at the pre-flowering stage.

The growth can comprise root and floral apical meristems, floral organ production, fruit development, fruit production, number of floral organs, size of floral organs, or a combination thereof.

In the methods, protecting the plant or the plant part from disease can comprise prophylactic treatment, treatment, prevention and decreased disease progression on or in the plant or plant part.

The disease can comprise Asian citrus greening disease (HLB), Citrus canker disease, Cercospora leaf blight or a bacteria causing disease.

The bacteria causing disease can comprise bacterial leaf blight, bacterial leaf streak, bacterial stalk rot, bacterial leaf spot, bacterial leaf scorch, bacterial top rot, bacterial stripe, chocolate spot, Goss's bacterial wilt and blight, Holcus spot, purple leaf sheath, seed rot, seedling blight, Stewart's disease (bacterial wilt), corn stunt, Fire Blight, Pierce's disease, citrus variegated chlorosis, citrus canker, Pseudomonas syringae serovars, or a combination thereof.

The methods can further comprise preventing or reducing callose deposition in or around phloem plasmodesmata in a tree infected with Canditus (Ca.) Liberibacter

The methods can further comprise decreasing fruit drop from a plant infected with a disease. For example, the disease can comprise a Canditus (Ca.) Liberibacter infection and/or Huanglongbing (HLB).

In the methods, the polypeptide, the composition, or the recombinant microorganism can be applied exogenously to the plant, the plant part, or the plant growth medium.

In the methods, the polypeptide, the composition, or the recombinant microorganism can be applied endogenously to the plant or the plant part. For example, the polypeptide, the composition or the recombinant microorganism can be applied to the vascular system of the plant (e.g., via injection into a plant trunk, stem, root, or vine).

The plant part can include a plant cell, a leaf, a branch, a trunk, a vine, a plant tissue (i.e., xylem or phloem), a stem, a flower, a foliage, a floral organ, a fruit, pollen, a vegetable, a tuber, a rhizome, a corm, a bulb, a pseudobulb, a pod, a root, a root ball, a root stock, a scion, or a seed.

In the methods, the isolated polypeptide or composition can be applied to a surface of the plant, a foliage of the plant or a surface of a seed of the plant.

In the methods, the isolated polypeptide or composition can be applied to the surface of the seed and the plant or the plant part is grown from the seed.

In the methods, the isolated polypeptide or composition can be injected into a branch, trunk, stem, vasculature, root, or vine of the plant.

In the methods, the isolated polypeptide or composition can be applied as a foliar application.

In the methods, the isolated polypeptide or composition can be injected into a branch, trunk, stem, vine, or root of the plant.

In the methods wherein a composition comprising a polypeptide and an inducer compound, or two polypeptides or two inducer compounds is used; the composition can be prepared in two separate compositions to allow for separate (e.g., sequential) application of the two components. That is the methods can comprise sequentially applying one or more components of the composition to the plant or plant part. For example, the method can comprise sequentially applying one or more of the polypeptides of the composition and one or more of the inducer compounds of the composition to the plant or plant part.

The sequential applications can be made within 100 hours, within 72 hours, within 48 hours, within 24 hours, within 12 hours, or within 4 hours.

For example, a composition comprising a polypeptide (e.g., a flagellin or flagellin associated polypeptide) and an inducer (e.g., a callose synthase inhibitor) can be prepared as two separate compositions and administered separately (e.g., sequentially) to the plant or plant part. Alternatively, the compositions can be combined and applied at the same time.

The plant can be a fruit plant or a vegetable plant, and the method provides increased yield of fruits or vegetables.

The plant can be a tree or a vine.

The plant can be a row crop (e.g., corn or soybean)

The plant can be a citrus plant (e.g., a citrus tree).

The plant can be a citrus plant and the method reduces disease symptoms in the citrus plant. For example, the improved disease symptoms can comprise a reduction in a pathogen titer (i.e., a bacterial titer), as described below.

### Methods for Quantifying CLas Titer in an Infected Citrus Plant

The presences of the CLas bacterial titers in the HLB infected citrus trees can be determined with quantitative real-time polymerase chain reaction (qPCR) methods using specific primers to confirm the presence of the disease (Li, W. B., Hartung, J. S. and Levy, L. 2008 “Optimized quantification of unculturable ‘Candidatus Liberibacter spp.’ In host plants using real-time PCR”, Plant Disease 92: 854-861). DNA extraction and quantitative PCR (qPCR) analysis on these leaves was performed at Southern Gardens Citrus (Clewiston, Fla.) using HLB primer set targeting the 16S DNA of C. liberibacter bacteria 5′» 3′ (forward): HLB as TCGAGCGCGTATGCAATACG; (SEQ ID NO: 742, forward) HLBr: GCGTTATCCCGTAGAAAAAGGTAG (SEQ ID NO: 743, reverse); HLBpc (probe): AGACGGFTGAGTAACGCG (SEQ ID NO: 744), where “F” represents a fluorescent reporter dye label intercalated in the probe sequence]. Forty cycles of qPCR were conducted and the fluorescent signal which is proportional to the amount of dsDNA in solution was measured. The qPCR analysis allows for the detection of the CLas bacteria in citrus tissue. The cycle threshold (Ct) values from the qPCR analysis were obtained per each treatment. The Ct measurement is equivalent to the number of PCR cycles required to produce a relative threshold level. As in common practice within the field of molecular biology, the change in Ct value is reported to indicate the relative quantity of CLas DNA either in treated vs untreated samples or in treated samples at one time point vs another time. The higher the Ct value, the greater or more effective the treatment effect, which is indicated by the reduction/elimination of CLas bacteria from the tree. A percentage reduction in bacterial load can be computed as:

% reduction in sample over time=(1−2[Ct (initial time)−Ct(later time)])*100%

or

% reduction in treated vs. control sample=(1−2[Ct (control sample)-Ct(treated sample)])*100%

Methods of Improving the Quality and Quantity of Juice Obtained from a Plant

The method can also comprise increasing fruit yield and/or improving the quality and/or quantity of the juice obtained from a plant. Juice quality is typically expressed in terms of the sugar (Brix) and acid content. A particularly useful measure of juice quality is the ratio of the two (e.g. a Brix:acid ratio). Methods for obtaining a Brix:acid ratio are described in the art (JBT Food Tech Laboratory Manual, “Procedures for Analysis of Citrus Products, Sixth edition). The methods, therefore, can comprise increasing juice content and/or improving sugar or acid content and/or improving a Brix:acid ratio in a juice obtained from a citrus plant or plant part.

Any isolated polypeptide described herein (e.g., an isolated RHPP or RI-RHPP or an isolated glucanase, amylase and/or chitinase) can be used in this method. For example, an isolated RHPP or RI-RHPP having an amino acid sequence comprising or consisting of any one of SEQ ID NOs: 745-766 can be used in this method. Alternatively, an isolated glucanase, amylase, or chitinase having an amino acid sequence comprising or consisting of any one of SEQ ID NOs: 732, 735 and 767-778 can be used in this method. For example, the isolated polypeptide can comprise a glucanase having an amino acid sequence comprising or consisting of any one of SEQ ID NO: 732 and 767-776. As another example, the isolated polypeptide can comprise an amylase having an amino acid sequence comprising or consisting of SEQ ID NO: 735. As another example, the isolated polypeptide can comprise a chitinase comprising or consisting of SEQ ID NO: 777 or SEQ ID NO: 778.

Any composition comprising at least one polypeptide as described herein can be used in this method. For example, the composition can comprise at least one flagellin or flagellin-associated polypeptide (e.g., having an amino acid sequence comprising any one of SEQ ID NO: 571, 1-375, 526, 528, 530, 532, 534, 536, 538, 540, 541, 572-585, 587, and 589-603) or at least one retro inverso flagellin or flagellin associated polypeptide (e.g., having an amino acid sequence comprising any one of SEQ ID NO: 376-525, 527, 529, 531, 533, 535, 537, 539, or 588, or 586); or at least one RHPP polypeptide (e.g., having an amino acid sequence comprising any one of SEQ ID NOs: 604, 607, 608, and 745-756), or at least one RI-RHPP polypeptide (e.g., having an amino acid sequence comprising any one of SEQ ID NOs: 605, 609, 610, and 757-766), or at least one thionin or thionin-like polypeptide (e.g., having an amino acid sequence comprising any one of SEQ ID NOs: 620-719), or at least one serine protease (e.g., having an amino acid sequence comprising any one of SEQ ID NOs: 721-722 and 794-796), or at least one glucanase (e.g., having an amino acid sequence comprising any one of SEQ ID NOs: 731-735 and 767-776), or at least one ACC deaminase (e.g., having an amino acid sequence comprising any one of SEQ ID NOs: 723-730) as described herein.

Any composition comprising at least one free polypeptide as described herein can be used in this method. For example, the composition can comprise at least one free polypeptide comprising a flagellin or flagellin-associated polypeptide (e.g., having an amino acid sequence comprising any one of SEQ ID NO: 571, 1-375, 526, 528, 530, 532, 534, 536, 538, 540, 541, 572-585, 587, and 589-603) or at least one free polypeptide comprising a retro inverso flagellin or flagellin associated polypeptide (e.g., having an amino acid sequence comprising any one of SEQ ID NO: 376-525, 527, 529, 531, 533, 535, 537, 539, or 588, or 586); or at least one free polypeptide comprising a RHPP polypeptide (e.g., having an amino acid sequence comprising any one of SEQ ID NOs: 604, 607, 608, and 745-756), or at least one free polypeptide comprising a RI-RHPP polypeptide (e.g., having an amino acid sequence comprising any one of SEQ ID NOs: 605, 609, 610, and 757-766), or at least one free polypeptide comprising a thionin or thionin-like polypeptide (e.g., having an amino acid sequence comprising any one of SEQ ID NOs: 620-719), or at least one free polypeptide comprising a serine protease (e.g., having an amino acid sequence comprising any one of SEQ ID NOs: 721-722 and 794-796), or at least one free polypeptide comprising a glucanase (e.g., having an amino acid sequence comprising any one of SEQ ID NOs: 731-735 and 767-776), or at least one free polypeptide comprising a ACC deaminase (e.g., having an amino acid sequence comprising any one of SEQ ID NOs: 723-730) as described herein

In the methods of improving juice quantity and/or quality the composition can also comprise any inducer described herein. Suitable inducers that can be used in combination or with a polypeptide to improve juice quantity include a callose synthase inhibitor, a bacteriocide, an amino acid or isomer thereof, a substituted or unsubstituted benzoic acid or derivative or salt thereof, a dicarboxylic acid or derivative or salt thereof, a benzothiadiazole, a betaine, a proline, a succinate dehydrogenase inhibitor (e.g., bixafen) or any combination thereof.

In the methods of improving juice quantity and/or quality, the plant can be a tree or a vine. The plant can be also be citrus plant (e.g., a citrus tree).

The citrus plant can comprise an orange, a lemon, a lime, a tangerine, a kumquat, a tangelo, or any variety, hybrid or cross thereof.

The plant can be a row crop. For example, the row crop can be corn or soybean.

In the methods wherein a composition comprising a polypeptide and an inducer compound, or two polypeptides or two inducer compounds is used; the composition can be prepared in two separate compositions to allow for separate (e.g., sequential) application of the two components. That is the methods can comprise sequentially applying one or more components of the composition to the plant or plant part. For example, the method can comprise sequentially applying one or more of the polypeptides of the composition and one or more of the inducer compounds of the composition to the plant or plant part.

The sequential applications can be made within 100 hours, within 72 hours, within 48 hours, within 24 hours, within 12 hours, or within 4 hours.

For example, a composition comprising a polypeptide (e.g., a flagellin or flagellin associated polypeptide) and an inducer (e.g., a callose synthase inhibitor) can be prepared as two separate compositions and administered separately (e.g., sequentially) to the plant or plant part. Alternatively, the compositions can be combined and applied at the same time.

Methods are also provided comprising applying to the plant or plant part a second composition, wherein the second composition comprises any polypeptide described herein and/or any inducer compound described herein.

In the methods, the isolated polypeptides or compositions can be applied exogenously or endogenously to the plant or plant part. When applied endogenously, the isolated polypeptide (e.g., a β-1,3-glucanase) or the composition can be injected into a trunk, root, or stem of the plant. The injection can be performed to ensure delivery of the isolated polypeptide or the composition directly into the vascular system of the plant or plant part—that is, into the xylem and/or phloem of the plant or plant part.

In methods where the compositions are applied two or more times during a growing season, the first application can occur at or before the V2 stage of development, and subsequent applications can occur before the plant flowers. For example, the first application can occur as a seed treatments, at/or before the VE stage of development, at or before the V1 stage of development, at or before the V2 stage of development, at or before the V3 stage of development, at or before the V4 stage of development, at or before the V5 stage of development, at or before the V6 stage of development, at or before the V7 stage of development, at or before the V8 stage of development, at or before the V9 stage of development, at or before the V10 stage of development, at or before the V11 stage of development, at or before the V12 stage of development, at or before the V13 stage of development, at or before the V14 stage of development, at or before the V15 stage of development, at or before the VT stage of development, at or before the R1 stage of development, at or before the R2 stage of development, at or before the R3 stage of development, at or before the R4 stage of development, at or before the R5 stage of development, at or before the R6 stage of development, at or before the R7 stage of development, or at or before the R8 stage of development. By way of example, the first application can occur at or before the germination stage, at or before the seedling stage, at or before the tillering stage, at or before the stem elongation stage, at or before the booting stage, or at or before the heading stage. For example, where the Feekes growth stage scale is used to identify the stage of growth of a cereal crop, the first application can occur at or before stage 1, at or before stage 2, at or before stage 3, at or before stage 4, at or before stage 5, at or before stage 6, at or before stage 7, at or before stage 8, at or before stage 9, at or before stage 10, at or before stage 10.1, at or before stage 10.2, at or before stage 10.3, at or before stage 10.4, or at or before stage 10.5.

### Abiotic Stress

Abiotic stress causes significant crop loss and can result in major reductions in crop production and yield potential. The bioactive priming polypeptides and compositions as described herein can be used as chemical priming agents to increase tolerance of a plant to one or more abiotic stresses. Thus, the flagellin polypeptides, flagellin-associated polypeptides of Flg22 or FlgII-28 derived from Bacillus species, Flg15 and Flg22 derived from E. coli and other organisms (Table 5) and the RHPP polypeptides derived from Glycine max (Tables 11 to 13) are useful for increasing the tolerance of a plant, group of plants, field of plants and/or the parts of plants to abiotic stress. The polypeptides and compositions as described herein impart abiotic stress tolerance to a plant or plant part. The abiotic stress tolerance imparted to a plant or plant part are to abiotic stresses that include, but are not limited to: temperature stress, radiation stress, drought stress, cold stress, salt stress, osmotic stress, nutrient-deficient or high metal stress, and water stress that results from water deficit, flooding or anoxia. Chemical priming using the bioactive priming polypeptides and compositions as described herein are applied to a plant or plant part offering a versatile approach to protect the plant or plant part against individual, multiple or combined abiotic stresses.

The polypeptides and compositions as described herein are effective to protect a plant against abiotic stressors when applied as an above ground foliar application to a plant, a plant part, a plant root, a plant seed, a plant growth medium, or the area surrounding a plant or the area surrounding a plant seed. For example, for trees, one or more applications can be applied at different growth timings of trees, including timings before, during or after flushes; before, during, or after fruit set; or before or after fruit harvest.

The methods described herein chemically prime the plant for protection against abiotic stress(es) in such a way that the plant has already prepared and initiated defense mechanisms that can be activated faster and increase tolerance to an abiotic stress or multiple stressors occurring simultaneously or at different times during the growing season.

The retro inverso forms of the Flg22 polypeptides as described herein can be applied externally as a foliar spray application (or using other application methods as well, for example as a root drench) during times of excessive heat, water, and drought stress and be used to protect a plant against drought, heat stress and/or other abiotic stresses that can affect stomatal aperture and oscillation that commonly occur with transpiration loss through a plant.

In the methods, the composition preferably comprises at least one of a proline, a betaine, an ACC deaminase or any combination thereof. In addition, the composition can comprise one or more bioactive priming polypeptide. For example, the composition can comprise a flagellin or flagellin associated polypeptide and at least one of a proline, a betaine, or an ACC deaminase. The abiotic stress can comprise heat stress, temperature stress, radiation stress, drought stress, cold stress, salt stress, nutrient-deficient stress, high metal stress, water stress, osmotic stress, or any combination thereof.

Balancing Immune Response with Plant Growth and Development

Although immune responses can provide protection of plants from pathogen attack, excessive immune responses can have negative impacts on plant growth. Therefore, balancing enhanced immunity or disease prevention and protection in a plant with an increased growth promoting response is a desired combination to optimize plant health.

Bioactive priming polypeptides that are useful for enhancing immune responses as described herein can be combined with polypeptides that provide positive impacts on plant growth and productivity. The polypeptide combinations are specifically selected for their distinct modes of action/regulation when applied to a plant or plant part. However, some of the bioactive priming polypeptides (Flg peptides such as Flg22, Flg15 and FlgII-28) are perceived by receptor-like proteins, followed by a process that initiates their entry and transport in the plant which results in functional outcomes while others are taken into the plant by active absorption. For example, the Flg-associated polypeptides such as Flg22, Flg15 and FlgII-28 are perceived by a leucine-rich receptor kinase located on the surface of the plasma membrane and involve a complex signaling pathway involved in the pathogen-triggered responses leading to immunity, disease resistance or disease prevention (Kutschmar et al. “PSKα promotes root growth in Arabidopsis,” New Phytologist 181: 820-831, 2009).

The bioactive priming polypeptides as described herein such as Flg22 polypeptides and thionins can act as elicitors and exhibit antimicrobial activity (e.g., anti-pesticide; bacterial, fungal, or viral activity). Specific combinations of polypeptides are provided, for example, the combination of flagellin and flagellin-associated bioactive priming polypeptides are useful for preventing and protecting plants from pathogenic diseases and serve a dual utility when they are applied together with those other polypeptides, for example, RHPP, serine proteases, glucanases and/or ACC deaminases, that enhance plant growth and productivity in a plant, plant part, and/or field of plants.

The bioactive priming compositions described herein can be applied exogenously as a foliar spray, in furrow treatment, soil application, seed treatment, drench or wash or endogenously to a plant to stimulate both the immune responsiveness and growth characteristics of the plant that collectively result in improved yield performance. They can also provide protection and growth benefits to the different parts of the plant (for example, leaves, roots, tubers, corms, rhizomes, bulbs, pseudobulbs, flowers, pods, fruits, and growing meristems).

Any of the bioactive priming compositions as described herein can be applied one or more times to a plant either in combination or individually to enhance growth and productivity of a plant. Multiple applications can be applied to promote yield benefits over the growing season with applications tailored to the conditions in the environment, for example if a period of hot and dry weather is expected during the growth season, an additional spray of bioactive priming polypeptides that promote growth under abiotic stress can alleviate negative impacts to the plant. Further, any of the individual components of a composition can be divided into separate compositions for separate application to the plant or plant part. For instance, when a composition comprises an inducer and a polypeptide, application of the “composition” to the plant or plant part does not require simultaneous administration. Instead, the inducer and the polypeptide can be applied separately in accordance with knowledge in the art.

For example, the methods herein comprise applying a composition comprising a polypeptide and an inducer. In certain instances, the inducer can be applied separately (e.g., before or after) the composition. For example, when the inducer comprises a bacteriocide (e.g., oxytetracycline), the inducer can be applied before or after the composition comprising the polypeptide.

### Bacterial Disease

Methods of using the bioactive priming compositions such as those containing the flagellin-associated polypeptides or the thionin-like polypeptides alone or in combination with an inducer compound as described herein are useful for the prevention, treatment and control of bacterial diseases in corn and particularly useful for the treatment of bacterial leaf streak disease in corn caused by Xanthomonas vasicola pv. vasculorum, also recognized as Xanthomnas campestris pv. vasculorum.

Surveys indicate that bacterial leaf streak disease has spread and may be widely distributed throughout the U.S. Corn Belt (Western Indiana, Illinois, Iowa, Missouri, Eastern Nebraska and Eastern Kansas). Disease spread is most prevalent where corn is planted on corn in crop rotation practices. The bacterial leaf streak disease can cause infection on dent corn (field) seed corn, popcorn and sweet corn. The symptoms on corn include narrow to brown yellow streaks and brown yellow strips between the leaf veins. Lesions usually develop on lower or older plant leaves and initially spread to the higher or younger leaves on the plant. Yellow discoloration also may be present around lesions.

The bacterial leaf streak disease of corn presumably survives in previously infected host debris. Bacterial exudates found on surfaces of infected leaf tissues can serve as secondary inocula. The bacterium is spread by wind, splashing rain, and possibly by irrigation water. The pathogen penetrates corn leaves through natural openings such as stomata, which can result in a banded pattern of lesions occurring across leaves. Colonization of leaf tissues apparently is restricted by main veins.

Because the disease is caused by a bacterial pathogen, the current use of bactericides is problematic to control it. For example, most bactericides act as contact products and are not systemic and thus they will not be absorbed or taken into the plant via other mechanisms. Bactericide treatments may require repeated applications as the bactericide may be washed off with rain or wind, thus rendering them uneconomical or impractical for use in some corn crops.

Current disease management practices to date recommend crop rotation practices (such as corn, soybean and then back to corn) and the implementation of sanitation practices, such as cleaning equipment between field usage to slow disease progression.

Foliar applications of bioactive priming compositions described herein that contain the Flg (Tables 4-5) and thionin polypeptides (Table 15) or combinations of the two classes provide an alternative approach for treating the disease. Foliar applications with these bioactive priming compositions provided as a spray to the leaf surface of either asymptomatic or symptomatic plants provides a means to prevent, treat, and control the bacterial leaf streak disease in corn.

Alternatively, bioactive priming compositions described herein that contain flagellin- and thionin bioactive priming polypeptides or combinations thereof can be useful for the prevention, treatment and control of other bacterial diseases that infect corn (Table 22). The compositions can comprise an inducer compound.

TABLE 22
Bacteria causing diseases in plants

| Crop(s) | Disease Name | Bacteria Causing Disease |
|---|---|---|
| Corn | Bacterial leaf blight and stalk rot | Pseudomonas avenae subsp. avenae |
| Corn | Bacterial leaf spot | Xanthomonas campestris pv. |
| | | holcicola |
| Corn | Bacterial leaf streak | Xanthomonas vasicola spp., |
| | | Xanthamonas vasicola pv. holcicola |
| Corn | Bacterial stalk rot | Enterobacter dissolvens; Erwinia |
| | | dissolvens |
| Corn | Bacterial stalk and top rot | Erwinia carotovora subsp. |
| | | carotovora |
| | | Erwinia chrysanthemi pv. zeae |
| Corn | Bacterial stripe | Pseudomonas andropogonis |
| Corn | Chocolate spot | Pseudomonas syringae pv. |
| | | coronafaciens |
| Corn | Goss's bacterial wilt and | Clavibacter michiganensis subsp. |
| | blight (leaf freckles and wilt) | nebraskensis; Corynebacterium |
| | | michiganense pv. nebraskense |
| Corn | Holcus leaf spot | Pseudomonas syringae pv. syringae |
| | | van Hall |
| Corn | Purple leaf sheath | Hemiparasitic bacteria |
| Corn | Seed rot-seedling blight | Bacillus subtilis |
| Corn | Stewart's disease (Stewart's | Pantoea stewartii |
| | bacterial wilt) | |
| Corn | Corn stunt (achapparramiento, | Spiroplasma kunkelii |
| | maize stunt, Mesa Central or | |
| | Rio Grande maize stunt) | |
| Citrus | Citrus greening; Huanglongbing | Candidatus liberibacter asiaticus |
| Citrus | Citrus canker | Xanthomonas citrii |
| Kiwi | Kiwi PSA disease | Pseudomonas syringae pv. actinidae |
| Flowering trees | Fireblight | Erwinia amylovora |
| (apple, pear, | | |
| almond, plum, | | |
| cherry) & | | |
| ornamental | | |
| plants | | |
| Grapevine, olive | Pierce's disease, bacterial leaf | Xylella fastidiosa |
| trees, | scorch | |
| Fruiting | Bacterial leaf spot, bacterial | Pseudomonas syringae spp., |
| vegetables, | speck disease, bunch rot, | pseudomonas syringae pv. tomato, |
| vines, stone | bacterial blossom | Pseudomonas syringae pv. |
| fruits, apple, pear | blast, blister spot, | Lachrymans 2, Pseudomonas |
| brassicacae, | bacterial blast | syringae pv. syringae, Pseudomonas |
| vegetables, nut | | syringae pv. maculicola |
| trees, peppers, | | |
| tomato | | |
| Banana | Blood disease | Ralstonia syzygii subsp. celebesenis |
| Cashew | Bacterial leaf and fruit spot, | Xanthamonas spp., Xanthamonas |
| | Bacterial leaf and nut spot | campestris pv. mangiferaeindicae, |
| | | Xanthomonas citri pv. anacardii) |
| Citrus | Citrus variegated chlorosis (CVC) | Xylella fastidiosa |
| Tomato, peppers | Bacterial leaf spot | Xanthomonas spp., Xanthamonas |
| | | campestris pv. vesicatoria |
| Walnuts | Walnut blight | Xanthomonas arboricola pv. |
| | | juglandis |
| Leafy green | Bacterial leaf spot, bacterial spot | Xanthamonas spp., Xanthamonas |
| vegetables, | | campestris pv. vesicatoria |
| lettuce, tomato, | | |
| peppers | | |

Alternatively, bioactive priming compositions and/or isolated polypeptides described herein can be useful for the prevention, treatment and control of fungal diseases that infect a wide variety of plants, such as those listed in Table 23 below.

TABLE 23
Fungal Diseases in Plants

| Crop(s) | Disease Name | Fungus Causing Disease |
|---|---|---|
| kiwifruit, grapes, citrus | Sooty mold | Cladosporium and |
| | | Alternaria species |
| soybean | Frog eye leaf spot | Cercospora sojina |
| soybean | Cercospora leaf blight; | Cercospora kikuchii |
| | Cercosporiosis, purple seed stain | |
| soybean | Asian soybean rust | Phakopsora pachyrhizi; |
| | | Phakopsora meibomiae |
| soybean | Target spot | Corynespora cassicola |
| soybean | Powdery mildew | Erysiphe diffusa |
| soybean | Septoria brown rot | Septoria glycines |
| soybean | Soybean stem canker | Diaporthe spp., Diaporthe |
| | | phasoleorum |
| soybean | Phomopsis pod, seed blight, seed | Phomopsis spp., Diaporthe |
| | decay | spp. |
| soybean | Charcoal rot | Macrophomina phaseolina |
| soybean | Leaf sheath blight, mela | Thanatephorus cucumeris |
| legumes, soybean, | White mold, Sclerotinia stem rot | Sclerotinia sclerotiorum |
| canola, leafy green | | |
| vegetables, fruiting | | |
| vegetables | | |
| corn | Grey leaf spot | Cercospora zeae-maydis; |
| | | Cercospora zeina |
| Corn, soybean | Aspergillus, Aspergillus rot | Aspergillus spp., Aspergillus |
| | | flavus, Aspergillus sojae |
| corn | Phaeosphaeria spot | Phaeosphaeria maydis |
| turf grass | Dollar spot | Sclerotinia homoeocarpa |
| citrus | Citrus black spot | Guignardia citricarpa; |
| | | Phyllosticta citricarpa |
| citrus, almond | Phytopthora root rot; Phytopthora | Phytopthora spp. |
| | foot rot; Phytopthora crown rot | Phytophthora citrophthora; |
| | | Phytopthora parasitica, |
| citrus | Citrus black rot | Alternaria citri |
| citrus | Post-bloom fruit drop | Colletotrichum acutatum; |
| | | Colletotrichum |
| | | gloeosporioides |
| turf grass | Gray leaf spot | Pyricularia grisea |
| almond, peach, cherry, | Brown rot; blossom blight | Monilinia laxa |
| apricot, plum, nectarine, | | |
| prune, stone fruits | | |
| almond, apricot, | Shot hole; coryneum disease; | Wilsonomyces carpophilus |
| nectarine, peach, prune, | gum spot | |
| cherry, stone fruits | | |
| potato, pulse crops, | White mold | Sclerotinia sclerotiorum |
| canola | | |
| canola, oilseed rape | Black leg | Leptosphaeria maculans |
| leafy greens | Cercospora leaf spot | Cercospora spp. |
| soybean | Sudden death syndrome | Fusarium virguliforme; |
| | | Fusarium brasilense; |
| | | Fusarium solani spp. |
| corn, soybean, leafy | Rhizoctonia seedling blight, | Rhizoctonia solani; |
| green vegetables, grains, | damping-off, root rot, seed rot | Rhizoctonia spp. |
| fruiting, vegetables, | | |
| potato | | |
| corn, soybean, leafy | Pythium seedling blight, | Pythium spp., Pythium |
| green vegetables, grains, | damping-off, root rot, seed rot, | sylvaticum, |
| fruiting, vegetables | stalk rot | |
| corn, soybean, leafy | Fusarium seedling blight, | Fusarium spp., Fusarium |
| green vegetables, grains, | damping-off, root rot, seed rot, | graminearum, Fusarium |
| fruiting, vegetables | stalk rot | moniliforme |
| sugarbeets | Cercospora leaf spot | Cercospora spp. |
| grapevine, pepper, | Powdery mildew | Erysiphe spp., Erysiphe |
| tomato, cucurbits, hops | | necator, Oidium |
| | | lycopersicum, Leveillula |
| | | taurica, Podosphaeria |
| | | macularis |
| leafy green vegetables | Downy mildew | Peronospora spp., Bremia |
| | | lactucae |
| wheat, barley, oats, | Fusarium head blight, scab | Fusarium spp., Fusarium |
| triticale | | graminearum |
| potato, tomato | Early blight | Alternaria solani |
| potato | Late blight | Phytopthora infestans |
| fruiting trees, nut trees, | Phytopthora root rot, crown rot | Phytopthora spp. |
| vines | | |
| corn, soybean, grains, | Phytopthora seedling wilt, root | Phytopthora spp. |
| leafy green vegetables, | rot, damping-off, stem rot | |
| fruiting vegetables | | |
| banana | Fusarium wilt, Panama disease, | Fusarium oxysporum; |
| | Panama disease tropical race 4 | Fusarium oxysporum f. sp. |
| | | cubense |
| banana | Black sigatoka, black leaf streak | Mycosphaerella fijiensis; |
| | disease | Pseudocercospora fijiensis |
| banana | Yellow sigatoka | Pseudocercospora musicola |
| rice | Rice blast, rice rotten neck, rice | Magnaporthe oryzae; |
| | seedling blight, pitting disease, | Magnaporthe grisea |
| | Johnson spot | |
| rice | Rice sheath blight | Rhizoctonia solani |
| rice | Seedling rot | Pythium spp. |
| hops | Downy mildew | Pseudoperonospora humuli |
| legumes, leafy green | Grey mold | Bortrytis spp., Bortrytis |
| vegetables, grapevine, | | cinerea |
| strawberry | | |
| corn, wheat | Rust, polishing rust. Southern rust | Puccinia spp., Puccinia |
| | | polysora, Puccinia sorghi, |
| | | Puccinia recondita |
| wheat, fruiting | Speckled leaf spot, Septoria leaf | Septoria spp. |
| vegetables | spot, Stagonospora nodorum | |
| | blotch, Septoria tritici blotch | |
| cotton, cucurbits | Target spot, Corynespora leaf | Corynespora spp., |
| | spot, Corynespora blight | Corynespora cassiicola |
| cucurbits | Gummy stem blight | Didymella bryoniae |
| corn | Southern corn leaf blight | Cochliobolus |
| | | heterostrophus |
| corn | Northern corn leaf blight | Setosphaeria turcica, |
| | | Exserohilum turcicum |
| corn, soybean, leafy | Anthracnose, Anthracnose leaf | Colletotrichum spp., |
| green vegetables, | blight, Anthracnose stalk rot, top- | Colletotrichum graminicola, |
| fruiting vegetables, | kill, die-back | Colletotrichum truncatum |
| legumes, cucurbits, | | |
| fruiting trees, nut trees, | | |
| grains | | |
| turf grass | Brown patch | Rhizoctonia spp., |
| | | Rhizoctonia solani |
| sunflower | Downy mildew | Plasmopara halstedii |
| coffee | Coffee rust, coffee leaf rust | Hemileia vastatrix |
| coffee | Cercospora Leaf Spot | Cercospora spp. |
| grapevine | Witches broom disease | Moniliopthora perniciosa, |
| apple | Alternaria blotch, leaf spot | Alternaria mali |
| apple | Glomerella leaf spot and fruit rot, | Colletotrichum spp. |
| | bitter rot | |
| cocoa | Witches broom disease | Moniliopthora perniciosa, |
| | | Moniliopthora roreri |
| cocoa | Black pod disease, cocao stem | Phytopthora spp., |
| | canker, seedling blight, nursery | Phytopthora megakarya, |
| | blight | Phytopthora palmivora, |
| | | Phytopthora capsici |
| cocoa | Anthracnose | Colletotrichum |
| | | aeschynomenes |
| cashew | Anthracnose, Anthracnose blight | Glomerella cingulata, |
| | | Colletotrichum spp. |
| cashew | Powdery mildew | Erysiphe quericcola |
| coconut | Stem bleeding disease | Chalara paradoxa, |
| | | Theilanopsis paradoxa, |
| | | Ceratocystis paradoxa |
| coconut | Postharvest stem-end rot | Pestalotiopsis adusta, |
| | | Lasiodiplodia spp. |
| grapevine | Anthracnose | Elsino\ddot{e} ampelina, |
| | | Sphaceloma ampelinum |
| grapevine | Botrytis, Noble rot | Botrytis cinereal, |
| | | Bortryotinia fuckeliana |
| grapevine | Downy mildew | Plasmopara viticola |
| grapevine | Powdery mildew | Erysiphe necator, Uncinula |
| | | necator |
| grapevine | Grape leaf rust | Phakopsora euvitis |
| Grapevine | Summer Sour Rot | Aspergillus niger, Alternaria |
| | | spp, Cladosporium |
| | | herbarum, Rhizopus |
| | | arrhizus, Penicillium spp. |
| mango | Anthracnose | Glomerella cingulata, |
| | | Colletotrichum |
| | | gloeosporioides, |
| | | Colletotrichum karstii |
| mango | Mango sudden decline, sudden | Ceratocystis fimbriata |
| | wilt | |
| mango | Mango malformation disease | Fusarium sterilihyphosum |
| mango | Powdery mildew | Oidium mangiferae |
| mango | Stem-end rot | Dothiorella dominicana, |
| | | Phomopsis spp., |
| | | Botryodiplodia theobromae, |
| | | Lasiodiplodia theobromae |
| Mango | Mango Scab | Elsino\ddot{e} mangiferae |
| Mango | Sooty Mold | Meliola mangiferae, |
| | | Capnodium mangiferae, |
| | | Capnodium ramosum, |
| | | Tricospermum acerinum |
| almond, nut trees | Hull rot | Rhizophus stolofiner, |
| | | Monthnia spp. |
| apple | Apple scab disease, Black spot | Venturia inaequalis |
| apple | Powdery mildew | Podosphaeria leucotricha |
| stone fruits | Peach leaf curl | Taphrina deformans |
| leafy green vegetables, | Lettuce leaf spot | Cercopsora spp., |
| lettuce | | Cercospora lactucae-sativae |
| leafy green vegetables, | Downy mildew | Bremia lactucae |
| lettuce | | |
| turf grass | Snow mold, Fusarium patch | Microdochium nivale |

### Cercospora Leaf Blight Disease of Soybean

Cercospora is a fungal pathogen that causes the disease Cercospora leaf blight of soybean. Cercospora leaf blight also referred to as the purple seed stain disease infects both the leaves and seeds of soybeans. Cercospora infection of soybean seeds diminishes seed appearance and quality. The causal organism of Cercospora leaf blight is Cercospora kikuchii, which overwinters in soybean residue and in the seed coats. Spread of the disease occurs when the spores from the fungus are spread to soybean plants from infected residue, weeds or other infected soybean plants. Disease spread and symptom development are accelerated during periods of warm and wet weather. Symptom development usually begins after flowering and appears as circular lesions on soybean leaves as reddish brown to purple spots that can merge to form lesions. Symptoms are apparent in the upper canopy, usually in the uppermost three or four trifoliate leaves. Infected soybean plants exhibit worsening symptoms as the crop matures, and premature defoliation of affected leaves may occur during pod-fill. Cercospora symptom development may also appear as lesions on stems, leaf petioles and pods. Seeds are infected through the attachment to the pod. Cercospora infected seeds show a purple discoloration, which can appear as specks or blotches covering the entire seed coat. Other Cercospora diseases of soybean are Frogeye leaf spot caused by Cercospora sojina which can cause premature leaf drop and yield loss.

Foliar applications of bioactive priming compositions containing flagellin or flagellin-associated polypeptides (Tables 4-5) and an inducer compound provide an alternative approach for treating the disease. Foliar applications with these bioactive priming compositions provided as a spray to the leaf surface of either asymptomatic or symptomatic plants provides a means to prevent, treat, and control Cercospora leaf blight in soybeans. Foliar applications of Flg22 derived from Bacillus thuringiensis, particularly at high use rates (e.g., 4.0 Fl. oz/Ac), can provide a means of managing early symptom development and provide healthier more vigorous soybean plants grown in field locations that have been impacted by Cercospora.

Specific compositions that can be useful for treating or reducing the symptoms of Cercospora can comprise a flagellin or flagellin-associated polypeptide having an amino acid sequence comprising SEQ ID NO 226, 571, 587 or 590; an RHPP polypeptide having a sequence comprising SEQ ID NO: 604; or a combination of a flagellin associated polypeptide having an amino acid sequence comprising any one of SEQ ID NOs 226, 587 and 590 and an RHPP polypeptide having the amino acid sequence comprising SEQ ID NO: 604. The compositions can further comprise an inducer compound. The inducer compound can comprise β-aminobutyric acid, a callose synthase inhibitor, salicylic acid, oxalic acid or any combination thereof. For example, the inducer compound can comprise β-aminobutyric acid or a callose synthase inhibitor. The callose synthase inhibitor can comprise 2-DDG.

For example, a useful combination of bioactive priming polypeptides for treating, or reducing the symptoms of Cercospora on a plant or plant part is a flagellin polypeptide having an amino acid sequence comprising SEQ ID NO: 226 alone or in combination with an RHPP polypeptide having an amino acid sequence comprising SEQ ID NO: 604. The compositions can further comprise an inducer compound. The inducer compound can comprise β-aminobutyric acid, a callose synthase inhibitor, salicylic acid, oxalic acid or any combination thereof. For example, the inducer compound can comprise β-aminobutyric acid or a callose synthase inhibitor. The callose synthase inhibitor can comprise 2-DDG. Additional treatments can further comprise a fungicide in combination with these bioactive priming polypeptides.

### Asian Soybean Rust Disease

Asian soybean rust is a fungal disease caused by Phakopsora pachyrhizi. Its etiology and symptoms are similar to Cercospora and the bioactive priming polypeptide combinations useful for treating it are similar as well. Specifically, combinations of bioactive priming polypeptides that can be useful for treating or reducing the symptoms of Asian soybean rust include: a flagellin or flagellin-associated polypeptide having an amino acid sequence comprising SEQ ID NO 226, 571, 587 or 590; an RHPP polypeptide having a sequence comprising SEQ ID NO: 604; or a combination of a flagellin associated polypeptide having an amino acid sequence comprising any one of SEQ ID NOs 226, 587, 571 and 572 and an RHPP polypeptide having the amino acid sequence comprising SEQ ID NO: 604. The compositions can further comprise an inducer compound.

For example, a useful combination of bioactive priming polypeptides for treating, or reducing the symptoms of Asian soybean rust on a plant or plant part is a flagellin polypeptide having an amino acid sequence comprising SEQ ID NO: 226 alone or in combination with an RHPP polypeptide having an amino acid sequence comprising SEQ ID NO: 604. The compositions can further comprise an inducer compound. The inducer compound can comprise 3-aminobutyric acid, a callose synthase inhibitor, salicylic acid, oxalic acid or any combination thereof. For example, the inducer compound can comprise β-aminobutyric acid or a callose synthase inhibitor. The callose synthase inhibitor can comprise 2-DDG. Additional treatments can further comprise a fungicide in combination with these bioactive priming polypeptides.

### Holcus Spot

Holcus spot is a bacterial disease caused by Pseudomonas syringae pv. actinidae. Methods are described herein for using flagellin or flagellin associated polypeptides to restrict growth of P. syringae and thus prevent or treat the disease of Holcus spot in a plant or a plant part. Compositions comprising a flagellin or flagellin associated polypeptides having amino acid sequences comprising any one of SEQ ID NOs: 226, 540, 587, 571 and 572 or any combination thereof are useful for the treatment of P. syringae. The compositions can further comprise an inducer compound. The inducer compound can comprise β-aminobutyric acid, a callose synthase inhibitor, salicylic acid, oxalic acid or any combination thereof. For example, the inducer compound can comprise β-aminobutyric acid or a callose synthase inhibitor. The callose synthase inhibitor can comprise 2-DDG.

### Sclerotinia Stem Rot (White Mold) Disease

Sclerotinia sclerotiorum is a plant pathogenic fungus that causes a disease caused white mold. It is also known as cottony rot, water soft rot, stem rot, drop, crown rotand blossom blight. Diagnostic symptoms of the white rot include black resting structures known as sclerotia and white fuzzy growths of mycelium on the infected plant. The sclerotia, in turn, produce a fruiting body that produces spores in a sac. Sclerotinia can affect herbaceous, succulent plants, particularly fruits and vegetables, or juvenile tissue on woody ornamentals. It can also affect legumes or tuberous plants like potatoes. White mold can affect a host at any stage of growth, including seedlings, mature plants, and harvested products. It is usually found on tissues with high water content and close proximity to soil. Left untreated, pale to dark brown lesions on the stem at the soil line are covered by a white, fluffy mycelial growth. This affects the xylem which leads to chlorosis, wilting, leaf drop, and death. White mold can also occur on fruit in the field or in storage and is characterized by white fungal mycelium covering the fruit and its subsequent decay. Compositions comprising a flagellin or flagellin associated polypeptides having amino acid sequences comprising any one of SEQ ID NOs: 226, 540, 571, 587, and 590 are useful for the treatment of Sclerotinia sclerotiorum. The compositions can further comprise an inducer compound. The inducer compound can comprise β-aminobutyric acid, a callose synthase inhibitor, salicylic acid, oxalic acid or any combination thereof. For example, the inducer compound can comprise β-aminobutyric acid or a callose synthase inhibitor. The callose synthase inhibitor can comprise 2-DDG.

### Pseudomonas Leaf Spot

Pseudomonas syringae pv. actinidiae (PSA) is a devastating plant pathogen causing bacterial canker of both green- (Actinidiae deliciosa) and yellow-flesh (Actinidiae chinesis) kiwi plants throughout zones of kiwi production, causing severe harvest loss in New Zealand, China, and Italy. In New Zealand alone, cumulative revenue losses to the most devastating biovar PSA-V are predicted to approach $740 million New Zealand leaves Dollars (NZD) by 2025 (Agribusiness and Economics Research Institute of Lincoln University “The Costs of Psa-V to the New Zealand Kiwifruit Industry and the Wider Community”; May 2012). PSA-V colonizes the outer and inner surfaces of the kiwi plant and can spread through the xylem and phloem tissues. Disease symptoms of PSA-V on kiwi include bacterial leaf spot, bacterial canker of the trunk, red exudates, blossom rot, discoloration of twigs, and ultimately dieback of kiwi vines. The standard method of control for PSA-V currently employs frequent foliar applications of metallic copper to kiwi vines which is predicted to lead to the selection of copper-resistant form of the pathogen and loss of disease control. Novel methods of control are urgently needed.

Compositions comprising a Flagellin or flagellin associated peptides having amino acid sequences comprising SEQ ID NO: 226, 540, 752, and/or 571 are useful for the treatment of Pseudomonas syringae, particularly in kiwis. The compositions can further comprise an inducer compound. The inducer compound can comprise β-aminobutyric acid, a callose synthase inhibitor, salicylic acid, oxalic acid or any combination thereof. For example, the inducer compound can comprise β-aminobutyric acid or a callose synthase inhibitor. The callose synthase inhibitor can comprise 2-DDG.

### Asian Citrus Greening (Huanglonging) Disease

The compositions described herein are particularly suited to treating Asian citrus greening (Huanglonging) Disease. The methods described herein incorporate a different approach to combating disease and additionally providing benefits of increasing the overall productivity of a plant. This approach is specifically directed to providing either exogenous or endogenous applications of the compositions comprising a polypeptide and/or inducer compound as described herein to combat disease in plants.

The compositions comprising a polypeptide and/or inducer compound as described herein are useful for the prevention, treatment and control of Asian citrus greening also referred to as Huanglonging (HLB) disease, a devastating disease for citrus. HLB disease is widely distributed and has been found in most commercial and residential sites in all counties that have commercial citrus orchards.

Methods are described herein for using compositions comprising a bioactive polypeptide described herein in combination with an inducer compound to prevent the spread of and in the treatment of HLB disease. For example, the method can comprise using a flagellin or flagellin associated polypeptide in combination with 2-DDG, β-aminobutyric acid, benzothiazole, oxytetracycline, cysteine, betaine, salicylic acid, oxalic acid or any combination thereof to prevent the spread of and in the treatment of HLB disease.

Asian citrus greening disease is transmitted by the Asian citrus psyllid, Diaphorina citri or the two-spotted citrus psyllid, Trioza erytreae Del Guercio, which are both characterized as sap-sucking, hemipteran bug(s) in the family Psyllidae and have been implicated in the spread of citrus greening, a disease caused by a highly fastidious phloem-inhabiting bacteria, Candidatus Liberibacter asiaticus (Halbert, S. E. and Manjunath, K. L, “Asian citrus psyllids Sternorrhyncha: Psyllidae and greening disease of citrus: A literature review and assessment of risk in Florida,” Florida Entomologist 87: 330-353, 2004). Asian citrus greening or Huanglongbing disease is considered fatal for a citrus tree once the tree becomes infected.

The early symptoms of the disease on leaves are vein yellowing and an asymmetrical chlorosis referred to as blotchy mottle, which is the most diagnostic symptom of the disease. Infected trees are stunted and sparsely foliated with a blotchy mottling appearing on the foliage. Early symptoms of yellowing may appear on a single shoot or branch and with disease progression, the yellowing can spread over the entire tree. Afflicted trees may show twig dieback, and fruit drop. Fruit are often few in number, small, deformed or lopsided and fail to color properly, remaining green at the end and display a yellow stain just beneath the peduncle (stem) on a cut fruit.

The Asian citrus greening disease may also be graft transmitted when citrus rootstocks are selected for and grafted to scion varieties.

Management of citrus greening disease has proven difficult and therefore current methods for control of HLB have taken a multi-tiered integrated disease and pest management approach using 1) the implementation of disease-free nursery stock and rootstock used in grafting, 2) the use of pesticides and systemic insecticides to control the psyllid vector, 3) the use of biological control agents such as antibiotics, 4) the use of beneficial insects, such as parasitic wasps that attack the psyllid, and 5) breeding for new citrus germplasm with increased resistance to the citrus greening causing bacteria (Candidatus Liberibacter spp.). The use of cultural and regulatory measures to prevent the spread of the disease is also part of the integrated management approach. Many aspects involved in the management of citrus greening are costly both monetarily and in respect to losses in citrus production.

Interveinal application of a thionin polypeptide or mixture of thionin polypeptides can be delivered directly into the phloem (e.g., phloem cells including phloem sap, phloem companion cells and phloem sieve tube elements) where Candidatus Liberibacter can reside.

The thionins can be produced using an expression system where they can be fused to a phloem targeting sequence(s) (Table 14) and then uniquely delivered to the same vicinity where the bacteria can reside in the citrus plant.

The phloem targeted thionin bioactive priming polypeptides are useful for treating citrus plants to prevent, reduce or eliminate the spread of the Asian citrus greening disease or Huanglonging (HLB) by directly targeting the bacterium, Candidatus Liberibacter asiaticus.

These phloem targeted thionins can be delivered by injection into the phloem of a shrub or tree. Additionally, they can be delivered by spraying, washing, or adding as a soak or a drench to the soil or area surrounding a plant.

Any of the phloem targeting sequences (Table 14; SEQ ID NOs: 611-619) can be used in combinations with the thionin and thionin-like polypeptides (Table 15; SEQ ID NOs: 620-719).

The bacteria that cause HLB, Candidatus Liberibacter asiaticus is difficult to isolate and culture. In order to test individual thionins and thionins with the phloem targeting sequences to determine if they are useful for the treatment of HLB disease, Agrobacterium tumefaciens can be used as a model organism to test the effectiveness on reducing the cell titer or growth of Agrobacterium prior to using the thionin or thionin combinations in an orchard setting.

The “peptide priming” methods provided herein with the thionins and/or thionin-like polypeptides (Table 15) can also be used in combination with flagellin and flagellin-associated polypeptides (Tables 1-5). Combinations of the thionin- and flagellin-associated bioactive priming polypeptides can be used to prophylactically pre-treat a citrus plant by applying the bioactive priming polypeptide or a composition containing the polypeptide prior to the onset or appearance of any infection-related symptoms on the citrus shrubs or trees. This pretreatment increases resistance to the disease pathogen that causes citrus greening (Candidatus Liberibacter spp.).

The thionins provided in combination with the flagellin associated bioactive priming polypeptides provide a more comprehensive approach to disease prevention and management. The thionin and flagellin associated bioactive priming polypeptides use two distinct modes of action to prevent disease and the spread of disease.

The thionin-flagellin bioactive priming polypeptide combinations can also be used with any other integrated management approach for disease control prescribed for HLB including, but are not limited to, (1) the use of disease-free nursery stock and/or rootstocks for grafting, (2) the use of pesticides and/or systemic insecticides to control the disease-causing psyllid, (3) the use of biological control agents such as injections of antibiotics or parasitic insects that controls the psyllid, (4) breeding new varieties of citrus germplasm with increased resistance to the bacteria responsible for Asian citrus greening disease, (5) controlling parasitic plants (for example, dodder) that may spread the disease, or (6) any combination thereof.

A synthetic version of a phloem targeting polypeptide (SEQ ID NO: 611) is particularly useful in targeting anti-microbial polypeptides to the phloem sieve tube and companion cells and can be useful for treating various bacterial diseases of plants, such as bacterial leaf streak, Asian citrus greening or Huanglonging and citrus canker.

In addition, flagellin or flagellin associated polypeptides are useful for treating Asian citrus greening, especially when used in combination with a bacteriocide. For instance, flagellin or flagellin associated polypeptides having amino acid sequences comprising any one of SEQ ID NOs: 226, 571 can be used. Preferably, the bacteriocide comprises oxytetracycline.

Other compositions that are useful against these diseases include “enzyme recovery mixes” comprising a β-1,3-endoglucanase, an α-amylase, an L-cysteine and 2-DDG with or without a flagellin or flagellin associated polypeptide. For example, a suitable composition can comprise a β-1,3-endoglucanase having an amino acid sequence comprising any one of SEQ ID NOs: 731-733 and 767-776, an α-amylase having an amino acid sequence comprising SEQ ID NO: 734 or 735, an L-cysteine and 2-DDG. The composition can further comprise a flagellin or flagellin associated polypeptide. The flagellin or flagellin associated polypeptide can have an amino acid sequence comprising SEQ ID NO: 226 or 571.

### Citrus Canker

“Peptide priming” methods were developed for use with the the compositions comprising a polypeptide and/or inducer compound as described herein to prophylactically treat citrus plants prior to any visible symptoms of the citrus canker disease or as a treatment once the onset of disease symptoms become apparent.

Citrus canker occurs primarily in tropical and sub-tropical climates and has been reported to occur in over thirty countries including spread of infection reported in Asia, Africa, the Pacific and Indian Oceans Islands, South America, Australia, Argentina, Uruguay, Paraguay, Brazil and the United States. Citrus canker is a disease caused by the bacterium, Xanthomonas axonopodis pv. citri or pv. aurantifolii (also referred as Xanthomonas citri subsp. citri) that infect foliage, fruit and young stems. Symptoms of citrus canker infection on leaves, and fruit of the citrus shrubs/trees can result in leaf-spotting, leaf lesions, defoliation, die back, deformation of fruit, fruit rind-blemishing, pre-mature fruit drop, and canker formation on leaves and fruits. Diagnostic symptoms of citrus canker include a characteristic yellow halo that surrounds the leaf lesions and a water-soaked margin that develops around the necrotic tissue on the leaves of the citrus plant. The citrus canker pathogen can spread through the transport of infected fruit, plants, and equipment. Dispersal can also be facilitated by the wind and rain. Overhead irrigation systems may also facilitate movement of the citrus canker causing pathogen. Infected stems can harbor the citrus canker causing bacteria (Xanthomonas axonopodis pv. citri) in the stem lesions for transmission to other citrus plants. Insects, such as the Asian leaf miner (Phyllocnistis citrella) also disseminate the disease.

In general, citrus plants susceptible to the citrus canker disease include orange, sweet orange, grapefruit, pummelo, mandarin tangerine, lemon, lime, swingle acid lime, palestine sweet lime, tangerine, tangelo, sour orange, rough lemon, citron, calamondin, trifoliate orange and kumquat. World-wide, millions of dollars are spent annually on prevention, sanitation, exclusion, quarantine and eradication programs to control citrus canker (Gottwald T. R. “Citrus Canker,” The American Phytopathological Society, The Plant Health Instructor 2000/updated in 2005). Treatment for the disease has included application of antibiotics or disinfectants, the use of copper-based bactericidal sprays, and pesticide applications for Asian leaf miner control.

The compositions comprising a polypeptide and/or inducer compound as described herein can be applied to a citrus plant or citrus plant part (e.g., rootstock, scion, leaves, roots, stems, fruit, and foliage) using application methods that can comprise: spraying, inoculating, injecting, soaking, infiltrating, washing, dipping and/or provided to the surrounding soil as an in furrow treatment.

The methods are provided using the compositions comprising a polypeptide and/or inducer compound as described herein to pre-treat citrus plants or citrus plant parts (e.g., root stock, scion, leaves, roots, stems, fruit, and foliage) prior to any visible occurrence of symptoms. They are also useful for providing an increase in resistance to the citrus canker pathogen resulting in a reduction in disease symptoms.

Additionally, the methods of using the compositions comprising a polypeptide and/or inducer compound as described herein are useful to treat citrus plants or citrus plant parts (e.g., root stock, scion, leaves, roots, stems, fruit, and foliage) once the early onset of citrus canker disease symptoms or when the symptoms of the disease become apparent.

Application of the compositions comprising a polypeptide and/or inducer compound as described herein for treating citrus plants to prevent, reduce or eliminate the spread of the citrus canker disease can be delivered by injecting into the phloem of a shrub, tree, or vine; and/or by spraying, washing, adding as a soak or a drench to the soil or soil area surrounding a plant or provided in furrow.

The compositions comprising a polypeptide and/or inducer compound as described herein can be applied as a foliar treatment or spray or as an injection and are useful for the prevention of infestation of citrus plants from insects such as the Asian leaf miner (Phyllocnistis citrella) that have been identified in the dissemination of the bacteria (Xanthomonas axonopodis pv. citri) that cause the citrus canker disease.

### Sooty Mold

Sooty mold infection can occur on plant surfaces including fruit, leaves or other plant parts exposed to various Ascomycete fungi, such as Cladosporium and Alternaria species. Symptoms include dark spots and stained areas on the surface of the plant or plant part, with possible visible mold growth, including filamentous or spore-laden patches. Fruit including but not limited to kiwifruit, oranges, grapes, as well as pecan and hickory trees and ornamental plants are particularly susceptible to sooty mold growth. These blemishes are primarily a cosmetic issue but reduce the marketability of fruit. Mold growth is often caused by sucking insects that feed on fruit or other plant parts and then excrete sugary secretions known as honeydew onto plant surfaces. The fungi are then able to colonize the surfaces with honeydew available. Sooty mold is estimated to result in a $50 million production loss to the New Zealand kiwifruit industry annually. While sooty mold can be washed off fruit post-harvest, processing limitations make it unfeasible to apply liquid products to fruit after they are picked. Thus, there is a need for treatments that can be applied pre-harvest to remove sooty mold from fruit or prevent it from growing. Compositions comprising a polypeptide and/or inducer compound as described herein can be applied as a foliar treatment or spray or fruit wash for prevention or sooty mold growth or removal of sooty mold. For instance, glucanases (SEQ ID NO: 731-733 and 767-776), chitinases (SEQ ID NO: 777-778), and serine proteases (SEQ ID NO: 721, 722, and 794-796) are useful for reducing sooty mold growth on kiwifruit.

### Citrus Plants

Any of the methods described herein to provide improved plant health, disease tolerance or disease treatment applications to treat or prevent Asian citrus greening (HLB) or citrus canker are suitable for use with any citrus plants and shrubs/trees.

The compositions comprising a polypeptide and/or inducer compound as described herein can be applied to any citrus shrub and/or tree and to any agronomically-important citrus hybrid or citrus non-hybrid plant and are useful for prophylactically treating the citrus to prevent the onset of an infection or providing treatment after an infection has occurred.

Citrus plant species for use of the methods described herein can comprise any plant of the genus Citrus, family Ruttaceae, and include, but are not limited to: Sweet orange also known as Hamlin or Valencia orange (Citrus sinensis, Citrus maxima x Citrus reticulata), Bergamot Orange (Citrus bergamia, Citrus limetta x Citrus aurantium), Bitter Orange, Sour Orange, or Seville Orange (Citrus aurantium, Citrus maxima x Citrus reticulata), Blood Orange (Citrus sinensis), Orangelo or Chironja (Citrus paradisi x Citrus sinensis), Mandarin Orange (Citrus reticulate), Trifoliate Orange (Citrus trifoliata), Tachibana Orange (Citrus tachibana), Alemow (Citrus macrophylla), Clementine (Citrus clementina), Cherry Orange (Citrus kinokuni), Lemon (Citrus limon, or hybrids with Citrus maxima x Citrus medica) or Citrus limonia, Indian Wild Orange (Citrus indica), Imperial Lemon (Citrus limon, Citrus medica x Citrus paradisi), Lime (Citrus latifoli, Citrus aurantifolia), Meyer Lemon (Citrus meyeri); hybrids of Citrus x meyeri with Citrus maxima, Citrus medica, Citrus paradisi and/or Citrus sinensis), Rough Lemon (Citrus jambhiri), Volkamer Lemon (Citrus volkameriana), Ponderosa Lemon (Citrus limon x Citrus medica), Key Lime (Citrus aurantiifolia), Kaffir Lime (Citrus hystrix or Mauritius papeda), Sweet Lemon, Sweet Lime, or Mosambi (Citrus limetta), Persian Lime or Tahiti Lime (Citrus latifolia), Palestine Sweet Lime (Citrus limettioides), Winged Lime (Citrus longispina), Australian Finger Lime (Citrus australasica), Australian Round Lime (Citrus australis), Australian Desert or Outback Lime (Citrus glauca), Mount White Lime (Citrus garrawayae), Jambola (Citrus grandis), Kakadu Lime or Humpty Doo Lime (Citrus gracilis), Russel River Lime (Citrus inodora), New Guinea Wild Lime (Citrus warburgiana), Brown River Finger Lime (Citrus wintersii), Mandarin Lime (Citrus limonia; (hybrids with Citrus reticulata x Citrus maxima x Citrus medica), Carabao Lime (Citrus pennivesiculata), Blood Lime (Citrus australasica x Citrus limonia) Limeberry (Triphasia brassii, Triphasia grandifolia, Triphasia trifolia), Lemon hybrid or Lumia (Citrus medica x Citrus limon), Omani Lime (Citrus aurantiifolia, Citrus medica x Citrus micrantha), Sour Lime or Nimbuka (Citrus acida), Grapefruit (Citrus paradisi; Citrus maxima x Citrus x sinensis), Tangarine (Citrus tangerina), Tangelo (Citrus tangelo; Citrus reticulata x Citrus maxima or Citrus paradisi), Minneola Tangelo (Citrus reticulata x Citrus paradisi), Orangelo (Citrus paradisi x Citrus sinensis), Tangor (Citrus nobilis; Citrus reticulata x Citrus sinensis), Pummelo or Pomelo (Citrus maxima or Citrus retkulata), Citron (Citrus medica), Mountain Citron (Citrus halimii), Kumquat (Citrus japonica or Fortunella species), Kumquat hybrids (Calamondin, Fortunella japonica; Citranqequat, Citrus ichangensis; Limequat, Citrofortunella floridana); Orangequat, hybrid between Satsuma mandarin x Citrus japonica or Fortunella species; Procimequat, Fortunella hirdsiie; Sunquat, hybrid between Citrus meyeri and Citrus japonica or Fortunella species; Yuzuquat, hybrid between Citrus ichangensis and Fortunella margarita), Papedas (Citrus halimii, Citrus indica, Citrus macroptera, Citrus micrantha), Ichang Papeda (Citrus ichangensis), Celebes Papeda (Citrus celebica), Khasi Papeda (Citrus latipes), Melanesian Papeda (Citrus macroptera), Ichang Lemon (Citrus ichangensis x Citrus maxima), Yuzu (Citrus ichangensis x Citrus reticulata), Cam sành (Citrus reticulata x Citrus maxima), Kabosu (Citrus sphaerocarpa), Sudachi (Citrus sudachi), Alemow (Citrus macrophylla), Biasong (Citrus micrantha), Samuyao (Citrus micrantha), Kalpi (Citrus webberi), Mikan (Citrus unshiu), Hyuganatsu (Citrus tamurana), Manyshanyegan (Citrus mangshanensis), Lush (Citrus crenatifolia), Amanatsu or Natsumikan (Citrus natsudaidai), Kinnow (Citrus nobilis x Citrus deliciosa), Kiyomi (Citrus sinensis x Citrus unshiu), Oroblanco (Citrus maxima x Citrus paradisi), Ugh (Citrus reticulata x Citrus maxima and/or Citrus x paradisi), Calamondin (Citrus reticulata x Citrus japonica), Chinotto (Citrus myrtifolia, Citrus aurantium or Citrus pumila), Cleopatra Mandarin (Citrus reshni), Daidai (Citrus aurantium or Citrus daidai), Laraha (Citrus aurantium), Satsuma (Citrus unshiu), Naartjie (Citrus reticulata x Citrus nobilis), Rangpur (Citrus limonia; or hybrid with Citrus sinensis x Citrus maxima x Citrus reticulata), Djeruk Limau (Citrus amblycarpa), Iyokan, anadomikan (Citrus iyo), Odichukuthi (Citrus odichukuthi), Ougonkan (Citrus flaviculpus), Pompia (Citrus monstruosa), Tangerine (Citrus tangerine), Taiwan Tangerine (Citrus depressa), Shonan gold (Citrus flaviculpus or Citrus unshiu), Sunki (Citrus sunki), Mangshanyen (Citrus mangshanensis, Citrus nobilis), Clymenia (Clymenia platypoda, Clymenia polyandra), Jabara (Citrus jabara), Mandora (Mandora cyprus), Melogold (Citrus grandis x Citrus paradisii/Citrus maxima/Citrus grandis), Shangjuan (Citrus ichangensis x Citrus maxima), Nanfengmiju (Citrus reticulata), and Shīkwāsaī (Citrus depressa)

The compositions comprising a polypeptide and/or inducer compound as described herein can be applied to any citrus plant, shrub/tree used for medicinal or cosmetic/health and beauty purposes, such as Bergamot Orange (Citrus bergamia), Sour or Bitter Orange (Citrus aurantium), Sweet Orange (Citrus macrophylla), Key Lime (Citrus aurantiifolia), Grapefruit (Citrus paradisi), Citron (Citrus medica), Mandarin Orange (Citrus reticulate), Lemon (Citrus limon, or hybrids with Citrus medica x Citrus maxima, Citrus limonia, Citrus medica x Citrus maxima x Citrus medica), Sweet Lime (Citrus limetta), Kaffir Lime, (Citrus hystrix or Mauritius papeda), Lemon hybrid or Lumia (Citrus medica x Citrus limon, Omani Lime (Citrus aurantiifolia, Citrus medica x Citrus micrantha), Jambola (Citrus grandis), Kakadu Lime or Humpty Doo Lime (Citrus gracilis), Pomelo (Citrus maxima), Tangor (Citrus nobilis), and Sour Lime or Nimbuka (Citrus acida).

Exemplary important citrus hybrids for fruit production are: Sweet Orange (Citrus sinensis), Bitter Orange (Citrus aurantium), Grapefruit (Citrus paradisi), Lemon (Citrus limon), Persian Lime (Citrus latifolia), Key Lime (Citrus aurantiifolia), Tangerine (Citrus tangerine) and Rangpur (Citrus limonia).

Additionally, any the compositions comprising a polypeptide and/or inducer compound as described herein can be applied to any citrus plant, shrub/tree used as a rootstock and/or a scion germplasm. The methods are particularly useful for rootstocks commonly used in grafting of citrus to enhance the merits of the scion varieties, which can include tolerance to drought, frost, disease or soil organisms (for example, nematodes). Such citrus plants that provide useful rootstocks include: Sour or Bitter Orange (Citrus aurantium), Sweet Orange (Citrus macrophylla), Trifoliate Orange (Poncirus trifoliata), Rough Lemon (Citrus jambhiri), Volkamer Lemon (Citrus volkameriana), Alemow (Citrus macrophylla), Cleopatra Mandarin (Citrus reshini), Citrumelo (hybrids with x citroncirus species), Grapefruit (Citrus paradisi), Rangpure Lime (Citrus limonia), Palestine Sweet Lime (Citrus limettioides) and Troyer Citrange (Citrus sinensis x Poncirus trifoliata or Citrus sinensis x Citrus trifoliata) and Citrange (Citrus sinensis x Poncirus trifoliata or C. sinensis x C. trifoliata). Citrus varieties can also be recombinant, engineered to additionally express higher levels of defensins, antimicrobial peptides, or recombinant virus particle.

### EXAMPLES

The following non-limiting examples are provided to further illustrate the present invention.

### Examples 1-5: Use of Flagellin Peptides in Combination with Other Inducers to Prevent and Treat Citrus Disease

Examples 1-5 describe the use of various compositions in the prevention and treatment of citrus disease. For ease of reference, the compositions tested, their mode of administration and application use rate are described in Table 24 below. Note that some compositions (e.g., composition 6) are described as having two parts (Part A and Part B). As will be described in the examples, these two parts were applied simultaneously or sequentially depending on the test.

TABLE 24
Compositions for the prevention and treatment of citrus disease

| | | | Application Use |
|---|---|---|---|
| | | | Rate |
| | | | Milliliters per tree |
| | | | (mL/tree) or Fluid |
| | | Treatment | ounce/acre (Fl. |
| Composition No: | Formulation | Method | oz/Ac) |
| Citrus Composition 1 | Bt.4Q7Flg22Syn01 (SEQ ID | Trunk Injection | 2.75 mL/tree |
| | NO: 571) 120 ppm | | (0.33 mg) |
| | 10 mM Sodium Phosphate | | |
| | Buffer, pH 5.7 | | |
| Citrus Composition 2 | Oxytetracycline-HCl | Trunk Injection | 20 mL/tree |
| | (48.6 mg/mL solution in | | (0.9278 g per tree) |
| | water) | | |
| Citrus Composition 3 | Oxytetracycline-HCl | Trunk Injection | 20 mL/tree |
| | (24.1 mg/mL solution in | | (0.4818 g per tree) |
| | water) | | |
| Citrus Composition 4 | Thionin-like Peptide | Trunk Injection | 80 mL/tree |
| | (SEQ ID NO: 620) | | |
| | (fermentation broth filtrate) | | |
| Citrus Composition 5 | Serine Protease 2 | Trunk Injection | 20 mL/tree |
| | Bacillus subtilis | | |
| | (SEQ ID NO: 795) | | |
| | (fermentation broth filtrate) | | |
| Citrus Composition 6 | Part A | Trunk Injection | 2.75 mL/tree |
| | Bt.4Q7Flg22Syn01 | | (0.33 mg/tree) |
| | (SEQ ID NO: 571) 120 ppm | | |
| | 10 mM Sodium Phosphate | | |
| | Buffer, pH 5.7 | | |
| | Part B | Trunk Injection | 20 mL/tree |
| | Oxytetracycline-HCl | | (0.4818 g per tree) |
| | (24.1 mg/mL solution in | | |
| | water) | | |
| Citrus Composition 7 | Part A | Foliar Spray | 3.0 mL/tree (0.36 |
| | Bt.4Q7Flg22Syn01 | | mg/tree) in a spray |
| | (SEQ ID NO: 571) 120 ppm | | carrier volume of 3 |
| | 10 mM Sodium Phosphate | | L water + 0.1% v/v |
| | Buffer, pH 5.7 | | non-ionic surfactant |
| | | | (alkyl phenol |
| | | | ethoxylate) |
| | Part B | Trunk Injection | 20 mL/tree |
| | Oxytetracycline-HCl | | (0.4818 g per tree) |
| | (24.1 mg/mL solution in | | |
| | water) | | |
| Citrus Composition 8 | Part A | Foliar Spray | 12.0 mL/tree (1.44 |
| | Bt.4Q7Flg22Syn01 | | mg/tree) in a spray |
| | (SEQ ID NO: 571) 120 ppm | | carrier volume of 3 |
| | 10 mM Sodium Phosphate | | L water + 0.1% v/v |
| | Buffer, pH 5.7 | | non-ionic surfactant |
| | | | (alkyl phenol |
| | | | ethoxylate) |
| | Part B | Trunk Injection | 20 mL/tree |
| | Oxytetracycline-HCl | | (0.4818 g per tree) |
| | (24.1 mg/mL solution in | | |
| | water) | | |
| Citrus Composition 9 | Part A | Trunk Injection | 2.75 mL/tree |
| | Bt.4Q7Flg22Syn01 | | (0.33 mg/tree) |
| | (SEQ ID NO: 571) 120 ppm | | |
| | 10 mM Sodium Phosphate | | |
| | Buffer, pH 5.7 | | |
| | Part B | Trunk Injection | 20 mL/tree |
| | L-Cysteine | | (62.86 mg per tree) |
| | (3.143 mg/mL solution in | | |
| | water) | | |
| Citrus Composition | Part A | Trunk Injection | 2.75 mL/tree |
| 10 | Bt.4Q7Flg22-Syn01 | | (0.33 mg/tree) |
| | (SEQ ID NO: 571) 120 ppm | | |
| | 10 mM Sodium Phosphate | | |
| | Buffer, pH 5.7 | | |
| | Part B | Trunk Injection | 20 mL/tree |
| | 2-Deoxy-D-Glucose (2-DDG) | | (111.1 mg per tree) |
| | (5.56 mg/mL solution in | | |
| | water) | | |
| Citrus Composition | Part A | Trunk Injection | 2.75 mL/tree |
| 11 | Bt.4Q7Flg22-Syn01 | | (0.33 mg/tree) |
| | (SEQ ID NO: 571) 120 ppm | | |
| | 10 mM Sodium Phosphate | | |
| | Buffer, pH 5.7 | | |
| | Part B | Trunk Injection | 20 mL/tree |
| | β-Aminobutyric acid (BABA) | | (2 g per tree) |
| | (100 mg/mL solution in | | |
| | water) | | |
| Citrus Composition | Part A | Trunk Injection | 2.75 mL/tree |
| 12 | Bt.4Q7Flg22Syn01 | | (0.33 mg/tree) |
| | (SEQ ID NO: 571) 120 ppm | | |
| | 10 mM Sodium Phosphate | | |
| | Buffer, pH 5.7 | | |
| | Part B | Trunk Injection | 20 mL/tree |
| | ACTIGARD WG | | (1 g per tree) |
| | (Active Ingredient: 50% | | |
| | Acibenzolar-S-methyl: | | |
| | Benzo (1,2,3) thiadiazole- | | |
| | 7-carbothioic acid-S-methyl | | |
| | ester; BTH) | | |
| | (50 mg/mL solution in water) | | |
| Citrus Composition | Part A | Trunk Injection | 2.75 mL/tree |
| 13 | Bt.4Q7Flg22Syn01 (SEQ ID | | (0.33 mg/tree) |
| | NO: 571) 120 ppm | | |
| | 10 mM Sodium Phosphate | | |
| | Buffer, pH 5.7 | | |
| | Part B | Trunk Injection | 20 mL/tree |
| | Serine Protease 2 | | |
| | Bacillus subtilis | | |
| | (SEQ ID NO: 795) | | |
| | (fermentation broth filtrate) | | |
| Citrus Composition | Part A | Trunk Injection | 2.75 mL/tree |
| 14 | Bt.4Q7Flg22Syn01 (SEQ ID | | (0.33 mg/tree) |
| | NO: 571) 120 ppm | | |
| | 10 mM Sodium Phosphate | | |
| | Buffer, pH 5.7 | | |
| | Part B | Trunk Injection | 80 mL/tree |
| | Thionin-like Peptide (SEQ ID | | |
| | NO: 620) | | |
| | (fermentation broth filtrate) | | |
| Citrus Composition | Part A | Trunk Injection | 20 mL/tree |
| 15 | L-Cysteine | | (62.86 mg per tree) |
| | (3.143 mg/mL solution in | | |
| | water) | | |
| | Part B | Trunk Injection | 20 mL/tree |
| | Oxytetracycline-HCl | | (0.9278 g per tree) |
| | (48.6 mg/mL solution in | | |
| | water) | | |
| Citrus Composition | Part A | Trunk Injection | 20 mL/tree |
| 16 | 2-Deoxy-D-Glucose (2-DDG) | | (111.1 mg per tree) |
| | (5.56 mg/mL solution in | | |
| | water) | | |
| | Part B | Trunk Injection | 20 mL/tree |
| | Oxytetracycline-HCl | | (0.4818 g per tree) |
| | (24.1 mg/mL solution in | | |
| | water) | | |
| Citrus Composition | Part A | Trunk Injection | 20 mL/tree |
| 17 | β-Aminobutyric acid (BABA) | | (2 g per tree) |
| | (100 mg/mL solution in | | |
| | water) | | |
| | Part B | Trunk Injection | 20 mL/tree |
| | Oxytetracycline-HCl | | (0.4818 g per tree) |
| | (24.1 mg/mL solution in | | |
| | water) | | |
| Citrus Composition | Part A | Trunk Injection | 20 mL/tree |
| 18 | ACTIGARD WG | | (1 g per tree) |
| | (Active Ingredient: 50% | | |
| | Acibenzolar-S-methyl: | | |
| | Benzo (1,2,3) thiadiazole-7- | | |
| | carbothioic acid-S-methyl | | |
| | ester; BTH) | | |
| | (50 mg/mL solution in water) | | |
| | Part B | Trunk Injection | 20 mL/tree |
| | Oxytetracycline-HCl | | (0.9278 g per tree) |
| | (48.6 mg/mL solution in | | |
| | water) | | |
| Citrus Composition | Part A | Trunk Injection | 20 mL/tree |
| 19 | Serine Protease 2 | | |
| | Bacillus subtilis | | |
| | (SEQ ID NO: 795) | | |
| | (fermentation broth filtrate) | | |
| | Part B | Trunk Injection | 20 mL/tree |
| | Oxytetracycline-HCl | | (0.4818 g per tree) |
| | (24.1 mg/mL solution in | | |
| | water) | | |
| Citrus Composition | Part A | Trunk Injection | 80 mL/tree |
| 20 | Thionin-like Peptide (SEQ ID | | |
| | NO: 620) | | |
| | (fermentation broth filtrate) | | |
| | Part B | Trunk Injection | 20 mL/tree |
| | Oxytetracycline-HCl | | (0.9278 g per tree) |
| | (48.6 mg/mL solution in | | |
| | water) | | |
| Citrus Composition | Bt.4Q7Flg22-Syn01 (SEQ ID | Foliar Spray | 3.0 mL/tree (0.36 |
| 21 | NO: 571) 120 ppm | | mg) in a spray |
| | 10 mM Sodium Phosphate | | carrier volume of 3 |
| | Buffer, pH 5.7 | | L water + 0.1% v/v |
| | | | non-ionic surfactant |
| | | | (akyl phenol |
| | | | ethoxylate) |
| Citrus Composition | Part A | Foliar Spray | 12.0 mL/tree (1.44 |
| 22 | Bt.4Q7Flg22Syn01 (SEQ ID | | mg) in a spray |
| | NO: 571) 120 ppm | | carrier volume of 3 |
| | 10 mM Sodium Phosphate | | L water + 0.1% v/v |
| | Buffer, pH 5.7 | | non-ionic surfactant |
| | | | (akyl phenol |
| | | | ethoxylate) |
| Citrus Composition | Bt.4Q7Flg22 (SEQ ID NO: | Trunk Injection | 2.75 mL/tree |
| 23 | 226) 120 ppm | | (0.33 mg) |
| | 10 mM Sodium Phosphate | | |
| | Buffer, pH 5.7 | | |

### Example 1: Treatment of Citrus Trees Infected with Candidatus Liberibacter Asiaticus (CLas) with Flg22 Peptide Combinations—Increased Fruit Yield —Hamlin Orange: Melvin Grove, Florida

Trees were treated at three separate sites in Florida sites that were selected due to a high prevalence of Citrus Greening Disease (Huanglongbing) caused by the bacterial pathogen Candidatus Liberibacter asiaticus (CLas). Five-year old Hamlin orange trees (Citrus sinensis) were treated at a commercial grove orchard located in central Florida (Okeechobee County), 6-year old Vernia orange trees on Swingle rootstock were treated at Lake Wales, Fla. (Polk County), and 9-year old Valencia orange trees were treated at Eustis, Fla. (Lake County). Citrus composition treatments were applied as listed in Table 24 above using a low-pressure injection device, BRANDT ENTREE (BRANDT) for trunk injection or a CO₂-pressurized backpack sprayer that produced a fine mist for foliar spray. Foliar compositions of Bt.4Q7Flg22 were diluted in water with a non-ionic surfactant (alkyl phenol ethoxylate; 0.1% v/v of spray tank volume) and evenly applied to the canopy of the tree at a spray rate of 3 Liters (L) per tree. Blocks of trees receiving a foliar treatment were spaced in the trial area with a gap (skipped tree) in between treatment blocks to avoid drift of treatment into neighboring treatment blocks. Treatments were applied during the early morning or late evening during a period of low wind (<5 mph), and conditions were such all spray treatments dried on leaves within a period of 4 hours. Combination treatments described in Table 25 were either co-injected in the same BRANDT ENTREE bottle (Citrus Composition 6 and 9-20) or applied separately as an oxytetracycline injection followed by a Bt.4Q7Flg22Syn01 foliar treatment on the same day (Citrus Composition 7 and 8). For all treatments, 10 trees were used per treatment, separated into two replicated blocks of five trees each. Citrus compositions 1-6 and 9-22 were applied at the Okeechobee, Polk, Fla. and Lake County, FL groves, while Citrus Compositions 7 and 8 were applied at the Okeechobee, Fla. grove alone.

To assess the effects of Citrus Compositions 1-23 on fruit yield and quality, Hamlin oranges (Okeechobee County, FL) were harvested 8.5 months post-treatment, and the Vernia (Polk County, FL) and Valencia (Lake County, FL) oranges will be harvested approximately 10-11 months post-treatment. All fruit with a diameter greater than or equal to 1.6 inches (40 mm) were hand-picked and collected for each tree. The total “Fruit Count” and “Fruit Weight” (in kilograms) per individual tree was measured and recorded. Trees with total fruit weights greater than 200%, or less than 50%, of the median fruit weight for the trial (all treatments included) were considered to be outliers and removed from the dataset. Fruit size was assessed as the 1) “Average Weight per Fruit” in grams (total fruit weight divided by total fruit count per tree=average weight per fruit) and 2) “Average Fruit Diameter” in millimeters. For diameter measurements, digital calipers accurate to 0.1 mm were used to measure 10 random fruit from each tree, for a total of 100 fruit per treatment (10 trees with 10 fruit each). The calipers were positioned perpendicular to the fruit blossom and stem ends, and diameter was measured at the widest point on the fruit. One of the symptoms of citrus greening is increased fruit drop prior to harvest; therefore, the number of recently dropped, non-rotting fruit was counted for each tree. Percent (%) fruit drop was calculated as the number of pre-harvest dropped fruit divided by the total fruit (dropped and picked) for each tree. Average yield, average fruit diameter and % fruit drop for each of the tested compositions are described in Table 25 below.

Harvest results from ‘Valencia’ orange trees (Table 25) indicated that trunk injections of flagellin polypeptide compositions including Bt.4Q7Flg22 (SEQ ID NO: 226) or Bt.4Q7Flg22Syn01 (SEQ ID NO: 571) were effective for increasing the number of fruit harvested per tree and the average fruit size (weight and diameter), resulting in 33% and 26% total increased yield (kg) per tree, respectively.

TABLE 25
Trunk injection of Bt.4Q7Flg22Syn01 (SEQ ID NO: 571)
and Bt.4Q7Flg22 (SEQ ID NO: 226) increase
‘Vernia’ yield relative to an untreated control

| | Average | Average | | | |
|---|---|---|---|---|---|
| | Yield | Yield | Calculated | | |
| | (kg | (Fruit | Average | Average | % |
| | fruit per | Count per | Fruit | Diameter | Fruit |
| Treatment* | tree) | Tree) | Weight | (mm) | Drop |
| Untreated | 53.5 | 345.0 | 155.2 | 66.7 | 3.0 |
| Citrus | 67.3 | 408.1 | 164.9 | 69.6 | 2.2 |
| Composition 1 | (126%) | (118%) | (106%) | (104% | (73%) |
| Composition 23 | 71.0 | 445.1 | 159.6 | 69.4 | 1.3 |
| | (133%) | (129%) | (103%) | (104%) | (43%) |

*Citrus compositions, administration route, and dosage are descnbed in Table 24.

Next, combination treatments of trunk-injected immune activators (BABA, BTH), callose synthase inhibitor 2-DDG, proteinogenic amino acid L-cysteine, and fermentation filtrates containing the antimicrobial compounds Serine Protease 2 (SP2) (SEQ ID NO:795) and thionin (SEQ ID NO: 620) were co-injected with Bt.4Q7Flg22Syn01 (SEQ ID NO: 571) to assess whether combination treatments would further increase yield. Average yield, average fruit diameter and % fruit drop for each of the tested compositions are described in Table 26 below.

TABLE 26
Trunk injection of Flg22-Syn01 combination treatments
increased ‘Hamlin’ fruit yield relative to Flg22-Syn01 alone

| | Average Yield | Average | Fruit Drop |
|---|---|---|---|
| | (Fruit Count per | Fruit | (%) |
| | Tree) (Relative to | Diameter | (Relative to |
| Treatment* | Composition 1) | (mm) | Composition 1) |
| Citrus Composition 1 | 63.8 fruit/tree | 60.9 mm | 10.5% |
| Citrus Composition 9 | 70.8 fruit/tree | 66.6 mm | 7.21% |
| | (111%) | (109%) | (−3.3%) |
| Citrus Composition 10 | 78.6 fruit/tree | 63.2 mm | 10.8% |
| | (123%) | (104%) | (+0.3%) |
| Citrus Composition 11 | 98.3 fruit/tree | 64.8 mm | 4.5% |
| | (154%) | (107%) | (−6.0%) |
| Citrus Composition 12 | 107.8 fruit/tree | 66.6 mm | 6.3% |
| | (169%) | (109%) | (−4.2%) |
| Citrus Composition 13 | 72.5 fruit/tree | 69.1 mm | 1.4% |
| | (114%) | (113%) | (−9.1%) |
| Citrus Composition 14 | 98.9 fruit/tree | 63.3 mm | 5.7% |
| | (155%) | (104%) | (−4.8%) |

*Citrus compositions, administration route, and doses are described in Table 24

Yield results indicate that Bt.4Q7Flg22Syn01 (SEQ ID NO: 571) is compatible with all tested co-injection treatments, and that the combination treatments increase the number of harvested fruit per tree relative to Bt.4Q7Flg22Syn01 injection alone, as well as increase average fruit size (diameter, mm) and either reduce or remain unchanged pre-harvest fruit drop relative to Bt.4Q7Flg22Syn01 injection alone. In a separate trial, compatibility (additive or synergistic) was tested using a combination treatment for Bt.4Q7Flg22Syn01 (trunk injection and/or foliar spray) treatment with a trunk injection of the antibiotic oxytetracycline. A low dose of oxytetracycline (0.45 g/tree) was delivered at the same time as Bt.4Q7Flg22Syn01 treatment.

TABLE 27
Bt.4Q7Flg22Syn01 (SEQ ID NO: 571) trunk injection or foliar
spray increased ‘Hamlin’ fruit yield in trees co-injected with
oxytetracycline relative to oxytetracycline alone

| | Average | Calculated | |
|---|---|---|---|
| | Yield | Average | Fruit Drop |
| | (kg fruit | Fruit | (%); |
| | per tree) | Weight (g) | (Relative |
| | (Relative | (Relative | to Citrus |
| | to Citrus | to Citrus | Composition |
| Treatment | Composition 3) | Composition 3) | 3) |
| Citrus Composition 3 | 12.65 kg/tree | 148.9 g | 6.1% |
| Citrus Composition 6 | 13.25 kg/tree | 152.5 | 5.8% |
| | (105%) | (102%) | (−0.3%) |
| Citrus Composition 7 | 16.01 kg/tree | 145.2 | 6.3% |
| | (127%) | (98%) | (+0.2%) |
| Citrus Composition 8 | 14.03 kg/tree | 145.3 | 3.5% |
| | (111%) | (98%) | (−2.6%) |
| AVERAGE for | +14.1% | −0.8% | −0.9% |
| Compositions 6, 7 and 8 | | | |
| relative to Composition 3 | | | |

*Citrus compositions, administration route, and doses are described in Table 24

Results in Table 27 above indicated that co-treatment of ‘Hamlin’ orange trees with Bt.4Q7Flg22Syn01 (SEQ ID NO: 571) and oxytetracyline injection increases yield (kg per tree), while fruit size and fruit drop remain relatively unchanged. A citrus management program that included co-injection of Flg22 with oxytetracyline would be expected to provide on average 14.1% increase in yield relative to injection of oxytetracycline alone (Table 27).

Co-injection treatments of oxytetracycline (0.45 g or 0.90 g) with either BABA, BTH, 2-DDG, L-cysteine, BTH (ACTIGARD WG), or fermentation filtrates containing serine protease 2 or thionin were next tested for ability to increase ‘Hamlin’ yield and/or decrease fruit drop. The results are summarized in Tables 28 (yield) and 29 (fruit drop) below.

TABLE 28
Trunk injection of oxytetracycline combination treatments increased
‘Hamlin’ fruit yield relative to oxytetracycline alone

| | Average Yield | Change in Yield |
|---|---|---|
| | (kg fruit per | relative to Citrus |
| Treatment | tree) | Composition 2 or 3 (%) |
| Citrus Composition 2 | 11.98 kg/tree | — |
| Citrus Composition 3 | 12.65 kg/tree | — |
| Citrus Composition 17 | 15.90 kg/tree | +25.7% relative to |
| | | Composition 3 |
| Citrus Composition 15 | 16.5 kg/tree | +37.7% relative to |
| | | Composition 2 |
| Citrus Composition 18 | 15.18 kg/tree | +26.7% relative to |
| | | Composition 2 |

*Citrus Compositions, application route, and doses are described in Table 24.

TABLE 29
Trunk injection of oxytetracycline combination treatments
decreased fruit drop relative to oxytetracycline alone

| | | Change in Fruit Drop |
|---|---|---|
| | Average Fruit Drop | relative to Citrus |
| Treatment | (percent of total fruit) | Composition 2 or 3 (%) |
| Citrus Composition 2 | 4.7% | — |
| Citrus Composition 3 | 6.1% | — |
| Citrus Composition 16 | 3.3% | 2.8% Decrease relative |
| | | to Composition 3 |
| Citrus Composition 19 | 4.3% | 1.8% Decrease relative |
| | | to Composition 3 |
| Citrus Composition 20 | 3.5% | 1.2% Decrease relative |
| | | to Composition 2 |

*Citrus Compositions, application route, and doses are described in Table 24.

In comparison to trees injected with oxytetracycline alone, increased yield and decreased fruit drop was observed for co-injection of oxytetracycline and an inducer compound or bioactive polypeptide. Increased total kg/tree was observed when oxytetracycline was combined with immune activators BABA or BTH (ACTIGARD WG) or L-cysteine; reduced fruit drop was observed when oxytetracycline was combined with Thionin (SEQ ID NO: 620), Serine Protease 2 (SEQ ID NO: 795), or the callose synthase inhibitor 2-DDG. These treatments could be further combined with a bioactive polypeptide such as Flg22.

### Example 2: Treatment of Citrus Trees Infected with Candidatus Liberibacter Asiaticus with Flg22 Combinations Increases Fruit Quality

As described in Example 1, oranges of the ‘Hamlin’, ‘Vernia’, and “Valencia’ varieties were harvested from trials designed to test the efficacy of Bt.4Q7Flg22Syn01 peptide and oxytetracycline combination treatments for increased yield and fruit quality. Trials were arranged with 10 trees per treatment, with two replicated blocks of five trees each. At the time of harvest, two representative fruit were collected per tree.

For juice quality analysis, one set of oranges consisted of 10 total fruit, each corresponding to a sampling from 5 trees of the same experimental treatment. The set of 10 oranges were weighed (gram; g) and then juiced together. Oranges were imaged as a set of whole fruit and then cut in half so the pedicel and the style were a part of separate halves and the interior half of the fruit resembled wedges in a wheel. After imaging the halved fruit, each half was juiced until no endocarp remained. The juice from all the fruit in the set was strained to remove bulk pulp and then combined and measured for juice volume (mL) and mass (g). Mean juice volume per fruit (mL), mean fruit weight (g), and % juice content (g juice in set/g comprising the whole fruit in set) were calculated and recorded. The bulk juice was strained through a mesh strainer and samples were retained for Brix and acidity analysis (1 mL and 5 mL, respectively).

Acid-corrected ^{o}Brix (^{o}Brix_{c}) values of juice were obtained from the juiced (squeezed) fruit following the USDA minimum standards for Brix laboratory analytical methods. A MA871 Refractometer (Milwaukee Instruments) was used for Brix analysis. For Brix analysis, 1 mL of the strained juice from each set was centrifuged at 13,300×g for 30 seconds to pellet pulp. 100 μL of distilled water was used to zero the instrument, and 100 μL each of 12.5% Brix and 25% Brix standards made with sucrose (Acros Organics, Belgium) were used to verify calibration between runs. A 100 μL volume of each sample was read for Brix content and recorded. Temperature was recorded using the output in the Brix analysis meter. (JBT FoodTech Laboratory Manual, “Procedures for Analysis of Citrus Products, Sixth Edition). For determination of citric acid content (% CA), 5 mL of strained juice was diluted 10-fold in distilled water. The diluted sample was titrated to pH 8.10 using a HI 84532 Titratable Acidity Minititrator & pH Meter for Fruit Juice (Hanna Instruments). The low range of citric acid was recorded from the displayed “% CA” value. The standard pH curve was set with provided standards at pH 4.01, pH 7.01, and pH 8.20. Temperature was recorded using the displayed temperature output from the temperature probe. After collecting Brix values and acidity data, the fruit brix to acidity ratio was calculated (Brix:CA). Increased Brix and Brix:CA values are indicative of a higher quality fruit with increased sugar content relative to acid content. Results are described in Table 30 below.

TABLE 30
Trunk injection of Bt.4Q7Flg22Syn01 (SEQ ID NO: 571) combination
treatments with inducer compounds increased ‘Hamlin’ fruit yield quality

| Treatment | Juice Content (%) | Brix value |
|---|---|---|
| Untreated | 43% | 9.2 |
| Citrus Composition 1 | 46% | 9.4 |
| Citrus Composition 9 | 42% | 9.8 |
| Citrus Composition 10 | 45% | 9.3 |
| Citrus Composition 11 | 47% | 9.6 |
| Citrus Composition 12 | 36% | 9.7 |
| Citrus Composition 13 | 47% | 9.3 |
| Citrus Composition 14 | 52% | 9.9 |

*Citrus Compositions, application route, and doses are described in Table 24.

In addition to increasing yield (see Example 1), combination treatments with Bt.4Q7Flg22Syn01-injected trees improve juice content. In comparison to the untreated control fruit with a measured juice content of 43%, Bt.4Q7Flg22Syn01-treated trees produced fruit with 46% juice content. Co-injection of 2-DDG, BABA, SP2, and thionin (Composition 14) with Bt.4Q7Flg22Syn01 increased juice content by 2-9%. Brix values are also improved in comparison to the untreated control (Brix=9.2) or compared to Bt.4Q7Flg22Syn01 alone (9.4) and for all combinations tested. The greatest increase in Brix was observed for Bt.4Q7Flg22Syn01 in combination with thionin (SEQ ID NO: 620), with a Brix value of 9.9.

### Example 3: Treatment of Citrus Trees Infected with Candidatus Liberibacter Asiaticus with Recovery-Promoting Enzyme Compositions to Restore Plant Health and Increase Fruit Yield

Described herein is a method for promoting tree recovery from the symptoms of Citrus Greening Disease or HLB using a multi-pronged approach to 1) alert the plant to the presence of pathogenic bacteria using trunk injection or foliar application of a Flg22 peptide from Bacillus thuringiensis, 2) clear excess callose and starch polymers from the plant vasculature through injection of callose- and starch-degrading enzymes, and/or the callose synthesis inhibitor 2-DDG, and 3) improve plant health through delivery of the sulfur-containing amino acid L-cysteine.

Trees were injected at three separate citrus grove sites that had a high prevalence of HLB disease. Treatment trees included 10-year old ‘Ruby Red’ grapefruit (Citrus x paradisi) located at a commercial grove orchard located in central Florida (Okeechobee County) and 8 to 10 year old ‘Valencia’ orange (Citrus sinesis) trees located at two sites, a grove in Eustis, Fla. (Lake County) and a grove in central Florida (Okeechobee County). Individual and combinations of citrus treatments in Table 31 below were injected using a low-pressure injection device, BRANDT ENTREE (BRANDT) into the trunk of the citrus trees using the methods as previously described in Example 1. For all treatments, 10 trees were used per treatment, separated into two replicated blocks of five trees each. To assess the effects of citrus recovery treatments on fruit yield and quality, ‘Ruby Red’ grapefruit were harvested 20 months post-treatment, and the ‘Valencia’ oranges (Okeechobee and Lake Counties) were harvested approximately 22 months post-treatment. Fruit were harvested and assessed according the same metrics of total “Fruit Count” and “Fruit Weight”, “Average Weight per Fruit”, “Average Fruit Diameter” and “% Drop” as described in Example 1, “Acid-corrected ^{o}Brix (^{o}Brix_{c})”, “Citric Acid %”, “% Juice Content” as described as in Example 2, with the exception that one set of grapefruit consisted of 25 total fruit, sampled as 5 fruit each from 5 trees. One set of 25 grapefruit were assessed per replicated treatment block, for a total of two sets per treatment.

TABLE 31
Enzyme and peptide sequences for citrus trunk injection treatments

| | | | Amount of |
|---|---|---|---|
| | Active Ingredient | | Active |
| Treatment | (Sequence or | Treatment | Ingredient |
| No: | CAS Number) | Method | per tree |
| Citrus | β-1,3-endoglucanase | Trunk Injection | 660-2640 |
| Recovery | from Hordium vulgare | | Units enzyme |
| Treatment 1 | (SEQ ID NO: 731) | | per tree |
| Citrus | β-1,3-endoglucanase | Trunk Injection | 660-2640 |
| Recovery | from Paenibacillus | | Units enzyme |
| Treatment 2 | (SEQ ID NO: 732) | | per tree |
| Citrus | Amylase (amyE) | Trunk Injection | 660 Units |
| Recovery | from Bacillus | | enzyme per |
| Treatment 4 | licheniformis | | tree |
| | (SEQ ID NO: 735) | | |
| Citrus | Bt.4Q7Flg22 | Trunk Injection | 0.33-3.3 mg |
| Recovery | from Bacillus | or Foliar | per tree |
| Treatment 5 | thuringiensis | Spray | |
| | strain 4Q7 | | |
| | SEQ ID NO: 226 | | |
| Citrus | L-cysteine | Trunk Injection | 0.266 mg per |
| Recovery | (CAS 52-90-4) | | tree |
| Treatment 6 | | | |
| Citrus | 2-Deoxy-D-Glucose | Trunk Injection | 111.1 mg per |
| Recovery | (2-DDG; | | tree |
| Treatment 7 | CAS 154-17-6) | | |

TABLE 32
Trunk injection of recovery compositions increased ‘Grapefruit’
fruit yield relative untreated trees

| | Average Yield |
|---|---|
| | (kg per tree) |
| | (Relative |
| Treatment | to Untreated) |
| Untreated | 34.6 |
| | — |
| Bt.4Q7Flg22 | 37.2 |
| (SEQ ID NO: 226) | (108%) |
| 0.33 mg/tree) | |
| β-1,3-Endoglucanase from Barley (SEQ ID NO: 731) | 38.7 |
| (2600 U/tree) | (112%) |
| Recovery Enzyme Mixture | 35.1 |
| β-1,3-Endoglucanase from Barley (SEQ ID NO: 731) | (102%) |
| (660 U/tree) + Amylase from Bacillus licheniformis (SEQ | |
| ID NO: 735) (660 U/tree) + Cysteine (0.27 mg/tree) | |

Harvest results from replicated ‘Ruby Red’ grapefruit trials (Table 32) indicated that individual trunk injection treatments to activate the plant immune system, degrade polysaccharides in and around sieve tube elements, and provide essential amino acids for sustained defense responses (L-cysteine) lead to increased fruit yield. Bt.4Q7Flg22 injection at 0.33 mg per tree increased yield 2.6 kg per tree compared to the untreated control, while the callose-degrading enzyme β,1-3-endoglucanase from Barley increased yield 4.1 kg per tree. A Recovery Enzyme Mixture including β,1-3-endoglucanase, starch-degrading Amylase, and L-Cysteine increased yield 0.5 kg per tree which was equivalent to a 2% increase compared to the untreated control.

TABLE 33
Trunk injection of recovery compositions increased ‘Grapefruit’ juice
quantity relative to untreated trees

| | | Average | | |
|---|---|---|---|---|
| | Average | Juice | | |
| | Fruit | Volume | | |
| | Diameter | per fruit | | |
| | (mm) | (mL) | Average | |
| | (Relative | (Relative | Juice | |
| | to | to | Content | Brix:CA |
| Treatment | Untreated) | Untreated | (%) | Ratio |
| Untreated | 82.17 | 134.9 | 59% | 8.90 |
| | — | — | | |
| Bt.4Q7Flg22 | 85.03 | 163.6 | 68% | 9.24 |
| (SEQ ID NO: 226) | (103%) | (121%) | | |
| 0.33 mg/tree) | | | | |
| Recovery Enzyme Mixture | 87.68 | 156.7 | 59% | 8.51 |
| B-1,3-Endoglucanase | (107%) | (116%) | | |
| Hordeum vulgare (SEQ ID | | | | |
| NO: 731) | | | | |
| (660 U/tree) + Amylase from | | | | |
| Bacillus licheniformis (SEQ | | | | |
| ID NO: 735) (660 U/tree) + | | | | |
| Cysteine (0.27 mg/tree) | | | | |

Fruit quality assessments of the ‘Ruby Red’ grapefruit harvested in December 2018 indicate that Bt.4Q7Flg22 (0.33 mg per tree) increased fruit size, juice volume, % juice content and ^{o}Brix_{c} to Citric Acid ratio (Brix:CA) relative to the untreated control. The Recovery Enzyme Mixture injection increased overall fruit size and juice volume relative to the untreated control (Table 33).

### Example 4: Reactive Oxygen Species (ROS) Production of a Flg22 Peptide with Inducer Compounds for Use in the Treatment of Citrus Disease

Combinations of Flg22 with inducer compounds that were used to restore plant health and increase fruit yield in HLB-infected citrus trees by the restriction of CLas bacterial growth and the reduction of phloem-blocking callose polysaccharides were further examined for the activation of the plant immune system using in reactive oxygen species (ROS) assays. To test for compatibility between Flg22 peptide combinations with inducer compounds, tank mixes between native Bt.4Q7Flg22 (SEQ ID NO: 226) and L-Cysteine or 2-Deoxy-D-Glucose (2-DDG) were tested for the ability to enhance Flg22-induced ROS production. Flg22 combinations with inducer compounds in the ROS activity assays were selected to model co-injection of the citrus trees used for Hamlin oranges (Examples 1 to 2). Combination concentrations of Bt.4Q7Flg22 with inducer compounds, L-Cysteine or 2-Deoxy-D-Glucose (2-DDG) were matched to injection volumes as used for the injection of Hamlin oranges in Table 30, such that the injection rates per tree were calculated using the assumption that the phloem content is 1 L in volume. Final concentrations for Bt.4Q7Flg22 (SEQ ID NO: 226) (0.12 ppm); L-cysteine (0.06 g/L) and 2-DDG (0.1 g/L) were used in the ROS activity assays. Fresh plant tissues from soybean (variety MorSoyXtra 38X52) leaves were cut into uniform samples and floated on 150 μL of sterile water in a 96-well white, low luminescence plate. For soybean samples, fully expanded trifoliate leaves were removed from V1-V3 stage plants. Leaf discs (12.6 mm²) were cut from the leaf blades using a 4-mm diameter clean, sharpened cork borer. Discs were cut in half using a clean razor blade, and each disc half was placed in an individual well of the 96-well plate. The plate was placed under growth lights that had a 16-hour light/8-hour dark cycles at a consistent temperature of 22° C. After 18-24 hours, the water was removed from each well of the 96 well plate. Plant tissue samples were treated with a 100 μL elicitation solution containing 34 μg/mL luminol, 20 μg/mL horseradish peroxidase, and the indicated concentration of Bt.4Q7 Flg22 (SEQ ID NO: 226) alone or in combination with L-Cysteine or 2-DDG. Recognition of the Flg22 polypeptide by the plant tissue resulted in activation of immune signaling and the production of apoplastic reactive oxygen species (ROS). In the presence of ROS (H₂O₂), horseradish peroxidase catalyzed the oxidation of luminol and production of visible light. Relative light units (RLUs) were recorded with a SpectraMaxL luminometer using a 0.5 s integration and 2.0 min intervals over a time course of 40 minutes. For data analysis, the average RLU value 14.5 min post-treatment is reported (n=4 samples per treatment).

TABLE 34
ROS activity assays of Bt.4Q7Flg22 peptide used in combination with
inducer compounds

| | Relative Light Units | Fold Change |
|---|---|---|
| Treatment | 14.5 min post- | relative to |
| (n = 4 leaf samples per treatment) | treatment | Treatment A |
| Untreated | 516 | 0.028 X |
| Treatment A | 18456 | 1 X |
| BL4Q7Flg22 | | |
| (SEQ ID NO: 226); 0.12 ppm | | |
| Phosphate Buffer 10 μM | | |
| Treatment B | 17183 | 0.93 X |
| Bt. 4Q7Flg22 | | |
| (SEQ ID NO: 226); 0.12 ppm | | |
| Phosphate Buffer 10 μM + | | |
| 2-deoxy-D-glucose CAS | | |
| 154-17-6 0.1 g/L | | |
| Treatment C | 39018 | 2.11 X |
| Bt. 4Q7Flg22 | | |
| (SEQ ID NO: 226); 0.12 ppm | | |
| Phosphate Buffer 10 μM + | | |
| L-Cysteine CAS 136743-62-9 | | |
| 0.06 g/L | | |

ROS activity assay results (Table 34) indicated that inducer compound, 2-DDG, which is used as a callose synthase inhibitor, provided in combination with the Bt.4Q7Flg22 peptide (Treatment B) resulted in a ROS response that was similar to the response output from the Bt.4Q7Flg22 peptide alone (Treatment A). This was an expected result as 2-DDG used to reduce callose content in the phloem did not contribute directly to increasing the ROS response but also did not hinder the response of the Bt.4Q7Flg22 peptide and therefore found compatible. However, inducer compound, L-Cysteine provided in combination with the Bt.4Q7Flg22 peptide (Treatment C) induced and contributed to the production of ROS in soybean leaf tissue and resulted in a more than 2-fold increase (2.11×) in the production of ROS. The increase in ROS activity for the combination of L-cysteine and Bt.4Q7Flg22 provides an indicator of activation of the plant immune system.

### Example 5: Reactive Oxygen Species (ROS) Production of a Flg22 Peptide with Osmoprotectants for Use in the Treatment of Citrus Disease or Improving Yield in Row Crops

In a separate study, the compatibility between Bt.4Q7Flg22 (SEQ ID NO: 226; Composition 1) and formulations that provide increased plant osmoprotection were tested for production of reactive oxygen species (ROS) and are further described in Table 35 below. Product concentrations in the ROS assay were selected to model co-treatment of Bt.4Q7Flg22 (Composition 1) and osmoprotectant compositions in a carrier volume of 10 gallons per acre for foliar application to crops. ROS production was measured in soybean (variety MorSoyXtra 38X52) using the same methods as previously described in Example 4. Plant tissue samples were treated with a 100 μL elicitation solution containing 34 μg/mL luminol, 20 μg/mL horseradish peroxidase, and the indicated concentration of Bt.4Q7Flg22 alone (Treatment A) or in combination with osmoprotectant (Treatments B, C & D) containing betaine and/or L-proline. Relative light units (RLUs) were recorded with a SpectraMaxL luminometer using a 0.5 s integration and 2.0 min intervals over a time course of 40 minutes. For data analysis, the average total RLU over a 40 minute time course is reported (n=4 samples per treatment) in Table 35.

TABLE 35
ROS activity assays with Bt. 4Q7Flg22 and Osmoprotectants

| | Average Total | Fold Change |
|---|---|---|
| | Relative | relative to |
| Treatment | Light Units | Treatment A |
| Untreated Control | 40372 | 0.01 |
| Treatment A | 275142 | 1 |
| BL4Q7Flg22 (SEQ ID NO: 226) | | |
| 0.12 ppm (Composition 1) | | |
| Phosphate Buffer 0.01 mM | | |
| PROXEL BC preservative: 1 μM | | |
| (BIT) | | |
| Treatment B | 308932 | 1.12 X |
| BL4Q7Flg22 (SEQ ID NO: 226) | | |
| 0.12 ppm | | |
| Phosphate Buffer 0.01 mM | | |
| PROXEL BC preservative: 1 μM | | |
| (BIT) + | | |
| Osmoprotectant 0.25% | | |
| [83.49 mM Betaine-HCl | | |
| 3.3% Tribasic Potassium Phosphate | | |
| 37.9% Non-ionic surfactant(alkyl | | |
| phenol ethoxylate); PROXEL BD20 | | |
| preservative: 6.4 mM (BIT)] | | |
| Treatment C | 323822 | 1.18 X |
| BL4Q7Flg22 (SEQ ID NO: 226) | | |
| 0.12 ppm | | |
| Phosphate Buffer 0.01 mM | | |
| PROXEL BC preservative: 1 μM | | |
| (BIT) + | | |
| Osmoprotectant 0.25% | | |
| [163.88 mM L-Proline | | |
| 3.3% Tribasic Potassium Phosphate | | |
| 37.9% Non-ionic surfactant(alkyl | | |
| phenol ethoxylate); PROXEL BD20 | | |
| preservative:6.4 mM (BIT)] | | |
| Treatment D | 287127 | 1.04 X |
| Bt. 4Q7Flg22 (SEQ ID NO: 226) | | |
| 0.12 ppm | | |
| Phosphate Buffer 0.01 mM | | |
| Proxel ™ BC preservative: 1 μM | | |
| (BIT + | | |
| Osmoprotectant 0.25% | | |
| [83.49 mM Betaine-HCl | | |
| 163.88 mM L-Proline | | |
| 3.3% Tribasic Potassium Phosphate | | |
| 37.9% Non-ionic surfactant(alkyl | | |
| phenol ethoxylate); PROXEL BD20 | | |
| preservative:6.4 mM (BIT)] | | |

ROS activity assay results indicated that formulated combinations with osmoprotectants (Treatments B, C and D) are compatible with the Bt.4Q7Flg22-induced production of ROS in soybean leaf tissues, which is an indicator of activation of the plant immune system. Co-treatment of soybean leaf tissue with Bt.4Q7Flg22 with the osmoprotectants as described in treatments B, C and increased the production of ROS 1.04- to 1.18-fold over the Bt.4Q7Flg22 (Treatment A) alone.

### Examples 6-9: Foliar Compositions of Flg22 Peptides Used in Combinations with Osmoprotectant and ACC Deaminase to Increase Yield in Row Crops, Corn and Soybean

In the following examples, a series of treatment compositions comprising a combination of a Flg22 peptide, osmoprotectant and/or ACC deaminase were tested on row crops (corn and soybean) to measure their effect on yield. For ease of reference, the compositions used and application method/use rate are summarized in Table 36 below.

TABLE 36
Compositions of row crop treatments

| | | | Application |
|---|---|---|---|
| | | | Use Rate Fluid |
| Composition | | Treatment | ounce/acre |
| No: | Formulation | Method | (Fl. oz/Ac) |
| Row Crop | Bt.4Q7Flg22 (SEQ ID NO: | Foliar | 4.0 oz/Ac |
| Composition 1 | 226) 20 ppm | Spray | |
| | 1.67 mM Sodium Phosphate | | |
| | Buffer, pH 5.7 | | |
| | PROXELBC preservative: | | |
| | 330.7 μM (BIT); 53.5 μM | | |
| | (CMIT); 26.1 μM (MIT) | | |
| Row Crop | Osmoprotectant | Foliar | 3.2 oz/Ac |
| Composition 2 | 83.49 mM Betaine-HCl | Spray | |
| | 3.3% Tribasic Potassium | | |
| | Phosphate | | |
| | 37.9% Non-ionic surfactant | | |
| | (alkyl phenol ethoxylate) | | |
| | PROXEL BD20 preservative: | | |
| | 6.38 mM (BIT) | | |
| Row Crop | ACC Deaminase [SEQ ID | Foliar | 8.0 oz/Ac |
| Composition 3 | NO: 730] | Spray | |
| | Fermentation broth filtrate | | |
| | with immobilized enzyme | | |
| | [PROXELBC preservative: | | |
| | 330.7 μM (BIT); 53.5 μM | | |
| | (CMIT); 26.1 μM (MIT) | | |
| Row Crop | Osmoprotectant | Foliar | 3.2 oz/Ac |
| Composition 4 | 163.88 mM Proline | Spray | |
| | 3.3% Tribasic Potassium | | |
| | Phosphate | | |
| | 40 mM Phosphoric Acid | | |
| | 37.9% Non-ionic surfactant | | |
| | (alkyl phenol ethoxylate) | | |
| | PROXEL BD20 preservative: | | |
| | 6.38 mM (BIT) | | |

### Example 6: Foliar Application of Bt.4Q7Flg22 (SEQ ID NO:1) (Composition 1) in Combination with Osmoprotectant (Composition 2) to V4-V7 Corn

Foliar treatments with Bt.4Q7Flg22 (SEQ ID NO: 226) (Row Composition 1), in combination with Osmoprotectant (Row Composition 2), were conducted to determine if synergistic effects resulted from the combination of the treatments. Yield impact of foliar spray applications of Bt.4Q7Flg22 (SEQ ID NO: 226) (Row Composition 1) provided in combination treatments with an Osmoprotectant (Row Composition 2) was assessed on corn plants (hybrids: DKC 60-88 RIB and DKC 58-08 RIB) at the V4-V7 stage of development.

Replicated trials were conducted at 10 locations throughout the US Midwest (IL, IA, NE) testing on two hybrids per location. Field seed beds at each location were prepared using conventional or conservation tillage methods for corn plantings. Fertilizer was applied as recommended by conventional farming practices and remained consistent between the US Midwest locations. Herbicides were applied for weed control and supplemented with cultivation when necessary. Four-row plots, 17.5 feet (5.3 meters) long were planted at all locations. Corn seed was planted 1.5 to 2 inches (3.8 to 5.1 cm) deep, to ensure normal root development, at 28,000 to 36,000 plants per acre with row widths of 30 inch (76.2 cm)-seed spacing of approximately 1.6 to 1.8 seeds per foot. Each hybrid was grown in at least three separate plots (replicates) per treatment at each location, to account for field variability. Plots were maintained using the individual grower's production practices.

Native Bt.4Q7Flg22 bioactive priming polypeptide (SEQ ID NO: 226) (Composition 1) was chemically synthesized via solid phase peptide synthesis and formulated at 4 Fl. oz/Ac (292.1 mL/hectare, Ha) use rate. The final concentration in the spray tank was 42 ppb after dilution in carrier rate of 15 gallons water/Ac, GPA (37.85 L/Ha). Native Bt.4Q7Flg22 bioactive priming polypeptide (SEQ ID NO: 226) (Composition 1) was applied alone and in combination with Osmoprotectant (Composition 2). Osmoprotectant (Composition 2) was formulated at a 3.2 Fl. oz/Ac (233.7 mL/Hectare, Ha) use rate. The combination was applied at the V4-V7 development stage with a non-ionic surfactant at 0.067% v/v (final concentration in spray tank).

Corn yield in bushels per acre (Bu/Ac) was reported at all locations as an average yield of each treatment's replicates across each hybrid. The effect of Bt.4Q7Flg22 (SEQ ID NO: 226) (Composition 1) in combination with Osmoprotectant (Composition 2) was normalized to the average yield for the surfactant control plots for the 10 locations (Table 37). Additionally, the win rate was calculated: the percentage of testing locations at which one treatment has a yield advantage over other treatments (in this case, as compared to the surfactant-control plants).

Foliar treatment Bt.4Q7Flg22 (SEQ ID NO: 226) (Composition 1) in combination with Osmoprotectant (Composition 2) provided yield benefits over the surfactant-treated control corn plants with an average+8.70 Bu/Ac (546 kg/Ha) increase observed across the 10 locations with a 60% win rate over surfactant-control. The Bt.4Q7Flg22 combination treatment with the Osmoprotectant resulted in yield increases in corn greater than the additive increases of these treatments applied individually (Table 37).

TABLE 37
Foliar treatment of corn with Bt.4Q7Flg22 (Composition 1), in
combination with Osmoprotectant (Composition 2) to increase
yield in corn

| | Application | Average Total | Average Bu/Ac |
|---|---|---|---|
| | Use Rate | Yield Bu/Ac | Increase compared |
| Treatment—Corn | Fl. oz/Ac | (10 locations) | to Control |
| Surfactant Control | — | 223.21 | — |
| BL4Q7Flg22 | 4.0 | 224.67 | +1.42 |
| (Composition 1) | | | |
| Osmoprotectant | 3.2 | 229.53 | +6.32 |
| (Composition 2) | | | |
| BL4Q7Flg22 | 4.0 | 231.91 | +8.70 |
| (Composition 1) + | | | |
| Osmoprotectant | 3.2 | | |
| (Composition 2) | | | |

### Example 7: Foliar Application of Bt4Q7Flg22 (Composition 1) in Combination with ACC Deaminase (Composition 3) to V4-V7 Corn

Foliar treatments with Bt.4Q7Flg22 (SEQ ID NO: 226) (Composition 1), in combination with ACC Deaminase (Composition 3), were conducted to determine if synergistic effects resulted from the combination of the treatments. Yield impact of foliar spray application of Bt.4Q7Flg22 in combination with ACC Deaminase (Composition 3) was assessed on corn plants (hybrids: DKC 60-88 RIB, DKC 58-08 RIB and DKC 64-35 RIB) at the V4-V7 stage of development.

Replicated trials were conducted at 12 locations throughout the US Midwest (IL, IA, NE). Corn plants were grown as described in Example 6.

Bt.4Q7Flg22 (SEQ ID NO: 226) (Composition 1) was prepared and applied as in Example 6, alone and in combination with ACC Deaminase (Composition 3). ACC Deaminase (Composition 3) was formulated at 8 Fl. oz/Ac (584.2 mL/Hectare, Ha) use rate. The combination was applied at the V4-V7 development stage with a non-ionic surfactant at 0.1% v/v (final concentration in spray tank).

Corn yield in bushels per acre (Bu/Ac) and win rate was calculated as for Example 6.

Foliar treatment Bt.4Q7Flg22 (SEQ ID NO: 226) (Composition 1) in combination with ACC Deaminase (SEQ ID NO: 730) (Composition 3) provided yield benefits over the untreated control corn plants with an average+10.64 Bu/Ac (667.9 kg/Ha) increase observed across the 12 locations with a 75% win rate over surfactant-control. The combination treatments of Bt.4Q7Flg22 and ACC deaminase resulted in a synergistic increase in yield of greater than the individual foliar treatments with Bt.4Q7Flg22 (+4.37 Bu/Ac) and ACC deaminase (+1.06 Bu/Ac), (Table 38).

TABLE 38
Foliar treatment of corn with Bt 4Q7Flg22 (SEQ ID NO: 226)
(Composition 1), in combination with ACC Deaminase (Composition
3) to increase yield in corn

| | Application | Average Total | Average Bu/Ac |
|---|---|---|---|
| | Use Rate | Yield Bu/Ac | Increase compared |
| Treatment—Corn | Fl. oz/Ac | (12 locations) | to Control |
| Surfactant Control | — | 225.51 | — |
| BL4Q7Flg22 | 4.0 | 229.88 | +4.37 |
| (SEQ ID NO: 226) | | | |
| (Composition 1) | | | |
| ACC Deaminase | 8.0 | 226.57 | +1.06 |
| (SEQ ID NO: 730) | | | |
| (Composition 3) | | | |
| BL4Q7Flg22 | 4.0 | 236.15 | +10.64 |
| (SEQ ID NO: 226) | | | |
| (Composition 1) + | | | |
| ACC Deaminase | 8.0 | | |
| (SEQ ID NO: 730) | | | |
| (Composition 3) | | | |

### Example 8: Foliar Application of Bt.4Q7Flg22 (Composition 1) in Combination with Osmoprotectant (Composition 2) to V3-V6 Soybean

Foliar applications using native Bt.4Q7Flg22 (SEQ ID NO: 226) (Composition 1), in combination with Osmoprotectant (Composition 2), were conducted to determine if synergistic effects resulted from the combination of the treatments. Yield impact of foliar spray application was assessed on soybean plants (varieties: AG35X7, AG41X8, AG27X7 and AG30X6), at the V3-V6 stage of development.

Replicated trials were conducted at 14 locations throughout the US Midwest (IL, IA, NE) Soybean seed varieties were planted 1.5 to 2 inches (3.8 to 5.1 cm) deep to assure normal root development. Soybean seed was planted at approximately on average 150,000 plants per acre with row widths of 30 inch (76.2 cm) rows—seed spacing of approximately 7 to 8 seeds per foot (0.3 meter). Plots were maintained using the individual grower's production practices.

Bt.4Q7Flg22 (SEQ ID NO:226) (Composition 1) was applied alone and in combination with Osmoprotectant (Composition 2) as for Example 6. The combination was applied at the V3-V6 development stage with a non-ionic surfactant at 0.067% v/v (final concentration in spray tank).

Soybean yield in bushels per acre (Bu/Ac) and win rate was calculated as for Example 6.

Foliar treatment Bt.4Q7Flg22 (SEQ ID NO:226) (Composition 1) in combination with Osmoprotectant (Composition 2) provided yield benefits over the surfactant-treated control soybean plants with an average+1.94 Bu/Ac (126.4 kg/Ha) increase observed across the 14 locations with an 86% win rate over surfactant-control. The Bt.4Q7Flg22 (Composition 1) combination treatment with the Osmoprotectant (Composition 2) resulted in increased yield in soybean over the Bt.4Q7Flg22 or Osmoprotectant treatments applied alone (Table 39).

TABLE 39
Foliar treatment of soybean with Bt.4Q7Flg22 (Composition 1),
in combination with Osmoprotectant (Composition 2) to
increase yield in soybean

| | Application | Average Total | Average Bu/Ac |
|---|---|---|---|
| | Use Rate | Yield Bu/Ac | Increase compared |
| Treatment—Soybean | Fl. oz/Ac | (14 locations) | to Control |
| Surfactant Control | — | 64.69 | — |
| BL4Q7Flg22 | 4.0 | 64.97 | +0.29 |
| (SEQ ID NO: 226) | | | |
| (Composition 1) | | | |
| Osmoprotectant | 3.2 | 65.97 | +1.28 |
| (Composition 2) | | | |
| BL4Q7Flg22 | 4.0 | 64.05 | +1.94 |
| (SEQ ID NO: 226) | | | |
| (Composition 1) + | | | |
| Osmoprotectant | 3.2 | | |
| (Composition 2) | | | |

### Example 9: Foliar Application of Bt.4Q7Flg22 (Composition 1) in Combination with a Herbicide to V3-V6 Soybean

Foliar applications using native Bt.4Q7Flg22 (SEQ ID NO: 226) (Composition 1), in combination with a broad weed control herbicide with the active ingredient lactofen (24% w/v) were conducted to determine if the application of the Bt.4Q7Flg22 peptide with a herbicide used for broad weed control and protection against white mold infection could provide a beneficial yield advantage compared to that achieved from the herbicide alone. Yield impact of foliar spray application of Bt.4Q7Flg22 (SEQ ID NO:226) (Composition 1) in combination with lactofen was determined on soybean plants (varieties: AG35X7, AG41X8) at the V3-V6 stage of development. Large-scale, replicated soybean trials were conducted at 4 locations in the Midwestern US (KS, MO, IL), with planting as for Example 8.

TABLE 40
Foliar treatment of soybean with Bt. 4Q7Flg22 (Composition 1), in
combination with an herbicide to increase yield in soybean

| | Application | Average Total | Average Bu/Ac |
|---|---|---|---|
| | Use Rate | Yield Bu/Ac | Increase compared |
| Treatment—Soybean | Fl. oz/Ac | (4 locations) | to Herbicide Alone |
| Lactofen (24% w/v) | 10 | 66.10 | — |
| Herbicide | | | |
| BL4Q7Flg22 | 4 | 67.73 | +1.62 |
| (SEQ ID NO:226) + | | | |
| Lactofen (24% w/v) | 10 | | |
| Herbicide | | | |

Foliar treatment Bt.4Q7Flg22 (Composition 1) provided in combination with a broad weed control herbicide with the active ingredient lactofen increased yield+1.62 Bu/Ac (109 kg/Ha) f as compared to the herbicide treatment applied alone (Table 40), indicating that Bt.4Q7Flg22 treatment is compatible with lactofen. Provided together, Bt.4Q7Flg22 and lactofen would be expected to improve soybean resistance to white mold.

### Example 10: Additive and Synergistic Effects Between Flagellins and Recovery Enzyme Mixture

To test for additive and/or synergistic effects between combinations of Bt.4Q7Flg22 and the Recovery Enzyme Mixture (Composition Table 41), trials will be established in 5 to 10-year old Navel, Hamlin and/or Valencia orange trees and Ruby red grapefruit trees. Ten trees will be treated for each composition (described in Table 41), arranged into two replicated blocks of 5 trees each, using methods described in Example 1 for tree injection and foliar spray. Fruit will be harvested and fruit quality assessed as described in Example 1 and Example 2.

TABLE 41
Synergistic effects between combinations of Bt.4Q7Flg22 and the
recovery enzyme mixtures

| | | | Application | |
|---|---|---|---|---|
| | | | Use Rate | |
| | | | Milliliters | |
| | | | per tree | |
| | | | (mL/tree) or | |
| | | | Fluid | |
| Composition | | Treatment | ounce/acre | |
| No: | Formulation | Method | (Fl. oz/Ac) | |
| Untreated | — | — | — | |
| Citrus | Bt.4Q7Flg22 | Foliar Spray or | 0.33 | mg/tree |
| Composition 1 | (SEQ ID NO: 226) | Trunk Injection | | |
| Citrus | Bt.4Q7Flg22 | Foliar Spray or | 0.33 | mg/tree |
| Composition 2 | (SEQ ID NO: 226) | Trunk Injection | | |
| | L-Cysteine | Trunk Injection | 60 | mg/tree |
| | B-1,3-Endoglucanase | Trunk Injection | 500 | U/tree |
| Citrus | Bt.4Q7Flg22 | Foliar Spray or | 0.33 | mg/tree |
| Composition 3 | (SEQ ID NO: 226) | Trunk Injection | | |
| | L-Cysteine | Trunk Injection | 60 | mg/tree |
| | Amylase | Trunk Injection | 500 | U/tree |
| Citrus | Bt.4Q7Flg22 | Foliar Spray or | 0.33 | mg/tree |
| Composition 4 | (SEQ ID NO: 226) | Trunk Injection | | |
| | L-Cysteine | Trunk Injection | 60 | mg/tree |
| | 2-DDG | Trunk Injection | 100 | mg/tree |
| Citrus | Bt.4Q7Flg22 | Foliar Spray or | 0.33 | mg/tree |
| Composition 5 | (SEQ ID NO: 226) | Trunk Injection | | |
| | L-Cysteine | Trunk Injection | 60 | mg/tree |
| | B-1,3-Endoglucanase | Trunk Injection | 500 | U/tree |
| | Amylase | Trunk Injection | 500 | U/tree |
| | L-Cysteine | Trunk Injection | 60 | mg/tree |
| Citrus | B-1,3-Endoglucanase | Trunk Injection | 500 | U/tree |
| Composition 6 | Amylase | Trunk Injection | 500 | U/tree |
| | 2-DDG | Trunk Injection | 100 | mg/tree |
| Citrus | Bt.4Q7Flg22 | Foliar Spray or | 0.33 | mg/tree |
| Composition 7 | (SEQ ID NO: 226) | Trunk Injection | | |
| | L-Cysteine | Trunk Injection | 60 | mg/tree |
| | B-1,3-Endoglucanase | Trunk Injection | 500 | U/tree |
| | Amylase | Trunk Injection | 500 | U/tree |
| | 2-DDG | Trunk Injection | 100 | mg/tree |

### Examples 11-12

In the following Examples 11-12, a series of treatment compositions comprising a combination of a Flg22 peptide, osmoprotectant and/or ACC deaminase were tested on row crops (corn and soybean) to measure their effect on yield. For ease of reference, the compositions used are the same compositions as described in Table 42 for compositions 1, 3, 5 and 7. The compositions used, application method, and usage rates are summarized in Table 42 below.

TABLE 42
Compositions of row crop treatments for corn and soy application

| | | | Application |
|---|---|---|---|
| | | | Use Rate |
| | | | Fluid |
| Composition | | Treatment | ounce/acre |
| No: | Formulation | Method | (Fl. oz/Ac) |
| Row Crop | Bt.4Q7Flg22 (SEQ ID NO: | Foliar Spray | 4.0 oz/Ac |
| Composition 1 | 226) 20 ppm | or | Foliar or |
| | 1.67 mM Sodium Phosphate | In Furrow | 4.0 oz/Ac |
| | Buffer, pH 5.7 | Treatment | In Furrow |
| | PROXELBC preservative: | | |
| | 330.7 μM (BIT); 53.5 μM | | |
| | (CMIT); 26.1 μM (MIT) | | |
| Row Crop | ACC Deaminase [SEQ ID | In furrow | 8 oz/Ac |
| Composition 3 | NO: 730] | treatment | In Furrow |
| | Fermentation broth filtrate | | |
| | with immobilized enzyme | | |
| | PROXELBC preservative: | | |
| | 330.7 μM (BIT); 53.5 μM | | |
| | (CMIT); 26.1 μM (MIT) | | |
| Row Crop | Osmoprotectant | Foliar Spray | 3.2 oz/Ac |
| Composition 5 | 83.49 mM Betaine-HCl | | Foliar |
| | 163.88 mM L-Proline | | |
| | 3.3% Tribasic Potassium | | |
| | Phosphate | | |
| | 37.9% Non-ionic surfactant | | |
| | (alkyl phenol ethoxylate) | | |
| | PROXEL BD20 preservative: | | |
| | 6.38 mM (BIT) | | |
| Row Crop | Bt.4Q7Flg22 (SEQ ID NO: | Seed | 0.14 fl |
| Composition 6 | 226) 48 ppm | Treatment | oz/unit |
| | 4 mM Sodium Phosphate | | seed |
| | Buffer, pH 5.7 | | |
| | PROXELBC preservative: | | |
| | 330.7 μM (BIT); 53.5 μM | | |
| | (CMIT); 26.1 μM (MIT) | | |
| Row Crop | ACC Deaminase [SEQ ID | Seed | 30 mU/unit |
| Composition 7 | NO: 730] | Treatment | seed |
| | Bacterial cell lysate with free | | |
| | enzyme | | |

### Example 11: Foliar Application of Bt.4Q7Flg22 (SEQ ID NO: 226) Peptide in Combination with Osmoprotectants to Increase Soybean Yield

Foliar application of Bt.4Q7Flg22 (SEQ ID NO: 226) peptide and an osmoprotectant formulation with two active ingredient, L-Proline and Betaine, were applied to soybeans as a foliar spray to test potential for the combined treatment to increase grain yield.

Replicated trials were conducted at 11 sites throughout the US Midwest (MN, IL, IA, MO), on the following soybean varieties, AG2733, AG3931, P21a28x, and P40a47x, with one variety per site. Soybean seeds were planted and test plots established as for Example 8.

Bt.4Q7Flg22 (SEQ ID NO: 226) (Table 42; Row Crop Composition 1) was applied alone and in combination with the Osmoprotectant formulation (Table 42; Row Crop Composition 5), at soybean R2 development stage with a non-ionic surfactant at 0.067% v/v (final concentration in spray tank). Soybean yield in bushels per acre (Bu/Ac) was reported for each experimental replicate, and average change in Bu/Ac and win rate (Table 43) were calculated across all sites as described in Example 6.

TABLE 43
Foliar treatment of soybean with Bt.4Q7Flg22 (SEQ ID No: 226)
and Osmoprotectants increased yield in soybean

| | | | Average | Average |
|---|---|---|---|---|
| | | | Total | Bu/Ac |
| | | Application | Yield | Increase |
| | Treatment | Use Rate | Bu/Ac | compared |
| Treatment—Soybean | Method | Fl. oz/Ac | (11 sites) | to Control |
| Untreated Control | — | — | 65.16 | — |
| Bt.4Q7Flg22 | Foliar | 4.0 | 66.60 | +1.44 |
| (SEQ ID NO: 226) | Spray | | | |
| (Row Crop | | | | |
| Composition 1) | | | | |
| Osmoprotectant | Foliar | 3.2 | 67.82 | +2.66 |
| (Row Crop | Spray | | | |
| Composition 5) | | | | |
| Bt. 4Q7Flg22 | Foliar | 4.0 | 68.16 | +3.00 |
| (SEQ ID NO: 226) | Spray | | | |
| (Row Crop | | | | |
| Composition 1) + | | | | |
| Osmoprotectant | | 3.2 | | |
| (Row Crop | | | | |
| Composition 5) | | | | |

Foliar treatment of Bt.4Q7Flg22 (SEQ ID NO: 226) (Row Crop Composition 1) in combination with Osmoprotectant (Row Crop Composition 5) provided yield benefits over the untreated control soybean plants with an average+3.00 Bu/Ac across the 11 sites, with a 73% win-rate over untreated control. The observed yield for the combination treatment exceeded the yield for each composition alone, +1.44 Bu/Ac and +2.66 Bu/Ac for Row Crop Composition 1 and 5, respectively, demonstrating increased benefit for combinations of the bioactive peptide provided with an osmoprotectant including a betaine or proline during reproductive stages (this example) and vegetative stages (Example 8).

### Example 12: In Furrow or Seed Treatment of Corn with Bt.4Q7Flg22 (SEQ ID NO:226) in Combination with ACC Deaminase (SEQ ID NO: 730) to Increase Yield

In furrow treatments with Bt.4Q7Flg22 (SEQ ID NO: 226) (Row Crop Composition 1), in combination with ACC deaminase (SEQ ID NO: 730) (Row Crop Composition 3), were conducted to determine if synergistic or additive effects resulted from the combination of the treatments. Yield impact of combination in furrow treatments was assessed on corn plants, hybrid P1197.

Replicated trials were conducted at 4 locations throughout the US Midwest (KS, MO, IL). Field seed beds at each location were prepared using conventional or conservation tillage methods for corn plantings. Fertilizer was applied as recommended by conventional farming practices and remained consistent between the US Midwest locations. A base treatment of 10-34-0 fertilizer at the rate of 2.5 gallons per acre was applied along with all treatments, including the control treatment. Herbicides were applied for weed control. Four-row plots, 39 feet (11.9 meters) long were planted at all locations. Corn seed was planted 1.75 to 2.25 inches (4.4 to 5.7 cm) deep, to ensure normal root development, at 30,000 to 35,000 plants per acre with row widths of 30 inches (76.2 cm). Three separate plots (replicates) per treatment were grown at each location, to account for field variability. Plots were maintained using the individual grower's production practices.

Bt.4Q7Flg22 bioactive priming polypeptide (SEQ ID NO: 226) (Row Crop Composition 1) or ACC deaminase (Row Crop Composition 3) were either applied alone, or in combination, at the rates indicated in Table 42. Corn yield in bushels per acre (Bu/Ac) was reported for each experimental replicate, and the average change in Bu/Ac and win rate relative to base fertilizer control were calculated across all sites as described in Example 6.

In furrow treatment with Bt.4Q7Flg22 (SEQ ID NO: 226) (Row Crop Composition 1) or ACC deaminase (Row Crop Composition 3) provided yield benefits over the control corn plants with an average of +1.39 Bu/Ac or +2.18 Bu/Ac, respectively (Table 44). Combined treatment resulted in an additive effect, with an observed+3.62 Bu/Ac increase in yield in comparison to the control, with an observed 75% win rate over the control plants. The Bt.4Q7Flg22 combination treatment with the ACC deaminase resulted in yield increases in corn greater than the increases of these treatments applied individually.

TABLE 44
In furrow treatment of corn with Bt.4Q7Flg22 (SEQ ID NO: 226) in
combination with ACC Deaminase to increase yield

| | | | | Average |
|---|---|---|---|---|
| | | | Average | Bu/Ac |
| | | Application | Total | Increase |
| | | Use | Yield | compared |
| Treatment—Corn | Treatment | Rate fl. | Bu/Ac | to |
| In Furrow | Method | oz/Ac | (4 sites) | Control |
| Base | — | — | 219.26 | — |
| (10-34-0 fertilizer; 2.5 | | | | |
| gallons per acre) | | | | |
| Base + Bt.4Q7Flg22 | In Furrow | 4 | 220.65 | +1.39 |
| (SEQ ID NO: 226) | | | | |
| (Row Crop Composition | | | | |
| 1) | | | | |
| Base + ACC Deaminase | In Furrow | 8 | 221.44 | +2.18 |
| [SEQ ID NO: 730] | | | | |
| Fermentation broth filtrate | | | | |
| with immobilized enzyme | | | | |
| (Row Crop Composition | | | | |
| 3) | | | | |
| Base + Bt.4Q7Flg22 | In Furrow | 4 | 222.88 | +3.62 |
| (SEQ ID NO: 226) | | | | |
| (Row Crop Composition | | | | |
| 1) + | | | | |
| ACC Deaminase | | 8 | | |
| [SEQ ID NO: 730] | | | | |
| Fermentation broth | | | | |
| filtrate with | | | | |
| immobilized enzyme | | | | |
| (Row Crop Composition | | | | |
| 3) | | | | |

Trials were established to further test the combination of ACC deaminase (ACCD) (SEQ ID NO: 730) and Bt.4Q7Flg22 (SEQ ID NO: 226) seed treatment applications to corn for increasing yield. For these trials, ACC deaminase (SEQ ID NO: 730) was expressed and purified, and enzyme activity was determined.

Cloning and Expression of pET28a-ACCD Free Enzyme Expression Construct.

The pET28a-ACCD plasmid was created by In Fusion cloning. Insert- and vector-specific PCR primers were used to amplify the pET28a vector and ACC deaminase insert sequences with overlapping ends. The PCR products were digested with the restriction enzyme DpnI for 1 hour at 37° C. Following purification, 20 ng of each DNA fragment was combined with In Fusion Premix (In Fusion Cloning Kit, Takara) and incubated for 15 minutes at 50° C. to create the plasmid sequence. This plasmid was then transformed into Stellar E. coli competent cells (Takara) by heat shock treatment at 42° C. followed by recovery at 37° C. in Luria-Bertani (LB) broth and plating on LB-ampillicin agar plates. The plates were incubated at 37° C. overnight. The construct sequence for transformant colonies was confirmed by PCR and sequencing. The pET28a-ACCD construct was then isolated from the Stellar E. coli cells using the Wizard SV Plus Minipreps DNA Purification System (Promega) and transformed into E. coli Bt.21 via electroporation in a 1 mM cuvette with the following parameters: 2.35 kV, 200 ohms, and 25 μFD. Transformants were selected on LB-kanamycin plates and the sequence of the construct was verified.

To express the ACC deaminase free enzyme in E. coli Bt.21, the cells were grown in LB auto-induction media containing lactose and a small concentration of glucose. Following initial depletion of the glucose, the cells were then induced automatically by the lactose in the culture. Cell lysis was performed with Bugbuster reagent (Millipore Sigma). The lysed cells were dialyzed against phosphate-buffered saline. Protein expression was verified by polyacrylamide gel electrophoresis.

### Determination of ACC Deaminase Enzyme Activity

ACCD enzyme activity was determined by a lactate dehydrogenase (LDH)-coupled assay. This enzyme converts 1-aminocyclopropane carboxylate (ACC) to α-ketobutyrate, which in turn can be reduced with NADH by LDH. The consumption of NADH was monitored by UV-VIS absorbance at 340 nm. The enzyme activity of ACC deaminase was then determined based on the absorbance change, following subtraction of the background.

Corn Seed Treatment with a Composition of ACC Deaminase and Flg22

Corn seed from two hybrids, Beck's 6127 BHMF and 6127 DVMF, were treated with Bt.4Q7Flg22 (SEQ ID NO: 226) (Row Crop Composition 6), ACC deaminase (SEQ ID NO: 730) (Row Crop Composition 7), or a combination of the two bioactive peptides in a standard seed treatment slurry at the rates indicated in Table 42. The standard slurry contained a fungicide, insecticide, beneficial bacteria, colorant and seed finisher (Prothioconazole 76.8 g/L, Metalazyl 61.4 g/L, Penflufen 38.4 g/L (0.031 mg ai/seed), Clothiandin (40.3%) with Bacillus firmus strain 1-1582 (8.10%) (0.6 mg ai/seed), Bacillus thuringiensis strain EX297512 (91.04% bacteria and carrier) (3 fl oz/unit) Peridium Quality 1006 (5 fl oz/cwt) and Pro-Ized Red Colorant (normal) (0.5 fl oz/cwt). Seed treatments were applied using a Wintersteiger HEGE II (Wintersteiger AG, Austria, Germany).

Seed was planted in 7 sites in the US Midwest (IL, IN), and each location was prepared using conventional or conservation tillage methods for corn plantings. Two-row plots, 17.5 feet long were planted at all locations. Corn seed was planted 1.75 to 2.25 inches (4.4 to 5.7 cm) deep, to ensure normal root development, at 30,000 to 35,000 plants per acre with row widths of 30 inches (76.2 cm). Three separate plots (replicates) per treatment were grown at each location, to account for field variability. Plots were maintained using the individual grower's production practices. Corn yield in bushels per acre (Bu/Ac) was reported for each experimental replicate, and the average change in Bu/Ac and win rate relative to base seed treatment control were calculated across all sites as described in Example 6.

Seed treatment with Bt.4Q7Flg22 (SEQ ID NO: 226) (Row Crop Composition 6) or ACC Deaminase (Row Crop Composition 7) provided yield benefits over the control corn plants with an average of +6.44 Bu/Ac or +7.16 Bu/Ac, respectively (Table 45). Combined treatment resulted in increased benefit, with an observed+9.16 Bu/Ac increase in yield in comparison to the seed treatment control, and an observed 71% win rate over the control plants. The Bt.4Q7Flg22 combination treatment with ACC deaminase resulted in yield increases in corn greater than the increases of these treatments applied individually.

TABLE 45
Corn seed treatment with ACC Deaminase free enzyme in combination
with Bt. 4Q7Flg22 to increase yield in corn

| | | | | Average |
|---|---|---|---|---|
| | | | Average | Bu/Ac |
| | | Application | Total | Increase |
| | | Use Rate | Yield | compared |
| Treatment—Corn | Treatment | per | Bu/Ac | to |
| Seed Treatment | Method | unit corn | (7 sites) | Control |
| Base Seed Treatment | — | — | 200.65 | — |
| Control | | | | |
| Base + Bt.4Q7Flg22 | Seed | 0.14 fl oz | 207.09 | +6.44 |
| (SEQ ID NO: 226) | Treatment | | | |
| (Row Crop Composition | | | | |
| 6) | | | | |
| Base + ACC Deaminase | Seed | 30 mU | 207.81 | +7.16 |
| [SEQ ID NO: 730] | Treatment | | | |
| Bacterial cell lysate with | | | | |
| free enzyme | | | | |
| (Row Crop Composition | | | | |
| 7) | | | | |
| Base + Bt.4Q7Flg22 | Seed | 0.14 fl oz | 209.81 | +9.16 |
| (SEQ ID NO: 226) | Treatment | | | |
| (Row Crop Composition | | | | |
| 6) + | | | | |
| ACC Deaminase | | 30 mU | | |
| [SEQ ID NO: 730] | | | | |
| Bacterial cell lysate with | | | | |
| free enzyme (Row Crop | | | | |
| Composition 7) | | | | |

### Example 13: Treatment with GmRHPP or RHPP Peptide Variants Increases Root Hair and Lateral Root Lengths

GmRHPP (SEQ ID NO:604) and RHPP-like peptide variants (SEQ ID NOs: 608, 607 and 745-755) were tested for ability to promote root hair elongation and lateral root elongation. To evaluate root architecture changes, field mustard (Brassica rapa) seeds were surface sterilized then added to bottles containing 10 μM peptide in Murashige and Skoog liquid culture with 0.25 g/L 2-(N-Morpholino) ethanesfulonic acid hydrate (pH 5.8). Field mustard seedlings were grown in liquid-culture bottles for approximately three days. After three days, seedling roots were stained with 0.1% methylene blue in 70% ethanol then imaged using a Dino-lite digital microscope. Lengths of root hairs and lateral roots were quantified in FIJI software using the “line measure tool”.

TABLE 46
Liquid culture treatment with GmRHPP or RHPP variants promotes
elongation of root hairs and lateral roots in liquid-culture grown field
mustard (Brassica rapa) seedlings

| | Experiment A: | Experiment B: Average |
|---|---|---|
| | Average Root Hair | Length of Three Longest |
| Treatment: | Length Per Plant (mm) | Lateral Roots Per Plant (mm) |
| Untreated Control | 0.137 | 0.632 |
| (UTC) | — | — |
| GmRHPP | 0.172 | 0.971 |
| SEQ ID NO: 604 | (125% of UTC) | (154% of UTC) |
| (10 μM) | | |
| RHPP-Gm1 | 0.185 | 1.034 |
| SEQ ID NO: 608 | (135% of UTC) | (164% of UTC) |
| (10 μM) | | |
| RHPP-Gm2 | 0.169 | 0.953 |
| SEQ ID NO: 607 | | |
| (10 μM) | (123% of UTC) | (151% of UTC) |
| RHPP-Pp | 0.145 | 0.853 |
| SEQ ID NO: 745 | (105% of UTC) | (135% of UTC) |
| (10 μM) | | |
| RHPP-Mc | 0.165 | 0.822 |
| SEQ ID NO: 746 | (120% of UTC) | (130% of UTC) |
| (10 μM) | | |
| RHPP-Bd | 0.137 | 0.959 |
| SEQ ID NO: 747 | (99% of UTC) | (152% of UTC) |
| (10 μM) | | |
| RHPP-Va | 0.151 | 0.929 |
| SEQ ID NO: 748 | (110% of UTC) | (147% of UTC) |
| (10 μM) | | |
| RHPP-Ls | 0.182 | 1.058 |
| SEQ ID NO: 749 | (132% of UTC) | (168% of UTC) |
| (10 μM) | | |
| RHPP-Vr | 0.147 | — |
| SEQ ID NO: 750 | (107% of UTC) | — |
| (10 μM) | | |
| Syn01 RHPP | 0.154 | 0.964 |
| SEQ ID NO: 751 | (112% of UTC) | (153%) |
| (10 μM) | | |
| Syn02 RHPP | 0.150 | 0.932 |
| SEQ ID NO: 752 | (109% of UTC) | (148% of UTC) |
| (10 μM) | | |
| Syn03 RHPP | 0.153 | 0.954 |
| SEQ ID NO: 753 | (112% of UTC) | (151% of UTC) |
| (10 μM) | | |
| Syn04 RHPP | 0.164 | — |
| SEQ ID NO: 754 | (120% of UTC) | — |
| (10 μM) | | |
| Syn05 RHPP | 0.152 | 0.932 |
| SEQ ID NO: 755 | (111% of UTC) | (147% of UTC) |
| (10 μM) | | |

Results (Table 46) indicate that GmRHPP (SEQ ID NO: 604) and almost all RHPP peptide variants tested (SEQ ID NOs: 607, 608, 745, 746, 748, 749, 750, 751, 752, 753, 754, 755) increased average root hair length compared to untreated control. The only exception was RHPP-Bd (SEQ ID NO: 747) which did not improve root hair elongation. Treatment with RHPP-Gm1, RHPP-Ls, and GmRHPP peptides correlated to the greatest increases in root hair length relative to control (+35%, +32% & +25%, respectively). Increased average lateral root length compared to untreated control was observed for seedlings treated with GmRHPP (SEQ ID NO: 604) or all tested RHPP peptide variants (SEQ ID NOs: 607, 608, 745, 746, 747, 748, 749, 750, 751, 752, 753, 754, 755). Treatment with RHPP-Ls, RHPP-Gm1, and GmRHPP peptides correlated to the greatest increases in average lateral root length relative to control (+68%, +64% & +54%, respectively). RHPP and RHPP-like peptides can be used to stimulate root growth of soil grown, hydroponic or aeroponic grown plants, to improve nutrient update and increase biomass. RHPP and RHPP-like peptides could be used in combination with other bioactive peptides to stimulate root growth, while also reducing abiotic and biotic stress, and increasing yield.

### Example 14: Gm.RHPP Foliar Application in Combination with Foliar Fungicides Containing a Succinate Dehydrogenase Inhibitor (SDHI) Provides Enhanced Plant Protection Against Phakopsora pachyrhizi

In January 2019, replicated field trials were conducted across three locations in Paraguay (Capitan Meza, Capitan Miranda, Natalio) using a foliar application comprising a Gm.RHPP peptide and a broad-spectrum fungicide Cripton XPro (12.5% Bixafen, 17.5% Prothioconazole, 15% Trifloxystrobin) with three modes of action. Cripton XPro is a commercially available foliar fungicide for preventative and curative treatment of Asian Soybean Rust (ASR) caused by Phakopsora pachyrhizi when applied as a foliar spray following the recommendation on the specimen label at a use rate of 6.84 fluid ounces per acre (Fl. oz/Ac) (500 mL/hectare). Due to rapidly evolving resistance of P. pachyrhizi and other pathogens to triazole class fungicides, new modes of action are needed to extend the efficacy of chemical fungicides and promote crop resistance and yield. Bixafen is an example of a highly effective succinate dehydrogenase inhibitor activity (SDHI) group fungicide, of the pyrazole carboxamide class, that disrupt fungal cell energy production. Trials were established to test co-treatment of plants with the Cripton XPro formulation of bixafen and bioactive peptides, such as Gm.RHPP (SEQ ID NO: 604), for control of ASR.

Beginning at the R1 stage of development, soybean plants received three foliar applications of the compositions described in Table 47 with an interval of 10-14 days between applications. Foliar applications were applied to soybeans, with four replicated plots (3×10 meters, 30 m2; 6 rows) per treatment at each independent sites (total of 12 replicates per treatment) Plants became naturally infected with P. pachyrhizi through environmental pressure, and plots were scored for severity of infection (0-100% foliage affected) for 10 plants within each plot at the late R6 stage of soy development with guidance from Godoy et al (1997; Journal of plant disease and protection 104: 336-345). At approximately the R7 stage of development, soybean plants were scored for defoliation (0-100% defoliated). Disease severity, defoliation and yield results are provided in Table 47.

TABLE 47
Yield and severity of Asian Soybean Rust disease symptoms after foliar
application of SDHI fungicide and Gm.RHPP compositions in Paraguay

| | Application | Asian Soybean | | |
|---|---|---|---|---|
| | Use Rate in | Rust Disease | Defoliation | |
| | Fluid | Scores | Percentage | |
| | Ounces/Acre | (% Severity) | (% of foliage) | Yield (kg/Ha) |
| Foliar | Milliliters/ | (Comparisons | (Comparisons | (Comparisons |
| Treatment | hectare | Below) | Below) | Below) |
| Untreated | n/a | 75.0% | 90.0% | 1,269 |
| Control | | — | — | — |
| (UTC) | | | | |
| Cripton | 6.84 Fl oz/Ac | 26.7% | 46.7% | 2.,039 |
| XPro | 500 mL/Ha | (−48.3% to | (−43.3% to | (+770 kg to |
| Fungicide | | UTC) | UTC) | UTC) |
| Cripton | 6.84 Fl oz/Ac | 20.0% | 35.0% | 2,603 |
| XPro | 500 mL/Ha + | (−55.0% to | (−55.0% to | (+1,334 to |
| Fungicide + | 4.11 Fl oz/Ac | UTC) | UTC) | UTC) |
| Gm.RHPP | 300 mL/Ha | (−6.7% to | (−11.7% to | (+564 to |
| (SEQ ID | | CriptonXPro) | Cripton | Cripton |
| NO: 604) | | | XPro) | XPro) |

Foliar application of Gm.RHPP in combination with the fungicide Cripton XPro during reproductive phases of soy development provided increased protection against ASR as compared to the Untreated Control or Cripton XPro alone. Combination treatment with Gm.RHPP+Cripton XPro decreased ASR disease severity scores to 27% of the Untreated control and 75% of Cripton)(Pro. Combination treatment with Gm.RHPP+Cripton XPro decreased defoliation percentages to 39% of the Untreated control and 75% of Cripton XPro. Combination treatment with Gm.RHPP+Cripton XPro increased yield to 205% of the Untreated control and 127% of Cripton XPro. Based on these results, a soybean disease management program that included Gm. RHPP would be expected to reduce ASR foliar symptoms and defoliation, thus increasing grain fill and yield compared to the standard fungicide alone.

### Example 15: Trunk Injection of Oxytetracycline+Bacillus subtilis Serine Protease 2 Increases Fruit Yield on Citrus Trees Infected with CLas

As described in Example 1, co-injection treatments of oxytetracycline (0.48 g, or 0.93 g) with fermentation filtrate containing Serine Protease 2 (BsSP2) (SEQ ID NO: 795) were tested for ability to increase ‘Hamlin’ orange yield and/or decrease fruit drop. Trials were arranged with 10 trees per treatment, with two replicated blocks of five trees each in Okeechobee County, FL. The results are summarized in Table 48 below.

TABLE 48
Trunk injection of oxytetracycline combination with BsSP2 increased ‘Hamlin’
fruit yield relative to oxytetracycline alone

| | Average Yield (kg | Average Yield (Fruit |
|---|---|---|
| Treatment | fruit per tree) | Count per Tree) |
| Citrus Composition 3 | 12.65 | 84.75 |
| Oxytetracycline-HCl | — | — |
| (24.1 mg/mL); 20 mL/tree injection (0.48 | | |
| g/tree) | | |
| Citrus Composition 19 | 15.68 | 105.00 |
| Oxytetracycline-HCl | (124% relative to | (124% relative to |
| (24.1 mg/mL); 20 mL/tree injection (0.48 | Composition 3) | Composition 3) |
| g/tree) + | | |
| BsSP2 (SEQ ID NO: 795); 20 mL/tree | | |
| injection | | |

Results indicate that a citrus management program that included co-injection of oxytetracycline with BsSP2 would be expected to provide, on average, a 24% increase in yield relative to oxytetracycline alone (Table 48).

### Example 16: Trunk Injection of Oxytetracycline and 2-Deoxy-D-Glucose Increases Fruit Yield and Juice Quality and Reduces Fruit Drop on Citrus Trees Infected with CLas

As described in Example 1, oranges of the ‘Vernia’ and ‘Valencia’ varieties were harvested from trials designed to test the efficacy of co-injection treatments of oxytetracycline (0.48 g or 0.93 g) with 2-Deoxy-D-Glucose (2-DDG), with respect to orange yield, fruit quality, and/or juice quality. Trials were arranged with 10 trees per treatment, with two replicated blocks of five trees each. At the time of harvest, two representative fruit were collected per tree for fruit and juice quality assessment, as described in Example 1. The results are summarized in Tables 49, 50, and 51 below.

TABLE 49
Trunk injection of oxytetracycline (0.93 g per tree) in combination with 2-deoxy-
D-glucose increased ‘Vernia’ and ‘Valencia’ fruit yield relative to untreated control

| | Average Yield | Average Yield | Calculated |
|---|---|---|---|
| | (kg fruit per | (Fruit Count per | Average Weight |
| Treatment | tree) | Tree) | per Fruit (g) |
| | Vernia | | |
| Untreated Control (UTC) | 50.88 | 327.2 | 157.93 |
| Citrus Composition 24 | 57.35 | 368.4 | 151.67 |
| Oxytetracycline-HCl | (113% of UTC) | (113% of UTC) | (96% of UTC) |
| (24.1 mg/mL); 20 mL/tree | | | |
| injection (0.93g/tree) + | | | |
| 2-DDG (5.56 mg/mL solution | | | |
| in water); 20 mL/tree injection | | | |
| (111.1 mg/tree) | | | |
| | Valencia | | |
| Untreated Control (UTC) | 37.05 | 239.4 | 157.92 |
| Citrus Composition 24 | 47.17 | 279.9 | 168.33 |
| Oxytetracycline-HCl | (127% of UTC) | (117% of UTC) | (107% of UTC) |
| (48.6 mg/mL); 20 mL/tree | | | |
| injection (0.93 g/tree) + | | | |
| 2-DDG (5.56 mg/mL solution | | | |
| in water); 20 mL/tree injection | | | |
| (111.1 mg/tree) | | | |

Increased yield (kg/tree) was observed after co-injection of oxytetracycline and 2-DDG, relative to the untreated control. Increased total yield was due in part to increased number of fruit per tree (Vernia and Valencia) and increased fruit size (Valencia).

TABLE 50
Trunk injection of oxytetracycline (0.48 g per tree) in combination with 2-deoxy-
D-glucose increased ‘Vernia’ fruit yield relative to untreated control and oxytetracycline
alone

| | | | | | Average |
|---|---|---|---|---|---|
| | | Average | | | Fruit |
| | Average | Yield | Calculated | | Drop |
| | Yield (kg | (Fruit | Average | Average | (percent |
| | fruit per | Count per | Weight per | Diameter | of total |
| Treatment | tree) | Tree) | Fruit (g) | (mm) | fruit) |
| Untreated Control | 51.33 | 325.2 | 162.56 | 65.64 | 3.58% |
| (UTC) | — | — | — | — | — |
| Citrus Composition 3 | 55.17 | 309.6 | 178.36 | 63.33 | 4.61% |
| Oxytetracycline-HCl | (107% of | (95% of | (110% of | (96% of | (Δ + 1.04% |
| (24.1 mg/mL); 20 | UTC) | UTC) | UTC) | UTC) | to UTC) |
| mL/tree injection (0.48 | — | — | — | — | — |
| g/tree) | | | | | |
| Citrus Composition 24 | 72.56 | 478.0 | 172.4 | 70.17 | 3.24% |
| Oxytetracycline-HCl | (141% of | (147% of | (106% of | (107% of | (Δ − 0.34% to |
| (24.1 mg/mL); 20 | UTC) | UTC) | UTC) | UTC) | UTC) |
| mL/tree injection (0.48 | (132% | (154% | (97% | (111% | (Δ − 1.37% to |
| g/tree) + | relative to | relative to | relative to | relative to | Composition |
| 2-DDG (5.56 mg/mL | Composition | Composition | Composition | Composition | 3) |
| solution in water); 20 | 3) | 3) | 3) | 3) | |
| mL/tree injection | | | | | |
| (111.1 mg/trees) | | | | | |

Increased yield (compared to plants that were untreated or treated with oxytetracycline) was observed for co-injection of oxytetracycline and 2-DDG. Results indicate that a citrus management program that included co-injection of oxytetracycline with 2-DDG could be expected to provide, on average, a 27% increase in yield, 26% increase in fruit count, and 2.9% increase in fruit size relative to untreated (Table 49 and Table 50). Table 50 above indicates an 11% increase in fruit diameter relative to injection of oxytetracycline alone, coupled with a 1.37% decrease in fruit drop, which contribute to increased yield.

Co-injection treatment of oxytetracycline (0.93 g) with 2-DDG was tested for ability to increase ‘Vernia’ and ‘Valencia’ juice quality. The results are summarized in Table 51 below.

TABLE 51
Trunk injection of oxytetracycline combination with 2-deoxy-D-glucose increased
‘Vernia’ and ‘Valencia’ juice quality relative to oxytetracycline alone

| Treatment | Brix value | Brix:CA Ratio |
|---|---|---|
| | ‘Vernia’ | |
| Citrus Composition 2 | 9.40 | 19.58 |
| Oxytetracycline-HCl | — | — |
| (48.6 mg/mL); 20 mL/tree injection (0.93 g/tree) | | |
| Citrus Composition 24 | 9.90 | 18.68 |
| Oxytetracycline-HCl | (+0.50 relative to | (−0.90 relative to |
| (48.6 mg/mL); 20 mL/tree injection (0.93 g/tree) + | Composition 2) | Composition 2) |
| 2-DDG (5.56 mg/mL solution in water); 20 | | |
| mL/tree injection (111.1 mg/tree) | | |
| | ‘Valencia’ | |
| Citrus Composition 2 | 11.73 | 10.30 |
| Oxytetracycline-HCl | — | — |
| (48.6 mg/mL); 20 mL/tree injection (0.93 g/tree) | | |
| Citrus Composition 16 | 12.30 | 12.42 |
| Oxytetracycline-HCl | (+0.58 relative to | (+2.12 relative to |
| (48.6 mg/mL); 20 mL/tree injection (0.93 g/tree) + | Composition 2) | Composition 2) |
| 2-DDG (5.56 mg/mL solution in water); 20 | | |
| mL/tree injection (62.9 mg/tree) | | |

Increased yield (compared to plants that were untreated or treated with oxytetracycline) was observed for co-injected treatments with oxytetracycline and 2-DDG. Results indicate that a citrus management program that included co-injection of oxytetracycline with 2-DDG would be expected to provide, on average, a +0.54 increase in Brix and a +0.61 increase in the Brix:CA Ratio relative to injection of oxytetracycline alone (Table 51; average of Valencia and Vernia juice quality results).

### Example 17: Polysaccharide Degradation from Recovery Enzyme Treatments with Inducer Compounds for Use in the Treatment of Citrus Disease

Combinations of recovery compositions that were used to restore plant health and increase fruit yield in HLB-infected citrus trees by the reduction of phloem-blocking callose polysaccharides were further examined for the degradation of β-1,3 linked glucan polymers in kinetic assays. To demonstrate enzyme activity, various β-1,3-D-glucanases (SEQ ID NO: 731, 732, 768, 770, 772, 773, 774, 775, 776) were tested for activity on β-1,3-glucan from Euglena gracilis (Table 52), and two α-amylases (SEQ ID NO: 734, 735) were tested for activity on starch.

Amounts of 0.05 g of β-1,3-glucan (unbranched 13-1,3-linked glucose residues) from Euglena gracilis weighed and dissolved in 8.75 mL 2.5 M NaOH sodium hydroxide (CAS #1310-73-2) and diluted to 10 mL in glacial acetic acid (64-19-7) to bring the pH to 7. For each enzyme, 8.0 μL of enzyme preparation was added, in duplicate, to a 96-well plate, containing 52 μL of 50 mM citrate buffer, pH 4.9, or 300 nL of enzyme preparation diluted in 59.7 μL 50 mM citrate buffer, pH 4.9 was added, in duplicate, to a 96-well plate. 80 μL of 5 g/L β-1,3-glucan from Euglena gracilis in 87.5 g/L sodium hydroxide, pH 7 was added to each well and kept at a consistent temperature of 37° C. After 30 to 60 minutes, 120 μL of coloring solution containing 10 g/L 3,5-dinitrosalicylic acid (CAS #609-99-4), 10 g/L NaOH (CAS #1310-73-2), 0.5 g/L Na₂SO₄ (CAS #7757-82-6), 2.0 g/L Phenol (CAS #108-95-2), 182 g/L potassium sodium tartrate (CAS #6381-59-5), and 0.18 g/L glucose (CAS #50-99-7) was added to each well of the 96-well plate. With the addition of heat (10 minutes at 99.9° C.), reduction of the 3,5-dinotrosalicylic acid to 3-amino-5-nitrosalicylic acid by the freed glucose resulted in a maximum absorbance change of the reaction mixture from 375 nm to 540 nm. Absorbances at 540 nm (A₅₄₀) from 200 μL of reaction mixture were recorded with a BioTek spectrophotometer. For data analysis, the average A_{540 nm} value 30 or 60 min post-treatment is reported (n=2-3 samples per treatment) in Table 52.

TABLE 52
Glucanase activity assays of β-1,3-D-glucanases on β-1,3-glucan from Euglena
gracilis

| Treatment | A₅₄₀ | |
|---|---|---|
| (n = 2 replicates per treatment) | 30 min post- treatment | Blank subtracted A₅₄₀ |
| Blank (mock treatment) | 0.381 | 0.000 |
| β-1,3-D-glucanase (BglH) from | 1.042 | 0.662 |
| Paenibacillus spp. (SEQ ID NO: 732) | | |
| β-1,3-D-glucanase (HvGii) from Hordeum | 0.426 | 0.045 |
| vulgare (SEQ ID NO: 731) | | |
| β-1,3-D-glucanase (LamA1) from | 0.448 | 0.067 |
| Paenibacillus sp. CCRC17245 (SEQ ID | | |
| NO: 768) | | |
| β-1,3-D-glucanase (CsPr2) from Citrus | 0.394 | 0.014 |
| sinensis (SEQ ID NO: 770) | | |
| β-1,3-D-glucanase (DK-1) from | 1.284 | 0.903 |
| Cellulosimicrobium cellulans strain .DK-1 | | |
| (SEQ ID NO: 772) | | |
| β-1,3-D-glucanase (QLK1) from | 0.687 | 0.307 |
| Kitasatospora phosalacinea strain | | |
| SYBCQL (SEQ ID NO: 773) | | |
| β-1,3-D-glucanase (17-W) from | 0.734 | 0.353 |
| Streptomyces sp. SYBC17 (SEQ ID NO: | | |
| 774) | | |
| β-1,3-D-glucanase (BglS27) from | 0.882 | 0.501 |
| Streptomyces sp. S27 (SEQ ID NO: 775) | | |
| β-1,3-D-glucanase (BglM) from | 0.716 | 0.336 |
| Paenibacillus sp IAM1165 (SEQ ID NO: | | |
| 776) | | |

Glucanase activity assay results (Table 52) indicated that glucanases from various sources, including plants, gram-negative bacteria, and gram-positive bacteria, have activity on β-1,3-glucan from Euglena gracilis, an unbranched polymer of glucose residues linked by β-1,3 glycosidic bonds. Euglena gracilis, a eukaryote, makes a glucan predicted to have a structure similar to the callose found in the phloem of a citrus tree.

To test for compatibility between β-1,3-D-glucanase in a recovery enzyme mixture, a tank mix of β-1,3-D-glucanase from Hordeum vulgare (SEQ ID NO: 731), L-Cysteine (Treatment D), and α-amylase from Bacillus licheniformis (Treatment C) SEQ ID NO: 735) were compared (Table 53) with β-1,3-D-glucanase alone (Treatment A). The β-1,3-D-glucanase combination with L-Cysteine and α-amylase (Treatment B) were assayed for glucanase activity which was specifically selected to model co-injection of the recovery enzyme mixture on citrus trees used for Ruby Red grapefruit and Valencia oranges (Examples 3 and 20). Combination concentrations of β-1,3-D-glucanase from Hordeum vulgare with L-Cysteine and α-amylase were matched to injection concentrations as used for the injection of Ruby Red grapefruit and Valencia oranges in Table 53. Final concentrations for β-1,3-D-glucanase from Hordeum vulgare (SEQ ID NO: 731) (33 U/mL); L-cysteine (0.0133 mg/mL) and α-amylase from Bacillus licheniformis (SEQ ID NO: 735) (33 U/mL) in 50 mM citrate buffer, pH 4.9 were used in the glucanase activity assays.

TABLE 53
Glucanase activity assays of β-1,3-D-glucanase from Hordeum vulgare
used in combination
with recovery citrus compounds

| | A₅₄₀ | Blank | Change |
|---|---|---|---|
| Treatment | 60 min post- | subtracted | relative to |
| (n = 3 replicates per treatment) | treatment | A₅₄₀ | Treatment A |
| Blank (buffer only) | 0.414 | 0.000 | 0.000 X |
| Treatment A | 0.522 | 0.108 | 1.0 X |
| β-1,3-D-glucanase from Hordeum vulgare | | | |
| (SEQ ID NO: 731), 33 U/mL | | | |
| Treatment B | 0.514 | 0.101 | 0.93 X |
| β-1,3-D-glucanase from Hordeum vulgare | | | |
| (SEQ ID NO: 731), 33 U/mL + | | | |
| α-amylase from Bacillus licheniformis | | | |
| (SEQ ID NO: 735), 33 U/mL + | | | |
| L-Cysteine CAS 136743-62-9 | | | |
| 0.013 mg/mL | | | |
| Treatment C | 0.383 | −0.031 | −0.29 X |
| α-amylase from Bacillus licheniformis | | | |
| (SEQ ID NO: 735), 33 U/mL | | | |
| Treatment D | 0.373 | −0.041 | −0.38 X |
| L-Cysteine CAS 136743-62-9 | | | |
| 0.013 mg/mL | | | |

Glucanase activity assay results (Table 53) indicated that L-Cysteine, an inducer compound, and α-amylase, a starch degrader, provided in combination with the β-1,3-D-glucanase (Treatment B) resulted in a glucanase response that was similar to the response output from β-1,3-D-glucanase alone (Treatment A). Additionally, L-Cysteine and α-amylase, provided alone (Treatments C & D) and in combination with the β-1,3-D-glucanase (Treatment B) did not show additional glucanase activity by synthesizing glucose or degrading glucans but also did not significantly hinder the response of the β-1,3-D-glucanase and are therefore found compatible in the recovery enzyme mixture.

For data in Table 54 below, an amount of 0.1 g of starch (unbranched α-1,4-linked glucose residues) from Solanum tuberosum was suspended in 1.0 mL room temperature 10× Phosphate Buffered Saline (PBS), pH 6.9 and then dissolved into 9 mL boiling water. For Treatments A-D, 0.165 nL of enzyme preparation diluted in 1×PBS, pH 6.9 were added, in triplicate, to a 96-well plate. For Treatments E-G, 9.4 nL of enzyme preparation in 1×PBS, pH 6.9 were added, in triplicate, to a 96-well plate. 40 μL of 10 g/L starch in 1×PBS, pH 6.9 was added to each well and kept at a consistent temperature of 37° C. After 10 minutes, 60 μL of coloring solution containing 10 g/L 3,5-dinitrosalicylic acid (CAS #609-99-4), 10 g/L NaOH (CAS #1310-73-2), 0.5 g/L Na2SO4 (CAS #7757-82-6), 2.0 g/L Phenol (CAS #108-95-2), and 182 g/L potassium sodium tartrate (CAS #6381-59-5) was added to each well of the 96 well plate. With the addition of heat (10 minutes at 99.9° C.), reduction of the 3,5-dinotrosalicylic acid to 3-amino-5-nitrosalicylic acid by the freed glucose resulted in a maximum absorbance change of the reaction mixture from 375 nm to 540 nm. Absorbances at 540 nm (A540) from 100 μL of reaction mixture were recorded with a BioTek spectrophotometer. For data analysis, the average A540 nm value 10 minutes post-treatment is reported (n=2-3 samples per treatment).

TABLE 54
Amylase activity assays of β-amylase from Bacillus licheniformis used in
combination with recovery citrus compounds

| | A₅₄₀ | Blank | Change |
|---|---|---|---|
| Treatment | 10 min post- | subtracted | relative to |
| (n = 2-3 replicates per treatment) | treatment | A₅₄₀ | Treatment A |
| Blank (buffer only) | 0.184 | 0.000 | 0.000 X |
| Treatment A | 0.590 | 0.406 | 1.0 X |
| α-amylase from Bacillus licheniformis | | | |
| (SEQ ID NO: 735), 33 U/mL | | | |
| Treatment B | 0.659 | 0.475 | 1.17X |
| β-1,3-D-glucanase from Hordeum vulgare | | | |
| (SEQ ID NO: 731), 33 U/mL + | | | |
| α-amylase from Bacillus licheniformis | | | |
| (SEQ ID NO: 735), 33 U/mL + | | | |
| L-Cysteine CAS 136743-62-9 | | | |
| 0.013 mg/mL | | | |
| Treatment C | 0.214 | 0.030 | 0.07 X |
| β-1,3-D-glucanase from Hordeum vulgare | | | |
| (SEQ ID NO: 731), 33 U/mL | | | |
| Treatment D | 0.207 | 0.023 | 0.06 X |
| L-Cysteine CAS 136743-62-9 | | | |
| 0.013 mg/mL | | | |
| Treatment E | 0.374 | 0.190 | 1.0 X |
| α-amylase from Bacillus subtilis 168 | | | |
| (SEQ ID NO: 734), 60 U/mL + | | | |
| cell supernatant from Bacillus subtilis | | | |
| 168 | | | |
| Treatment F | 0.465 | 0.281 | 1.48 X |
| α-amylase from Bacillus subtilis 168 | | | |
| (SEQ ID NO: 734), 60 U/mL + | | | |
| 2-DDG CAS 154-17-6 (6.94 mg/mL) + | | | |
| cell supernatant from Bacillus subtilis | | | |
| 168 | | | |
| Treatment G | 0.257 | 0.073 | 0.38 X |
| 2-DDG CAS 154-17-6 | | | |
| 6.94 mg/mL + cell supernatant from | | | |
| Bacillus subtilis 168 | | | |

Amylase activity assay results (Table 54) indicated that L-Cysteine, an inducer compound, and β-1,3-D-glucanase, a callose degrader, provided in combination with the α-amylase (Treatment B) resulted in an increased amylase activity response that was similar to the response output from the α-amylase and 2-DDG (Treatment F). Additionally, L-Cysteine and β-1,3-D-glucanase, provided alone (Treatments C & D) and in combination with the α-amylase (Treatment B) did not show significant amylase activity by synthesizing glucose or degrading starch but also did not hinder the response of the α-amylase and are therefore found compatible (Table 54).

Amylase activity assay results (Table 54) indicated that 2-DDG, which is used as a callose synthase inhibitor, provided in combination with α-amylase (Treatment F), did not show significant additional amylase activity beyond the additions of Treatments E and G (p=0.23) (Treatment G is a reducing sugar that acts on 3,5-dinitrosalicylic acid but does not have enzymatic activity on starch or other polysaccharides). This was an expected result as 2-DDG, used to reduce callose content in the phloem, does not synthesize glucose or degrade starch, but also did not hinder the response (activity) of the α-amylase and therefore is compatible in this recovery enzyme mixture.

Example 4 (Table 34) demonstrates how L-cysteine cysteine assists other actives to activate the plant immune system outside of glucanase and amylase activity. The Recovery Enzyme Mixture described in Example 10 (Table 42) and Example 17 (Tables 53, and 54), describe a combinatorial synergistic approach to clear the callose (a β-1,3 glucan polymer) and starch (an α-1,4-glucose polymer) buildup in citrus phloem to improve nutrient transport while helping to induce immune system activation to combat a Clas infection.

### Example 18: Polysaccharide Degradation from β-1,3-Endoglucanase for Use in the Treatment of Fungal Disease

Combinations of recovery compositions that were used to restore plant health and increase fruit yield in HLB-infected citrus trees by the reduction of phloem-blocking callose polysaccharides were further examined for the degradation of β-1,3 linked glucan polymers in kinetic assays. To demonstrate enzyme activity, various β-1,3-D-glucanases (SEQ ID NO: 731, 732, 767, 772, 773, 774, 775, 776) were tested for activity on carboxymethyl-pachyman (CM-pachyman) (CAS 69552-83-6), derived from a 1,3-β-D-glucan from the sclerotia of Poria cocos, a wood-decay fungus (Table 55). CM-pachyman was carboxymethylated with chloroacetic acid to increase solubility in aqueous solutions.

An amount of 0.125 g of CM-pachyman from Poria cocos was dissolved in 23.5 mL water, at 90° C., and diluted to 25 mL in 0.5 M sodium citrate buffer, pH 4.9. For each enzyme, 5.0 μL of enzyme preparation was added, in duplicate, to a 96-well plate, containing 55 μL of 50 mM citrate buffer, pH 4.9. 80 μL of 5 g/L CM-pachyman from Poria cocos in 50 mM citrate buffer, pH 4.9 was added to each well and kept at a consistent temperature of 37° C. After 30 minutes, 120 μL of coloring solution containing 10 g/L 3,5-dinitrosalicylic acid (CAS #609-99-4), 10 g/L NaOH (CAS #1310-73-2), 0.5 g/L Na2SO4 (CAS #7757-82-6), 2.0 g/L Phenol (CAS #108-95-2), 182 g/L potassium sodium tartrate (CAS #6381-59-5), and 0.18 g/L glucose (CAS #50-99-7) was added to each well of the 96-well plate. With the addition of heat (10 minutes at 99.9° C.), reduction of the 3,5-dinotrosalicylic acid to 3-amino-5-nitrosalicylic acid by the freed glucose resulted in a maximum absorbance change of the reaction mixture from 375 nm to 540 nm. Absorbances at 540 nm (A540) from 200 μL of reaction mixture were recorded with a BioTek spectrophotometer. For data analysis, the average A540 nm value 30 min post-treatment is reported (n=2 samples per treatment).

TABLE 55
Glucanase activity assays of β-1,3-D-glucanases on CM-pachyman
from Poria cocos

| Treatment | A₅₄₀ | |
|---|---|---|
| (n = 2 replicates per treatment) | 30 min post- treatment | Blank subtracted A₅₄₀ |
| Blank (mock treatment) | 0.350 | 0.000 |
| β-1,3-D-glucanase (HvGii) from Hordeum | 0.626 | 0.276 |
| vulgare (SEQ ID NO: 731) | | |
| β-1,3-D-glucanase (BglH) from | 0.765 | 0.415 |
| Paenibacillus spp. (SEQ ID NO: 732) | | |
| β-1,3-D-glucanase (LamA1) from | 0.402 | 0.052 |
| Paenibacillus sp. CCRC17245 (SEQ ID | | |
| NO: 767) | | |
| β-1,3-D-glucanase (DK-1) from | 0.853 | 0.503 |
| Cellulosimicrobium cellulans strain .DK-1 | | |
| (SEQ ID NO: 772) | | |
| β-1,3-D-glucanase (QLK1) from | 0.592 | 0.242 |
| Kitasatospora phosalacinea strain | | |
| SYBCQL (SEQ ID NO: 773) | | |
| β-1,3-D-glucanase (17-W) from | 0.556 | 0.206 |
| Streptomyces sp. SYBC17 (SEQ ID NO: | | |
| 774) | | |
| β-1,3-D-glucanase (BglS27) from | 0.637 | 0.287 |
| Streptomyces sp. S27 (SEQ ID NO: 775) | | |
| β-1,3-D-glucanase (BglM) from | 0.705 | 0.268 |
| Paenibacillus sp IAM1165 (SEQ ID NO: | | |
| 776) | | |

Glucanase activity assay results (Table 55) indicated that glucanases from various sources (β-1,3-D-glucanase listed as SEQ ID Nos: 731, 732, 767, 772, 773, 774, 775 and 776) which include glucanases from gram-negative bacteria and gram-positive bacteria, have activity on CM-pachyman from Poria cocos, an unbranched polymer of glucose residues linked by β-1,3 glycosidic bonds with glucanases of SEQ ID Nos: 732 and 772 having the highest activities. Polysaccharides, including β-1,3-linked polymers, are a key component of fungal cell walls and any of the above enzymes demonstrating activity degrading β-1,3-linked glucose-based polysaccharides would have activity on the fungal cell wall and lead to lysis of fungal pathogens.

### Example 19: Trunk Injection of Recovery-Promoting Enzyme Compositions Increase Fruit Yield and Juice Quality on Citrus Trees Infected with Clas

Treatments as previously described in Example 3; β-1,3-Endoglucanase from barley SEQ ID NO: 731 (Treatment 1) and Bt.4Q7Flg22 from Bacillus thurigiensis SEQ ID NO: 226 (Treatment 5) increased fruit yield (kg/tree) as described in Table 56. Fruit quality, and juice quality of ‘Valencia’ oranges are described in Table 57.

TABLE 56
Trunk injection of recovery compositions increased ‘Grapefruit’ and ‘Valencia’
fruit yield relative to untreated trees

| | Average Yield (kg per tree) | | |
|---|---|---|---|
| | (Relative to Untreated) | | |
| Treatment | ‘Ruby Red’ | ‘Valencia’ | Average of 2 sites |
| Untreated | 34.6 | 40.3 | |
| | — | — | — |
| Bt.4Q7Flg22 | 37.2 | 48.1 | |
| (SEQ ID NO: 226) (0.33 mg/tree) | (108%) | (119%) | (113%) |
| B-1,3-Endoglucanase | 38.7 | 54.8 | |
| from Barley (SEQ ID NO: 731) (2660 U/tree) | (112%) | (136%) | (124%) |
| L-Cysteine | 34.8 | 42.3 | |
| (0.27 mg/tree) | (101%) | (105%) | (103%) |
| Recovery Enzyme Mixture | 35.1 | 46.8 | |
| B-1,3-Endoglucanase from Barley (SEQ ID | (102%) | (116%) | (109%) |
| NO: 731) (660 U/tree) + Amylase from Bacillus | | | |
| licheniformis (SEQ ID NO: 735) (660 U/tree) + | | | |
| Cysteine (0.27 mg/tree) | | | |

For direct comparison of the treatment results between grapefruit (‘Ruby Red’) and orange (‘Valencia’) are referenced in Table 56 above. Harvest results from replicated ‘Ruby Red’ grapefruit trials and described individually in Table 32 indicated that trunk injection treatments to activate the plant immune system, degrade polysaccharides in and around sieve tube elements, and provide essential amino acids for sustained defense responses (L-cysteine) resulted in increased fruit yield. Table 56 provides a summary of the harvest results from “Ruby Red” grapefruit trials and ‘Valencia’ orange trials and an average of 2 sites for each citrus variety. On ‘Valencia’ trees, Bt.4Q7Flg22 injection at 0.33 mg per tree increased yield 7.8 kg per tree compared to the untreated control (combined average 113%), while the callose-degrading enzyme β-1-3-endoglucanase from Barley increased yield 14.5 kg per tree (average 124% relative to control). L-Cysteine injection at 0.27 per tree increased yield+2.0 kg (average 103%). A Recovery Enzyme Mixture including β,1-3-endoglucanase, starch-degrading Amylase, and L-Cysteine increased yield 6.5 kg per tree compared to the untreated control (average 109%), and a citrus management program including injection of one or more of these components in a range of concentrations, would be expected to improve tree health and yield.

TABLE 57
Trunk injection of recovery compositions increased ‘Valencia’ fruit size and juice
quality relative to untreated trees

| | Average Fruit | |
|---|---|---|
| | Diameter (mm) | Brix:CA Ratio |
| | (Relative to | (Relative to |
| Treatment | Untreated) | Untreated) |
| Untreated | 60.13 | 11.25 |
| | — | |
| Bt.4Q7Flg22 | 60.72 | 12.11 |
| (SEQ ID NO: 226) (0.33 mg/tree) | (101%) | (+0.86) |
| Treatment 5 | | |
| B-1,3-Endoglucanase | 62.76 | 12.01 |
| from Barley (SEQ ID NO: 731) (660 U/tree) | (104%) | (+0.76) |
| Treatment 1 | | |
| L-Cysteine | 62.52 | 12.76 |
| (0.27 mg/tree) | (104%) | (+1.51) |
| Recovery Enzyme Mixture | 65.57 | 12.89 |
| B-1,3-Endoglucanase Hordeum vulgare (SEQ ID NO: 731) | (109%) | (+1.63) |
| (660 U/tree) +Amylase from Bacillus licheniformis (SEQ ID | | |
| NO: 735) (660 U/tree) + Cysteine (0.27 mg/tree) | | |

Fruit quality assessments of the ‘Valencia’ oranges harvested in Spring 2019 indicate that trunk injection of Bt.4Q7Flg22 (0.33 mg per tree), the callose-degrading enzyme β,1-3-endoglucanase from Barley, L-cysteine, an essential amino acid for sustained defense responses, and the Recovery Enzyme Mixture increased both fruit size and ^{o}Brix_{c} to Citric Acid ratio (Brix:CA) relative to the untreated control (Table 57). According to the Food and Agriculture Organization of the United Nations (FAO), Brix:CA should be 10-14, with an ideal target of 12 for oranges. Trunk injection of Bt.4Q7Flg22, β,1-3-endoglucanase from Barley, L-cysteine, and the Recovery Enzyme Mixture all resulted a Brix:CA ratio near the target value for fruit quality.

### Examples: 20-24

In Examples 20-24, a series of treatment compositions were tested by citrus tree injection or foliar spray to measure their effect on fruit yield and quality. For ease of reference, the compositions used and application method and use rate are summarized in Table 58 below.

TABLE 58
Compositions for the prevention and treatment of citrus disease

| Composition | | Treatment | Application | # of Applications |
|---|---|---|---|---|
| No: | Formulation | Method | Use Rate | (Timing) |
| Citrus | Bt.4Q7Flg22 (SEQ ID | Trunk | 6.875 mL/tree | 1 Application |
| Composition | NO: 226) 48 ppm | Injection | (0.33 mg) | (Spring 2019) |
| 25 | 4 mM Sodium Phosphate | | | |
| | Buffer, pH 5.7 | | | |
| Citrus | Part A | Trunk | 6.875 mL/tree | 1 Application |
| Composition | Bt.4Q7Flg22 (SEQ ID | Injection | (0.33 mg) | (Spring 2019) |
| 26 | NO: 226) 48 ppm | | | |
| | 4 mM Sodium Phosphate | | | |
| | Buffer, pH 5.7 | | | |
| | Part B | Trunk | 2640 Enzyme | |
| | β-1,3-endoglucanase from | Injection | Units per tree | |
| | Hordeum vulgare (SEQ | | | |
| | ID NO: 731) | | | |
| Citrus | Amylase (amyE) | Trunk | 92 Enzyme Units | 1 Application |
| Composition | from Bacillus subtilis 168 | Injection | per tree | (Spring 2019) |
| 27 | (SEQ ID NO: 734) | | | |
| Citrus | Part A | Trunk | 92 Enzyme Units | 1 Application |
| Composition | Amylase (amyE) | Injection | per tree | (Spring 2019) |
| 28 | from Bacillus subtilis 168 | | | |
| | (SEQ ID | NO: 734) | | |
| | Part B | Trunk | 1.0 mL/tree | |
| | 2-Deoxy-D-Glucose (2- | Injection | (111.1 mg per | |
| | DDG) (111.1 mg/mL | | tree) | |
| | solution in water) | | | |
| Citrus | Part A | Trunk | 1.0 mL/tree | 1 Application |
| Composition | 2-Deoxy-D-Glucose (2- | Injection | (111.1 mg per | (Spring 2019) |
| 29 | DDG) (111.1 mg/mL | | tree) | |
| | solution in water) | | | |
| | Part B | Trunk | 9.3 mL/tree | |
| | Oxytetracycline-HCl | Injection | (0.875 g per | |
| | (94.5 mg/mL solution in | | tree) | |
| | water) | | | |
| Citrus | Bt.4Q7Flg22 (SEQ ID | Foliar | 12.0 mL/tree | 1 Application |
| Composition | NO: 226) 120 ppm | Spray | (1.44 mg) | (Spring 2019) |
| 30 | 10 mM Sodium | | | |
| | Phosphate Buffer, pH 5.7 | | | |
| Citrus | Part A | Foliar | 12.0 mL/tree | 2 Applications |
| Composition | Bt.4Q7Flg22 (SEQ ID | Spray | (1.44 mg) | (Spring 2019 |
| 31 | NO: 226) 120 ppm | | | and Fall 2019) |
| | 10 mM Sodium | | | |
| | Phosphate Buffer, pH 5.7 | | | |
| | Part B “Osmoprotectant | Foliar | 4.21 mL/tree | |
| | 1” | Spray | | |
| | 12.8 g/L Betaine HCl + | | | |
| | 3.3% (w/v) tribasic | | | |
| | potassium phosphate | | | |
| Citrus | Gm.RHPP (SEQ ID NO: | Foliar | 3.0 mL/tree (1.5 | 1 Application |
| Composition | 604) | Spray | mg) | (Spring 2019) |
| 32 | 500 ppm in water | | | |
| Citrus | β-1,3-endoglucanase | Trunk | 2625 Enzyme | 1 Application |
| Composition | (DK-1) | Injection | Units | (Fall 2019) |
| 33 | from Cellulosimicrobium | | per tree | |
| | cellulans (SEQ ID NO: | | | |
| | 760) | | | |

For Examples 20-24, trees were treated at three separate sites in Florida that were selected due to a high prevalence of HLB (See Examples 1, 2, 3). Five-year-old Ruby Red Grapefruit trees (Citrus paradisi) and 5-year old Hamlin orange (Citrus sinesis) trees were treated at Lake Wales, Fla. (Polk County), and 10-year old Valencia orange trees were treated at Eustis, Fla. (Lake County). Citrus composition treatments were applied as listed in Table 58 using a low-pressure injection device, BRANDT ENTREE for trunk injection with methods as described in Example 1, or a CO₂-pressurized backpack sprayer that produced a fine mist for foliar spray. Foliar compositions diluted in water with a non-ionic surfactant (alkyl phenol ethoxylate; 0.1% v/v of spray tank volume) and evenly applied to the canopy of the tree at a spray rate of 3 Liters (L) per tree. Blocks of trees receiving a foliar treatment were spaced in the trial area with a gap or a skipped tree between treatment blocks to avoid drift of treatment into neighboring treatment blocks. Treatments were applied during a period of low wind (<5 mph), and conditions were such all spray treatments dried on leaves within a period of 4 hours. Combination treatments described in Table 58 with two parts (A & B) were either co-injected in the same BRANDT ENTREE pouch (Citrus Compositions 26, 28, or 29) or tank mixed into the same foliar treatment tank (Citrus Composition 31). For all treatments, 10 trees were used per treatment per site, separated into two replicated blocks of five trees each.

To assess the effects of Citrus Compositions 25-33 on CLas bacterial titer, leaf samples were collected prior to application (TO) and 8-weeks post-application (T8) and processed for quantitative PCR (qPCR) at Southern Gardens Citrus (Clewiston, Fla.). For qPCR analysis, 10 leaves were collected per tree, randomly distributed over the tree canopy from the most recently hardened-off flush. Leaf mid-ribs were isolated from each set of 10 leaves and combined into a single sample for DNA extraction and qPCR analysis as described in previously in paragraph [0567] (Methods for quantifying CLas Titer in an Infected Citrus Plant). Ct values for each tree sample were used to calculate the CLas titer per 100 mg of leaf tissue. CLas titer results are summarized in Tables 60, 65 and 70. Leaf samples were again collected in August 2019 at either 15 weeks (T15; Valencia oranges) or 18 weeks (T18; Hamlin oranges and Ruby Red Grapefruit) post-application for nutrient content analysis at the University of Florida Institute of Food and Agriculture Sciences (UF IFAS) Extension (Gainesville, Fla.). Two samples were analyzed per treatment at each site, with a single sample consisting of 20 leaves randomly selected from each treatment block (5 trees per block×4 leaves per tree=20 leaves). A full nutrient panel was reported by UF IFAS Extension, including nitrogen, phosphorus, potassium, iron, calcium, copper, manganese, magnesium, boron and zinc. Selected average nutrient contents at each site (N=2 tree blocks per treatment) are reported in Tables 61, 64, 66, 71, and 72.

To assess fruit yield and quality, Hamlin oranges and Ruby Red Grapefruit (Polk County, FL) were harvested 7.5 months post-treatment. All fruit with a diameter greater than or equal to 1.6 inches (40 mm) were hand-picked and collected for each tree. The total “Fruit Weight” (in kilograms) per individual tree was measured and recorded. Trees with total fruit weights less than or greater than 2 standard deviations (2STDEV) from the trial mean were considered to be outliers and removed from the dataset. Fruit size was assessed as the “Average Weight per Fruit” in grams (weight of 20 random fruit per tree, divided by 20) and “Average Fruit Diameter” in millimeters, as described in Example 1. The “Average Fruit Count” per tree was assessed as the total “Fruit Weight” divided by “Average Weight per Fruit”. “Fruit Drop” was assessed as for Example 1. Fruit quality was assessed as for Example 2, with the exception that one set of fruit consisted for 15 total fruit, each corresponding to a sampling of 3 fruit from 5 trees of the same experimental treatment block. Each set of 15 fruit were weighed (gram; g) and then juiced together. Average yield, % fruit drop, fruit size, and juice quality for each of the tested compositions are described in Examples 20-24.

### Examples 20: Trunk Injection of Oxytetracycline and 2-Deoxy-D-Glucose Increases Fruit Yield in ‘Ruby Red’ Grapefruit and ‘Hamlin’ Orange

Harvest results from replicated Ruby Red grapefruit and Hamlin orange trials (Table 59) indicate that trunk injection of Oxytetracycline-HCl and 2-Deoxy-D-Glucose (2-DDG) together increased yield by 8.5% (kg fresh fruit) on average in comparison to the untreated control. Together with yield results in Example 16 (Table 49) for Citrus Composition 24, co-injection of Oxytetracycline-HCl and 2-DDG resulted in an average increase in yield of 14% over 4 independent trials (Table 49: 13% increase yield in ‘Vernia’ orange and a 27% increase yield in Valencia orange; Table 59: 8% in Ruby Red′ grapefruit and 9% and in ‘Hamlin’ orange).

TABLE 59
Trunk Injection of Oxytetracycline and 2-Deoxy-D-Glucose
increased fresh fruit yield in citrus

| | Yield, Average kg fruit per tree | | |
|---|---|---|---|
| | (relative to control) | | |
| Treatment | ‘Ruby Red’ Grapefruit | ‘Hamlin’ Orange | Average |
| Untreated Control | 32.57 kg | 49.4 kg | — |
| Citrus Composition 29 | 35.20 kg | 53.8 kg | |
| 2-Deoxy-D-Glucose (111.1 | (+8%) | (+9%) | (+8.5%) |
| mg/tree) + | | | |
| Oxytetracycline-HCl | | | |
| (0.875 g/tree) | | | |

### Examples 21: Trunk Injection of Bt.4Q7Flg22+β1,3 Endoglucanase Reduces Bacterial Titer, Improves Nutrient Content, and Increases Yield in Citrus

qPCR results from replicated ‘Valencia’ orange trials (Table 60) indicate that injection treatments to activate the plant immune system and degrade polysaccharides in and around sieve tube elements lead to decreased CLas bacterial titer levels 26 weeks after application. The Bt.4Q7Flg22 injection at 0.33 mg per tree decreased bacterial titers 20% relative to the untreated control, while the combination including Bt.4Q7Flg22 injection together with the callose-degrading enzyme β,1-3-endoglucanase from Barley decreased bacterial titers 97% relative to untreated control.

TABLE 60
Trunk injection of Bt.4Q7Flg22 and β-1,3-Endoglucanase decreased CLas
bacterial titers in HLB-infected ‘Ruby Red’ grapefruit 26 weeks post-application

| | Average CLas Titer | | |
|---|---|---|---|
| | (copy number per 100 mg leaf tissue) | | Fold Change in |
| | Pre-application | 26 Weeks Post- | CLas Titer |
| Treatment | T₀ | Application (T26) | (T₂₆/T₀) |
| Untreated Control | 6.23 × 10⁶ | 7.13 × 10⁶ | 1.14× |
| Citrus Composition 25 | 2.51 × 10⁶ | 2.01 × 10⁶ | 0.80× |
| Bt.4Q7Flg22 | | | |
| (SEQ ID NO: 226) | | | |
| (0.33 mg/tree) | | | |
| Citrus Composition 26 | 3.09 × 10⁵ | 9.04 × 10³ | 0.03× |
| Bt.4Q7Flg22 | | | |
| (SEQ ID NO: 226) | | | |
| (0.33 mg/tree) + | | | |
| B-1,3-Endoglucanase | | | |
| from Barley (SEQ ID NO: | | | |
| 731) (2660 U/tree) | | | |

Nutrient analysis results from replicated ‘Valencia’ and ‘Hamlin’ orange trials (Table 61) indicate that co-injection of Bt.4Q7Flg22 and β,1-3-endoglucanase increased manganese levels. Manganese is an important micronutrient involved in citrus tree metabolism and photosynthesis, and manganese deficiency can cause reduced yield, fruit size, and tree growth. Bt.4Q7Flg22 injection at 0.33 mg per tree increased manganese levels an average of 120% relative to untreated control, while the combination including Bt.4Q7Flg22 injection together with the callose-degrading enzyme β,1-3-endoglucanase from barley increased foliar manganese levels an average of 136% relative to untreated control.

TABLE 61
Trunk injection of recovery compositions
increased manganese levels in
manganese-deficient ‘Valencia’ and
‘Hamlin’ oranges relative to control

| | Manganese (ppm) | | |
|---|---|---|---|
| | (relative to untreated) | | |
| Treatment | ‘Valencia’ | ‘Hamlin’ | Average |
| Untreated Control | 6.32 | 14.18 | |
| | — | — | — |
| Citrus Composition 25 Bt.4Q7Flg22 | 7.54 | 17.05 | |
| (SEQ ID NO: 226) (0.33 mg/tree) | (119%) | (120%) | (120%) |
| Citrus Composition 26 Bt.4Q7Flg22 | 9.27 | 17.62 | |
| (SEQ ID NO: 226) (0.33 mg/tree) + | (147%) | (124%) | (136%) |
| β-1,3-Endoglucanase | | | |
| from Barley (SEQ ID NO: 731) | | | |
| (2660 U/tree) | | | |

Harvest results from replicated Ruby Red grapefruit trials (Table 62) indicate that trunk injection of Bt4Q7Flg22 alone in Spring 2019 increased yield by 14.4% relative to the untreated control. In a citrus disease management program including Bt4Q7Flg22 trunk injection (Spring 2019) and foliar application (Fall 2019), yield was further increased to 39.7% relative to untreated control. Trunk injection of β-1,3-Endoglucanase from C. cellulans to degrade polysaccharides in and around sieve tube elements increased yield by 22.4% relative to the untreated control. Based on synergistic reduction in CLas titers (Table 60), combination treatments between Bt4Q7Flg22 (Foliar and/or Trunk Injection) and β-1,3-Endoglucanase (Trunk Injection) are predicted to provide additive or synergistic yield increase and improvement in tree health.

TABLE 62
Trunk Injection of Bt4Q7Flg22 or β-1,3-Endoglucanase increased fresh fruit
yield in Ruby Red grapefruit trees relative to control

| | Yield, Average kg fruit per tree |
|---|---|
| Treatment | (relative to control) |
| Untreated Control | 32.57 kg |
| Citrus Composition 25 | 37.27 kg |
| Bt.4Q7Flg22 | (+14.4%) |
| (SEQ ID NO: 226) | |
| (0.33 mg/tree) | |
| Spring 2019 application | |
| Citrus Composition 25 | 45.49 kg |
| Bt.4Q7Flg22 (SEQ ID NO: 226) | (+39.7%) |
| (0.33 mg/tree) | |
| Spring 2019 application + | |
| Citrus Composition 30 | |
| Bt.4Q7Flg22 (SEQ ID NO: 226) | |
| (12 mL/tree) | |
| Fall 2019 application | |
| Citrus Composition 33 | 39.85 kg |
| β-1,3-endoglucanase (DK-1) | (+22.4%) |
| from Cellulosimicrobium cellulans | |
| (SEQ ID NO: 760) (2625 U/tree) | |
| Fall 2019 application | |

Fruit quality results from replicated Ruby Red grapefruit trials (Table 63) indicate that trunk injection of Bt.4Q7Flg22 and β-1,3-Endoglucanase from barley increases fruit size, as measured by an 11.4% increase in fruit diameter relative to control, and 5.4% increase relative to Bt.4Q7Flg22 trunk injection alone. Larger fruit provide more value to the grower, with a 40-count box of fruit for Bt.4Q7Flg22 and β-1,3-Endoglucanase co-treatment having higher value per box than the 56-count box for Bt.4Q7Flg22 alone, or 64-count box for the untreated control.

TABLE 63
Trunk Injection of Bt.4Q7Flg22, alone, or with β-1,3-Endoglucanase
improved fruit quality in Ruby Red grapefruit trees relative to control

| | Average fruit | |
|---|---|---|
| | diameter, mm | Box Count |
| Treatment | (relative to control) | (Fruit per box) |
| Untreated Control | 89.6 | 64-count |
| Citrus Composition 25 | 94.7 | 56-count |
| Bt.4Q7Flg22 | (+5.7%) | |
| (SEQ ID NO: 226) (0.33 mg/tree) | | |
| Citrus Composition 26 | 99.8 | 40-count |
| Bt.4Q7Flg22 + | (+11.4%) | |
| β-1,3-Endoglucanase from | | |
| Barley (SEQ ID NO: 731) (2660 | | |
| U/tree) | | |

### Example 22: Trunk Injection of Recovery Enzyme Compositions Improves Leaf Micronutrient Content

Nutrient analysis results from replicated ‘Ruby Red’ grapefruit and ‘Valencia’ and ‘Hamlin’ orange trials (Table 64) indicate that injection treatments to degrade polysaccharides in and around sieve tube elements and decrease the production of those polysaccharides lead to increased iron and zinc levels. Iron is an important micronutrient involved in chlorophyll production in citrus trees, and iron deficiency can cause leaf dieback, reduced yield, and reduced fruit and juice quality. Zinc is an important micronutrient involved in citrus tree metabolism, photosynthesis, and growth regulation, and zinc deficiency, a widespread nutrient deficiency in citrus, can cause reduced blossoming, fruit set, fruit size, fruit quality, and juice content. Trunk injection of starch-degrading Amylase from Bacillus subtilis 168 at 92 U per tree increased iron and zinc levels an average of 102% and 105% relative to untreated control, respectively, while the combination including Amylase injection together with 2-DDG, a callose synthase inhibitor, increased foliar iron and zinc levels an average of 122% and 126% relative to untreated control, respectively.

TABLE 64
Trunk injection of recovery compositions increased iron and zinc levels in
nutrient-deficient citrus leaves and improved citrus fruit quality relative to control

| | Iron (ppm) Content, Leaves | | | |
|---|---|---|---|---|
| | (relative to untreated) | | | |
| Treatment | ‘Ruby Red’ | ‘Valencia’ | ‘Hamlin’ | Average |
| Untreated Control | 47.52 | 45.71 | 57.64 | — |
| | — | — | — | |
| Citrus Composition 27 | 56.39 | 38.70 | 59.40 | (102%) |
| Amylase (amyE) | (119%) | (85%) | (103%) | |
| from Bacillus subtilis 168 | | | | |
| (SEQ ID NO: 734) (92 U/tree) | | | | |
| Citrus Composition 28 | 69.83 | 50.68 | 62.75 | (122%) |
| Amylase (amyE) | (147%) | (111%) | (109%) | |
| from Bacillus subtilis 168 | | | | |
| (SEQ ID NO: 734) (92 U/tree) + | | | | |
| 2-deoxy-D-glucose (2-DDG) | | | | |
| 5.6 mg/mL (111.1 mg/tree) | | | | |
| | Zinc (ppm) Content, Leaves | | | |
| | (relative to untreated) | | | |
| Treatment | ‘Ruby Red’ | ‘Valencia’ | ‘Hamlin’ | Average |
| Untreated Control | 63.4 | 10.82 | 12.87 | — |
| | — | — | — | |
| Citrus Composition 27 | 60.37 | 11.51 | 14.57 | (105%) |
| Amylase (amyE) | (95%) | (106%) | (113%) | |
| from Bacillus subtilis 168 | | | | |
| (SEQ ID NO: 734) (92 U/tree) | | | | |
| Citrus Composition 28 | 71.18 | 12.10 | 19.94 | (126%) |
| Amylase (amyE) | (112%) | (112%) | (155%) | |
| from Bacillus subtilis 168 | | | | |
| (SEQ ID NO: 734) (92 U/tree) + | | | | |
| 2-deoxy-D-glucose (2-DDG) | | | | |
| 5.6 mg/mL (111.1 mg/tree) | | | | |

| | Average fruit weight, grams | | |
|---|---|---|---|
| | (relative to untreated) | | |
| Treatment | ‘Ruby Red’ | ‘Hamlin’ | Average |
| Untreated Control | 297.3 | 155.8 | |
| Citrus Composition 27 | 299.8 | 170.6 | (+5%) |
| Amylase (amyE) | (+1%) | (+9%) | |
| from Bacillus subtilis 168 | | | |
| (SEQ ID NO: 734) (92 U/tree) | | | |
| Citrus Composition 28 | 306.1 | 215.2 | (+21%) |
| Amylase (amyE) | (+3%) | (38%) | |
| from Bacillus subtilis 168 | | | |
| (SEQ ID NO: 734) (92 U/tree) + | | | |
| 2-deoxy-D-glucose (2-DDG) | | | |
| 5.6 mg/mL (111.1 mg/tree) | | | |

Increased iron and zinc micronutrient levels in leaves sampled from trees injected with Amylase and 2-DDG were predicted to have a positive impact on the fruit quality at harvest. ‘Ruby Red’ Grapefruit and ‘Hamlin” fruit was harvested from untreated, Amylase injected, and Amylase+2-DDG injected trees. Fruit quality results (Table 64) indicate that trunk injection of treatments to degrade polysaccharides in and around sieve tube elements and decrease the production of those polysaccharides lead to increased fruit weight, as measured by an average 5% increase from application of starch-degrading Amylase from Bacillus subtilis 168 at 92 U per tree. Amylase injection together with 2-DDG, a callose synthase inhibitor, increased fruit weight an average 21% relative to control.

### Example 23: Foliar Application of Bt4Q7Flg22 and Osmoprotectants Decreases Bacterial Titer, Improves Nutrient Content, and Increases Yield in Citrus

qPCR results from replicated ‘Valencia’ orange trials (Table 65) indicate that foliar treatments to activate the plant immune system and protect the plant from abiotic stress lead to decreased CLas bacterial titers. Over the 8-week time course CLas bacterial titers in the untreated control increased 491% compared to initial levels, whereas bacterial titers in Bt.4Q7Flg22 spray treated trees were reduced 16%, compared to initial levels. A synergistic effect was observed for treatment with both Bt.4Q7Flg22+Osmoprotectant 1, for which a 90% reduction in CLas titers was observed in comparison to the initial bacterial titers. Osmoprotectant 1 (Citrus composition 31) contains Betaine, also known as trimethylglycine, which is a naturally occurring amino acid that is utilized by plants to hold osmotic pressure within cells. Foliarly applied betaine is taken up by plants through the stoma and is utilized by plants to hold an osmotic gradient to keep water inside tissues, and to minimize water vapor loss during transpiration. Osmoprotectant 1 additionally contains potassium, which is an essential micronutrient for plant growth and required for flower and fruit formation. Foliarly applied potassium is also used by the plant to help close the stoma under stressful conditions, such as drought or heat stress, and assists in the movement of nutrients throughout the plant through regulation and assistance in osmosis.

TABLE 65
Foliar application of citrus compositions including Bt4Q7Flg22 and
Osmoprotectants decreased CLas bacterial titers in HLB-infected
‘Valencia’ 8 weeks post-application relative to control

| | Average CLas Titer | | |
|---|---|---|---|
| | (copy number | | Fold |
| | per 100 mg leaf tissue) | | Change in |
| | Pre-application | 8 Weeks Post- | CLas Titer |
| Treatment | (T0) | Application (T8) | (T₈/T₀) |
| Untreated Control | 6.06 × 10⁵ | 3.58 × 10⁶ | 5.91× |
| Citrus Composition 30 | 1.85 × 10⁵ | 1.56 × 10⁵ | 0.84× |
| Bt.4Q7Flg22 | | | |
| (SEQ ID NO: 226) | | | |
| (12 mL/tree) | | | |
| Citrus Composition 31 | 1.08 × 10⁶ | 1.04 × 10⁵ | 0.10× |
| Bt.4Q7Flg22 | | | |
| (SEQ ID NO: 226) | | | |
| (12 mL/tree) + | | | |
| Osmoprotectant 1 | | | |
| (4.21 mL/tree) | | | |

Nutrient analysis results from replicated citrus trials (Table 66) indicate that foliar treatments to activate the plant immune system and protect the plant from abiotic stress lead to increased calcium levels. Calcium is an essential nutrient that has important roles in cell division, root development, plant growth, and fruit yield in citrus trees. On average, Bt.4Q7Flg22 spray at 12 mL/tree increased calcium levels 0.8% relative to control, while the combination including Bt.4Q7Flg22 spray and Osmoprotectant 1 increased foliar calcium levels 4.6% relative to control.

TABLE 66
Foliar application of citrus compositions including Bt4Q7Flg22 and
Osmoprotectant increased calcium levels in citrus trees relative to control

| | % Calcium (w/w) | | |
|---|---|---|---|
| | (relative to control) | | |
| | ‘Hamlin’ | ‘Ruby Red’ | |
| Treatment | Orange | Grapefruit | Average |
| Surfactant Control | 2.53 | 2.20 | 2.37 |
| | — | — | — |
| Citrus Composition 30 | 2.64 | 2.14 | 2.39 |
| Bt.4Q7Flg22 (SEQ ID NO: 226) | (104.3%) | (97.3%) | (100.8%) |
| (12 mL/tree) | | | |
| Citrus Composition 31 | 2.71 | 2.25 | 2.48 |
| Bt.4Q7Flg22 (SEQ ID NO: 226) | (107.1%) | (102.3% | (104.6%) |
| (12 mL/tree) + | | | |
| Osmoprotectant 1 | | | |
| (4.21 mL/tree) | | | |

Harvest results from replicated Hamlin orange and Ruby Red grapefruit trials (Table 67) indicate that foliar treatment of Bt.4Q7Flg22 and Osmoprotectants lead to increased yield per tree (kg fresh fruit). On average, co-treatment with Bt.4Q7Flg22 (12 mL/tree) and Osmoprotectant 1 increased yield by 21% relative to the control trees, and 10% relative to Bt.4Q7Flg22 (12 mL/tree) alone. Thus, combination treatments of Bt.4Q7Flg22 and Osmoprotectants are predicted to promote additive or synergistic yield increase.

TABLE 67
Foliar application of Bt.4Q7Flg22 and Osmoprotectants
increased fresh fruit yield in Hamlin orange and-Ruby
Red grapefruit trees relative to control

| | Yield, Average pounds fruit per tree | | |
|---|---|---|---|
| | (relative to control) | | |
| | Hamlin | Ruby Red | |
| Treatment | Orange | Grapefruit | Average |
| Surfactant Control | 39.31 kgs. | 50.75 kgs. | — |
| | — | — | |
| Citrus Composition 30 | 45.62 kgs. | 53.32 kgs. | (+11%) |
| Bt.4Q7Flg22 (SEQ ID NO: 226) | (+16%) | (+5%) | |
| (12 mL/tree) | | | |
| Citrus Composition 31 | 51.57 kgs. | 56.53 kgs. | (+21%) |
| Bt.4Q7Flg22 (SEQ ID NO: 226) | (+31%) | (+11%) | |
| (12 mL/tree) + | | | |
| Osmoprotectant 1 | | | |
| (4.21 mL/tree) | | | |

Fruit quality results from replicated Hamlin orange and Ruby Red grapefruit trials (Table 68) indicate that increased yield (Table 68) after foliar treatment with Bt.4Q7Flg22 and Osmoprotectant 1 is due in part to increased fruit size (diameter and weight) and fruit retention (higher fruit count harvested, reduced pre-harvest dropped fruit) in comparison to the control.

TABLE 68
Foliar application of Bt.4Q7Flg22 and Osmoprotectant improved fruit quality and
retention in Hamlin orange and Ruby Red grapefruit trees relative to control

| | Average fruit diameter, mm | | | Average fruit weight, grams | | |
|---|---|---|---|---|---|---|
| | (relative to control) | | | (relative to control) | | |
| | Hamlin | Ruby Red | | Hamlin | Ruby Red | |
| Treatment | Orange | Grapefruit | Average | Orange | Grapefruit | Average |
| Surfactant | 66.4 | 91.3 | — | 144.5 | 329.0 | — |
| Control | | | | | | |
| Citrus | 69.0 | 95.6 | | 166.1 | 341.0 | |
| Composition | | | | | | |
| 31 | | | | | | |
| Bt.4Q7Flg22 | (+3.9%) | (+4.7%) | (+4.3%) | (+14.9%) | (+3.6%) | (+9.3%) |
| (SEQ ID NO: | | | | | | |
| 226) | | | | | | |
| (12 mL/tree) + | | | | | | |
| Osmoprotectant | | | | | | |
| 1 | | | | | | |
| (4.21 mL/tree) | | | | | | |

| | Average Fruit Count per Tree | | | Average % Fruit Drop | | |
|---|---|---|---|---|---|---|
| | (relative to control) | | | (relative to control) | | |
| | Hamlin | Ruby Red | | Hamlin | Ruby Red | |
| Treatment | Orange | Grapefruit | Average | Orange | Grapefruit | Average |
| Surfactant | 271 | 151 | — | 6.0% | 11.7% | — |
| Control | | | | | | |
| Citrus | 314 | 165 | — | 4.2% | 10.4% | — |
| Composition | | | | | | |
| 31 | | | | | | |
| Bt.4Q7Flg22 | (+15.9%) | (+9.3%) | (+12.6%) | (−29.1%) | (−10.4%) | (−19.7%) |
| (SEQ ID NO: | | | | | | |
| 226) | | | | | | |
| (12 mL/tree) + | | | | | | |
| Osmoprotectant | | | | | | |
| 1 | | | | | | |
| (4.21 mL/tree) | | | | | | |

Juice quality results from replicated Hamlin orange and Ruby Red grapefruit trials (Table 69) indicate that foliar treatment of Bt.4Q7Flg22 and Osmoprotectant 1 leads to an increased average juice volume per fruit, ^{o}Brix_{c}:Citric Acid Ratio, and pounds-solids per tree. The Ratio is an indicator of good flavor and high juice quality. Per USDA standards, the ideal Ratio ranges from 15.0-20.5 for orange juice and from 7.0-16.0 for grapefruit juice. In the citrus industry, a higher Ratio is accepted to give a better flavor juice. On average compared to the control, combined foliar treatment of Bt4Q7Flg22 and Osmoprotectant 1 increases juice volume per fruit by 33%, ^{o}Brix_{c}:Citric Acid Ratio by 10%, and pounds-solids per tree by 25%.

TABLE 69
Foliar application of Bt.4Q7Flg22 and Osmoprotectant improved juice quality
and yield in ‘Ruby Red’ grapefruit trees relative to control

| | Average Juice Volume per Fruit, mL (relative to control) | | |
|---|---|---|---|
| Treatment | Hamlin orange | Ruby Red Grapefruit | Average |
| Surfactant Control | 45 mL | 110 mL | — |
| Citrus Composition 31 | 55 mL | 159.2 mL | (+33%) |
| Bt.4Q7Flg22 | (+22%) | (+45%) | |
| (SEQ ID NO: 226) | | | |
| (12 mL/tree) + | | | |
| Osmoprotectant 1 | | | |
| (4.21 mL/tree) | | | |

| | Average °Brix_{c}:Citric Acid Ratio | | | Average pounds-solids | | |
|---|---|---|---|---|---|---|
| | (relative to control) | | | per tree (relative to control) | | |
| | Hamlin | Ruby Red | | Hamlin | Ruby Red | |
| Treatment | orange | Grapefruit | Average | orange | Grapefruit | Average |
| Surfactant Control | 17.2 | 5.9 | — | 2.49 | 3.42 | — |
| Citrus Composition 31 | 17.9 | 6.9 | (+10%) | 3.13 | 4.24 | (+25%) |
| Bt.4Q7Flg22 | (+4%) | (+17%) | | (+26%) | (+24%) | |
| (SEQ ID NO: 226) | | | | | | |
| (12 mL/tree) + | | | | | | |
| Osmoprotectant 1 | | | | | | |
| (4.21 mL/tree) | | | | | | |

### Example 24: Foliar Application of GmRHPP Improves Citrus Tree Health and Yield

qPCR results from replicated Valencia orange trials (Table 70) indicate that foliar treatment of GmRHPP leads to decreased CLas bacterial titer 8-weeks post-application. Over the 8-week time course, CLas bacterial titers in untreated control trees increased 491% compared to initial levels, whereas bacterial titers in GmRHPP treated trees were reduced 17% compared to initial levels.

TABLE 70
Foliar application of GmRHPP decreased CLas bacterial titers in
HLB-infected Valencia 8 weeks post-application relative to controls

| | Average CLas Titer | | |
|---|---|---|---|
| | (copy number | | Fold |
| | per 100 mg leaf tissue) | | Change in |
| | Pre-application | 8 Weeks Post- | CLas Titer |
| Treatment | T0 | Application (T8) | (T₈/T₀) |
| Untreated Control | 6.06 × 10⁵ | 3.58 × 10⁶ | 5.91 |
| Citrus Composition 33 | 7.44 × 10⁵ | 6.17 × 10⁵ | 0.83 |
| GmRHPP (SEQ ID | | | |
| NO: 604) | | | |
| (1.5 mg/tree) (3.0 | | | |
| mL/tree) | | | |

Nutrient analysis results from replicated Valencia orange and Ruby Red grapefruit trials (Table 71) indicate that foliar treatment with GmRHPP leads to increased iron levels in the leaves. Iron is an important micronutrient required for chlorophyll production in leaves, and iron deficiency can cause citrus leaf dieback, reduced yield, and reduced fruit and juice quality. GmRHPP sprayed at a rate of 3.0 mL/tree per tree increased foliar iron levels an average of 28% relative to untreated.

TABLE 71
Foliar application of GmRHPP increased iron levels in iron-low
Valencia orange and Ruby Red grapefruit trees relative to control

| | Iron, ppm | | |
|---|---|---|---|
| | (relative to untreated) | | |
| | Valencia | Ruby Red | |
| Treatment | orange | grapefruit | Average |
| Untreated Control | 40.14 | 42.17 | 41.17 |
| | — | — | — |
| Citrus Composition 33 | 48.09 | 57.56 | 52.83 |
| GmRHPP (SEQ ID NO: 604) | (120%) | (136%) | (128%) |
| (3.0 mL/tree) | | | |

Nutrient analysis results from replicated Valencia and Hamlin orange and Ruby Red grapefruit trials (Table 72) indicate that foliar treatment of GmRHPP leads to increased boron levels in the leaves. Boron is an important micronutrient involved in citrus tree metabolism, growth, nutrient transport, flowering, fruiting, and hormone regulation. Boron deficiency, which is associated with HLB, can cause leaf chlorosis and defoliation, reduced yield, and reduced fruit size and quality. GmRHPP spray at 3.0 mL/tree per tree increased foliar boron levels an average of 12% relative to untreated across three trials.

TABLE 72
Foliar application of GmRHPP increased boron levels in Valencia orange,
Hamlin orange, and Ruby Red grapefruit trees relative to control

| | Boron, ppm | | | |
|---|---|---|---|---|
| | (relative to untreated) | | | |
| | Valencia | Hamlin | Ruby Red | |
| Treatment | orange | orange | grapefruit | Average |
| Untreated Control | 104.61 | 106.75 | 80.26 | 97.21 |
| | — | — | — | — |
| Citrus Composition 33 | 119.31 | 123.79 | 84.48 | 109.19 |
| GmRHPP (SEQ ID NO: 604) | (114%) | (116%) | (105%) | (112%) |
| (3.0 mL/tree) | | | | |

Harvest results from replicated Hamlin orange and Ruby Red grapefruit trials (Table 73) indicate that foliar treatment of GmRHPP leads to increased yield per tree (kg fresh fruit). On average, GmRHPP treated trees increases yield by 13.9% relative to the control trees treated with surfactant control alone.

TABLE 73
Foliar application of GmRHPP increased fresh fruit yield in
Hamlin orange and Ruby Red grapefruit trees relative to control

| | Yield, Average kg fruit per tree | | |
|---|---|---|---|
| | (relative to control) | | |
| | Hamlin | Ruby Red | |
| Treatment | orange | Grapefruit | Average |
| Surfactant Control | 39.31 kg | 50.75 kg | — |
| Citrus Composition 33 | 46.05 kg | 56.18 kg | (+13.9%) |
| GmRHPP (SEQ ID NO: 604) | (+17.1%) | (+10.7%) | |
| (3.0 mL/tree) | | | |

Harvest results from replicated Hamlin orange and Ruby Red grapefruit trials (Table 74) indicate that foliar treatment of GmRHPP leads to increased fruit count per tree and decreased fruit drop. On average, GmRHPP treated trees retain 19% more fruit at harvest, with an observed 22% decrease in fruit drop relative to the control trees treated with surfactant control alone.

TABLE 74
Foliar application of GmRHPP improved fruit retention in Hamlin
orange and Ruby Red grapefruit trees relative to control

| | Yield, Average fruit count | | | | | |
|---|---|---|---|---|---|---|
| | per tree | | | Fruit Drop, Average % | | |
| | (relative to control) | | | (relative to control) | | |
| | Hamlin | Ruby Red | | Hamlin | Ruby Red | |
| Treatment | orange | Grapefruit | Average | orange | Grapefruit | Average |
| Surfactant Control | 271 | 151 | — | 6.0% | 11.7% | — |
| Citrus Composition 33 | 297 | 193 | — | 5.2% | 8.2% | — |
| GmRHPP (SEQ ID NO: 604) | (+10%) | (+28%) | (+19%) | (−14%) | (−30%) | (−22%) |
| (3.0 mL/tree) | | | | | | |

Fruit quality results from replicated Hamlin orange and Ruby Red grapefruit trials (Table 75) indicate that foliar treatment of GmRHPP had no effect on fruit size and leads to increased fruit weight (density). On average, fruit harvested from GmRHPP treated trees weigh 6.2% more than fruit harvested from control trees treated with surfactant alone.

TABLE 75
Foliar application of GmRHPP improved fruit quality in
Hamlin orange trees relative to control

| | Average fruit | Average fruit |
|---|---|---|
| | diameter, mm | weight, grams |
| Treatment | (relative to control) | (relative to control) |
| Surfactant Control | 66.4 | 144.5 |
| Citrus Composition 33 | 65.9 | 153.3 |
| GmRHPP (SEQ ID NO: 604) | (−0.7%) | (+6.1%) |
| (3.0 mL/tree) | | |

Juice quality results from replicated Hamlin orange and Ruby Red grapefruit trials (Table 76) indicate that foliar treatment of GmRHPP leads to an increased ^{o}Brix_{c}:Citric Acid Ratio, and increased pounds-solids per tree. On average, juice harvested from GmRHPP treated citrus trees produce 13.5% more total sugar than control trees treated with surfactant alone.

TABLE 76
Foliar application of GmRHPP improved juice quality and yield in
Hamlin orange and Ruby Red grapefruit trees relative to control

| | Average °Brix_{c}:Citric Acid | | | Average pounds-solids | | |
|---|---|---|---|---|---|---|
| | Ratio | | | per tree | | |
| | (relative to control) | | | (relative to control) | | |
| | Hamlin | Ruby Red | | Hamlin | Ruby Red | |
| Treatment | orange | Grapefruit | Average | orange | Grapefruit | Average |
| Surfactant Control | 17.2 | 5.9 | — | 2.49 | 3.42 | — |
| Citrus Composition 33 | 17.9 | 6.9 | (+10%) | 2.97 | 3.71 | (+13.5%) |
| GmRHPP (SEQ ID NO: 604) | (+4%) | (+16%) | | (+19%) | (+8%) | |
| (3.0 mL/tree) | | | | | | |

### Example 25: Application of Chitinase and Glucanase Enzymes for Prevention of Sooty Mold (Ascomycete Fungi) Growth on Kiwifruit

β-1,3-D-glucanases (SEQ ID NO: 732 and SEQ ID NO: 772) and endochitinase (SEQ ID NO: 777) enzymes were investigated for their ability to prevent sooty mold growth on kiwifruit. The ascomycete fungus Cladosporium cladosporioides was grown on V8 agar (a sterol containing medium that allows for the growth and viewing of morphological features of the fungus) for 6 days in the dark to induce sporulation. Kiwifruit (Actinidia deliciosa) were marked to designate areas for spray treatments and inoculation with the fungus. Enzyme treatments comprising: β-1,3 endoglucanase (Paenibacillus species; SEQ ID NO: 732), β-1,3 endoglucanase (Cellulosimicrobium cellulans DK1; SEQ ID NO: 772) and endochitinase (Bacillus thuringiensis ChiC; SEQ ID NO: 767) were used in spray applications. Spray treatments were prepared with 0.1% non-ionic surfactant (alkyl phenol ethoxylate) and adjusted to final enzyme formulations consisting of 10% (v/v) or 33% (v/v) using water. The enzyme spray treatments were compared to a 0.1% non-ionic surfactant only control (same as described above with no enzyme added). For each treatment, 3 kiwifruits were placed on a paper towel and the marked areas were sprayed four times with the each of the treatments. The fruit were then allowed to dry before applying the mold spores.

A sterile cotton applicator was dipped into V8 broth and used to collect the C. cladosporioides mold spores from the agar V8 plate. Mold spores were then applied to the kiwifruit treated with the enzymes and surfactant control as described above. A volume of 40 μL of V8 broth containing the mold spores was applied to 6 marked spots on each of 3 kiwifruits. The spores were spread onto the marked sports for each fruit. Each treatment was also compared to a no mold control. For each treatment, the 3 kiwifruits were placed in a Ziplock bag with the spore spots facing upward and incubated in a growth chamber with 60% humidity, a natural light cycle, and ˜25° C. for 6 days.

After 6 days, images of each kiwifruit were taken. Each fruit was scored for darkened discoloration or obvious mold growth (fuzzy, filamentous, or spore-laden spots) within the 6 spots in comparison to the uninoculated surroundings. At the 6 designated spots on each kiwifruit, the mold growth was determined for a total of N=18 spots per treatment. In the first experiment (Table 78), β-1,3-endoglucanase (SEQ ID NO: 732) and endochitinase (SEQ ID NO: 777) were expressed as immobilized enzymes on a protein matrix. The β-1,3-endoglucanase (SEQ ID NO: 732) treatment reduced mold growth to 61%, relative to the surfactant only control, while endochitinase (SEQ ID NO: 778) reduced growth to 83% of control. The combined treatment with both the β-1,3-endoglucanase (SEQ ID NO: 732) and endochitinase (SEQ ID NO: 777) enzymes resulted in mold growth reduced to 78% of control. In the second experiment (Table 79), a β-1,3-endoglucanase (SEQ ID NO: 772) was expressed as a free enzyme and combined with endochitinase (SEQ ID NO: 777) expressed as an immobilized enzyme. In this experiment, the β-1,3-endoglucanase (SEQ ID NO: 772) applied alone reduced mold growth to 85% compared to the surfactant control treatment. The combination of β-1,3-endoglucanase (SEQ ID NO:772) free enzyme and immobilized endochitinase (SEQ ID NO: 777 reduced mold growth to 29% of control. Thus, the combination treatment of β-1,3-endoglucanase (SEQ ID NO: 772) free enzyme and immobilized endochitinase (SEQ ID NO: 777) had an overall greater benefit in reducing mold growth than treatment with the β-1,3-endoglucanase (SEQ ID NO: 760) enzyme when applied alone.

TABLE 78
Application of immobilized enzyme for
prevention of sooty mold growth on kiwifruit

| | Observed | Percent of |
|---|---|---|
| | mold growth | mold growth |
| | spots (out of 18 | relative to surfactant |
| Treatment (10% spray rate) | inoculated) | only control |
| Surfactant only control | 18 | — |
| Negative control (no mold) | 0 | 0% |
| β-1,3-endoglucanase (bglH) | 11 | 61% |
| Paenibacillus immobilized | | |
| enzyme (SEQ ID NO: 732) | | |
| Endochitinase (ChiC) Bacillus | 15 | 83% |
| thuringiensis immobilized enzyme | | |
| (SEQ ID NO: 777)) | | |
| β-1,3-endoglucanase (bglH) | 14 | 78% |
| Paenibacillus immobilized enzyme | | |
| (SEQ ID NO: 732) + | | |
| Endochitinase (ChiC) Bacillus | | |
| thuringiensis immobilized enzyme | | |
| (SEQ ID NO: 777) | | |

TABLE 79
Application of free enzymes for
prevention of sooty mold growth on kiwifruit

| | Observed | Percent of |
|---|---|---|
| | mold growth | mold growth |
| | spots (out of 18 | relative to control |
| Treatment (33% spray rate) | inoculated) | (mold only) |
| Surfactant only control | 14 | — |
| β-1,3-endoglucanase (DK-1) | 12 | 85% |
| Cellulosimicrobium cellulans | | |
| (SEQ ID NO: 772) | | |
| β-1,3-endoglucanase (DK-1) | 4 | 29% |
| Cellulosimicrobium cellulans | | |
| (SEQ ID NO: 772) + | | |
| Endochitinase (ChiC) | | |
| Bacillus thuringiensis immobilized | | |
| enzyme (SEQ ID NO: 777) | | |

### Example 26: Flg22-PSA and Serine Protease Foliar Application on Kiwi Protects Plants from Pseudomonas syringae pv. actinidiae (PSA-V)

Pseudomonas syringae pv. actinidiae (PSA) is a devastating plant pathogen causing bacterial canker of both green-flesh (Actinidiae deliciosa) and yellow-flesh (Actinidiae chinesis) kiwi plants throughout zones of kiwi production, causing severe harvest loss in New Zealand, China, and Italy. PSA-V colonizes the outer and inner surfaces of the kiwi plant, forming biofilms that promote virulence, and can spread through the xylem and phloem tissues. Disease symptoms of PSA-V on kiwi include bacterial leaf spot, bacterial canker of the trunk, red exudates, blossom rot, discoloration of twigs, and ultimately dieback of kiwi vines. The standard method of control for PSA-V currently employs frequent foliar applications of metallic copper to kiwi vines which is predicted to lead to the selection of copper-resistant form of the pathogen and loss of disease control. Novel methods of control are urgently needed. To assess the efficacy of Flg22-PSA (SEQ ID NO: 540) and Serine Protease 2 from Bacillus subtilis (SEQ ID NO: 795) for control of PSA-V, a potted kiwi disease trial was conducted in the Bay of Plenty area of Te Puka, New Zealand. Flg22-PSA (SEQ ID NO: 540) activates the kiwi innate immune system to restrict bacterial growth and symptom progression, and Serine Protease 2 (SEQ ID NO: 795) disrupts biofilm formation, thus decreasing bacterial virulence. Copper hydroxide was included in the trial as a comparative industry standard for control of PSA-V. Prior to inoculation, PSA-V symptom-free potted kiwi Actinidiae deliciosa ‘Hayward’ plants were evenly distributed between 6 treatment groups (Table 80), with 12 potted plants per group. Treatments were applied as described in Table 80, with Flg22-PSA (SEQ ID NO: 540) applications prior to inoculation with PSA-V for priming of plant defenses, and Serine Protease 2 (SEQ ID NO: 795) applications 48 hours after inoculation for prevention of PSA-V biofilm formation and breakdown of existing biofilms. All treatments were applied with a non-ionic surfactant for penetration of the leaf cuticle through the stoma.

TABLE 80
Treatments applied to potted kiwi trial

| | | Product dilution | | |
|---|---|---|---|---|
| Treat- | | for spray | | |
| ment | Foliar Formulation* | application | Timing | Inoculum |
| 1 | Uninfected (no | — | — | None |
| | PSA-V) | | | |
| 2 | Untreated | — | — | PSA-V |
| 3 | ChampION++ ™ | 0.9 g | 1 day | PSA-V |
| | (46.1% Copper | ChampION++ ™/ | before | |
| | Hydroxide; 30% | L water | inoculation | |
| | metallic copper | | with PSA- | |
| | equivalent) | | V | |
| 4 | Flg22-PSA (SEQ ID | 4 mL/L water | 1 day | PSA-V |
| | NO: 540) 100 μM; | | before | |
| | 10 mM Sodium | | inoculation | |
| | Phosphate | | with PSA- | |
| | Buffer, pH 5.7 | | V | |
| 5 | Serine Protease 2 | 20 mL/L water | Post | PSA-V |
| | [SEQ ID NO: 795] | | inoculation | |
| | Fermentation broth | | on dry | |
| | filtrate with | | plants | |
| | immobilized enzyme | | | |
| 6 | Flg22-PSA (SEQ ID | 4 mL/L water + | 1 day | PSA-V |
| | NO: 540) 100 μM; | 20 mL/L water | before | |
| | 10 mM Sodium | | inoculation | |
| | Phosphate Buffer, pH | | with PSA- | |
| | 5.7 + | | V + | |
| | Serine Protease 2 | | Post | |
| | [SEQ ID NO: 795] | | inoculation | |
| | Fermentation broth | | on dry | |
| | filtrate with | | plants | |
| | immobilized enzyme | | | |

*Foliar compositions contained 0.1% (v/v) Proxel ™ BC preservative, an aqueous dispersion of a blend of 330.7 mM 1,2-benzisothiazolin (BIT), 53.5 mM 5-chloro-2-methyl-4-isolthiazolin-3-one (CMIT), and 26.1 mM 2-methyl-4-isothiazolin-3-one (MIT). Foliar compositions were diluted to the indicated concentrations in water (g/L water or mL/L water) with 0.05% (v/v) Contact Xcel ™ (980 g/L linear alcohol ethoxylate) non-ionic surfactant. The diluted products were applied in fine droplets with a pressurized backpack sprayer to the entire canopy of each plant, until thoroughly covered.

24-hours after the initial treatments, all plants except for the uninfected controls were sprayed with 1×10⁸ cfu/mL PSA-V inoculum using a 5 L hand-held pressurized sprayer aimed at the underside of leaves until thoroughly covered. The uninfected control was sprayed with water alone. Potted plants were then transported to Pukehina and placed in an area with overhead misting for 48 hours to mimic environmental conditions for PSA-V infection, with uninfected control plants separated from infected plants. After 48 hours, a subset of plants was then removed from the misting area and allowed to briefly dry before application of Serine Protease 2 (SEQ ID NO: 795). After the final treatments, all plants were moved to their final outdoor trial site, randomized positions in Pukehina. Average daily temperature at the trial site was 20.75° C. with a total rainfall of 277 mm over 34 days. Additionally, each plant was watered twice a day for two hours at a time by drip irrigation. Environmental conditions were favorable for progression of PSA-V disease symptoms. Plants were visually monitored throughout the trial period for PSA-V disease assessments, with the same assessor recording the % of leaf area covered in spots at 6 days after inoculation (6 DAI), 16 DAI, 23 DAI and 29 DAI. Additionally, each plant was assessed for treatment phytotoxicity effects at 29 DAI on a scale of 0-10, with 0=no leaf phytotoxicity and 10=very severe leaf phytotoxicity symptoms. The average disease scores at 6, 16, 23, and 29 DAI and phytotoxicity score at 29 DAI are reported in Tables 81 and 81 for each treatment (n=12 plants per treatment). P-values were calculated for each treatment vs. the untreated control.

TABLE 81
Flg22-PSA and Serine Protease 2 foliar applications
significantly reduce PSA-V disease symptoms in kiwi plants

| Treatment | Foliage Affected (% leaf surface area); | | | |
|---|---|---|---|---|
| group #/Foliar | p-values vs. untreated control | | | |
| Formulation | 6 DAI | 16 DAI | 23 DAI | 29 DAI |
| Treatment 1 | 0.00% | 1.66% | 7.89% | 18.14% |
| Uninfected plants | | | | |
| Treatment 2 | 15.12% | 40.36% | 54.64% | 67.82% |
| Untreated Control | | | | |
| Treatment 3 | 3.23% | 12.48% | 16.57% | 25.20% |
| ChampION++ ™ | (p < 0.001) | (p < 0.001) | (p < 0.001) | (p < 0.001) |
| Treatment 4 | 7.31% | 29.41% | 45.97% | 61.91% |
| Flg22-PSA (SEQ ID | (p < 0.001) | (p = 0.013) | (p = 0.085) | (p = 0.190) |
| NO: 540) | | | | |
| Treatment 5 | 3.28% | 20.26% | 42.76% | 64.90% |
| Serine Protease 2 | (p < 0.001) | (p < 0.001) | (p = 0.019) | (p = 512) |
| (SEQ ID NO: 795) | | | | |
| Treatment 6 | 5.85% | 20.01% | 35.51% | 53.52% |
| Flg22-PSA (SEQ ID | (p < 0.001) | (p < 0.001) | (p < 0.001) | (p = 0.002) |
| NO: 540) + | | | | |
| Serine Protease 2 | | | | |
| (SEQ ID NO: 722) | | | | |

Application of Flg22-PSA (SEQ ID NO: 540) or Serine Protease 2 (SEQ ID NO: 795) alone significantly reduced PSA-V leaf spot symptoms (P<0.1; 90% confidence interval) at 6, 16 and 23 DAI in comparison to the untreated control. Combination of Flg22-PSA pretreatment and Serine Protease 2 post-inoculum treatment further decreased the severity of leaf spot compared to either treatment alone at 16, 23 and 29 DAI and prolongs the period of significant protection to 29 DAI (35.4% less leaf spot compared to untreated control; P=0.002). In conclusion, Flg22-PSA and Serine Protease 2 can be used both as stand-alone treatments and in combination with other treatments aimed at restricting pathogen growth. While the industry standard ChampION++TM which is the currently used copper containing treatment to treat PSA causes mild leaf phytotoxicity (AVE score=1.6), no significant phytotoxicity was observed for Treatments 4-6 (Table 82).

TABLE 82
FLG22-PSA and Serine Protease 2 foliar applications do not
cause leaf phytotoxicity of kiwi plants

| | Average Phytotoxicity |
|---|---|
| | Score (0-10); |
| Treatment group #/Foliar Formulation | 29 DAI |
| Treatment 1 | 0.0 (±0.0) |
| Uninfected plants | |
| Treatment 2 | 0.0 (±0.0) |
| Untreated Control | |
| Treatment 3 | 1.6 (±0.9) |
| ChampION++ ™ | |
| Treatment 4 | 0.1 (±0.3) |
| Flg22-PSA (SEQ ID NO: 540) | |
| Treatment 5 | 0.0 (±0.0) |
| Serine Protease 2 (SEQ ID NO: 795) | |
| Treatment 6 | 0.0 (±0.0) |
| Flg22-PSA (SEQ ID NO: 540) + | |
| Serine Protease 2 (SEQ ID NO: 795) | |

In view of the above, it will be seen that the several objects of the invention are achieved and other advantageous results attained.

As various changes could be made in the above polypeptides, recombinant organisms, methods, and seeds, without departing from the scope of the invention, it is intended that all matter contained in the above description shall be interpreted as illustrative and not in a limiting sense.

1. A composition for bioactive priming of a plant or a plant part to increase growth, yield, health, longevity, productivity, and/or vigor of a plant or a plant part and/or protect the plant or the plant part from disease, and/or increase the innate immune response of the plant or the plant part and/or improve the quality of a fruit, juice obtained from a fruit, or a harvest obtained from a plant or plant part, wherein the composition comprises (A) at least one bioactive priming polypeptide and an inducer compound; or (B) at least two bioactive priming polypeptides, optionally, with an inducer compound; or (C) a callose synthase inhibitor and at least one inducer compound comprising a bacteriocide, an amino acid, a substituted or unsubstituted benzoic acid or derivative or salt thereof, a dicarboxylic acid or derivative or salt thereof, a betaine, a proline, a benzothiadiazole or any combination thereof; or (D) a bacteriocide and at least one inducer compound comprising β amino butyric acid (BABA), a betaine, a proline, a benzothiadiazole, salicylic acid, oxalic acid, or any combination thereof, wherein:
the polypeptide or polypeptides of (A) or (B) comprise:
(i) a flagellin or flagellin-associated polypeptide; or
(ii) a retro inverso flagellin or flagellin-associated polypeptide
(iii) a root hair promoting polypeptide (RHPP); or
(iv) a retro inverso root hair promoting polypeptide (RI RHPP); or
(v) a thionin or thionin-like polypeptide; or
(vi) a glucanase polypeptide; or
(vii) a serine protease polypeptide; or
(viii) an ACC deaminase (1-aminocyclopropane-1-carboxylate deaminase) polypeptide; or
(ix) an amylase; or
(x) a chitinase; or
(xi) any combination thereof;
with the provisos that:
the inducer compound comprises a callose synthase inhibitor, β-amino butyric acid (BABA), a betaine, a proline, a benzothiadiazole, salicylic acid, oxalic acid, or any combination thereof when the polypeptide of (A) comprises any polypeptide from groups (i)-(v) but not polypeptides from groups (vi) to (x); and
the inducer compound comprises a bacteriocide, an amino acid or isomer thereof, a callose synthase inhibitor, a substituted or unsubstituted benzoic acid or derivative thereof, a dicarboxylic acid or a derivative thereof, a betaine, a proline, a benzothiadiazole, or any combination thereof when the polypeptide of (A) comprises any polypeptide from the groups (vi) to (x); and
the composition comprises the inducer compound and the inducer compound comprises a callose synthase inhibitor, β-amino butyric acid (BABA), a betaine, a proline, a benzothiadiazole, salicylic acid, oxalic acid, or any combination thereof when the two or more polypeptides of (B) comprise polypeptides from the groups (i)-(v) but not polypeptides from the groups (vi) to (x).

2. An isolated peptide for bioactive priming of a plant or a plant part to increase growth, yield, health, longevity, productivity, and/or vigor of a plant or a plant part and/or decrease abiotic stress in the plant or the plant part and/or protect the plant or the plant part from disease, insects and/or nematodes, and/or increase the innate immune response of the plant or the plant part and/or change plant architecture, wherein the peptide comprises the amino acid sequence of any one of SEQ ID NOs: 732, 735, 746-755 and 757-778; or the peptide consists of the amino acid sequence of any one of SEQ ID NOs: 732, 735, 745-778.

3-6. (canceled)

7. A composition for bioactive priming of a plant or a plant part to increase growth, yield, health, longevity, productivity, and/or vigor of a plant or a plant part and/or protect the plant or the plant part from disease, and/or increase the innate immune response of the plant or the plant part and/or improve the quality of a fruit, juice obtained from a fruit, or a harvest obtained from a plant or plant part, wherein the composition comprises bixafen and at least one free polypeptide comprising:
(i) a flagellin or flagellin-associated polypeptide; or
(ii) a retro inverso flagellin or flagellin-associated polypeptide
(iii) a root hair promoting polypeptide (RHPP); or
(iv) a retro inverso root hair promoting polypeptide (RI RHPP); or
(v) a thionin or thionin-like polypeptide; or
(vi) a glucanase polypeptide; or
(vii) a serine protease polypeptide; or
(viii) an ACC deaminase (1-aminocyclopropane-1-carboxylate deaminase) polypeptide; or
(ix) an amylase; or
(x) a chitinase; or
(xi) any combination thereof;
wherein the free polypeptide is not bound to an exosporium of a Bacillus cereus family member or an intact Bacillus cereus family member spore.

8-10. (canceled)

11. A method for increasing growth, yield, health, longevity, productivity, and/or vigor of a plant or plant part and/or protecting the plant or plant part from disease and/or increasing the innate immune response of the plant or the plant part and/or increasing juice content and/or improving juice, sugar or acid content and/or improving a Brix:acid ratio of juice obtained from a plant, the method comprising applying the composition of claim 1 to a plant, plant part, or a plant growth medium in which the plant or plant part will be grown, or a rhizosphere in an area surrounding the plant or the plant part to increase growth, yield, health, longevity, productivity, and/or vigor of the plant or plant part and/or protect the plant or the plant part from disease and/or increase the innate immune response of the plant or plant part and/or increase juice content and/or improve juice, sugar or acid content and/or improve a Brix:acid ratio of juice obtained from a plant.

12-13. (canceled)

14. The composition of claim 1, wherein the composition comprises at least one flagellin or flagellin-associated polypeptide.

15. The composition of claim 14, wherein the flagellin or flagellin-associated polypeptide (a) is modified chemically on its N or C terminus, (b) is modified via crosslinking or cyclization, or (c) is from a Bacillus, a Lysinibacillus, a Paenibacillus, an Aneurinibacillus genus bacterium, or any combination thereof.

16-21. (canceled)

22. The composition of claim 14, wherein the amino acid sequence of the flagellin or flagellin-associated polypeptide comprises any one of SEQ ID NOs: 226-375, 526, 528, 530, 532, 534, 536, 538, 540, 571-587, and 589-590 or any combination thereof.

23-26. (canceled)

27. The composition of claim 14, wherein the amino acid sequence of the flagellin or flagellin-associated polypeptide comprises SEQ ID NO: 226.

28-70. (canceled)

71. The composition of claim 1, wherein the composition comprises a polypeptide further comprising a core sequence, wherein the core sequence comprises any one of SEQ ID NOs: 591-603.

72. (canceled)

73. The composition of claim 1, wherein at least one of the polypeptide of (A) or the polypeptides of (B) comprises a polypeptide that contains a chemical modification; is a variant having an amino acid insertion, deletion, inversion, repeat, duplication, extension, or substitution within the amino acid; is part of a fusion protein; or contains a protease recognition sequence.

74. The composition or method of claim 73, wherein:
(a) the chemical modification comprises acetylation, acid addition, acylation, ADP-ribosylation, aldehyde addition, alkylamide addition, amidation, amination, biotinylation, carbamate addition, chloromethyl ketone addition, covalent attachment of a nucleotide or nucleotide derivative, cross-linking, cyclization, disulfide bond formation, demethylation, ester addition, formation of covalent cross-links, formation of cysteine-cysteine disulfide bonds, formation of pyroglutamate, formylation, gamma-carboxylation, glycosylation, GPI anchor formation, hydrazide addition, hydroxyamic acid addition, hydroxylation, iodination, lipid addition, methylation, myristoylation, oxidation, PEGylation, proteolytic processing, phosphorylation, prenylation, palmitoylation, addition of a purification tag, pyroglutamyl addition, racemization, selenoylation, sulfonamide addition, sulfation, transfer-RNA mediated addition of amino acids to proteins, ubiquitination, or urea addition; and/or
(b) the chemical modification comprises an N-terminal modification or a C-terminal modification; and/or
(c) the amino acid substitution within the amino acid of the variant comprises substitution of a β-amino acid, a D-amino acid, or a non-natural amino acid.

75-77. (canceled)

78. The composition of claim 73 wherein the composition comprises the fusion protein and the fusion protein comprises an assistance peptide.

79. (canceled)

80. The composition of claim 78, wherein the assistance polypeptide comprises a signature polypeptide, and an amino acid sequence of the signature polypeptide comprises any one of SEQ ID NOs: 542-548, or any combination thereof.

81. (canceled)

82. The composition of claim 78, wherein the assistance polypeptide comprises: (a) a signal anchor sorting polypeptide, and an amino acid sequence of the signal anchor sorting polypeptide comprises any one of SEQ ID NOs: 549-562, or any combination thereof; or (b) a C-terminal secretion peptide, and an amino acid sequence of the C-terminal comprises any one of SEQ ID NOs: 563-570, or any combination thereof.

83-84. (canceled)

85. The composition of claim 1, wherein the composition comprises a polypeptide comprising an amino acid sequence having at least 70% identity to any one of SEQ ID NOs. 1-735, 745-787, and 794-797 and the composition has bioactive priming activity.

86-92. (canceled)

93. The composition of claim 1 wherein the composition comprises the polypeptide of (A) or the polypeptides of (B) and the inducer compound, wherein the inducer compound comprises a betaine, a betaine homolog, a betaine analog, a proline, a proline homolog, a proline analog, or any combination thereof.

94-98. (canceled)

99. The composition of claim 93 wherein the composition comprises a betaine, a betaine homolog or a betaine analog.

100. The composition of claim 99 wherein:
(a) the betaine comprises glycine betaine, glycine betaine aldehyde, β-alanine betaine, betaine hydrochloride, cetyl betaine, proline betaine, choline-O-sulfate betaine, cocaamidopropyl betaine, oleyl betaine, sulfobetaine, lauryl betaine, octyl betaine, caprylamidopropyl betaine, lauramidopropyl betaine, isostearamidopropyl betaine, or a combination, homolog, or analog of any thereof and/or
(b) the betaine homolog or analog comprises ectoine, choline, phosphatidylcholine, acetylcholine, cytidine disphosphate choline, dimethylethanolamine, choline chloride, choline salicylate, glycerophosphocholine, phosphocholine, a sphingomyelin, choline bitartrate, propio betaine, deanol betaine, homodeanol betaine, homoglycerol betaine, diethanol homobetaine, triethanol homobetaine, or a combination of any thereof.

101-104. (canceled)

105. The composition of claim 1, wherein the composition comprises a proline, a proline homolog or a proline analog.

106. The composition of claim 105 wherein
(a) the proline comprises L-proline, D-proline, hydroxyproline, hydroxyproline derivatives, proline betaine, or a combination, derivative, homolog, or analog of any thereof and/or
(b) the proline homolog or analog comprises α-methyl-L-proline, α-benzyl-Lproline, trans-4-hydroxy-L-proline, cis-4-hydroxy-L-proline, trans-3-hydroxy-L-proline, cis-3-hydroxy-L-proline, trans-4-amino-L-proline, 3,4-dehydro-α-proline, (2S)-aziridine-2-carboxylic acid, (2S)-azetidine-2-carboxylic acid, L-pipecolic acid, proline betaine, 4-oxo-L-proline, thiazolidine-2-carboxylic acid, (4R)-thiazolidine-4-carboxylic acid, or a combination of any thereof.

107-142. (canceled)

143. The composition of claim 1, wherein the composition further comprises a pesticide and the pesticide comprises an insecticide, a herbicide, a fungicide, a bacteriocide, a nematicide, a miticide, a biological control agent, or any combination thereof.

144-219. (canceled)

220. A seed coated with the composition of claim 1.
